(19) United States
(12) Patent Application Publication
(10) Pub. No.: US 2016/0244437 A1
(43) Pub. Date: Aug. 25, 2016

(54) SUBSTITUTED PHENYLALANINE DERIVATIVES

(71) Applicant: BAYER PHARMA AKTIENGESELLSCHAFT, Berlin (DE)

(72) Inventors: Ulrike RÖHN, Berlin (DE); Manuel ELLERMANN, Berlin (DE); Julia STRASSBURGER, Wuppertal (DE); Astrid WENDT, Syke (DE); Susanne RÖHRIG, Hilden (DE); Robert Alan WEBSTER, Wuppertal (DE); Martina Victoria SCHMIDT, Köln (DE); Adrian TERSTEEGEN, Wuppertal (DE); Kristin BEYER, Kensington, CA (US); Martina SCHÄFER, Berlin (DE); Anja BUCHMÜLLER, Essen (DE); Christoph GERDES, Köln (DE); Michael SPERZEL, Kierspe (DE); Steffen SANDMANN, Essen-Heisingen (DE); Stefan HEITMEIER, Wülfrath (DE); Alexander HILLISCH, Solingen (DE); Jens ACKERSTAFF, Düsseldorf (DE); Carsten TERJUNG, Bochum (DE)

(73) Assignee: Bayer Pharma Aktiengesellschaft, Berlin (DE)

(21) Appl. No.: 15/024,923

(22) PCT Filed: Sep. 24, 2014
(86) PCT No.: PCT/EP2014/070308

§ 371 Date: Mar. 25, 2016

(30) Foreign Application Priority Data
Sep. 26, 2013 (EP) 13186063.7

(51) Int. Cl. C07D 413/12; C07D 249/12; C07D 235/26; C07D 401/12; C07D 401/14; C07D 271/07; C07D 249/10; C07D 231/56; C07D 235/10; C07D 257/04; C07D 403/12

(52) U.S. Cl. CPC C07D 413/12; C07D 257/04; C07D 249/12; C07D 235/26; C07D 401/12; C07D 403/12; C07D 271/07; C07D 249/10; C07D 231/56; C07D 235/10; C07D 401/14

(57) ABSTRACT

The invention relates to substituted phenylalanine derivatives and to processes for preparation thereof, and to the use thereof for production of medicaments for the treatment and/or prophylaxis of diseases, especially of cardiovascular disorders and/or severe perioperative blood loss.

The invention relates to substituted phenylalanine derivatives and to processes for preparation thereof, and to the use thereof for production of medicaments for the treatment and/or prophylaxis of diseases, especially of cardiovascular disorders and/or severe perioperative blood loss.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood.

In the more recent past, the traditional theory of two separate regions of the coagulation cascade (extrinsic and intrinsic path) has been modified owing to new findings: In these models, coagulation is initiated by binding of activated factor VIIa to tissue factor (TF). The resulting complex activates factor X, which in turn leads to generation of thrombin with subsequent production of fibrin and platelet activation (via PAR-1) as injury-sealing end products of haemostasis. Compared to the subsequent amplification/propagation phase, the thrombin production rate is low and as a result of the occurrence of TFPI as inhibitor of the TF-FVIIa-FX complex is limited in time.

A central component of the transition from initiation to amplification and propagation of coagulation is factor XIa. In positive feedback loops, thrombin activates, in addition to factor V and factor VIII, also factor XI to factor XIa, whereby factor IX is converted into factor IXa, thus, via the factor IXa/factor VIIIa complex generated in this manner, rapidly producing relatively large amounts of factor Xa. This triggers the production of large amounts of thrombin, leading to strong thrombus growth and stabilizing the thrombus.

The formation of a thrombus or blood clot is counter-regulated by fibrinolysis. Activation of plasminogen by tissue plasminogen activator (tPA) results in formation of the active serine protease, plasmin, which cleaves polymerized fibrin and thus breaks down the thrombus. This process is referred to as fibrinolysis—with plasmin as key enzyme.

Uncontrolled activation of the coagulation system or defects in the inhibition of the activation processes may cause formation of local thromboses or embolisms in vessels (arteries, veins, lymph vessels) or heart chambers. This may lead to serious thrombotic or thromboembolic disorders. In addition, systemic hypercoagulability may lead to consumption coagulopathy in the context of a disseminated intravasal coagulation.

In the course of many cardiovascular and metabolic disorders, there is an increased tendency for coagulation and platelet activation owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis. This unwanted and excessive haemostasis may, by formation of fibrin- and platelet-rich thrombi, lead to thromboembolic disorders and thrombotic complications with life-threatening conditions.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants known from the prior art, for example substances for inhibiting or preventing blood coagulation, have various, frequently grave disadvantages. Accordingly, in practice, efficient treatment methods or the prophylaxis of thrombotic/thromboembolic disorders are found to be very difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicomentosa or osteoporosis [Pschyrembel, Klinisches Worterbuch [clinical dictionary], 257th edition, 1994, Walter de Gruyter Verlag, page 610, keyword “Heparin”; Rompp Lexikon Chemie, Version 1.5, 1998, Georg Thieme Verlag Stuttgart, keyword “Heparin” ]. Low-molecular-weight heparins do have a lower probability of leading to the development of heparin-induced thrombocytopaenia; however, they can also only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of certain vitamin K-dependent coagulation factors in the liver. Owing to the mechanism of action, the onset of action is only very slow (latency to the onset of action 36 to 48 hours). The compounds can be administered orally; however, owing to the high risk of bleeding and the narrow therapeutic index complicated individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683]. In addition, other side-effects such as gastrointestinal problems, hair loss and skin necroses have been described.

More recent approaches for oral anticoagulants are in various phases of clinical evaluation or in clinical use, but they have also shown disadvantages, for example highly variable bioavailability, liver damage and bleeding complications.

For antithrombotic medicaments, the therapeutic width is of central importance: The interval between the therapeutically active dose for coagulation inhibition and the dose where bleeding may occur should be as large as possible so that maximum therapeutic activity is achieved at a minimum risk profile.

In various in vivo models with, for example, antibodies as factor XIa inhibitors, but also in factor XIa knock-out models, the antithrombotic effect with small/no prolongation of bleeding time or extension of blood volume was confirmed. In clinical studies, elevated factor XIa concentrations were associated with an increased event rate. However, factor XI deficiency (haemophilia C), in contrast to factor VIIIa or factor IXa (haemophilia A and B, respectively), did not lead to spontaneous bleeding and was only noticed during surgical interventions and traumata. Instead, protection against certain thromboembolic events was found.

In the event of hyperfibrinolytic states, there is inadequate wound closure, which causes severe, sometimes life-threatening, bleeding. This bleeding can be stopped by the inhibition of fibrinolysis with antifibrinolytics, by which plasmin activity is reduced. Corresponding effects with the plasminogen inhibitor tranexamic acid have been shown in various clinical studies.

It is therefore an object of the present invention to provide novel compounds for the treatment and/or prophylaxis of cardiovascular disorders and/or severe perioperative blood loss in man and animals, said compounds having a wide therapeutic range.

WO 89/11852 describes, inter alia, substituted phenylalanine derivatives for treatment of pancreatitis, and WO 2007/070816 describes substituted thiophene derivatives as factor XIa inhibitors.

The invention provides compounds of the formula

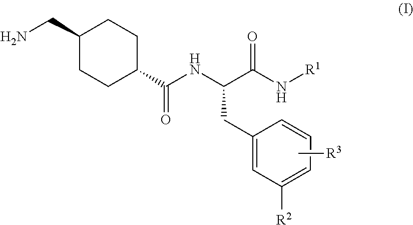

in which
R¹ represents a group of the formula

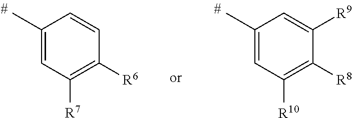

where # is the point of attachment to the nitrogen atom,
R⁶ represents 5-membered heteroaryl,
   where heteroaryl may be substituted by a substituent selected from the group consisting of oxo, chlorine, cyano, hydroxy and C₁-C₃-alkyl,
      in which alkyl may be substituted by 1 to 3 substituents independently of one another selected from the group consisting of hydroxy, amino, hydroxycarbonyl and methoxy,
      or
      in which alkyl may be substituted by 1 to 7 fluorine substituents,
      or
      in which alkyl is substituted by a substituent selected from the group consisting of hydroxy, amino, hydroxycarbonyl and methoxy, and in which alkyl is additionally substituted by 1 to 6 fluorine substituents,
R⁷ represents hydrogen, fluorine or chlorine,
R⁸ and R⁹ together with the carbon atoms to which they are attached form a 5-membered heterocycle,
   where the heterocycle may be substituted by 1 to 2 substituents independently of one another selected from the group consisting of oxo, chlorine, cyano, hydroxy, C₁-C₃-alkyl, pyrazolyl and pyridyl,
      in which alkyl may be substituted by 1 to 3 substituents independently of one another selected from the group consisting of hydroxy, amino, hydroxycarbonyl and methoxy,
      or
      in which alkyl may be substituted by 1 to 7 fluorine substituents,
      or
      in which alkyl is substituted by a substituent selected from the group consisting of hydroxy, amino, hydroxycarbonyl and methoxy, and in which alkyl is additionally substituted by 1 to 6 fluorine substituents,
R¹⁰ represents hydrogen, fluorine or chlorine,
   R² represents a group of the formula

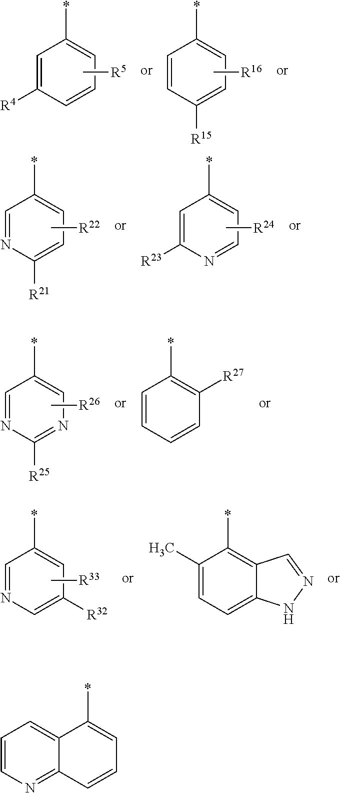

where * is the point of attachment to the phenyl ring,
R⁴ represents amino, C₁-C₄-alkylcarbonylamino, —S(O)₂NR¹¹R¹² or —C(O)NR¹³R¹⁴
   where
   R¹¹ represents hydrogen, C₁-C₃-alkyl, C₃-C₆-cycloalkyl, benzyl or 4- to 8-membered heterocyclyl which is attached via a carbon atom,
   R¹² represents hydrogen or C₁-C₃-alkyl,
   or
   R¹¹ and R¹² together with the nitrogen atom to which they are attached form a 4- to 7-membered heterocycle,
   R¹³ represents hydrogen, C₁-C₃-alkyl, C₁-C₃-alkoxy, C₃-C₆-cycloalkyl, benzyl or
      4- to 8-membered heterocyclyl which is attached via a carbon atom, in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
         in which n is a number from 1 to 6,
         in which m is a number from 1 to 6,
      and
      in which cycloalkyl may be substituted by 1 to 2 substituents independently of one another selected from the group consisting of oxo, fluorine, hydroxy, amino, C₁-C₄-alkyl and C₁-C₃-alkylamino,
         in which alkyl and alkylamino for their part may be substituted by 1 to 5 fluorine substituents,
      and
      in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
   R¹⁴ represents hydrogen or C₁-C₃-alkyl,
   R¹³ and R¹⁴ together with the nitrogen atom to which they are attached form a 4- to 7-membered heterocycle,
      in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
R⁵ represents hydrogen, fluorine, chlorine, C₁-C₄-alkyl, methoxy or trifluoromethyl,
R¹⁵ represents amino, —S(O)₂NR¹⁷R¹⁸ or —C(O)NR¹⁹R²⁰,
   where
   R¹⁷ represents hydrogen, C₁-C₃-alkyl, C₃-C₆-cycloalkyl, benzyl or 4- to 8-membered heterocyclyl which is attached via a carbon atom,
   R¹⁸ represents hydrogen or C₁-C₃-alkyl,
   or
   R¹⁷ and R¹⁸ together with the nitrogen atom to which they are attached form a 4- to 7-membered heterocycle,
   R¹⁹ represents hydrogen, C₁-C₃-alkyl, C₁-C₃-alkoxy, C₃-C₆-cycloalkyl, benzyl or 4- to 8-membered heterocyclyl which is attached via a carbon atom,
      in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
         in which n is a number from 1 to 6,
         in which m is a number from 1 to 6,
      and
      in which cycloalkyl may be substituted by 1 to 2 substituents independently of one another selected from the group consisting of oxo, fluorine, hydroxy, amino, C₁-C₄-alkyl and C₁-C₃-alkylamino,
         in which alkyl and alkylamino for their part may be substituted by 1 to 5 fluorine substituents,
      and
      in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
   R²⁰ represents hydrogen or C₁-C₃-alkyl,
   R¹⁹ and R²⁰ together with the nitrogen atom to which they are attached form a 4- to 7-membered heterocycle,
      in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
         in which alkyl and alkylamino for their part may be substituted by 1 to 5 substituents independently of one another selected from the group consisting of hydroxy and fluorine,
      and in which heterocyclyl may additionally be substituted by 1 to 4 substituents independently of one another selected from the group consisting of fluorine and methyl,
R¹⁶ represents hydrogen, fluorine, chlorine, C₁-C₄-alkyl, methoxy or trifluoromethyl,
R²¹ represents hydrogen, hydroxy, amino, C₁-C₄-alkoxy, C₁-C₃-alkylamino or 5- or 6-membered heterocyclyl which is attached via a nitrogen atom,
   where alkoxy may be substituted by 1 to 2 substituents independently of one another selected from the group consisting of amino and C₁-C₃-alkylamino,
   and
   where heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R²² represents hydrogen, fluorine, chlorine, amino, C₁-C₄-alkyl, methoxy or trifluoromethyl,
R²³ represents hydrogen, hydroxy, amino, C₁-C₄-alkoxy, C₁-C₃-alkylamino or 5- or 6-membered heterocyclyl which is attached via a nitrogen atom,
   where alkoxy may be substituted by 1 to 2 substituents independently of one another selected from the group consisting of amino and C₁-C₃-alkylamino,
   and
   where heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R²⁴ represents hydrogen, fluorine, chlorine, C₁-C₄-alkyl, methoxy or trifluoromethyl,
R²⁵ represents hydrogen, hydroxy, amino, C₁-C₄-alkoxy, C₁-C₃-alkylamino, C₁-C₄-alkylcarbonylamino, C₃-C₆-cycloalkylamino or 5- or 6-membered heterocyclyl which is attached via a nitrogen atom,
   where alkoxy may be substituted by 1 to 2 substituents independently of one another selected from the group consisting of amino and C₁-C₃-alkylamino,
   and
   where heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R²⁶ represents hydrogen, C₁-C₄-alkyl, methoxy, trifluoromethyl or benzyloxy,
R²⁷ represents C₁-C₄-alkyl, trifluoromethyl, —S(O)₂NR²⁸R²⁹ or —C(O)NR³⁰R³¹
   where
   R²⁸ represents hydrogen, C₁-C₃-alkyl, C₃-C₆-cycloalkyl, benzyl or 4- to 8-membered heterocyclyl which is attached via a carbon atom,
   R²⁹ represents hydrogen or C₁-C₃-alkyl,
   or
   R²⁸ and R²⁹ together with the nitrogen atom to which they are attached form a 4- to 7-membered heterocycle,
   R³⁰ represents hydrogen, C₁-C₃-alkyl, C₁-C₃-alkoxy, C₃-C₆-cycloalkyl, benzyl or 4- to 8-membered heterocyclyl which is attached via a carbon atom,
      in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
         in which n is a number from 1 to 6,
         in which m is a number from 1 to 6,
      and
      in which cycloalkyl may be substituted by 1 to 2 substituents independently of one another selected from the group consisting of oxo, fluorine, hydroxy, amino, C₁-C₄-alkyl and C₁-C₃-alkylamino,
         in which alkyl and alkylamino for their part may be substituted by 1 to 5 fluorine substituents,
      and
      in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
   R³¹ represents hydrogen or C₁-C₃-alkyl,
   R³⁰ and R³¹ together with the nitrogen atom to which they are attached form a 4- to 7-membered heterocycle,
      in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
R³² represents —S(O)₂NR³⁴R³⁵ or —C(O)NR³⁶R³⁷
   where
   R³⁴ represents hydrogen, C₁-C₃-alkyl, C₃-C₆-cycloalkyl, benzyl or 4- to 8-membered heterocyclyl which is attached via a carbon atom,
   R³⁵ represents hydrogen or C₁-C₃-alkyl,
   or
   R³⁴ and R³⁵ together with the nitrogen atom to which they are attached form a 4- to 7-membered heterocycle,
   R³⁶ represents hydrogen, C₁-C₃-alkyl, C₁-C₃-alkoxy, C₃-C₆-cycloalkyl, benzyl or 4- to 8-membered heterocyclyl which is attached via a carbon atom,
      in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
         in which n is a number from 1 to 6,
         in which m is a number from 1 to 6,
      and
      in which cycloalkyl may be substituted by 1 to 2 substituents independently of one another selected from the group consisting of oxo, fluorine, hydroxy, amino, C₁-C₄-alkyl and C₁-C₃-alkylamino,
         in which alkyl and alkylamino for their part may be substituted by 1 to 5 fluorine substituents,
      and
      in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
   R³⁷ represents hydrogen or C₁-C₃-alkyl,
   R³⁶ and R³⁷ together with the nitrogen atom to which they are attached form a 4- to 7-membered heterocycle,
      in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
R³³ represents hydrogen, C₁-C₄-alkyl, methoxy or trifluoromethyl,
   R³ represents hydrogen, fluorine, chlorine, methyl or methoxy,
   and the salts thereof, the solvates thereof and the solvates of the salts thereof.

Compounds according to the invention are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The compounds of the invention may, depending on their structure, exist in different stereoisomeric forms, i.e. in the form of configurational isomers or else, if appropriate, of conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the compounds of the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting compounds.

Preferred salts in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates in the context of the invention are described as those forms of the compounds according to the invention which form a complex in the solid or liquid state by coordination with solvent molecules. Hydrates are a specific form of the solvates in which the coordination is with water.

The present invention additionally also encompasses prodrugs of the compounds of the invention. The term “prodrugs” encompasses compounds which for their part may be biologically active or inactive but are converted during their residence time in the body into compounds according to the invention (for example by metabolism or hydrolysis).

The two ways (A) and (B) of representing a 1,4-disubstituted cyclohexyl derivative shown below are equivalent to one another and identical, and in both cases describe a trans-1,4-disubstituted cyclohexyl derivative.

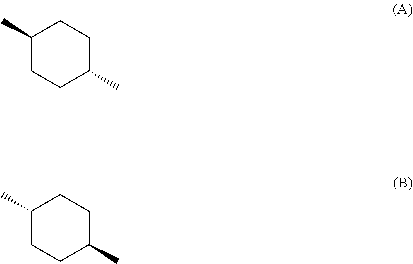

This applies especially to the structural element of tranexamamide, for example N-[(trans-4-{[(tert-butoxycarbonyl)amino]methyl}cyclohexyl)carbonyl and trans-4-(aminomethyl)-cyclohexyl]carbonyl}. In the present invention, representation (A) is used.

The three ways (C), (D) and (E) of representing tautomers of a triazole derivative shown below are equivalent to one another and identical and in all cases describe a 1,4-disubstituted triazole derivative.

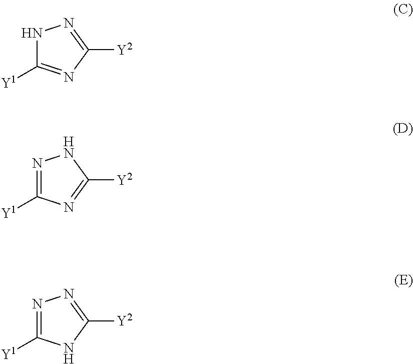

This applies especially to the following structural elements: 1H-1,2,4-triazol-3-yl, 1H-1,2,4-triazol-5-yl, 4H-1,2,4-triazol-3-yl and 4H-1,2,4-triazol-5-yl. Y¹ and Y² here are different substituents.

The two ways (F) and (G) of representing tautomers of a tetrazole derivative shown below are equivalent to one another and identical and in all cases describe a tetrazole derivative.

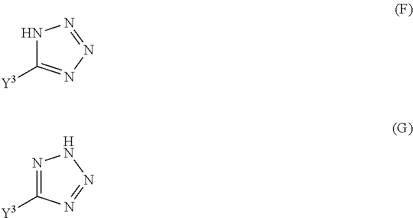

This applies especially to the following structural elements: 1H-tetrazol-5-yl and 2H-tetrazol-5-yl. Y³ here is the remainder of the compound.

The compounds according to the invention of the formula

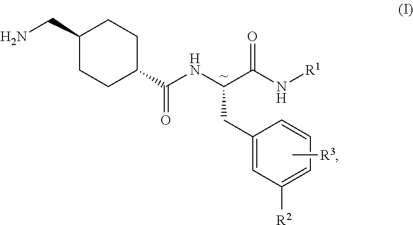

and all L-phenylalanine intermediates are described as the (S) configuration at the stereocentre marked with a “˜” in the above formula, since L-phenylalanine derivatives are introduced into the synthesis as central units. In the preparation of the compounds according to the invention, the coupling of the L-phenylalanine intermediates with the amine H₂N—R¹ can result in partial epimerization at the stereocentre marked with a “˜”. Thus, a mixture of the compounds according to the invention of (S) enantiomer and (R) enantiomer can arise. The main component is the (S) enantiomer depicted in each case. The mixtures of (S) enantiomer and (R) enantiomer can be separated into their enantiomers by methods known to those skilled in the art, for example by chromatography on a chiral phase.

The enantiomers can be separated either directly after the coupling of the L-phenylalanine intermediates with the amine H₂N—R¹ or at a later synthesis intermediate stage, or else the inventive compounds can be separated themselves. Preference is given to the separation of the enantiomers directly after the coupling of the L-phenylalanine intermediates with the amine H₂N—R¹.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is used here synonymously with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, unless specified otherwise, the substituents are defined as follows:

Alkyl represents a straight-chain or branched alkyl radical having 1 to 4 carbon atoms, preferably 1 to 3 carbon atoms, by way of example and with preference methyl, ethyl, n-propyl, isopropyl, 2-methylprop-1-yl, n-butyl and tert-butyl.

Alkoxy represents a straight-chain or branched alkoxy radical having 1 to 4 carbon atoms, preferably 1 to 3 carbon atoms, by way of example and with preference methoxy, ethoxy, n-propoxy, isopropoxy, 2-methylprop-1-oxy, n-butoxy and tert-butoxy.

Alkylamino represents an amino group having one or two independently selected, identical or different, straight-chain or branched alkyl radicals each having 1 to 3 carbon atoms, for example and with preference methylamino, ethylamino, n-propylamino, isopropylamino, N,N-dimethylamino, N,N-diethylamino, N-ethyl-N-methylamino, N-methyl-N-n-propylamino, N-isopropyl-N-n-propylamino and N,N-diisopropylamino. C₁-C₃-Alkylamino represents, for example, a monoalkylamino radical having 1 to 3 carbon atoms or a dialkylamino radical having 1 to 3 carbon atoms in each alkyl radical.

Alkoxycarbonyl represents a straight-chain or branched alkoxy radical attached via a carbonyl group and having 1 to 4 carbon atoms, preferably 1 to 3 carbon atoms, for example and with preference methoxycarbonyl, ethoxycarbonyl, n-propoxycarbonyl, isopropoxycarbonyl, n-butoxycarbonyl and tert-butoxycarbonyl.

Alkylaminocarbonyl represents an amino group having one or two independently selected, identical or different, straight-chain or branched alkyl substituents each having 1 to 3 carbon atoms, attached via a carbonyl group, for example and with preference methylaminocarbonyl, ethylaminocarbonyl, n-propylaminocarbonyl, isopropylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N,N-diisopropylaminocarbonyl. C₁-C₃-Alkylaminocarbonyl represents, for example, a monoalkylaminocarbonyl radical having 1 to 3 carbon atoms or a dialkylaminocarbonyl radical having 1 to 3 carbon atoms in each alkyl substituent.

Alkylcarbonylamino is a straight-chain or branched alkyl radical which is attached via a carbonylamino group and has 1 to 4 carbon atoms, preferably 1 to 3 carbon atoms, for example and with preference methylcarbonylamino, ethylcarbonylamino, n-propylcarbonylamino, isopropylcarbonylamino, n-butylcarbonylamino and tert-butylcarbonylamino.

Cycloalkyl represents a monocyclic cycloalkyl group having 3 to 6 carbon atoms, cycloalkyl which may be mentioned by way of example and with preference being cyclopropyl, cyclobutyl, cyclopentyl and cyclohexyl.

Cycloalkylamino represents a monocyclic cycloalkyl group having 3 to 6 carbon atoms which is attached via an amino group; cycloalkylamino groups which may be mentioned by way of example and with preference are cyclopropylamino, cyclobutylamino, cyclopentylamino and cyclohexylamino.

5-membered heteroaryl in the definition of the R⁶ radical represents an aromatic monocyclic radical having 5 ring atoms and up to 4 heteroatoms and/or hetero groups from the group consisting of S, O, N, SO and SO₂, where one nitrogen atom may also form an N-oxide, for example and with preference thienyl, furyl, pyrrolyl, thiazolyl, oxazolyl, isoxazolyl, oxadiazolyl, pyrazolyl, imidazolyl, triazolyl and tetrazolyl, particularly preferably oxadiazolyl, triazolyl and tetrazolyl.

5-membered heterocycle in the definition of the R⁸ and R⁹ radicals represents a saturated, partly unsaturated or aromatic monocyclic radical having 5 ring atoms and up to 2 heteroatoms and/or hetero groups from the group consisting of S, O, N, SO and SO₂, where one nitrogen atom may also form an N-oxide. This 5-membered heterocycle together with the phenyl ring to which it is attached represents, for example and with preference, 2,3-dihydro-1-benzothiophen-5-yl, 1,3-dihydro-2-benzothiophen-5-yl, 2,3-dihydro-1-benzofuran-5-yl, 1,3-dihydro-2-benzofuran-5-yl, indolin-5-yl, isoindolin-5-yl, 2,3-dihydro-1H-indazol-5-yl, 2,3-dihydro-1H-benzimidazol-5-yl, 1,3-dihydro-2,1-benzoxazol-5-yl, 2,3-dihydro-1,3-benzoxazol-5-yl, 1,3-dihydro-2,1-benzothiazol-5-yl, 2,3-dihydro-1,3-benzothiazol-5-yl, 1H-benzimidazol-5-yl, 1H-indazol-5-yl, 1,2-benzoxazol-5-yl, indol-5-yl, isoindol-5-yl, benzofuran-5-yl, benzothiophen-5-yl, 2,3-dihydro-1-benzothiophen-6-yl, 1,3-dihydro-2-benzothiophen-6-yl, 2,3-dihydro-1-benzofuran-6-yl, 1,3-dihydro-2-benzofuran-6-yl, indolin-6-yl, isoindolin-6-yl, 2,3-dihydro-1H-indazol-6-yl, 2,3-dihydro-1H-benzimidazol-6-yl, 1,3-dihydro-2,1-benzoxazol-6-yl, 2,3-dihydro-1,3-benzoxazol-6-yl, 1,3-dihydro-2,1-benzothiazol-6-yl, 2,3-dihydro-1,3-benzothiazol-6-yl, 1H-benzimidazol-6-yl, 1H-indazol-6-yl, 1,2-benzoxazol-6-yl, indol-6-yl, isoindol-6-yl, benzofuran-6-yl and benzothiophen-6-yl, particularly preferably 2,3-dihydro-1H-indazol-6-yl, 1H-benzimidazol-6-yl and 1H-indazol-6-yl.

Heterocycle in the definition of the radicals R¹¹ and R¹², the radicals R¹³ and R¹⁴, the radicals R¹⁷ and R¹⁸, the radicals R¹⁹ and R²⁰, the radicals R²⁸ and R²⁹, the radicals R³⁰ and R³¹, the radicals R³⁴ and R³⁵ and the radicals R³⁶ and R³⁷ represents a saturated or partly unsaturated monocyclic or bicyclic radical which is attached via a nitrogen atom and which has 4 to 7 ring atoms, preferably 5 or 6 ring atoms, and up to 3 heteroatoms and/or hetero groups, preferably 1 or 2 heteroatoms and/or hetero groups from the series S, O, N, SO and SO₂, where a nitrogen atom may also form an N-oxide, by way of example and with preference azetidinyl, pyrrolidinyl, morpholinyl, thiomorpholinyl, piperidinyl, piperazinyl, 3-azabicyclo[3.1.0]hex-6-yl, 8-azabicyclo[3.2.1]oct-3-yl and azepanyl, particularly preferably pyrrolidinyl, morpholinyl and piperazinyl.

Heterocycle in the definition of the radicals R²¹, R²³ and R²⁵ represents a saturated or partly unsaturated monocyclic radical which is attached via a nitrogen atom and which has 5 or 6 ring atoms and up to 3 heteroatoms and/or hetero groups, preferably 1 or 2 heteroatoms and/or hetero groups from the series S, O, N, SO and SO₂, where a nitrogen atom may also form an N-oxide, by way of example and with preference pyrrolidinyl, morpholinyl, thiomorpholinyl, piperidinyl and piperazinyl, particularly preferably morpholinyl and piperazinyl.

4- to 8-membered heterocyclyl attached via a carbon atom in the definition of the radicals R¹¹, R¹³, R¹⁷, R¹⁹, R²⁸, R³⁰, R³⁴ and R³⁶ represents a saturated or partly unsaturated monocyclic or bicyclic radical which is attached via a carbon atom and which has 4 to 8 ring atoms, preferably 5 or 6 ring atoms, and up to 3 heteroatoms and/or hetero groups, preferably 1 or 2 heteroatoms and/or hetero groups from the series S, O, N, SO and SO₂, where a nitrogen atom may also form an N-oxide, by way of example and with preference azetidinyl, pyrrolidinyl, piperidinyl, tetrahydropranyl, 3-azabicyclo[3.1.0]hex-6-yl, 8-azabicyclo[3.2.1]oct-3-yl and azepanyl, particularly preferably pyrrolidinyl and piperidinyl.

In the formulae of the group which may represent R¹, the end point of the line marked by # in each case does not represent a carbon atom or a CH₂ group, but is part of the bond to the atom to which R¹ is attached.

In the formulae of the group which may represent R², the end point of the line marked by * in each case does not represent a carbon atom or a CH₂ group, but is part of the bond to the atom to which R² is attached.

Preference is given to compounds of the formula (I) in which

R¹ represents a group of the formula

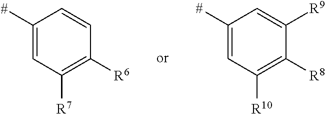

where # is the point of attachment to the nitrogen atom,
R⁶ represents 5-membered heteroaryl,
   where heteroaryl may be substituted by a substituent selected from the group consisting of oxo, chlorine and C₁-C₃-alkyl,
      in which alkyl may be substituted by 1 to 2 substituents independently of one another selected from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁷ represents hydrogen or fluorine,
R⁸ and R⁹ together with the carbon atoms to which they are attached form a 5-membered heterocycle,
   where the heterocycle may be substituted by 1 to 2 substituents independently of one another selected from the group consisting of oxo, chlorine, hydroxy, C₁-C₃-alkyl, pyrazolyl and pyridyl,
      in which alkyl may be substituted by 1 to 2 substituents independently of one another selected from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R¹⁰ represents hydrogen or fluorine,
   R² represents a group of the formula

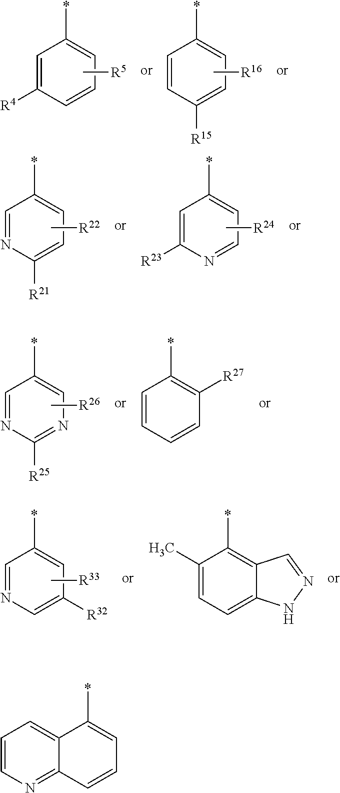

where * is the point of attachment to the phenyl ring,
R⁴ represents amino, C₁-C₄-alkylcarbonylamino, —S(O)₂NR¹¹R¹² or —C(O)NR¹³R¹⁴
   where
   R¹¹ represents hydrogen, methyl, ethyl, C₃-C₆-cycloalkyl or benzyl,
   R¹² represents hydrogen, methyl or ethyl,
   or
   R¹¹ and R¹² together with the nitrogen atom to which they are attached form a 4- to 7-membered heterocycle,
   R¹³ represents hydrogen, C₁-C₃-alkyl, C₁-C₃-alkoxy, C₃-C₆-cycloalkyl or 4- to 8-membered heterocyclyl which is attached via a carbon atom,
      in which alkyl may be substituted by 1 to 2 substituents independently of one another selected from the group consisting of amino, C₁-C₃-alkylamino, —(OCH₂CH₂)ₙ—OCH₃, —(OCH₂CH₂)ₘ—OH, morpholinyl and pyrrolidinyl,
         in which n is a number from 1 to 6,
         in which m is a number from 1 to 6,
      and
      in which cycloalkyl may be substituted by 1 to 2 substituents independently of one another selected from the group consisting of amino, methyl and C₁-C₃-alkylamino,
      and
      in which heterocyclyl may be substituted by 1 to 2 substituents independently of one another selected from the group consisting of C₁-C₄-alkyl,
   R¹⁴ represents hydrogen or methyl,
   R¹³ and R¹⁴ together with the nitrogen atom to which they are attached form a 4- to 7-membered heterocycle,
      in which the heterocycle may be substituted by 1 to 2 substituents independently of one another selected from the group consisting of C₁-C₄-alkyl,
R⁵ represents hydrogen, fluorine, chlorine, methyl, ethyl, methoxy or trifluoromethyl,
R¹⁵ represents amino, —S(O)₂NR¹⁷R¹⁸ or —C(O)NR¹⁹R²⁰,
   where
   R¹⁷ represents hydrogen, methyl, ethyl, C₃-C₆-cycloalkyl or 4- to 8-membered heterocyclyl which is attached via a carbon atom,
   R¹⁸ represents hydrogen, methyl or ethyl,
   or
   R¹⁷ and R¹⁸ together with the nitrogen atom to which they are attached form a 4- to 7-membered heterocycle,
   R¹⁹ represents hydrogen, C₁-C₃-alkyl, C₁-C₃-alkoxy, C₃-C₆-cycloalkyl or 4- to 8-membered heterocyclyl which is attached via a carbon atom,
      in which alkyl may be substituted by 1 to 2 substituents independently of one another selected from the group consisting of amino, C₁-C₃-alkylamino, —(OCH₂CH₂)ₘ—OH, piperidinyl and pyrrolidinyl,
         in which m is a number from 1 to 6,
      and
      in which cycloalkyl may be substituted by 1 to 2 substituents independently of one another selected from the group consisting of amino, methyl and C₁-C₃-alkylamino,
      and
      in which heterocyclyl may be substituted by 1 to 2 substituents independently of one another selected from the group consisting of oxo and C₁-C₄-alkyl,
   R²⁰ represents hydrogen or methyl,
   R¹⁹ and R²⁰ together with the nitrogen atom to which they are attached form a 4- to 7-membered heterocycle,
      in which the heterocycle may be substituted by 1 to 2 substituents independently of one another selected from the group consisting of C₁-C₄-alkyl,
         in which alkyl for its part may be substituted by a hydroxy substituent,
R¹⁶ represents hydrogen, fluorine, chlorine, methyl, ethyl, methoxy or trifluoromethyl,
R²¹ represents hydrogen, hydroxy, amino, C₁-C₄-alkoxy, C₁-C₃-alkylamino or 5- or 6-membered heterocyclyl which is attached via a nitrogen atom,
   where alkoxy may be substituted by 1 to 2 substituents independently of one another selected from the group consisting of amino and C₁-C₃-alkylamino,
   and
   where heterocyclyl may be substituted by 1 to 2 substituents independently of one another selected from the group consisting of C₁-C₄-alkyl,
R²² represents hydrogen, amino, methyl, ethyl, methoxy or trifluoromethyl,
R²³ represents hydrogen or 5- or 6-membered heterocyclyl which is attached via a nitrogen atom,
   where heterocyclyl may be substituted by 1 to 2 substituents independently of one another selected from the group consisting of C₁-C₄-alkyl,
R²⁴ represents hydrogen, methyl, ethyl, methoxy or trifluoromethyl,
R²⁵ represents hydrogen, hydroxy, amino, C₁-C₃-alkylamino, C₁-C₄-alkylcarbonylamino, C₃-C₆-cycloalkylamino or 5- or 6-membered heterocyclyl which is attached via a nitrogen atom,
   where heterocyclyl may be substituted by 1 to 2 substituents independently of one another selected from the group consisting of C₁-C₄-alkyl,
R²⁶ represents hydrogen, methyl, ethyl, methoxy, trifluoromethyl or benzyloxy,
R²⁷ represents C₁-C₄-alkyl, trifluoromethyl or —S(O)₂NR²⁸R²⁹
   where
   R²⁸ represents hydrogen, C₁-C₃-alkyl, C₃-C₆-cycloalkyl or 4- to 8-membered heterocyclyl which is attached via a carbon atom,
   R²⁹ represents hydrogen, methyl or ethyl,
   or
   R²⁸ and R²⁹ together with the nitrogen atom to which they are attached form a 4- to 7-membered heterocycle,
R³² represents —S(O)₂NR³⁴R³⁵
   where
   R³⁴ represents hydrogen, C₁-C₃-alkyl, C₃-C₆-cycloalkyl or 4- to 8-membered heterocyclyl which is attached via a carbon atom,
   R³⁵ represents hydrogen, methyl or ethyl,
   or
   R³⁴ and R³⁵ together with the nitrogen atom to which they are attached form a 4- to 7-membered heterocycle,
R³³ represents hydrogen, methyl, ethyl, methoxy or trifluoromethyl,
   R³ represents hydrogen, fluorine, methyl or methoxy,
   and the salts thereof, the solvates thereof and the solvates of the salts thereof.

Preference is also given to compounds of the formula (I) in which

R¹ represents a group of the formula

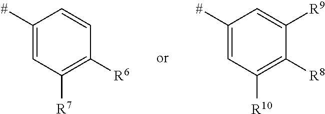

where # is the point of attachment to the nitrogen atom,
R⁶ represents 5-membered heteroaryl,
   where heteroaryl may be substituted by a substituent selected from the group consisting of oxo, chlorine and C₁-C₃-alkyl,
      in which alkyl may be substituted by a hydroxycarbonyl substituent,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁷ represents hydrogen or fluorine,
R⁸ and R⁹ together with the carbon atoms to which they are attached form a 5-membered heterocycle,
   where the heterocycle may be substituted by 1 to 2 substituents independently of one another selected from the group consisting of oxo, hydroxy, methyl, ethyl and n-propyl,
      in which methyl, ethyl and n-propyl may be substituted by a hydroxycarbonyl substituent,
      or
      in which ethyl and n-propyl may be substituted by 4 to 7 fluorine substituents,
      or
      in which ethyl and n-propyl are substituted by a hydroxycarbonyl substituent and in which ethyl and n-propyl are additionally substituted by 4 to 6 fluorine substituents,
R¹⁰ represents hydrogen,
   R² represents a group of the formula

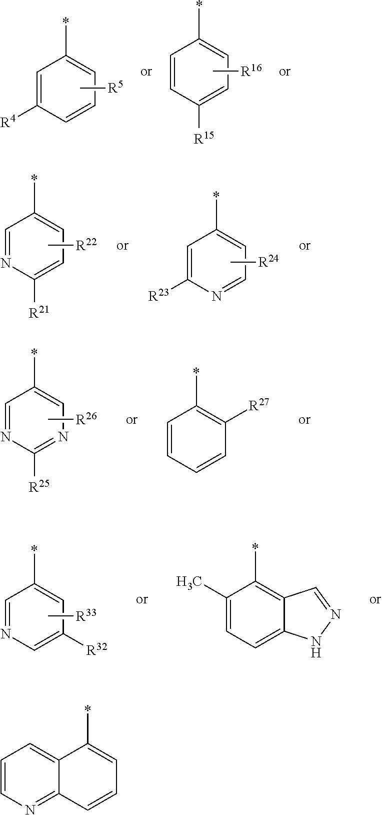

where * is the point of attachment to the phenyl ring,
R⁴ represents amino, C₁-C₄-alkylcarbonylamino, —S(O)₂NR¹¹R¹² or —C(O)NR¹³R¹⁴
   where
   R¹¹ represents hydrogen, methyl, C₃-C₆-cycloalkyl or benzyl,
   R¹² represents hydrogen or methyl,
   or
   R¹¹ and R¹² together with the nitrogen atom to which they are attached form a 4- to 7-membered heterocycle,
   R¹³ represents hydrogen, C₁-C₃-alkyl, methoxy, C₃-C₆-cycloalkyl or 4- to 8-membered heterocyclyl which is attached via a carbon atom,
      in which alkyl may be substituted by 1 to 2 substituents independently of one another selected from the group consisting of amino, C₁-C₃-alkylamino, —(OCH₂CH₂)ₙ—OCH₃, —(OCH₂CH₂)ₘ—OH, morpholinyl and pyrrolidinyl,
         in which n is a number from 1 to 6,
         in which m is a number from 1 to 6,
      and
      in which cycloalkyl may be substituted by 1 to 2 substituents independently of one another selected from the group consisting of amino, methyl and C₁-C₃-alkylamino,
      and
      in which heterocyclyl may be substituted by 1 to 2 substituents independently of one another selected from the group consisting of C₁-C₄-alkyl,
   R¹⁴ represents hydrogen or methyl,
   R¹³ and R¹⁴ together with the nitrogen atom to which they are attached form a 4- to 7-membered heterocycle,
      in which the heterocycle may be substituted by 1 to 2 substituents independently of one another selected from the group consisting of C₁-C₄-alkyl,
R⁵ represents hydrogen, fluorine, chlorine, methyl, ethyl, methoxy or trifluoromethyl,
R¹⁵ represents amino, —S(O)₂NR¹⁷R¹⁸ or —C(O)NR¹⁹R²⁰,
   where
   R¹⁷ represents methyl or 4- to 8-membered heterocyclyl which is attached via a carbon atom,
   R¹⁸ represents hydrogen or methyl,
   R¹⁹ represents hydrogen, C₁-C₃-alkyl, C₃-C₆-cycloalkyl or 4- to 8-membered heterocyclyl which is attached via a carbon atom,
      in which alkyl may be substituted by 1 to 2 substituents independently of one another selected from the group consisting of amino, C₁-C₃-alkylamino, —(OCH₂CH₂)ₘ—OH, piperidinyl and pyrrolidinyl,
         in which m is a number from 1 to 6,
      and
      in which cycloalkyl may be substituted by 1 to 2 substituents independently of one another selected from the group consisting of amino, methyl and C₁-C₃-alkylamino,
      and
      in which heterocyclyl may be substituted by 1 to 2 substituents independently of one another selected from the group consisting of oxo and C₁-C₄-alkyl,
   R²⁰ represents hydrogen or methyl,
   R¹⁹ and R²⁰ together with the nitrogen atom to which they are attached form a 4- to 7-membered heterocycle,
      in which the heterocycle may be substituted by 1 to 2 substituents independently of one another selected from the group consisting of C₁-C₄-alkyl,
         in which alkyl for its part may be substituted by a hydroxy substituent,
R¹⁶ represents hydrogen or methyl,
R²¹ represents hydrogen, hydroxy, amino, C₁-C₄-alkoxy, C₁-C₃-alkylamino or 5- or 6-membered heterocyclyl which is attached via a nitrogen atom,
   where alkoxy may be substituted by 1 to 2 substituents independently of one another selected from the group consisting of amino and C₁-C₃-alkylamino,
   and
   where heterocyclyl may be substituted by 1 to 2 substituents independently of one another selected from the group consisting of C₁-C₄-alkyl,
R²² represents hydrogen, amino, methyl or trifluoromethyl,
R²³ represents hydrogen or 5- or 6-membered heterocyclyl which is attached via a nitrogen atom,
   where heterocyclyl may be substituted by 1 to 2 substituents independently of one another selected from the group consisting of C₁-C₄-alkyl,
R²⁴ represents hydrogen or methyl,
R²⁵ represents hydrogen, hydroxy, amino, C₁-C₃-alkylamino, C₁-C₄-alkylcarbonylamino, C₃-C₆-cycloalkylamino or 5- or 6-membered heterocyclyl which is attached via a nitrogen atom,
   where heterocyclyl may be substituted by 1 to 2 substituents independently of one another selected from the group consisting of C₁-C₄-alkyl,
R²⁶ represents hydrogen, methyl or benzyloxy,
R²⁷ represents C₁-C₄-alkyl, trifluoromethyl or —S(O)₂NR²⁸R²⁹
   where
   R²⁸ and R²⁹ together with the nitrogen atom to which they are attached form a 4- to 7-membered heterocycle,
R³² represents —S(O)₂NR³⁴R³⁵
   where
   R³⁴ represents 4- to 8-membered heterocyclyl which is attached via a carbon atom,
   R³⁵ represents hydrogen or methyl,
R³³ represents hydrogen,
   R³ represents hydrogen, fluorine, methyl or methoxy,
   and the salts thereof, the solvates thereof and the solvates of the salts thereof.

Preference is also given to compounds of the formula (I) in which

R¹ represents a group of the formula

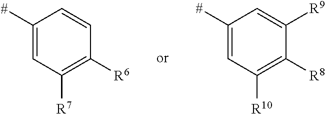

where # is the point of attachment to the nitrogen atom,
R⁶ represents 5-membered heteroaryl,
   where heteroaryl may be substituted by a substituent selected from the group consisting of oxo, chlorine and C₁-C₃-alkyl,
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁷ represents hydrogen or fluorine,
R⁸ and R⁹ together with the carbon atoms to which they are attached form a 5-membered heterocycle,
   where the heterocycle may be substituted by a substituent independently selected from the group consisting of oxo, methyl, ethyl and n-propyl,
      in which ethyl and n-propyl may be substituted by 4 to 7 fluorine substituents,
R¹⁰ represents hydrogen,
   R² represents a group of the formula

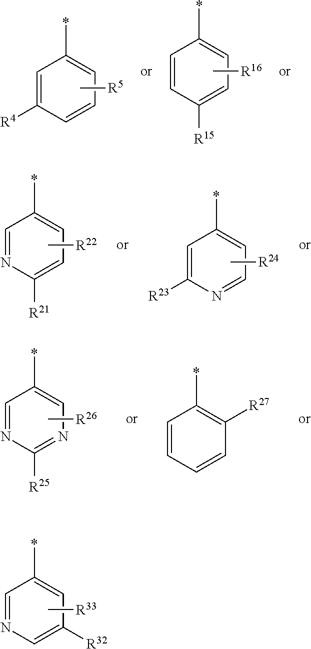

where * is the point of attachment to the phenyl ring,
R⁴ represents amino, C₁-C₄-alkylcarbonylamino, —S(O)₂NR¹¹R¹² or —C(O)NR¹³R¹⁴
   where
   R¹¹ represents hydrogen, methyl, cyclopropyl or benzyl,
   R¹² represents hydrogen or methyl,
   or
   R¹¹ and R¹² together with the nitrogen atom to which they are attached form a 4- to 7-membered heterocycle,
   R¹³ represents hydrogen, C₁-C₃-alkyl, methoxy or 4- to 8-membered heterocyclyl which is attached via a carbon atom,
      in which alkyl may be substituted by 1 to 2 substituents independently of one another selected from the group consisting of C₁-C₃-alkylamino, —(OCH₂CH₂)ₙ—OCH₃, —(OCH₂CH₂)ₘ—OH, morpholinyl and pyrrolidinyl,
         in which n is a number from 1 to 6,
         in which m is a number from 1 to 6,
      and
      in which heterocyclyl may be substituted by 1 to 2 substituents independently of one another selected from the group consisting of C₁-C₄-alkyl,
   R¹⁴ represents hydrogen or methyl,
   R¹³ and R¹⁴ together with the nitrogen atom to which they are attached form a 4- to 7-membered heterocycle,
      in which the heterocycle may be substituted by 1 to 2 substituents independently of one another selected from the group consisting of C₁-C₄-alkyl,
R⁵ represents hydrogen, fluorine, chlorine, methyl or methoxy,
R¹⁵ represents amino, —S(O)₂NR¹⁷R¹⁸ or —C(O)NR¹⁹R²⁰,
   where
   R¹⁷ represents methyl or 4- to 8-membered heterocyclyl which is attached via a carbon atom,
   R¹⁸ represents methyl,
   R¹⁹ represents C₁-C₃-alkyl, C₃-C₆-cycloalkyl or 4- to 8-membered heterocyclyl which is attached via a carbon atom,
      in which alkyl may be substituted by 1 to 2 substituents independently of one another selected from the group consisting of amino, C₁-C₃-alkylamino, —(OCH₂CH₂)ₘ—OH, piperidinyl and pyrrolidinyl,
         in which m is a number from 1 to 6,
      and
      in which cycloalkyl may be substituted by a C₁-C₃-alkylamino substituent
      and
      in which heterocyclyl may be substituted by an oxo substituent,
   R²⁰ represents hydrogen,
   R¹⁹ and R²⁰ together with the nitrogen atom to which they are attached form a 4- to 7-membered heterocycle,
      in which the heterocycle may be substituted by 1 to 2 substituents independently of one another selected from the group consisting of C₁-C₄-alkyl,
         in which alkyl for its part may be substituted by a hydroxy substituent,
R¹⁶ represents hydrogen or methyl,
R²¹ represents hydrogen, hydroxy, amino, C₁-C₄-alkoxy, C₁-C₃-alkylamino or 5- or 6-membered heterocyclyl which is attached via a nitrogen atom,
   where alkoxy may be substituted by a C₁-C₃-alkylamino substituent
   and
   where heterocyclyl may be substituted by 1 to 2 methyl substituents,
R²² represents hydrogen, amino, methyl or trifluoromethyl,
R²³ represents hydrogen or 5- or 6-membered heterocyclyl which is attached via a nitrogen atom,
   where heterocyclyl may be substituted by 1 to 2 methyl substituents,
R²⁴ represents hydrogen or methyl,
R²⁵ represents hydroxy, amino, C₁-C₃-alkylamino, C₁-C₄-alkylcarbonylamino, C₃-C₆-cycloalkylamino or 5- or 6-membered heterocyclyl which is attached via a nitrogen atom,
   where heterocyclyl may be substituted by 1 to 2 methyl substituents,
R²⁶ represents hydrogen, methyl or benzyloxy,
R²⁷ represents C₁-C₄-alkyl, trifluoromethyl or —S(O)₂NR²⁸R²⁹,
   where
   R²⁸ and R²⁹ together with the nitrogen atom to which they are attached form a 4- to 7-membered heterocycle,
R³² represents —S(O)₂NR³⁴R³⁵,
   where
   R³⁴ represents 4- to 8-membered heterocyclyl which is attached via a carbon atom,
   R³⁵ represents hydrogen,
R³³ represents hydrogen,
   R³ represents hydrogen, methyl or methoxy,
   and the salts thereof, the solvates thereof and the solvates of the salts thereof.

Preference is also given to compounds of the formula (I) in which

R¹ represents a group of the formula

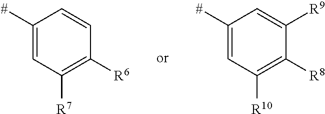

where # is the point of attachment to the nitrogen atom,
R⁶ represents 5-membered heteroaryl,
   where heteroaryl may be substituted by a substituent selected from the group consisting of C₁-C₃-alkyl,
      in which alkyl is substituted by a hydroxycarbonyl substituent and in which alkyl is additionally substituted by 1 to 6 fluorine substituents,
R⁷ represents hydrogen or fluorine,
R⁸ and R⁹ together with the carbon atoms to which they are attached form a 5-membered heterocycle,
   where the heterocycle may be substituted by a substituent independently selected from the group consisting of oxo, methyl, ethyl and n-propyl,
      in which ethyl may be substituted by 4 or 5 fluorine substituents,
R¹⁰ represents hydrogen,
   R² represents a group of the formula

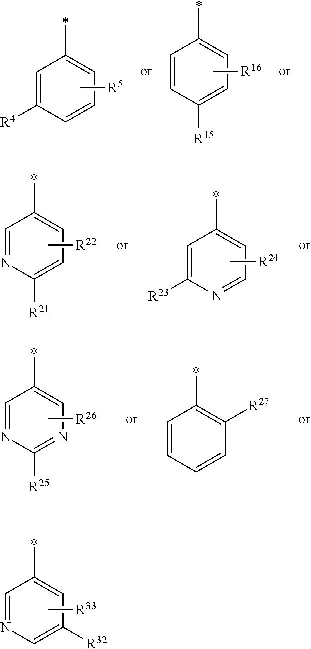

where * is the point of attachment to the phenyl ring,
R⁴ represents —S(O)₂NR¹¹R¹² or —C(O)NR¹³R¹⁴
   where
   R¹¹ represents hydrogen, methyl, cyclopropyl or benzyl,
   R¹² represents hydrogen or methyl,
   or
   R¹¹ and R¹² together with the nitrogen atom to which they are attached form a 4- to 7-membered heterocycle,
   R¹³ represents hydrogen, C₁-C₃-alkyl, methoxy or 4- to 8-membered heterocyclyl which is attached via a carbon atom,
      in which alkyl may be substituted by 1 to 2 substituents independently of one another selected from the group consisting of C₁-C₃-alkylamino, —(OCH₂CH₂)ₙ—OCH₃, —(OCH₂CH₂)ₘ—OH, morpholinyl and pyrrolidinyl,
         in which n is a number from 1 to 6,
         in which m is a number from 1 to 6,
   and
      in which heterocyclyl may be substituted by 1 to 2 substituents independently of one another selected from the group consisting of C₁-C₄-alkyl,
   R¹⁴ represents hydrogen or methyl,
   R¹³ and R¹⁴ together with the nitrogen atom to which they are attached form a 4- to 7-membered heterocycle,
      in which the heterocycle may be substituted by 1 to 2 substituents independently of one another selected from the group consisting of C₁-C₄-alkyl,
R⁵ represents hydrogen, fluorine, chlorine, methyl or methoxy,
R¹⁵ represents amino, —S(O)₂NR¹⁷R¹⁸ or —C(O)NR¹⁹R²⁰,
   where
   R¹⁷ represents methyl or 4- to 8-membered heterocyclyl which is attached via a carbon atom,
   R¹⁸ represents methyl,
   R¹⁹ represents C₁-C₃-alkyl, C₃-C₆-cycloalkyl or 4- to 8-membered heterocyclyl which is attached via a carbon atom,
      in which alkyl may be substituted by 1 to 2 substituents independently of one another selected from the group consisting of amino, C₁-C₃-alkylamino, —(OCH₂CH₂)ₘ—OH, piperidinyl and pyrrolidinyl,
         in which m is a number from 1 to 6,
      and
      in which cycloalkyl may be substituted by a C₁-C₃-alkylamino substituent
      and
      in which heterocyclyl may be substituted by an oxo substituent,
   R²⁰ represents hydrogen,
   R¹⁹ and R²⁰ together with the nitrogen atom to which they are attached form a 4- to 7-membered heterocycle,
      in which the heterocycle may be substituted by 1 to 2 substituents independently of one another selected from the group consisting of C₁-C₄-alkyl,
         in which alkyl for its part may be substituted by a hydroxy substituent,
R¹⁶ represents hydrogen or methyl,
R²¹ represents hydrogen, hydroxy, amino, C₁-C₄-alkoxy, C₁-C₃-alkylamino or 5- or 6-membered heterocyclyl which is attached via a nitrogen atom,
   where alkoxy may be substituted by a C₁-C₃-alkylamino substituent
   and
   where heterocyclyl may be substituted by 1 to 2 methyl substituents,
R²² represents hydrogen, amino, methyl or trifluoromethyl,
R²³ represents hydrogen or 5- or 6-membered heterocyclyl which is attached via a nitrogen atom,
   where heterocyclyl may be substituted by 1 to 2 methyl substituents,
R²⁴ represents hydrogen or methyl,
R²⁵ represents hydroxy, amino, C₁-C₃-alkylamino, C₁-C₄-alkylcarbonylamino, C₃-C₆-cycloalkylamino or 5- or 6-membered heterocyclyl which is attached via a nitrogen atom,
   where heterocyclyl may be substituted by 1 to 2 methyl substituents,
R²⁶ represents hydrogen, methyl or benzyloxy,
R²⁷ represents C₁-C₄-alkyl, trifluoromethyl or —S(O)₂NR²⁸R²⁹,
   where
   R²⁸ and R²⁹ together with the nitrogen atom to which they are attached form a 4- to 7-membered heterocycle,
R³² represents —S(O)₂NR³⁴R³⁵
   where
   R³⁴ represents 4- to 8-membered heterocyclyl which is attached via a carbon atom,
   R³⁵ represents hydrogen,
R³³ represents hydrogen,
   R³ represents hydrogen, methyl or methoxy,
   and the salts thereof, the solvates thereof and the solvates of the salts thereof.

Preference is also given to compounds of the formula (I) in which

R¹ represents a group of the formula

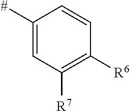

where # is the point of attachment to the nitrogen atom,
R⁶ represents oxadiazolyl, triazolyl and tetrazolyl,
   where oxadiazolyl, triazolyl and tetrazolyl may be substituted by a substituent selected from the group consisting of C₁-C₃-alkyl,
      in which alkyl is substituted by a hydroxycarbonyl substituent and in which alkyl is additionally substituted by 1 to 6 fluorine substituents,
R⁷ represents hydrogen,
   or
   R¹ represents 2,3-dihydro-1H-indazol-6-yl, 1H-benzimidazol-6-yl or 1H-indazol-6-yl,
where 2,3-dihydro-1H-indazol-6-yl, 1H-benzimidazol-6-yl and 1H-indazol-6-yl may be substituted by a substituent independently selected from the group consisting of oxo, methyl, ethyl and n-propyl,
   in which ethyl may be substituted by 4 or 5 fluorine substituents,
      R² represents a group of the formula

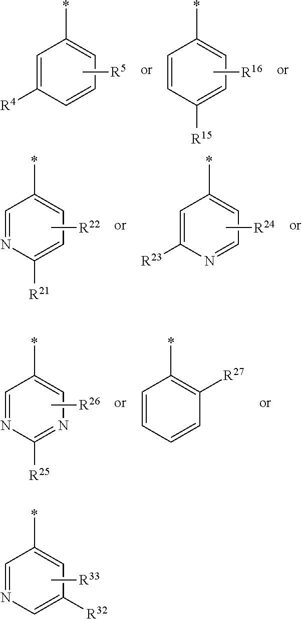

where * is the point of attachment to the phenyl ring,
R⁴ represents —S(O)₂NR¹¹R¹² or —C(O)NR¹³R¹⁴,
   where
   R¹¹ represents hydrogen, methyl, cyclopropyl or benzyl,
   R¹² represents hydrogen or methyl,
   or
   R¹¹ and R¹² together with the nitrogen atom to which they are attached form a pyrrolidinyl, morpholinyl or piperazinyl,
   R¹³ represents hydrogen, C₁-C₃-alkyl, methoxy or heterocyclyl which is attached via a carbon atom and selected from the group consisting of pyrrolidinyl and piperidinyl,
      in which alkyl may be substituted by 1 to 2 substituents independently of one another selected from the group consisting of C₁-C₃-alkylamino, —(OCH₂CH₂)ₙ—OCH₃, —(OCH₂CH₂)ₘ—OH, morpholinyl and pyrrolidinyl,
         in which n is a number from 1 to 6,
         in which m is a number from 1 to 6,
      and
      in which pyrrolidinyl and piperidinyl may be substituted by 1 to 2 substituents independently of one another selected from the group consisting of C₁-C₄-alkyl,
   R¹⁴ represents hydrogen or methyl,
   R¹³ and R¹⁴ together with the nitrogen atom to which they are attached form a pyrrolidinyl, morpholinyl or piperazinyl,
      in which pyrrolidinyl, morpholinyl and piperazinyl may be substituted by 1 to 2 substituents independently of one another selected from the group consisting of C₁-C₄-alkyl,
R⁵ represents hydrogen, fluorine, chlorine, methyl or methoxy,
R¹⁵ represents amino, —S(O)₂NR¹⁷R¹⁸ or —C(O)NR¹⁹R²⁰,
   where
   R¹⁷ represents methyl or heterocyclyl which is attached via a carbon atom and selected from the group consisting of pyrrolidinyl and piperidinyl,
   R¹⁸ represents methyl,
   R¹⁹ represents C₁-C₃-alkyl, C₃-C₆-cycloalkyl or heterocyclyl which is attached via a carbon atom and selected from the group consisting of pyrrolidinyl and piperidinyl,
      in which alkyl may be substituted by 1 to 2 substituents independently of one another selected from the group consisting of amino, C₁-C₃-alkylamino, —(OCH₂CH₂)ₘ—OH, piperidinyl and pyrrolidinyl,
         in which m is a number from 1 to 6,
      and
      in which cycloalkyl may be substituted by a C₁-C₃-alkylamino substituent
      and
      in which pyrrolidinyl and piperidinyl may be substituted by an oxo substituent,
   R²⁰ represents hydrogen,
   R¹⁹ and R²⁰ together with the nitrogen atom to which they are attached form a pyrrolidinyl, morpholinyl or piperazinyl,
      in which pyrrolidinyl, morpholinyl and piperazinyl may be substituted by 1 to 2 substituents independently of one another selected from the group consisting of C₁-C₄-alkyl,
         in which alkyl for its part may be substituted by a hydroxy substituent,
R¹⁶ represents hydrogen or methyl,
R²¹ represents hydrogen, hydroxy, amino, C₁-C₄-alkoxy, C₁-C₃-alkylamino or heterocyclyl which is attached via a nitrogen atom and selected from the group consisting of morpholinyl and piperazinyl,
   where alkoxy may be substituted by a C₁-C₃-alkylamino substituent
   and
   where morpholinyl and piperazinyl may be substituted by 1 to 2 methyl substituents,
R²² represents hydrogen, amino, methyl or trifluoromethyl,
R²³ represents hydrogen or heterocyclyl which is attached via a nitrogen atom and selected from the group consisting of morpholinyl and piperazinyl,
   where morpholinyl and piperazinyl may be substituted by 1 to 2 methyl substituents,
R²⁴ represents hydrogen or methyl,
R²⁵ represents hydroxy, amino, C₁-C₃-alkylamino, C₁-C₄-alkylcarbonylamino, C₃-C₆-cycloalkylamino or heterocyclyl which is attached via a nitrogen atom and selected from the group consisting of morpholinyl and piperazinyl,
   where morpholinyl and piperazinyl may be substituted by 1 to 2 methyl substituents,
R²⁶ represents hydrogen, methyl or benzyloxy,
R²⁷ represents C₁-C₄-alkyl, trifluoromethyl or —S(O)₂NR²⁸R²⁹
   where
   R²⁸ and R²⁹ together with the nitrogen atom to which they are attached form a pyrrolidinyl, morpholinyl or piperazinyl,
R³² represents —S(O)₂NR³⁴R³⁵,
   where
   R³⁴ represents heterocyclyl which is attached via a carbon atom and selected from the group consisting of pyrrolidinyl and piperidinyl,
   R³⁵ represents hydrogen,
R³³ represents hydrogen,
   R³ represents hydrogen, methyl or methoxy,
   and the salts thereof, the solvates thereof and the solvates of the salts thereof.

Preference is also given to compounds of the formula (I) in which

R¹ represents a group of the formula

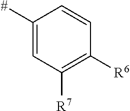

where # is the point of attachment to the nitrogen atom,
R⁶ represents oxadiazolyl, triazolyl and tetrazolyl,
   where oxadiazolyl, triazolyl and tetrazolyl may be substituted by a substituent selected from the group consisting of C₁-C₃-alkyl,
      in which alkyl is substituted by a hydroxycarbonyl substituent and in which alkyl is additionally substituted by 1 to 6 fluorine substituents,
and
R⁷ represents hydrogen.

Preference is also given to compounds of the formula (I) in which

R¹ represents 2,3-dihydro-1H-indazol-6-yl, 1H-benzimidazol-6-yl or 1H-indazol-6-yl,
where 2,3-dihydro-1H-indazol-6-yl, 1H-benzimidazol-6-yl and 1H-indazol-6-yl may be substituted by a substituent independently selected from the group consisting of oxo, methyl, ethyl and n-propyl,
   in which ethyl may be substituted by 4 or 5 fluorine substituents.

Preference is also given to compounds of the formula (I) in which

R² represents a group of the formula

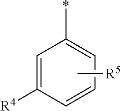

where * is the point of attachment to the phenyl ring,
R⁴ represents —S(O)₂NR¹¹R¹² or —C(O)NR¹³R¹⁴
   where
   R¹¹ represents hydrogen, methyl, cyclopropyl or benzyl,
   R¹² represents hydrogen or methyl,
   or
   R¹¹ and R¹² together with the nitrogen atom to which they are attached form a pyrrolidinyl, morpholinyl or piperazinyl,
   R¹³ represents hydrogen, C₁-C₃-alkyl, methoxy or heterocyclyl which is attached via a carbon atom and selected from the group consisting of pyrrolidinyl and piperidinyl,
      in which alkyl may be substituted by 1 to 2 substituents independently of one another selected from the group consisting of C₁-C₃-alkylamino, —(OCH₂CH₂)ₙ—OCH₃, —(OCH₂CH₂)ₘ—OH, morpholinyl and pyrrolidinyl,
         in which n is a number from 1 to 6,
         in which m is a number from 1 to 6,
      and
      in which pyrrolidinyl and piperidinyl may be substituted by 1 to 2 substituents independently of one another selected from the group consisting of C₁-C₄-alkyl,
   R¹⁴ represents hydrogen or methyl,
   R¹³ and R¹⁴ together with the nitrogen atom to which they are attached form a pyrrolidinyl, morpholinyl or piperazinyl,
      in which pyrrolidinyl, morpholinyl and piperazinyl may be substituted by 1 to 2 substituents independently of one another selected from the group consisting of C₁-C₄-alkyl,
and
R⁵ represents hydrogen, fluorine, chlorine, methyl or methoxy.

Preference is also given to compounds of the formula (I) in which

R² represents a group of the formula

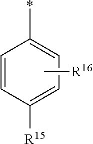

where * is the point of attachment to the phenyl ring,
R¹⁵ represents amino, —S(O)₂NR¹⁷R¹⁸ or —C(O)NR¹⁹R²⁰,
   where
   R¹⁷ represents methyl or heterocyclyl which is attached via a carbon atom and selected from the group consisting of pyrrolidinyl and piperidinyl,
   R¹⁸ represents methyl,
   R¹⁹ represents C₁-C₃-alkyl, C₃-C₆-cycloalkyl or heterocyclyl which is attached via a carbon atom and selected from the group consisting of pyrrolidinyl and piperidinyl,
      in which alkyl may be substituted by 1 to 2 substituents independently of one another selected from the group consisting of amino, C₁-C₃-alkylamino, —(OCH₂CH₂)ₘ—OH, piperidinyl and pyrrolidinyl,
         in which m is a number from 1 to 6,
      and
      in which cycloalkyl may be substituted by a C₁-C₃-alkylamino substituent
      and
      in which pyrrolidinyl and piperidinyl may be substituted by an oxo substituent,
   R²⁰ represents hydrogen,
   R¹⁹ and R²⁰ together with the nitrogen atom to which they are attached form a pyrrolidinyl, morpholinyl or piperazinyl,
      in which pyrrolidinyl, morpholinyl and piperazinyl may be substituted by 1 to 2 substituents independently of one another selected from the group consisting of C₁-C₄-alkyl,
         in which alkyl for its part may be substituted by a hydroxy substituent,
and
R¹⁶ represents hydrogen or methyl.

Preference is also given to compounds of the formula (I) in which

R² represents a group of the formula

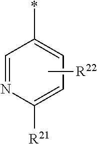

where * is the point of attachment to the phenyl ring,
R²¹ represents hydrogen, hydroxy, amino, C₁-C₄-alkoxy, C₁-C₃-alkylamino or heterocyclyl which is attached via a nitrogen atom and selected from the group consisting of morpholinyl and piperazinyl,
   where alkoxy may be substituted by a C₁-C₃-alkylamino substituent
   and
   where morpholinyl and piperazinyl may be substituted by 1 to 2 methyl substituents,
and
R²² represents hydrogen, amino, methyl or trifluoromethyl.

Preference is also given to compounds of the formula (I) in which

R² represents a group of the formula

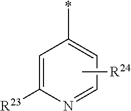

where * is the point of attachment to the phenyl ring,
R²³ represents hydrogen or heterocyclyl which is attached via a nitrogen atom and selected from the group consisting of morpholinyl and piperazinyl,
   where morpholinyl and piperazinyl may be substituted by 1 to 2 methyl substituents,
and
R²⁴ represents hydrogen or methyl.

Preference is also given to compounds of the formula (I) in which

R² represents a group of the formula

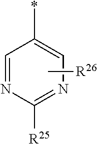

where * is the point of attachment to the phenyl ring,
R²⁵ represents hydroxy, amino, C₁-C₃-alkylamino, C₁-C₄-alkylcarbonylamino, C₃-C₆-cycloalkylamino or heterocyclyl which is attached via a nitrogen atom and selected from the group consisting of morpholinyl and piperazinyl,
   where morpholinyl and piperazinyl may be substituted by 1 to 2 methyl substituents,
and
R²⁶ represents hydrogen, methyl or benzyloxy.

Preference is also given to compounds of the formula (I) in which

R² represents a group of the formula

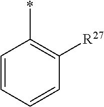

where * is the point of attachment to the phenyl ring,
R²⁷ represents C₁-C₄-alkyl, trifluoromethyl or —S(O)₂NR²⁸R²⁹
   where
   R²⁸ and R²⁹ together with the nitrogen atom to which they are attached form a pyrrolidinyl, morpholinyl or piperazinyl.

Preference is also given to compounds of the formula (I) in which

R² represents a group of the formula

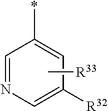

where * is the point of attachment to the phenyl ring,
R³² represents —S(O)₂NR³⁴R³⁵
   where
   R³⁴ represents heterocyclyl which is attached via a carbon atom and selected from the group consisting of pyrrolidinyl and piperidinyl,
   R³⁵ represents hydrogen
and
R³³ represents hydrogen.

Preference is also given to compounds of the formula (I) in which R³ represents hydrogen, methyl or methoxy.

Irrespective of the particular combinations of the radicals specified, the individual radical definitions specified in the particular combinations or preferred combinations of radicals are also replaced as desired by radical definitions from other combinations.

Very particular preference is given to combinations of two or more of the abovementioned preferred ranges.

The invention further provides a process for preparing the compounds of the formula (I), or the salts thereof, solvates thereof or the solvates of the salts thereof, wherein the compounds of the formula

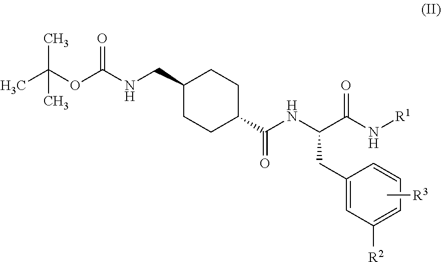

in which
R¹, R² and R³ have the meaning given above,
are reacted with an acid.

The reaction is generally effected in inert solvents, preferably within a temperature range from room temperature to 60° C. at standard pressure.

Inert solvents are, for example, halogenated hydrocarbons such as dichloromethane, trichloromethane, tetrachloromethane or 1,2-dichloroethane, or ethers such as tetrahydrofuran or dioxane, preference being given to dioxane.

Acids are, for example, trifluoroacetic acid or hydrogen chloride in dioxane, preference being given to hydrogen chloride in dioxane.

The compounds of the formula (II) are known or can be prepared by

[A] converting compounds of the formula

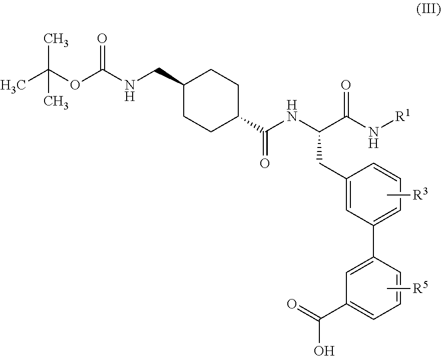

in which
R¹, R³ and R⁵ have the meaning given above
with compounds of the formula

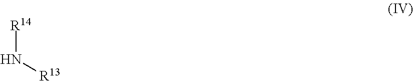

in which
R¹³ and R¹⁴ have the meaning given above
in the presence of dehydrating agents into compounds of the formula

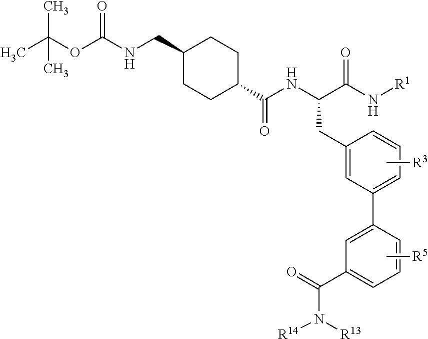

or
[B] converting compounds of the formula

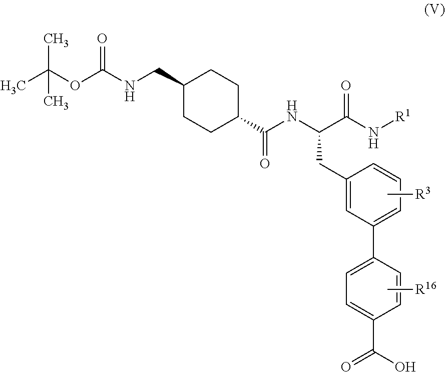

in which
R¹, R³ and R¹⁶ have the meaning given above
with compounds of the formula

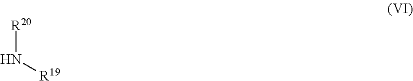

in which
R¹⁹ and R²⁰ have the meaning given above
in the presence of dehydrating agents into compounds of the formula

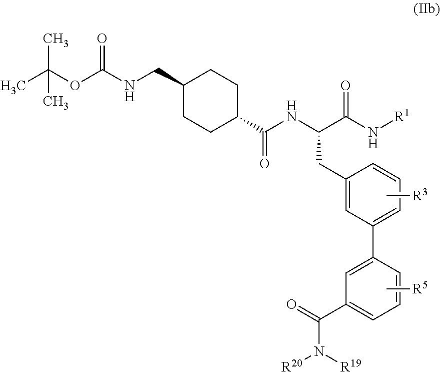

or
[C] converting compounds of the formula

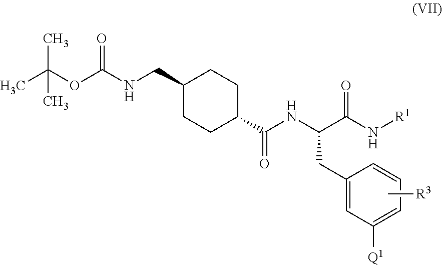

in which
R¹ and R³ have the meaning given above and
Q¹ represents —B(OH)₂, a boronic ester, preferably pinacol boronate, or —BF₃⁻K⁺,
with compounds of the formula

X¹—R² (VIII)

in which
R² has the meaning given above and
X¹ represents bromine or iodine,
under Suzuki coupling conditions,
or
[D] converting compounds of the formula

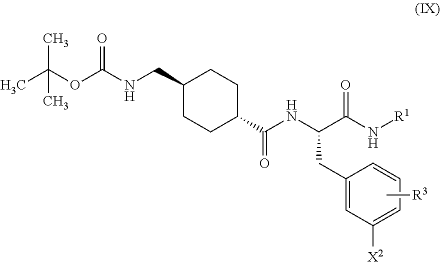

in which
R¹ and R³ have the meaning given above and
X² represents bromine or iodine,
with compounds of the formula

Q²-R² (X)

in which
R² has the meaning given above and
Q² represents —B(OH)₂, a boronic ester, preferably pinacol boronate, or —BF₃⁻K⁺,
under Suzuki coupling conditions,
or
[E] converting compounds of the formula

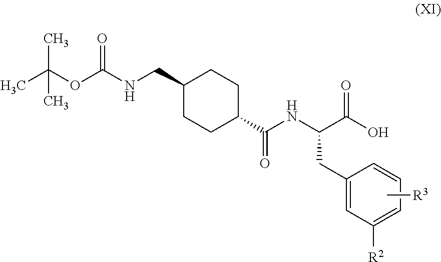

in which
R² and R³ have the meaning given above
with compounds of the formula

H₂N—R¹ (XII)

in which
R¹ has the meaning given above,
in the presence of dehydrating agents.

The compounds of the formulae (IIa) and (IIb) are a subset of the compounds of the formula (II).

The reaction in process [A] is generally effected in inert solvents, optionally in the presence of a base, preferably within a temperature range from 0° C. to the reflux of the solvents at standard pressure.

Suitable dehydrating agents here are, for example, carbodiimides such as N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or ethyl cyano(hydroxyimino)acetate (Oxyma), or (1-cyano-2-ethoxy-2-oxoethylideneaminooxy)dimethylaminomorpholinocarbenium hexafluorophosphate (COMU), or N-[(dimethylamino)(3H-[1,2,3]triazolo [4,5-b]pyridin-3-yloxy)methylidene]-N-methylmethanaminium hexafluorophosphate, or 2,4,6-tripropyl-1,3,5,2,4,6-trioxatriphosphinane 2,4,6-trioxide (T3P), or mixtures of these, with preference being given to N-[(dimethylamino)(3H-[1,2,3]triazolo[4,5-b]pyridin-3-yloxy)methylidene]-N-methylmethanaminium hexafluorophosphate or 2,4,6-tripropyl-1,3,5,2,4,6-trioxatriphosphinane 2,4,6-trioxide (T3P).

Bases are, for example, alkali metal carbonates such as sodium carbonate or potassium carbonate, or sodium bicarbonate or potassium bicarbonate, or organic bases such as trialkylamines, for example triethylamine, N-methylmorpholine, N-methylpiperidine, 4-dimethylaminopyridine or diisopropylethylamine; preference is given to diisopropylethylamine.

Inert solvents are, for example, halogenated hydrocarbons such as dichloromethane or trichloromethane, hydrocarbons such as benzene, or other solvents such as nitromethane, tetrahydrofuran, dioxane, dimethylformamide, dimethyl sulphoxide, acetonitrile or pyridine, or mixtures of the solvents, preference being given to tetrahydrofuran or dimethylformamide or a mixture of dimethylformamide and pyridine.

The compounds of the formula (IV) are known, can be synthesized from the corresponding starting compounds by known processes or can be prepared analogously to the processes described in the Examples section.

The reaction in process [B] is effected as described for process [A].

The compounds of the formula (VI) are known, can be synthesized from the corresponding starting compounds by known processes or can be prepared analogously to the processes described in the Examples section.

The reaction in process [C] is generally effected in inert solvents, in the presence of a catalyst, optionally in the presence of an additional reagent, optionally in a microwave, preferably within a temperature range from room temperature to 150° C. at standard pressure to 3 bar.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphaneferrocenyl)palladium(II) chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)], preference being given to tetrakistriphenylphosphinepalladium(0), [1,1-bis-(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)].

Additional reagents are, for example, potassium acetate, caesium carbonate, potassium carbonate or sodium carbonate, potassium tert-butoxide, caesium fluoride or potassium phosphate, which may be present in aqueous solution; preferred additional reagents are those such as potassium acetate or a mixture of potassium acetate and sodium carbonate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, or mixtures of the solvents with alcohols such as methanol or ethanol and/or water, preference being given to toluene, dimethylformamide or dimethyl sulphoxide.

The compounds of the formula (VIII) are known, can be synthesized from the corresponding starting compounds by known processes or can be prepared analogously to the processes described in the Examples section.

The reaction according to process [D] is carried out as described for process [C].

The compounds of the formula (X) are known, can be synthesized from the corresponding starting compounds by known processes or can be prepared analogously to the processes described in the Examples section.

The reaction according to process [E] is carried out as described for process [A].

The compounds of the formula (XII) are known, can be synthesized from the corresponding starting compounds by known processes or can be prepared analogously to the processes described in the Examples section.

The compounds of the formula (III) are known or can be prepared by

[F] reacting compounds of the formula

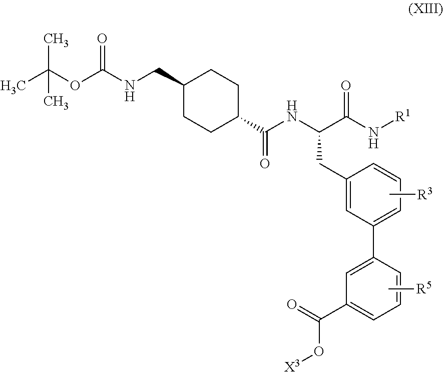

in which
R¹, R³ and R⁵ have the meaning given above and
X³ represents methyl or ethyl,
with a base,
or
[G] reacting compounds of the formula (IX) with compounds of the formula

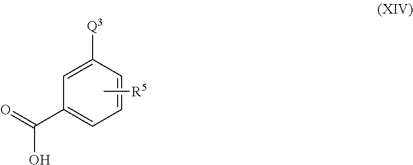

in which
R⁵ has the meaning given above and
Q³ is —B(OH)₂, a boronic ester, preferably pinacol boronate, or —BF₃⁻K⁺,
under Suzuki coupling conditions.

The reaction according to process [F] is generally carried out in inert solvents, preferably in a temperature range from room temperature up to reflux of the solvents at atmospheric pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water, preference being given to a mixture of tetrahydrofuran and water.

Bases are, for example, alkali metal hydroxides such as sodium hydroxide, lithium hydroxide or potassium hydroxide, or alkali metal carbonates such as caesium carbonate, sodium carbonate or potassium carbonate, or alkoxides such as potassium tert-butoxide or sodium tert-butoxide, preference being given to sodium hydroxide and lithium hydroxide.

The reaction according to process [G] is carried out as described for process [C].

The compounds of the formula (XIV) are known, can be synthesized from the corresponding starting compounds by known processes or can be prepared analogously to the processes described in the Examples section.

The compounds of the formula (XIII) are known or can be prepared by reacting compounds of the formula (IX) with compounds of the formula

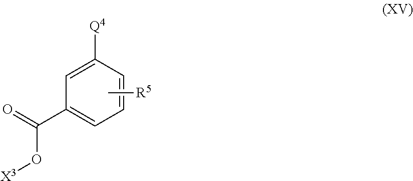

in which
R⁵ has the meaning given above,
X³ represents methyl or ethyl and
Q⁴ is —B(OH)₂, a boronic ester, preferably pinacol boronate, or —BF₃⁻K⁺,
under Suzuki coupling conditions.

The reaction is carried out as described for process [C].

The compounds of the formula (XV) are known, can be synthesized from the corresponding starting compounds by known processes or can be prepared analogously to the processes described in the Examples section.

The compounds of the formula (V) are known or can be prepared by

[H] reacting compounds of the formula

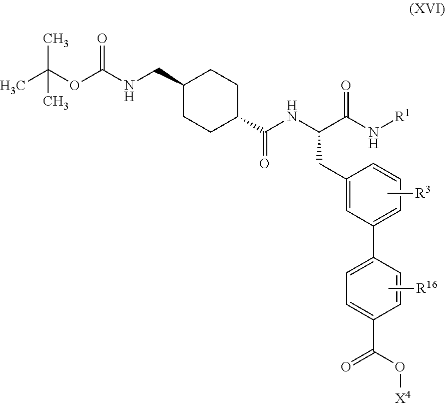

in which
R¹, R³ and R¹⁶ have the meaning given above and
X⁴ represents methyl or ethyl,
with a base,
or
[I] reacting compounds of the formula (IX) with compounds of the formula

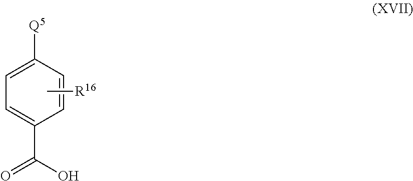

in which
R¹⁶ has the meaning given above and
Q⁵ is —B(OH)₂, a boronic ester, preferably pinacol boronate, or —BF₃⁻K⁺,
under Suzuki coupling conditions.

The reaction according to process [H] is carried out as described for process [F].

The reaction according to process [I] is carried out as described for process [C].

The compounds of the formula (XVII) are known, can be synthesized from the corresponding starting compounds by known processes or can be prepared analogously to the processes described in the Examples section.

The compounds of the formula (XVI) are known or can be prepared by reacting compounds of the formula (IX) with compounds of the formula

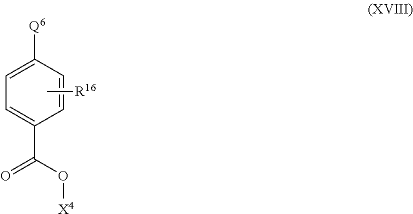

in which
R¹⁶ has the meaning given above,
X⁴ represents methyl or ethyl and
Q⁶ represents —B(OH)₂, a boronic ester, preferably pinacol boronate, or —BF₃⁻K⁺,
under Suzuki coupling conditions.

The reaction is carried out as described for process [C].

The compounds of the formula (XVIII) are known, can be synthesized from the corresponding starting compounds by known processes or can be prepared analogously to the processes described in the Examples section.

The compounds of the formula (VII) are known or can be prepared by reacting compounds of the formula (IX) with 4,4,4′,4′,5,5,5′,5′-octamethyl-2,2′-bi-1,3,2-dioxaborolane.

The reaction is generally effected in inert solvents, in the presence of a catalyst, optionally in the presence of an additional reagent, optionally in a microwave, preferably within a temperature range from room temperature to 150° C. at standard pressure to 3 bar. Hydrolysis in an acidic medium affords the corresponding boronic acids. Workup with potassium dihydrogenfluoride solution (KHF₂ solution) affords the corresponding trifluoroborates.

Catalysts are, for example, palladium catalysts customary for the borylation of aryl halides, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphaneferrocenyl)palladium(II) chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)], preference being given to tetrakistriphenylphosphinepalladium(0) and [1,1-bis-(diphenylphosphino)ferrocene]palladium(II) chloride.

Additional reagents are, for example, potassium acetate, caesium carbonate, potassium carbonate or sodium carbonate, potassium tert-butoxide or sodium tert-butoxide, caesium fluoride, potassium phosphate or potassium phenoxide, preference being given to potassium acetate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, preference being given to dioxane, dimethylformamide or dimethyl sulphoxide.

Literature: K. L. Billingslay, T. E. Barde, S. L Buchwald, Angew. Chem. 2007, 119, 5455 or T. Graening, Nachrichten aus der Chemie, January 2009, 57, 34.

The compounds of the formula (IX) are known or can be prepared by reacting compounds of the formula

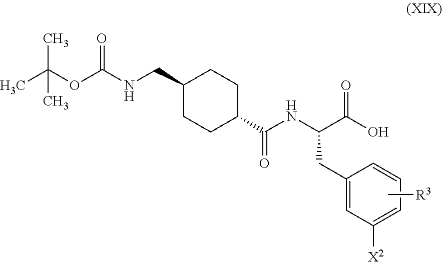

in which
R³ has the meaning given above and
X² is bromine or iodine,
with compounds of the formula (XII) in the presence of dehydrating agents.

The reaction is effected as described for process [A].

The compounds of the formula (XIX) are known, can be synthesized from the corresponding starting compounds by known processes or can be prepared analogously to the processes described in the Examples section.

The compounds of the formula (XI) are known or can be prepared by reacting compounds of the formula (XIX) with compounds of the formula (X) under Suzuki coupling conditions.

The reaction is carried out as described for process [C].

The preparation of the starting compounds and of the compounds of the formula (I) can be illustrated by the synthesis scheme below.

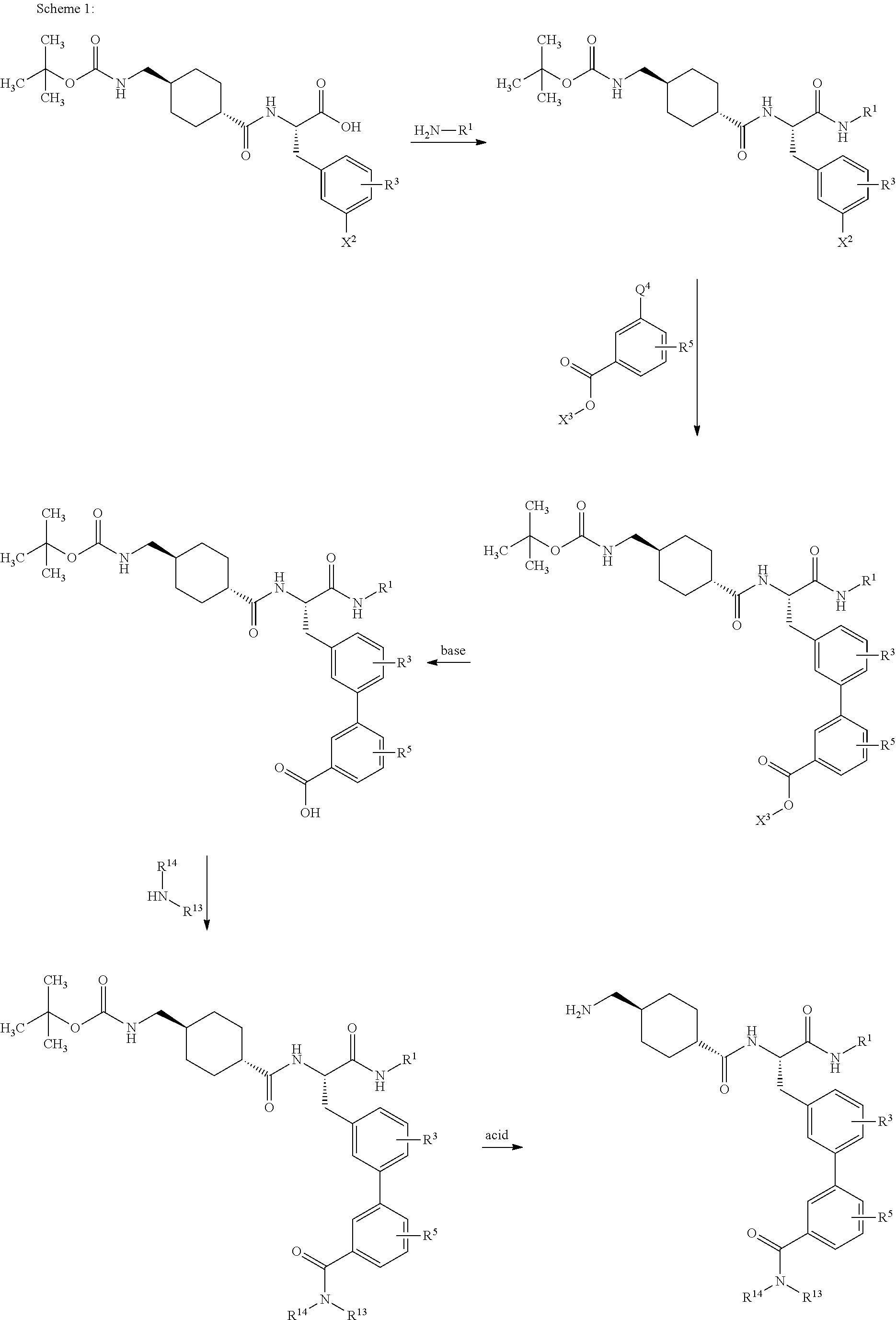

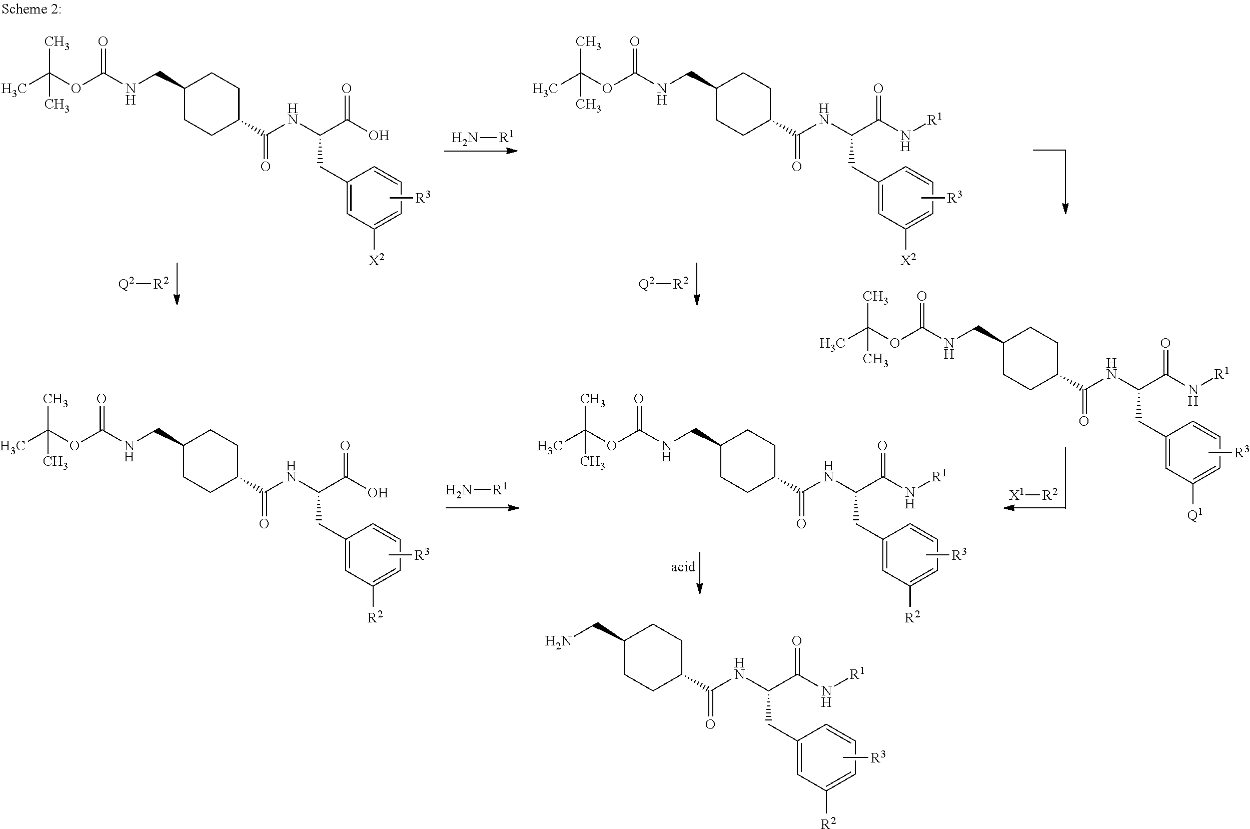

The compounds according to the invention have an unforeseeable useful pharmacological activity spectrum and good pharmacokinetic behaviour. They are compounds that influence the proteolytic activity of the serine proteases FXIa and kallikrein, and possibly plasmin. The inventive compounds inhibit the enzymatic cleavage of substrates that assume a major role in the activation of the blood coagulation cascade and platelet aggregation. If the inventive compounds inhibit plasmin activity, the result is inhibition of fibrinolysis.

They are therefore suitable for use as medicaments for the treatment and/or prophylaxis of diseases in man and animals.

The present invention further provides for the use of the compounds according to the invention for the treatment and/or prophylaxis of disorders, in particular cardiovascular disorders, preferably thrombotic or thromboembolic disorders and/or thrombotic or thromboembolic complications.

“Thromboembolic disorders” in the sense of the present invention include in particular disorders such as acute coronary syndrome (ACS), ST-segment elevation myocardial infarction (STEMI) and non-ST-segment elevation myocardial infarction (non-STEMI), stable angina pectoris, unstable angina pectoris, reocclusions and restenoses after coronary interventions such as angioplasty, stent implantation or aortocoronary bypass, peripheral arterial occlusion diseases, pulmonary embolisms, implantation or aortoooaybps, prpeareilocuindsae, ploayeblss venous thromboses, especially in deep leg veins and renal veins, transitory ischaemic attacks and also thrombotic and thromboembolic stroke.

The inventive compounds are therefore also suitable for the prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with artificial heart valves.

In addition, the inventive compounds are suitable for the treatment and prevention of disseminated intravascular coagulation (DIC) which may occur in connection with sepsis inter alia, but also owing to surgical interventions, neoplastic disorders, burns or other injuries and may lead to severe organ damage through microthromboses.

Thromboembolic complications are also encountered in microangiopathic haemolytic anaemias, extracorporeal circulatory systems, such as haemodialysis, and also prosthetic heart valves.

In addition, the inventive compounds are also suitable for influencing wound healing, for the prophylaxis and/or treatment of atherosclerotic vascular disorders and inflammatory disorders, such as rheumatic disorders of the locomotive system, coronary heart diseases, of heart failure, of hypertension, of inflammatory disorders, for example asthma, inflammatory pulmonary disorders, glomerulonephritis and inflammatory intestinal disorders, for example Crohn's disease or ulcerative colitis or acute renal failure, and additionally likewise for the prophylaxis and/or treatment of dementia disorders, for example Alzheimer's disease. In addition, the inventive compounds can be used for inhibiting tumour growth and the formation of metastases, for microangiopathies, age-related macular degeneration, diabetic retinopathy, diabetic nephropathy and other microvascular disorders, and also for the prevention and treatment of thromboembolic complications, for example venous thromboembolisms, for tumour patients, especially those undergoing major surgery or chemo- or radiotherapy.

In addition, the inventive compounds are also suitable for the prophylaxis and/or treatment of pulmonary hypertension.

The term “pulmonary hypertension” includes certain forms of pulmonary hypertension, as determined, for example, by the World Health Organization (WHO). Examples include pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hypertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for the treatment and/or prophylaxis of pulmonary hypertension associated with sarcoidosis, histiocytosis X and lymphangiomatosis.

In addition, the substances according to the invention are also useful for the treatment of pulmonary and hepatic fibroses.

In addition, the compounds according to the invention are also suitable for the treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications. Moreover, there may be endothelial damage with increased permeability of the vessels and seeping of fluids and proteins into the extravasal lumen. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or injured extravascular tissue. As a consequence, there is coagulation in small vessels of various organs with hypoxia and subsequent organ dysfunction. This can be prevented by the inventive compounds. A secondary effect is the consumption of coagulation factors (for example factor X, prothrombin and fibrinogen) and platelets, which reduces the coagulability of the blood and may result in heavy bleeding.

In addition, the inventive compounds are also useful for the prophylaxis and/or treatment of hyperfibrinolysis. The prophylaxis and/or treatment may reduce or eliminate severe perioperative blood loss. Severe bleeding occurs in major operations, for example coronary artery bypass surgery, transplants or hysterectomy, and in the event of trauma, in the event of haemorrhagic shock or in the event of postpartum haemorrhage. In the aforementioned indications, there may be perioperative use of extracorporeal circulation systems or filter systems, for example heart and lung machines, haemofiltration, haemodialysis, extracorporeal membrane oxygenation or a ventricular support system, for example artificial heart. This additionally requires anticoagulation, for which the inventive compounds can also be used.

The inventive compounds are also suitable for anticoagulation during kidney replacement procedures, for example in the case of continuous veno-venous haemofiltration or intermittent haemodialysis.

In addition, the compounds according to the invention can also be used for preventing coagulation ex vivo, for example for preserving blood and plasma products, for cleaning/pretreating catheters and other medical auxiliaries and instruments, for coating synthetic surfaces of medical auxiliaries and instruments used in vivo or ex vivo or for biological samples which could contain factor XIa.

The present invention further provides for the use of the compounds according to the invention for the treatment and/or prophylaxis of disorders, especially the disorders mentioned above.

The present invention further provides for the use of the compounds according to the invention for production of a medicament for the treatment and/or prophylaxis of disorders, especially the disorders mentioned above.

The present invention further provides a method for the treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

The present invention further provides medicaments comprising a compound according to the invention and one or more further active compounds.

The present invention further provides a method for preventing the coagulation of blood in vitro, especially in banked blood or biological samples which could contain factor XIa, which is characterized in that an anticoagulatory amount of the inventive compound is added.

The present invention further provides medicaments comprising a compound according to the invention and one or more further active compounds, in particular for the treatment and/or prophylaxis of the disorders mentioned above. Preferred examples of active compounds suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors), for example tissue plasminogen activator (t-PA), streptokinase, reteplase and urokinase;
anticoagulatory substances (anticoagulants), for example heparin (UFH), low-molecular-weight heparins (LMWH), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI), for example Pradaxa (dabigatran), atecegatran (AZD-0837), DP-4088 and SSR-182289A, argatroban, bivalirudin and tanogitran (BIBT-986 and prodrug BIBT-1011), hirudin;
direct factor Xa inhibitors, for example rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux;
platelet aggregation-inhibiting substances (platelet aggregation inhibitors, thrombocyte aggregation inhibitors), for example acetylsalicylic acid (for example Aspirin), ticlopidine (Ticlid), clopidogrel (Plavix), prasugrel, ticagrelor, cangrelor, elinogrel, vorapaxar;
fibrinogen receptor antagonists (glycoprotein-IIb/IIIa antagonists), for example abciximab, eptifibatide, tirofiban, lamifiban, lefradafiban and fradafiban;
and also antiarrhythmics;
various antibiotics or antifungal medicaments, either as calculated therapy (prior to the presence of the microbial diagnosis) or as specific therapy;
vasopressors, for example norepinephrine, dopamine and vasopressin;
inotropic therapy, for example dobutamine;
corticosteroids, for example hydrocortisone and fludrocortisone;
recombinant human activated protein C such as, for example, Xigris;
blood products, for example erythrocyte concentrates, thrombocyte concentrates, erythropietin and fresh frozen plasma.

“Combinations” for the purpose of the invention mean not only dosage forms which contain all the components (so-called fixed combinations) and combination packs which contain the components separate from one another, but also components which are administered simultaneously or sequentially, provided that they are used for the prophylaxis and/or treatment of the same disease. It is likewise possible to combine two or more active ingredients with one another, meaning that they are thus each in two-component or multicomponent combinations.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

Parenteral administration is preferred.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds of the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

In spite of this, it may be necessary, if appropriate, to deviate from the amounts specified, specifically depending on body weight, administration route, individual behaviour towards the active ingredient, type of formulation, and time or interval of administration.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### A) EXAMPLES

### Abbreviations

bs/br. s. broad singlet (in NMR)
bd broad doublet (in NMR)
cat. catalytic
CI chemical ionization (in MS)
dd doublet of doublet (in NMR)
DMF dimethylformamide
DMSO dimethyl sulphoxide
dt doublet of triplet (in NMR)
EI electron impact ionization (in MS)
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure, high-performance liquid chromatography
LC-MS liquid chromatography-coupled mass spectrometry
m multiplet (in NMR)
M molar
min minute(s)
MS mass spectrometry
N normal
NMR nuclear magnetic resonance spectrometry
q quartet (in NMR)
quant. quantitative
quint quintet (in NMR)
RT room temperature
Rₜ retention time (in HPLC)
s singlet (in NMR)
TFA trifluoroacetic acid
THF tetrahydrofuran
UV ultraviolet spectrometry

### HPLC and LC/MS Methods:

Method 1 (LC-MS): Instrument: Waters ACQUITY SQD UPLC system; column: Waters Acquity UPLC HSS T3 1.8 μ 50 mm×1 mm; mobile phase A: 11 of water+0.25 ml of 99% strength formic acid, mobile phase B: 11 of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

Method 2 (LC-MS): Instrument: Micromass Quattro Premier with Waters UPLC Acquity; column: Thermo Hypersil GOLD 1.9 μ 50 mm×1 mm; mobile phase A: 11 of water+0.5 ml of 50% strength formic acid, mobile phase B: 11 of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

Method 3 (LC-MS): Instrument: Waters ACQUITY SQD UPLC system; column: Waters Acquity UPLC HSS T3 1.8 g 30 mm×2 mm; mobile phase A: 11 of water+0.25 ml of 99% strength formic acid, mobile phase B: 11 of acetonitrile+0.25 ml of 99% strength formic acid; gradient: 0.0 min 90% A→1.2 min 5% A→2.0 min 5% A; oven: 50° C.; flow rate: 0.60 ml/min; UV detection: 208-400 nm.

Method 4 (LC-MS): Instrument: Waters Acquity UPLC-MS SQD 3001; column: Acquity UPLC BEH C18 1.7 50 mm×2.1 mm; mobile phase A: water+0.1% formic acid, mobile phase B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.

Method 5 (LC-MS): Instrument: Waters Acquity UPLC-MS SQD 3001; column: Acquity UPLC BEH C18 1.7 50 mm×2.1 mm; mobile phase A: water+0.2% ammonia, mobile phase B: acetonitrile; gradient: 0-1.6 min 1-99% B, 1.6-2.0 min 99% B; flow rate: 0.8 ml/min, temperature: 60° C.; injection: 2 μl; DAD scan: 210-400 nm; ELSD.

Method 6 (HPLC): System: Labomatic HD-3000 HPLC gradient pump, Labomatic Labocol Vario-2000 fraction collector, standard UV detector: 254 nm; column: Chromatorex C-18 125 mm×30 mm; flow rate: 150 ml/min; mobile phase A: 0.1% formic acid in water, mobile phase B: acetonitrile; gradient: A 95%/B 5%→A 55%/B 45%.

Method 7 (HPLC): System: Labomatic HD-3000 HPLC gradient pump, Labomatic Labocol Vario-2000 fraction collector, standard UV detector: 254 nm; column: Chromatorex C-18 125 mm×30 mm; flow rate: 150 ml/min; mobile phase A: 0.1% formic acid in water, mobile phase B: acetonitrile; gradient: A 90%/B 10%→A 50%/B 50%.

Method 8 (HPLC): System: Labomatic HD-3000 HPLC gradient pump, Labomatic Labocol Vario-2000 fraction collector, standard UV detector: 254 nm; column: Chromatorex C-18 125 mm×30 mm; flow rate: 150 ml/min; mobile phase A: 0.1% formic acid in water, mobile phase B: acetonitrile; gradient: A 85%/B 15%→A 45%/B 55%.

Method 9 (HPLC): System: Labomatic HD-3000 HPLC gradient pump, Labomatic Labocol Vario-2000 fraction collector, standard UV detector: 254 nm; column: Chromatorex C-18 125 mm×30 mm; flow rate: 150 ml/min; mobile phase A: 0.1% formic acid in water, mobile phase B: acetonitrile; gradient: A 80%/B 20%→A 40%/B 60%.

Method 10 (HPLC): Instrument: Waters autopurification system SQD; column: Waters XBridge C18 5 μ 100 mm×30 mm; mobile phase A: water+0.1% formic acid (99% strength), mobile phase B: acetonitrile; gradient: 0-8.0 min 1-100% B, 8.0-10.0 min 100% B; flow rate: 50.0 ml/min, temperature: RT; injection: 2500 μl; DAD scan: 210-400 nm.

Method 11 (HPLC): Instrument: Waters autopurification system SQD; column: Waters XBridge C18 5 μ 100 mm×30 mm; mobile phase A: water+0.2% ammonia (32% strength), mobile phase B: acetonitrile; gradient: 0-8.0 min 1-100% B, 8.0-10.0 min 100% B; flow rate: 50.0 ml/min; temperature: RT; injection: 2500 μl; DAD scan: 210-400 nm.

Method 12 (LC-MS): MS instrument: Waters (Micromass) QM; HPLC instrument: Agilent 1100 series; column: Agient ZORBAX Extend-C18 3.0 mm×50 mm 3.5 micron; mobile phase A: 11 of water+0.01 mol of ammonium carbonate, mobile phase B: 11 of acetonitrile; gradient: 0.0 min 98% A→0.2 min 98% A→3.0 min 5% A→4.5 min 5% A; oven: 40° C.; flow rate: 1.75 ml/min; UV detection: 210 nm.

Method 13 (LC-MS): Instrument: Waters ACQUITY SQD UPLC system; column: Waters Acquity UPLC HSS T3 1.8 μ 50 mm×1 mm; mobile phase A: 11 of water+0.25 ml of 99% strength formic acid, mobile phase B: 11 of acetonitrile+0.25 ml of 99% strength formic acid; gradient: 0.0 min 95% A→6.0 min 5% A→7.5 min 5% A; oven: 50° C.; flow rate: 0.35 ml/min; UV detection: 210-400 nm.

Method 14 (LC-MS): MS instrument: Waters (Micromass) Quattro Micro; HPLC instrument: Agilent 1100 series; column: YMC-Triart C18 3 μ 50 mm×3 mm; mobile phase A: 11 of water+0.01 mol of ammonium carbonate, mobile phase B: 11 of acetonitrile; gradient: 0.0 min 100% A→2.75 min 5% A→4.5 min 5% A; oven: 40° C.; flow rate: 1.25 ml/min; UV detection: 210 nm.

Method 15 (HPLC): System: Labomatic HD-3000 HPLC gradient pump, Labomatic Labocol Vario-2000 fraction collector, standard UV detector: 254 nm; column: Chromatorex C-18 125 mm×30 mm; flow rate: 150 ml/min; mobile phase A: 0.1% formic acid in water, mobile phase B: acetonitrile, gradient: A 60%/B 40%→A 20%/B 80%.

Method 16 (HPLC): Column: Reprosil C18, 10 μm, Spring Column, 470 mm×50 mm; mobile phase: methanol/water: 0-6 min 20/80; 6-57 min 77/23; 57-76 min 90/10; 76-82 min 20/80; flow rate: 125 ml/min, temperature: RT; UV detection: 210 nm.

Method 17 (HPLC): twice Labomatic pump HD-3000, Labomatic AS-3000, Knauer DAD 2600, Labomatic Labcol Vario 4000 Plus; column: XBridge C18 5 μm 150 mm×50 mm; mobile phase A: water+0.1% formic acid (99% strength), mobile phase B: acetonitrile, gradient: 0-12 min 30-70% B; flow rate: 150 ml/min, temperature: RT; solution: 2354 mg/21 ml DMF; injection: 11×2 ml; UV detection: 254 nm.

Method 18 (HPLC): Waters autopurification system: Pump 254, Sample Manager 2767, CFO, DAD 2996, ELSD 2424, SQD 3100; column: XBridge C18 5 μm 100×30 mm, mobile phase A: water+0.1% formic acid (99% strength). Mobile phase B: methanol, gradient: 0-8 min 10-60% B; flow rate: 50 ml/min; temperature: RT; solution: 76 mg/1.5 ml of DMSO/methanol 1:1; injection: 1×1.5 ml; detection: DAD scan 210-400 nm.

Microwave: The microwave reactor used was an instrument of the Biotage™ Initiator type.

When compounds according to the invention are purified by preparative HPLC by the above-described methods in which the eluents contain additives, for example trifluoroacetic acid, formic acid or ammonia, the compounds according to the invention may be obtained in salt form, for example as trifluoroacetate, formate or ammonium salt, if the compounds according to the invention contain a sufficiently basic or acidic functionality. Such a salt can be converted to the corresponding free base or acid by various methods known to the person skilled in the art. Weaker salts can be converted to the corresponding chlorides by addition of a little hydrochloride.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

If the starting compounds and examples contain an L-phenylalanine derivative as the central unit, the corresponding stereocentre is described as the (S) configuration. In the absence of further information, there was no check in individual cases as to whether partial epimerization of the stereocentre took place in the coupling of the L-phenylalanine intermediate with the amine H₂N—R¹. Thus, a mixture of the inventive compounds of (S) enantiomer and (R) enantiomer may be present. The main component is the (S) enantiomer depicted in each case.

### Starting Materials

### Example 1A

### Methyl 3-bromo-L-phenylalaninate hydrochloride

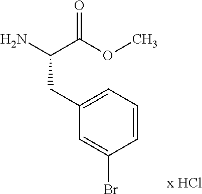

3-Bromo-L-phenylalanine (5 g, 19.46 mmol) was dissolved in 25 ml of methanol, the solution was cooled to 0° C. and thionyl chloride (9.26 g, 77.8 mmol) was added. The reaction mixture was stirred at RT for 48 h and then concentrated to dryness. This gave 5.7 g (100% of theory) of the title compound.

¹H NMR (400 MHz, DMSO-d₆): δ=ppm 3.11 (dd, 4.55 Hz, 2H), 3.66 (s, 3H), 4.29 (dd, 1H), 7.19-7.30 (m, 2H), 7.43-7.48 (m, 2H), 8.58 (br. s., 3H).

LC-MS (Method 4): Rₜ=0.65 min; MS (ESIpos): m/z=258 [M+H−HCl]⁺.

### Example 2A

### Methyl 3-bromo-N-[(trans-4-{[(tert-butoxycarbonyl)amino]methyl}cyclohexyl)carbonyl]-L-phenylalaninate

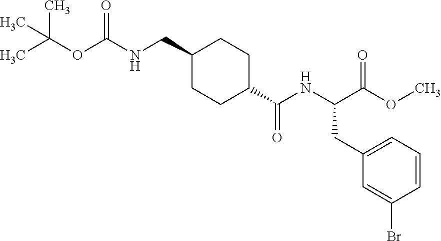

Methyl 3-bromo-L-phenylalaninate hydrochloride (4.6 g, 15.7 mmol), trans-4-{[(tert-butoxycarbonyl)amino]methyl}cyclohexanecarboxylic acid (4.1 g, 15.75 mmol) and N,N-diisopropylethylamine (11 ml, 63 mmol) were suspended in 85 ml of ethyl acetate. The solution was cooled to 0° C. Subsequently, 2,4,6-tripropyl-1,3,5,2,4,6-trioxatriphosphinane 2,4,6-trioxide (50% in ethyl acetate, 25 g, 39.4 mmol) was added dropwise, and the mixture was stirred at 0° C. for 30 minutes and at RT overnight. The mixture was quenched with water and extracted three times with ethyl acetate. The combined organic phases were washed once with saturated aqueous ammonium chloride solution and once with saturated aqueous sodium chloride solution, dried over magnesium sulphate, filtered and concentrated to dryness. The residue was recrystallized from acetonitrile. This gave 4.7 g (60% of theory) of the title compound.

¹H NMR (300 MHz, DMSO-d₆): δ=ppm 0.69-0.86 (m, 2H), 0.99-1.27 (m, 3H), 1.33 (s, 9H), 1.45-1.55 (m, 1H), 1.61 (m, 3H), 1.92-2.05 (m, 1H), 2.70 (m, 2H), 2.82 (dd, 1H), 3.01 (dd, 1H), 4.34-4.46 (m, 1H), 6.69-6.79 (m, 1H), 7.14-7.25 (m, 2H), 7.32-7.41 (m, 2H), 8.12 (d, 1H).

LC-MS (Method 4): Rₜ=1.30 min; MS (ESIpos): m/z=499 [M+H]⁺.

### Example 3A

### Methyl 3-bromo-N-[(trans-4-{[(tert-butoxycarbonyl)amino]methyl}cyclohexyl)carbonyl]-4-methyl-L-phenylalaninate

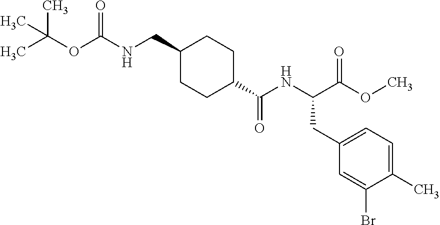

Methyl 3-bromo-4-methyl-L-phenylalaninate hydrochloride (3.5 g, 13 mmol), trans-4-{[(tert-butoxycarbonyl)amino]methyl}cyclohexanecarboxylic acid (3.3 g, 13 mmol) and N,N-diisopropylethylamine (6.7 ml, 39 mmol) were suspended in 100 ml of ethyl acetate. Subsequently, 2,4,6-tripropyl-1,3,5,2,4,6-trioxatriphosphinane 2,4,6-trioxide (50% in ethyl acetate, 20.5 g, 32 mmol) was added dropwise, and the mixture was stirred at RT overnight. The mixture was quenched with water and the aqueous phase was extracted three times with ethyl acetate. The combined organic phases were washed with saturated aqueous sodium bicarbonate solution and with saturated aqueous sodium chloride solution, dried over sodium sulphate, filtered and concentrated to dryness. This gave 5.8 g (88% of theory) of the title compound.

¹H NMR (300 MHz, DMSO-d₆): δ=ppm 0.72-0.87 (m, 2H), 1.01-1.24 (m, 3H), 1.33 (s, 9H), 1.48-1.69 (m, 4H), 1.93-2.06 (m, 1H), 2.25 (s, 3H), 2.65-2.83 (m, 3H), 2.96 (dd, 1H), 3.57 (s, 3H), 4.26-4.45 (m, 1H), 6.70-6.81 (m, 1H), 7.01-7.12 (m, 1H), 7.21 (d, 1H), 7.39 (s, 1H), 8.08-8.17 (m, 1H).

LC-MS (Method 4): Rₜ=1.36 min; MS (ESIpos): m/z=513 [M+H]⁺.

### Example 4A

### 3-Bromo-N-[(trans-4-{[(tert-butoxycarbonyl)amino]methyl}cyclohexyl)carbonyl]-L-phenylalanine

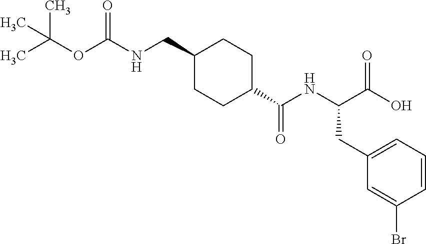

Methyl 3-bromo-N-[(trans-4-{[(tert-butoxycarbonyl)amino]methyl}cyclohexyl)carbonyl]-L-phenylalaninate (5.9 g, 11.8 mmol) was dissolved in 87 ml of tetrahydrofuran, the mixture was cooled to 0° C. and 8.9 ml of 2N aqueous sodium hydroxide solution were added. The mixture was allowed to come to RT and stirred at RT overnight. Subsequently, the tetrahydrofuran was drawn off and the aqueous phase was washed twice with methyl tert-butyl ether. The aqueous phase was then adjusted to pH 3 with 1N hydrochloric acid and the precipitated solid was filtered off. The aqueous phase was extracted three times with dichloromethane and the organic phase was concentrated. The residue from the organic phase was combined with the solid and dried under high vacuum. This gave 5.36 g (94% of theory) of the title compound.

¹H NMR (300 MHz, DMSO-d₆): δ=ppm 0.77 (m, 2H), 1.00-1.27 (m, 3H), 1.33 (s, 9H), 1.61 (m, 5H), 1.92-2.05 (m, 1H), 2.70 (dd, 2H), 2.82 (dd, 1H), 3.01 (dd, 1H), 4.21-4.31 (m, 1H), 6.74 (dd, 1H), 7.12-7.22 (m, 2H), 7.29-7.38 (m, 2H), 7.79 (d, 1H).

LC-MS (Method 4): Rₜ=1.18 min; MS (ESIpos): m/z=481 [M+H]⁺.

### Example 5A

### 3-Bromo-N-[(trans-4-{[(tert-butoxycarbonyl)amino]methyl}cyclohexyl)carbonyl]-4-methyl-L-phenylalanine

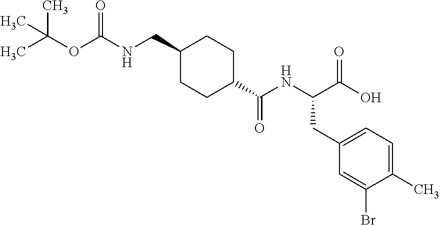

Methyl 3-bromo-N-[(trans-4-{[(tert-butoxycarbonyl)amino]methyl}cyclohexyl)carbonyl]-4-methyl-L-phenylalaninate (5.8 g, 11.3 mmol) was dissolved in 70 ml of tetrahydrofuran, the mixture was cooled to 0° C. and 8.5 ml of 2N aqueous sodium hydroxide solution were added. The mixture was allowed to come to RT and stirred at RT for 48 h. The tetrahydrofuran was then drawn off and the precipitated solid was filtered off with suction and washed with methyl tert-butyl ether and dried under high vacuum. This gave 4.8 g (85% of theory) of the title compound.

¹H NMR (300 MHz, DMSO-d₆): δ=ppm 0.69-0.88 (m, 2H), 1.08-1.27 (m, 3H), 1.33 (s, 9H), 1.56-1.69 (m, 4H), 1.85-2.00 (m, 1H), 2.22 (s, 3H), 2.65-2.74 (m, 2H), 2.84 (dd, 1H), 2.98 (dd, 1H), 3.75-3.84 (m, 1H), 6.70-6.79 (m, 1H), 6.87-6.95 (m, 2H), 7.10 (d, 1H), 7.15-7.21 (m, 1H).

LC-MS (Method 4): Rₜ=1.18 min; MS (ESIpos): m/z=495 [M+H]⁺.

### Example 6A

### 3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide

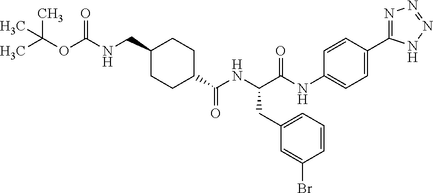

3-Bromo-N-[(trans-4-{[(tert-butoxycarbonyl)amino]methyl}cyclohexyl)carbonyl]-L-phenylalanine (10 g, 20.7 mmol), 4-(1H-tetrazol-5-yl)aniline (3.4 g, 20.7 mmol) and N,N-diisopropylethylamine (11 ml, 62 mmol) were suspended in 400 ml of ethyl acetate, and 2,4,6-tripropyl-1,3,5,2,4,6-trioxatriphosphinane 2,4,6-trioxide (50% in ethyl acetate, 30.5 ml, 52 mmol) was added. The mixture was subsequently stirred at RT overnight and then heated under reflux for 3 h. Water was added to the reaction mixture and the precipitated solid was filtered off with suction, washed with a little ethyl acetate and water and dried under high vacuum. This gave 7.23 g (56% of theory) of the title compound.

¹H NMR (300 MHz, DMSO-d₆): δ=ppm 0.79 (m, 2H), 0.98-1.26 (m, 3H), 1.33 (s, 9H), 1.45-1.72 (m, 4H), 1.98-2.14 (m, 1H), 2.71 (t, 2H), 2.84 (dd, 1H), 3.02 (dd, 2H), 4.55-4.70 (m, 1H), 6.67-6.85 (m, 1H), 7.16-7.32 (m, 2H), 7.36 (d, 1H), 7.50 (s, 1H), 7.76 (d, 2H), 7.95 (d, 2H), 8.13 (d, 1H), 10.41 (s, 1H).

LC-MS (Method 4): Rₜ=1.18 min; MS (ESIpos): m/z=628 [M+H]⁺.

### Example 7A

### 3-Bromo-N-alpha-[(trans-4-{[(tert-butoxycarbonyl)amino]methyl}cyclohexyl)carbonyl]-4-methyl-N-[4-(1H-tetrazol-5-yl)phenyl]-L-phenylalaninamide

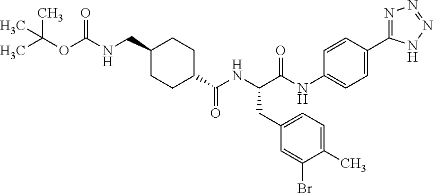

3-Bromo-N-[(trans-4-{[(tert-butoxycarbonyl)amino]methyl}cyclohexyl)carbonyl]-4-methyl-L-phenylalanine (3 g, 6.0 mmol), 4-(1H-tetrazol-5-yl)aniline (1.0 g, 6.0 mmol) and N,N-diisopropylethylamine (3.1 ml, 18 mmol) were suspended in 100 ml of ethyl acetate, and 2,4,6-tripropyl-1,3,5,2,4,6-trioxatriphosphinane 2,4,6-trioxide (50% in ethyl acetate, 9.6 g, 15 mmol) was added. The reaction mixture was heated under reflux for 3 h, water was added and the precipitated solid was filtered off with suction. The solid was washed with a little ethyl acetate and water and dried under high vacuum. This gave 2.65 g (69% of theory) of the title compound.

¹H NMR (300 MHz, DMSO-d₆): δ=ppm 0.71-0.93 (m, 2H), 1.22 (d, 3H), 1.33 (s, 9H), 1.51-1.69 (m, 4H), 2.00-2.14 (m, 1H), 2.25 (s, 3H), 2.64-2.74 (m, 2H), 2.79 (dd, 1H), 2.97 (dd, 1H), 4.54-4.65 (m, 1H), 6.67-6.77 (m, 1H), 7.19 (m, 2H), 7.50 (s, 1H), 7.75 (d, 2H), 7.95 (d, 2H), 8.03-8.11 (m, 1H), 10.36 (s, 1H).

LC-MS (Method 4): Rₜ=1.25 min; MS (ESIpos): m/z=640 [M+H]⁺.

### Example 8A

### N-alpha-[(trans-4-{[(tert-Butoxycarbonyl)amino]methyl}cyclohexyl)carbonyl]-3-(4,4,5,5-tetramethyl-1,3,2-dioxaborolan-2-yl)-N-[4-(1H-tetrazol-5-yl)phenyl]-L-phenylalaninamide

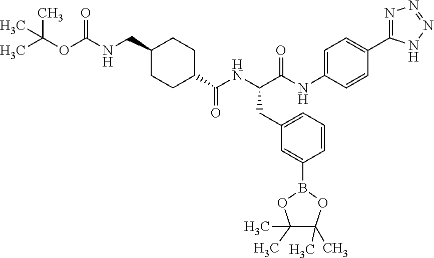

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (1 g, 1.6 mmol) and bis(pinacolato)diborane (729 mg, 2.9 mmol) were dissolved in DMSO, freed of air with argon and blanketed. 1,1′-Bis(diphenylphosphino)ferrocenedichloropalladium(II) (130 mg, 0.16 mmol) and potassium acetate (470 mg, 47 mmol) were added to the reaction mixture, and the reaction mixture was stirred in a microwave (Biotage Initiator) at 110° C. for 6 h and then converted further as the crude product.

LC-MS (Method 4): Rₜ=1.28 min; MS (ESIpos): m/z=674.6 [M+H]⁺.

### Example 9A

### 3-Bromo-N-alpha-[(trans-4-{[(tert-butoxycarbonyl)amino]methyl}cyclohexyl)carbonyl]-N-[3-fluoro-4-(1H-tetrazol-5-yl)phenyl]-L-phenylalaninamide

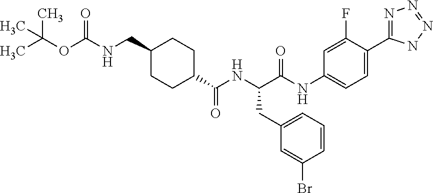

3-Bromo-N-[(trans-4-{[(tert-butoxycarbonyl)amino]methyl}cyclohexyl)carbonyl]-L-phenylalanine (1.2 g, 2.5 mmol), 3-fluoro-4-(2H-tetrazol-5-yl)aniline (0.5 g, 2.8 mmol) and N,N-diisopropylethylamine (1.33 ml, 7.6 mmol) were suspended in 14 ml of ethyl acetate, and 2,4,6-tripropyl-1,3,5,2,4,6-trioxatriphosphinane 2,4,6-trioxide (50% in ethyl acetate, 4.0 g, 6.3 mmol) was added. The reaction mixture was heated under reflux for 4 h, water was added and the precipitated solid was filtered off with suction. The solid was washed with a little ethyl acetate and water and dried under high vacuum. This gave 840 mg (51% of theory) of the title compound.

¹H NMR (300 MHz, DMSO-d₆): δ=ppm 0.73-0.90 (m, 2H), 1.02-1.29 (m, 4H), 1.35 (s, 9H), 1.66 (m, 4H), 2.01-2.15 (m, 1H), 2.73 (m, 2H), 2.80-2.90 (m, 1H), 3.01-3.09 (m, 1H), 4.58-4.69 (m, 1H), 6.73-6.81 (m, 1H), 7.25 (m, 2H), 7.39 (m, 1H), 7.45-7.54 (m, 1H), 7.84 (dd, 1H), 8.01 (t, 1H), 8.20 (d, 1H), 10.65 (s, 1H).

LC-MS (Method 4): Rₜ=0.83 min; MS (ESIpos): m/z=643 [M+H]⁺.

### Example 10A

### Methyl 3′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]biphenyl-3-carboxylate

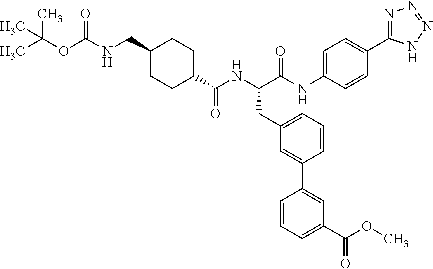

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and methyl 3-(4,4,5,5-tetramethyl-1,3,2-dioxaborolan-2-yl)benzoate (94 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 39 mg (24% of theory) of the title compound.

LC-MS (Method 4): Rₜ=1.30 min; MS (ESIpos): m/z=682.4 [M+H]⁺.

### Example 11A

### 3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-6-methylbiphenyl-3-carboxylic acid

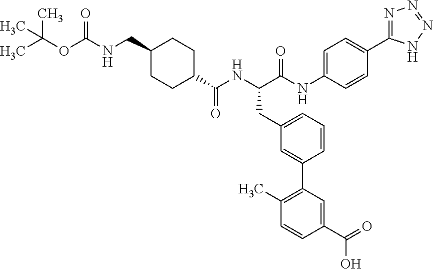

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (1.0 g, 1.6 mmol) and 4-methyl-3-(4,4,5,5-tetramethyl-1,3,2-dioxaborolan-2-yl)benzoic acid (627 mg, 2.4 mmol) were dissolved in dimethyl sulphoxide (10 ml), and tetrakis(triphenylphosphine)palladium(0) (184 mg, 160 μmol), sodium carbonate (507 mg, 4.8 mmol) and water (2.4 ml, 133 mmol) were added. The reaction mixture was stirred at 110° C. in a microwave (Biotage Initiator) for 240 min, cooled, filtered and, in several portions, purified chromatographically via HPLC (Method 9). This gave 399 mg (37% of theory) of the title compound.

LC-MS (Method 4): Rₜ=1.15 min; MS (ESIpos): m/z=682.5 [M+H]⁺.

### Example 12A

### 3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2-methylbiphenyl-4-carboxylic acid

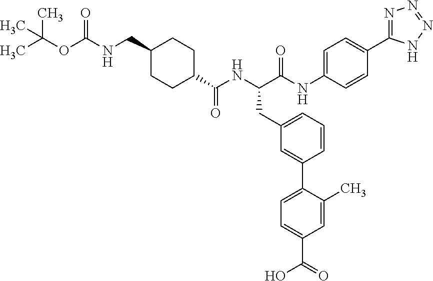

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (1.0 g, 1.6 mmol) and 4-carboxy-2-methylphenylboronic acid (627 mg, 2.4 mmol) were dissolved in dimethyl sulphoxide (10 ml), and tetrakis(triphenylphosphine)palladium(0) (184 mg, 160 μmol), sodium carbonate (507 mg, 4.8 mmol) and water (2.4 ml, 133 mmol) were added. The reaction mixture was stirred at 110° C. in a microwave (Biotage Initiator) for 240 min, cooled, filtered and, in several portions, purified chromatographically via HPLC (Method 9). This gave 468 mg (43% of theory) of the title compound.

LC-MS (Method 4): Rₜ=1.14 min; MS (ESIpos): m/z=682.5 [M+H]⁺.

### Example 13A

### 5′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2′-methylbiphenyl-3-carboxylic acid

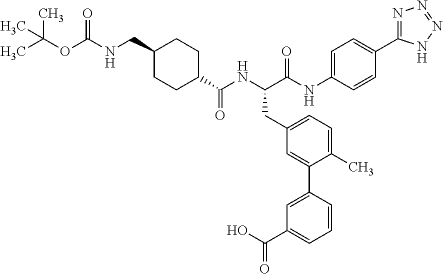

3-Bromo-N-alpha-[(trans-4-{[(tert-butoxycarbonyl)amino]methyl}cyclohexyl)carbonyl]-4-methyl-N-[4-(1H-tetrazol-5-yl)phenyl]-L-phenylalaninamide (500 mg, 0.78 mmol) and 3-carboxyphenylboronic acid (159 mg, 1.6 mmol) were dissolved in dimethyl sulphoxide (6 ml), and tetrakis(triphenylphosphine)palladium(0) (135 mg, 118 μmol), sodium carbonate (248 mg, 2.3 mmol) and water (1.2 ml, 65 mmol) were added. The reaction mixture was stirred at 110° C. in a microwave (Biotage Initiator) for 300 min, cooled, filtered and, in several portions, purified chromatographically via HPLC (Method 9). This gave 344 mg (65% of theory) of the title compound.

LC-MS (Method 4): Rₜ=1.20 min; MS (ESIpos): m/z=682.6 [M+H]⁺.

### Example 14A

### 5′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2′-methylbiphenyl-4-carboxylic acid

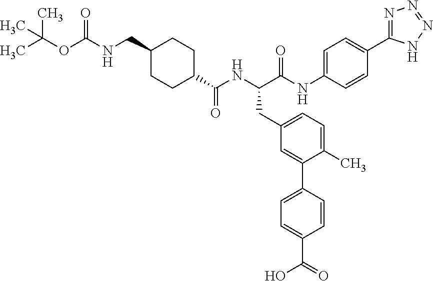

3-Bromo-N-alpha-[(trans-4-{[(tert-butoxycarbonyl)amino]methyl}cyclohexyl)carbonyl]-4-methyl-N-[4-(1H-tetrazol-5-yl)phenyl]-L-phenylalaninamide (350 mg, 0.55 mmol) and 4-(4,4,5,5-tetramethyl-1,3,2-dioxaborolan-2-yl)benzoic acid (270 mg, 1.1 mmol) were dissolved in dimethyl sulphoxide (4 ml), and tetrakis(triphenylphosphine)palladium(0) (126 mg, 110 μmol), sodium carbonate (174 mg, 1.64 mmol) and water (0.82 ml, 46 mmol) were added. The reaction mixture was stirred at 110° C. in a microwave (Biotage Initiator) for 450 min, cooled, filtered and, in two portions, purified chromatographically via HPLC (Method 11). This gave 199 mg (54% of theory) of the title compound.

LC-MS (Method 5): Rₜ=0.62 min; MS (ESIpos): m/z=682.5 [M+H]⁺.

### Example 15A

### 3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide

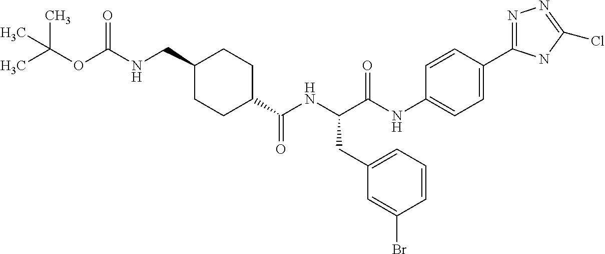

3-Bromo-N-[(trans-4-{[(tert-butoxycarbonyl)amino]methyl}cyclohexyl)carbonyl]-L-phenylalanine (2.5 g, 5.2 mmol), 4-(3-chloro-4H-1,2,4-triazol-5-yl)aniline (50%, 2.2 g, 5.7 mmol) and triethylamine (1.8 ml, 13 mmol) were suspended in 50 ml of ethyl acetate, and 2,4,6-tripropyl-1,3,5,2,4,6-trioxatriphosphinane 2,4,6-trioxide (50% in ethyl acetate, 6 ml, 10.4 mmol) was added. The mixture was then heated under reflux for 2 h and stirred at RT for a further 48 h. Water was added to the reaction mixture and the precipitated solid was filtered off with suction, washed with a little ethyl acetate and water and dried under high vacuum. This gave 2.16 g (63% of theory) of the title compound.

¹H NMR (300 MHz, DMSO-d₆): δ=ppm 0.68-0.88 (m, 2H), 0.99-1.27 (m, 4H), 1.33 (s, 9H), 1.47-1.69 (m, 4H), 1.99-2.13 (m, 1H), 2.66-2.74 (m, 2H), 2.81 (dd, 1H), 3.01 (dd, 1H), 4.56-4.68 (m, 1H), 6.70-6.79 (m, 1H), 7.22 (d, 1H), 7.25 (s, 1H), 7.33-7.40 (m, 1H), 7.49 (s, 1H), 7.70 (d, 2H), 7.86 (d, 2H), 8.12 (d, 1H), 10.36 (s, 1H).

LC-MS (Method 4): Rₜ=1.28 min; MS (ESIpos): m/z=661 [M+H]⁺.

### Example 16A

### N-alpha-[(trans-4-{[(tert-Butoxycarbonyl)amino]methyl}cyclohexyl)carbonyl]-N-[4-(3-chloro-4H-1,2,4-triazol-5-yl)phenyl]-3-(4,4,5,5-tetramethyl-1,3,2-dioxaborolan-2-yl)-L-phenylalaninamide

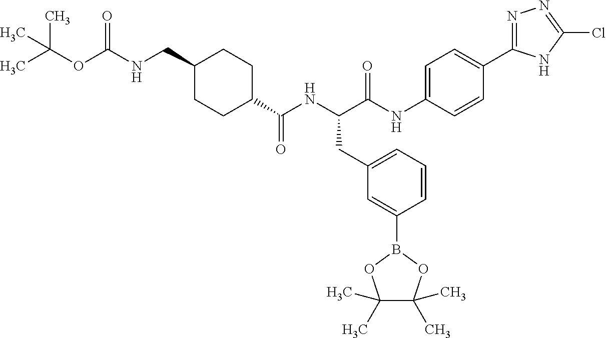

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (360 mg, 0.54 mmol) and bis(pinacolato)diborane (208 mg, 0.82 mmol) were dissolved in dimethyl sulphoxide (4 ml), the mixture was flushed with argon and 1,1′-bis(diphenylphosphine)ferrocenedichloropalladium(II) (22.3 mg, 27.3 μmol) and potassium acetate (160.6 mg, 1.6 mmol) were added. The reaction mixture was stirred in a microwave (Biotage Initiator) at 110° C. for 6 h. More bis(pinacolacto)diborane (69 mg, 0.27 mmol) and 1,1′-bis(diphenylphosphine)ferrocenedichloropalladium(II) (9 mg, 10.9 μmol) were then added, and the mixture was stirred at 110° C. for a further 6 h. The reaction mixture was converted further as the crude product.

LC-MS (Method 4): Rₜ=1.38 min; MS (ESIpos): m/z=707.5 [M+H]⁺.

### Example 17A

### 3-Bromo-N-alpha-[(trans-4-{[(tert-butoxycarbonyl)amino]methyl}cyclohexyl)carbonyl]-N-{4-[3-(trifluoromethyl)-4H-1,2,4-triazol-5-yl]phenyl}-L-phenylalaninamide

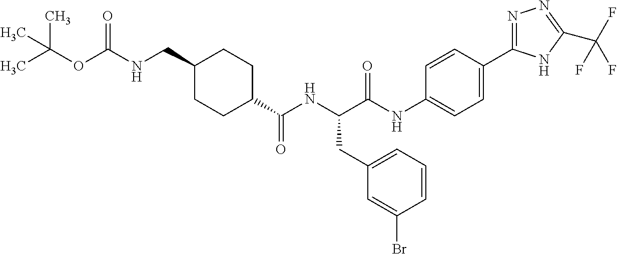

3-Bromo-N-[(trans-4-{[(tert-butoxycarbonyl)amino]methyl}cyclohexyl)carbonyl]-L-phenylalanine (2.6 g, 5.4 mmol), 2-(4-aminophenyl)-5-trifluoromethyl-1,3,4-triazole (1.45 g, 5.4 mmol) and diisopropylethylamine (3.8 ml, 22 mmol) were suspended in 50 ml of ethyl acetate, and 2,4,6-tripropyl-1,3,5,2,4,6-trioxatriphosphinane 2,4,6-trioxide (50% in ethyl acetate, 8 ml, 13.6 mmol) was added. The mixture was then heated under reflux for 6 h. Water was added to the reaction mixture, and the reaction mixture was extracted three times with ethyl acetate. The combined organic phases were washed with saturated aqueous ammonium chloride solution and saturated aqueous sodium chloride solution, dried over sodium sulphate, filtered and concentrated. This gave 3.8 g (100% of theory) of the title compound.

¹H NMR (300 MHz, DMSO-d₆): δ=ppm 0.79 (m, 2H), 1.19 (m, 2H), 1.33 (s, 9H), 1.50-1.67 (m, 4H), 1.99-2.12 (m, 1H), 2.71 (m, 2H), 2.83 (d, 1H), 2.99 (dd, 1H), 4.57-4.68 (m, 1H), 6.70-6.78 (m, 1H), 7.17-7.24 (m, 1H), 7.24-7.29 (m, 1H), 7.36 (d, 1H), 7.50 (s, 1H), 7.74 (d, 2H), 7.95 (d, 2H), 8.13 (d, 1H), 10.40 (s, 1H).

LC-MS (Method 4): Rₜ=1.35 min; MS (ESIpos): m/z=693.3 [M+H]⁺.

### Example 18A

### 3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide

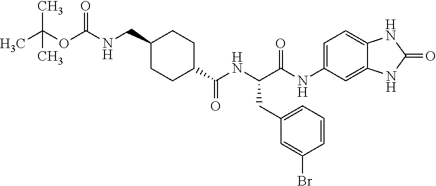

3-Bromo-N-[(trans-4-{[(tert-butoxycarbonyl)amino]methyl}cyclohexyl)carbonyl]-L-phenylalanine (5 g, 10.4 mmol), 5-amino-1,3-dihydro-2H-benzimidazol-2-one (1.6 g, 10.4 mmol) and N,N-diisopropylethylamine (5.4 ml, 31 mmol) were suspended in 175 ml of ethyl acetate, and 2,4,6-tripropyl-1,3,5,2,4,6-trioxatriphosphinane 2,4,6-trioxide (50% in ethyl acetate, 15 ml, 25.6 mmol) was added. The reaction mixture was then heated under reflux for 2 h and stirred at RT for a further 48 h. Water was added to the reaction mixture and the precipitated solid was filtered off with suction, washed with a little ethyl acetate and water and dried under high vacuum. This gave 2.1 g (33% of theory) of the title compound.

¹H NMR (400 MHz, DMSO-d₆): δ=ppm 0.71-0.85 (m, 2H), 1.17-1.25 (m, 4H), 1.33 (s, 9H), 1.47-1.56 (m, 1H), 1.57-1.67 (m, 3H), 2.00-2.11 (m, 1H), 2.68-2.73 (m, 2H), 2.80 (dd, 1H), 2.98 (dd, 1H), 4.53-4.62 (m, 1H), 6.68-6.75 (m, 1H), 6.81 (d, 1H), 6.96 (dd, 1H), 7.20 (d, 1H), 7.25 (d, 1H), 7.34 (d, 1H), 7.38 (d, 1H), 7.47 (s, 1H), 8.01 (d, 1H), 9.90 (s, 1H), 10.40-10.46 (m, 1H), 10.50 (s, 1H).

LC-MS (Method 4): Rₜ=1.20 min; MS (ESIpos): m/z=612 [M+H]⁺.

### Example 19A

### 3-Bromo-N-alpha-[(trans-4-{[(tert-butoxycarbonyl)amino]methyl}cyclohexyl)carbonyl]-N-(3-chloro-1H-indazol-6-yl)-L-phenylalaninamide

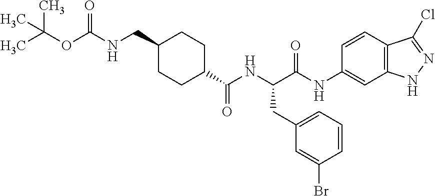

3-Bromo-N-[(trans-4-{[(tert-butoxycarbonyl)amino]methyl}cyclohexyl)carbonyl]-L-phenylalanine (1 g, 2.1 mmol), 3-chloro-1H-indazole-6-amine (368 mg, 2.1 mmol) and N,N-diisopropylethylamine (1.1 ml, 6.4 mmol) were suspended in 15 ml of ethyl acetate, and 2,4,6-tripropyl-1,3,5,2,4,6-trioxatriphosphinane 2,4,6-trioxide (50% in ethyl acetate, 3.2 ml, 6.4 mmol) was added. The reaction mixture was then heated under reflux for 2 h and stirred at RT for a further 48 h. Water was added to the reaction mixture, the phases were separated and the aqueous phase was extracted three times with ethyl acetate. The combined organic phases were washed with saturated aqueous ammonium chloride solution, dried over sodium sulphate, filtered and concentrated. This gave 1.4 g (93% of theory, 90% pure) of the title compound.

¹H NMR (300 MHz, DMSO-d₆): δ=ppm 0.70-0.93 (m, 2H), 1.17-1.25 (m, 4H), 1.30-1.37 (m, 9H), 1.63 (m, 3H), 1.99-2.11 (m, 1H), 2.69 (m, 2H), 2.77-2.86 (m, 1H), 2.97-3.06 (m, 1H), 4.55-4.70 (m, 1H), 6.71-6.79 (m, 1H), 7.18 (m, 2H), 7.21 (d, 1H), 7.27 (d, 1H), 7.36 (d, 2H), 7.50 (s, 1H), 7.54 (d, 1H), 8.09-8.17 (m, 2H), 10.37 (s, 1H), 13.09 (s, 1H).

LC-MS (Method 4): Rₜ=1.33 min; MS (ESIpos): m/z=634 [M+H]⁺.

### Example 20A

### 3-Bromo-N-alpha-[(trans-4-{[(tert-butoxycarbonyl)amino]methyl}cyclohexyl)carbonyl]-N-[2-(pentafluoroethyl)-1H-benzimidazol-6-yl]-L-phenylalaninamide

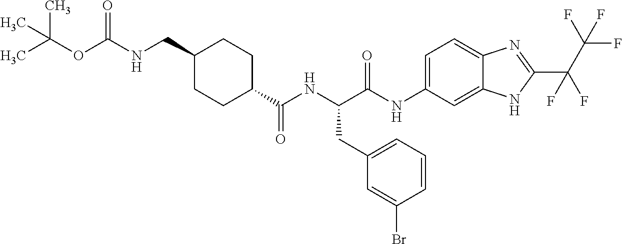 [SMILES: CC(C)(C)OC(=O)NC[C@H]1CC[C@H](C(=O)N[C@@H](Cc2cccc(Br)c2)C(=O)Nc2ccc3nc(C(F)(F)C(F)(F)F)[nH]c3c2)CC1]

3-Bromo-N-[(trans-4-{[(tert-butoxycarbonyl)amino]methyl}cyclohexyl)carbonyl]-L-phenylalanine (5.25 g, 2.1 mmol), 2-(pentafluoroethyl)-1H-benzimidazole-6-amine (3.0 g, 12 mmol) and N,N-diisopropylethylamine (4.5 ml, 32.6 mmol) were suspended in 145 ml of ethyl acetate, and 2,4,6-tripropyl-1,3,5,2,4,6-trioxatriphosphinane 2,4,6-trioxide (50% in ethyl acetate, 19.2 ml, 32.6 mmol) was added. The reaction mixture was then heated under reflux for 4 h and cooled. Water was added to the reaction mixture, the phases were separated and the aqueous phase was extracted three times with ethyl acetate. The combined organic phases were washed with saturated aqueous ammonium chloride solution, dried over sodium sulphate, filtered and concentrated. This gave 8.5 g (quant.) of the title compound.

LC-MS (Method 4): Rₜ=1.33 min; MS (ESIpos): m/z=718.2 [M+H]⁺.

### Example 21A

### 3′-[(2S)-2-{[(trans-4-{[(tert-Butoxycarbonyl)amino]methyl}cyclohexyl)carbonyl]amino}-3-oxo-3-{[2-(pentafluoroethyl)-1H-benzimidazol-6-yl]amino}propyl]-2-methylbiphenyl-4-carboxylic acid

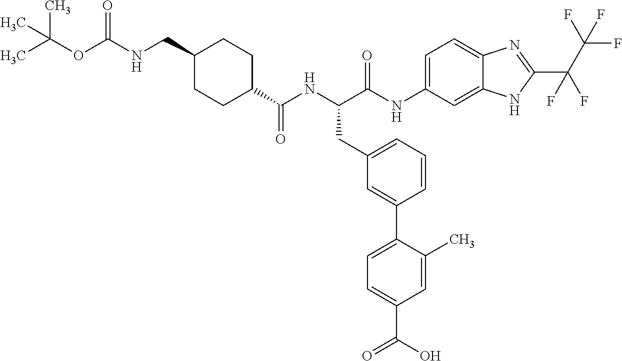

3-Bromo-N-alpha-[(trans-4-{[(tert-butoxycarbonyl)amino]methyl}cyclohexyl)carbonyl]-N-[2-(pentafluoroethyl)-1H-benzimidazol-6-yl]-L-phenylalaninamide (500 mg, 0.7 mmol) and 4-(4,4,5,5-tetramethyl-1,3,2-dioxaborolan-2-yl)benzoic acid (274 mg, 1.0 mmol) were dissolved in dimethyl sulphoxide (5 ml), and tetrakis(triphenylphosphine)palladium(0) (81 mg, 70 μmol), sodium carbonate (222 mg, 2.1 mmol) and water (1.05 ml, 58 mmol) were added. The reaction mixture was stirred at 110° C. in a microwave (Biotage Initiator) for 2 h, cooled, filtered and, in two portions, purified chromatographically via HPLC (Method 9). This gave 230 mg (43% of theory) of the title compound.

LC-MS (Method 4): Rₜ=1.25 min; MS (ESIpos): m/z=772.4 [M+H]⁺.

### Example 22A

### 3-Bromo-N-alpha-[(trans-4-{[(tert-butoxycarbonyl)amino]methyl}cyclohexyl)carbonyl]-N-[4-(5-oxo-4,5-dihydro-1H-1,2,4-triazol-3-yl)phenyl]-L-phenylalaninamide

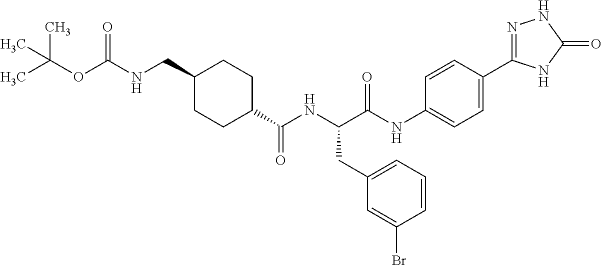

3-Bromo-N-[(trans-4-{[(tert-butoxycarbonyl)amino]methyl}cyclohexyl)carbonyl]-L-phenylalanine (2 g, 4.2 mmol), 5-(4-aminophenyl)-2,4-dihydro-3H-1,2,4-triazol-3-one (766 mg, 4.2 mmol) and N,N-diisopropylethylamine (2.2 ml, 12.6 mmol) were suspended in 100 ml of ethyl acetate, and 2,4,6-tripropyl-1,3,5,2,4,6-trioxatriphosphinane 2,4,6-trioxide (50% in ethyl acetate, 6.2 ml, 10.5 mmol) was added. The reaction mixture was then heated under reflux for 3 h and stirred at RT for a further 48 h. Water was added to the reaction mixture and the precipitated solid was filtered off with suction, washed with a little ethyl acetate and water and dried under high vacuum. This gave 2.7 g (100% of theory) of the title compound.

¹H NMR (400 MHz, DMSO-d₆): δ=ppm 0.72-0.85 (m, 2H), 1.01-1.11 (m, 1H), 1.16-1.26 (m, 2H), 1.33 (s, 9H), 1.49-1.57 (m, 1H), 1.63 (m, 3H), 2.01-2.10 (m, 1H), 2.68-2.74 (m, 2H), 2.81 (dd, 1H), 3.00 (dd, 1H), 4.57-4.66 (m, 1H), 6.69-6.74 (m, 1H), 7.21 (m, 1H), 7.25 (d, 1 H), 7.33-7.38 (m, 1H), 7.48 (br. s, 1H), 7.64 (d, 2H), 7.69 (d, 2H), 8.08 (d, 1H), 10.27 (s, 1H), 11.53 (s, 1H), 11.86 (br. s, 1H).

LC-MS (Method 4): Rₜ=1.22 min; MS (ESIpos): m/z=642 [M+H]⁺.

### Example 23A

### 3-Bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide

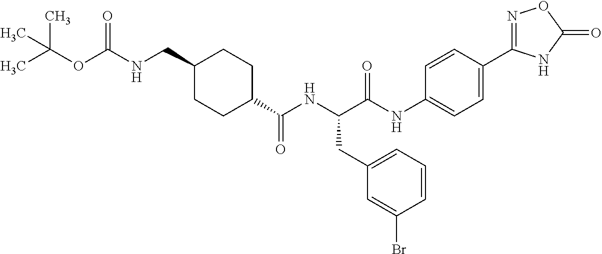

3-Bromo-N-[(trans-4-{[(tert-butoxycarbonyl)amino]methyl}cyclohexyl)carbonyl]-L-phenylalanine (10 g, 20.7 mmol), 3-(4-aminophenyl)-1,2,4-oxadiazol-5(4H)-one (3.8 g, 20.7 mmol) and N,N-diisopropylethylamine (11 ml, 62 mmol) were suspended in 375 ml of ethyl acetate, and 2,4,6-tripropyl-1,3,5,2,4,6-trioxatriphosphinane 2,4,6-trioxide (50% in ethyl acetate, 30.5 ml, 51.7 mmol) was added. The reaction mixture was then heated under reflux for 1 h, water was then added and the precipitated solid was filtered off with suction, washed with a little ethyl acetate and water and dried under high vacuum. This gave 6.3 g (47% of theory) of the title compound.

¹H NMR (300 MHz, DMSO-d₆): δ=ppm 0.78 (m, 2H), 0.99-1.25 (m, 3H), 1.33 (s, 9H), 1.62 (m, 4H), 2.05 (m, 1H), 2.70 (m, 2H), 2.82 (dd, 1H), 2.99 (dd, 1H), 4.55-4.67 (m, 1H), 6.75 (s, 1H), 7.22 (m, 2H), 7.36 (d, 1H), 7.49 (s, 1H), 7.73 (s, 4H), 8.14 (d, 1H), 10.45 (s, 1H).

LC-MS (Method 4): Rₜ=1.29 min; MS (ESIpos): m/z=644 [M+H]⁺.

### Example 24A

### Methyl (2Z)-3-(3-bromo-4-methoxyphenyl)-2-[(tert-butoxycarbonyl)amino]acrylate

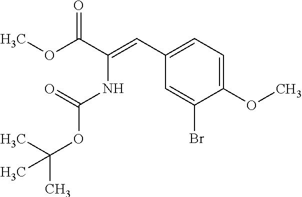

Under an argon atmosphere, methyl [(tert-butoxycarbonyl)amino](dimethoxyphosphoryl)acetate (7129 mg, 23 mmol) was initially charged in dichloromethane (150 ml), 1,8-diazabicyclo[5.4.0]undec-7-ene (7129 mg, 25 mmol) was added and the mixture was stirred at RT for 10 min. A solution of 3-bromo-4-methoxybenzaldehyde (5000 mg, 23 mmol) in dichloromethane (40 ml) was added and stirred at RT for 90 min. Ethyl acetate was added to the reaction mixture and the solution was adjusted to pH 4 using 1N hydrochloric acid solution. The mixture was washed with saturated aqueous sodium chloride solution. The organic phase was dried with sodium sulphate and the solvent was removed. The crude product was applied to silica gel and purified chromatographically (silica gel, cyclohexane/ethyl acetate 7.5:1, then 5:1, then 3:1), and the solvent was removed. This gave 7300 mg (83% of theory) of the title compound.

¹H NMR (400 MHz, DMSO-d₆): δ=ppm 1.43 (br. s., 9H), 3.72 (s, 3H), 3.88 (s, 3H), 7.17 (d, 2H), 7.66 (d, 1H), 7.96 (d, 1H), 8.67 (br. s, 1H).

LC-MS (Method 1): Rₜ=1.11 min; MS (ESIneg): m/z=384 [M−H]⁻.

### Example 25A

### Methyl 3-bromo-N-(tert-butoxycarbonyl)-O-methyl-L-tyrosinate

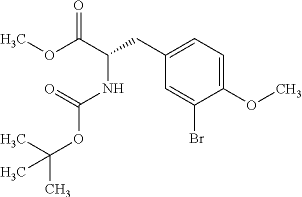

Methyl (2Z)-3-(3-bromo-4-methoxyphenyl)-2-[(tert-butoxycarbonyl)amino]acrylate (7292 mg, 19 mmol) was initially charged in ethanol (400 ml), the mixture was degassed, (+)-1,2-bis((2S,5S)-2,5-diethylphospholano)benzene(1,5-cyclooctadiene)rhodium(I) tetrafluoroborate (295 mg, 0.4 mmol) was added and the mixture was stirred under a hydrogen atmosphere (3 bar) at RT for 3 days. The reaction mixture was filtered through kieselguhr, washed with ethanol and dried under high vacuum. This gave 8045 mg (100% of theory) of the title compound.

α−D=+0.035°(23° C.,c=0.44 g/100 ml)

¹H NMR (400 MHz, DMSO-d₆): δ=ppm 1.32 (s, 9H), 2.72-2.83 (m, 1H), 2.90-2.98 (m, 1H), 3.62 (s, 3H), 3.81 (s, 3H), 4.08-4.18 (m, 1H), 7.03 (d, 1H), 7.22 (dd, 1H), 7.30 (d, 1H), 7.44 (d, 1H).

LC-MS (Method 1): Rₜ=1.13 min; MS (ESIpos): m/z=389 [M+H]⁺.

### Example 26A

### 3-Bromo-N-(tert-butoxycarbonyl)-O-methyl-L-tyrosine

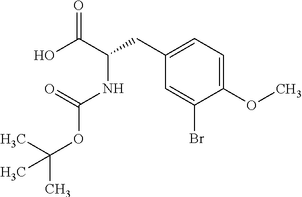 [SMILES: COc1ccc(C[C@H](NC(=O)OC(C)(C)C)C(=O)O)cc1Br]

A solution of lithium hydroxide monohydrate (4322 mg, 103 mmol) in water (60 ml) was added to a solution of methyl 3-bromo-N-(tert-butoxycarbonyl)-O-methyl-L-tyrosinate (4000 mg, 10 mmol) in tetrahydrofuran (180 ml). The suspension was stirred at RT for 16 h. The reaction mixture was acidified with 1N hydrochloric acid solution, and ethyl acetate was added. The organic phase was washed three times with water and once with saturated aqueous sodium chloride solution and dried over sodium sulphate, and the solvent was removed. This gave 3653 mg (95% of theory) of the title compound.

α−D=+0.088°(19° C.,c=0.49 g/100 ml)

¹H NMR (400 MHz, DMSO-d₆): δ=ppm 1.32 (s, 9H), 2.69-2.78 (m, 1H), 2.89-2.99 (m, 1H), 3.81 (s, 3H), 3.96-4.07 (m, 1H), 7.03 (d, 1H), 7.11 (d, 1H), 7.22 (dd, 1H), 7.45 (d, 1H), 12.53 (br. s, 1H).

LC-MS (Method 1): Rₜ=0.98 min; MS (ESIneg): m/z=372 [M−H]⁻.

### Example 27A

### 3-Bromo-N-alpha-(tert-butoxycarbonyl)-O-methyl-N-[4-(2H-tetrazol-5-yl)phenyl]-L-tyrosinamide

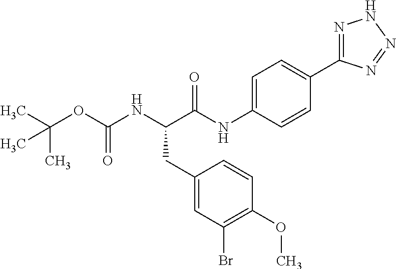 [SMILES: COc1ccc(C[C@H](NC(=O)OC(C)(C)C)C(=O)Nc2ccc(-c3nn[nH]n3)cc2)cc1Br]

N,N-Diisopropylethylamine (2.3 ml, 13 mmol) was added to a solution of 3-bromo-N-(tert-butoxycarbonyl)-O-methyl-L-tyrosine (2000 mg, 5 mmol) and 4-(2H-tetrazol-5-yl)aniline (1034 mg, 6.4 mmol) in ethyl acetate (40 ml). A 2,4,6-tripropyl-1,3,5,2,4,6-trioxatriphosphinane 2,4,6-trioxide solution (50% in DMF, 4081 mg, 6.4 mmol) and, until dissolution, DMF were added to the suspension, and the mixture was then stirred at RT for 16 h. The reaction mixture was stirred into ethyl acetate, and washed three times with water and once with saturated aqueous sodium chloride solution. The organic phase was dried with sodium sulphate and the solvent was removed. The crude product was stirred with acetonitrile and filtered off with suction. This gave 1820 mg (62% of theory, 93% pure) of the title compound.

¹H NMR (400 MHz, DMSO-d₆): δ=ppm 1.33 (s, 9H), 2.64-3.02 (m, 2H), 3.81 (s, 3H), 4.23-4.36 (m, 1H), 7.05 (d, 1H), 7.16-7.24 (d, 1H), 7.28-7.39 (dd, 1H), 7.58 (dd, 1H), 7.82 (d, 2H), 8.00 (d, 2H), 10.39 (s, 1H), 16.68 (br. s, 1H).

LC-MS (Method 1): Rₜ=1.01 min; MS (ESIpos): m/z=517 [M+H]⁺.

### Example 28A

### 3-Bromo-O-methyl-N-[4-(2H-tetrazol-5-yl)phenyl]-L-tyrosinamide hydrochloride

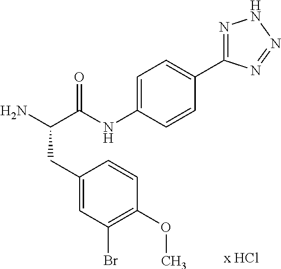 [SMILES: COc1ccc(C[C@H](N)C(=O)Nc2ccc(-c3nn[nH]n3)cc2)cc1Br]

13 ml (52 mmol) of a hydrochloride solution (4M in 1,4-dioxane) were added to a solution of 1805 mg (3.4 mmol) of 3-bromo-N-alpha-(tert-butoxycarbonyl)-O-methyl-N-[4-(2H-tetrazol-5-yl)phenyl]-L-tyrosinamide in 54 ml of 1,4-dioxane, and the mixture was stirred at RT for 20 h. The residue was filtered off, washed with acetonitrile and dried under high vacuum. This gave 1634 mg (99% of theory) of the title compound.

α−D=+0.555°(26° C.,c=0.49 g/100 ml)

¹H NMR (400 MHz, DMSO-d₆): δ=ppm 3.04 (dd, 1H), 3.23 (dd, 1H), 3.82 (s, 3H), 4.24-4.36 (m, 1H), 7.08 (d, 1H), 7.31 (dd, 1H), 7.59 (d, 1H), 7.84 (d, 2H), 8.08 (d, 2H), 8.38 (br. s, 3H), 11.30 (s, 1H).

LC-MS (Method 1): Rₜ=0.62 min; MS (ESIneg): m/z=415 [M−H−HCl]⁻.

### Example 29A

### 3-Bromo-N-alpha-[(trans-4-{[(tert-butoxycarbonyl)amino]methyl}cyclohexyl)carbonyl]-O-methyl-N-[4-(2H-tetrazol-5-yl)phenyl]-L-tyrosinamide

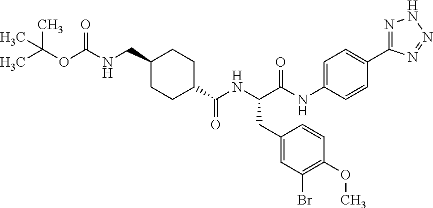

N,N-Diisopropylethylamine (1.9 ml, 11 mmol) was added to a solution of 3-bromo-O-methyl-N-[4-(2H-tetrazol-5-yl)phenyl]-L-tyrosinamide hydrochloride (1400 mg, 3 mmol) and trans-4-{[(tert-butoxycarbonyl)amino]methyl}cyclohexanecarboxylic acid (949 mg, 3.7 mmol) in ethyl acetate (24 ml). A 2,4,6-tripropyl-1,3,5,2,4,6-trioxatriphosphinane 2,4,6-trioxide solution (50% in DMF, 2356 mg, 3.7 mmol) was added dropwise to the suspension, and the suspension was then stirred at RT for 16 h. The reaction mixture was stirred into ethyl acetate, and washed three times with water and once with saturated aqueous sodium chloride solution. The organic phase was dried with sodium sulphate and the solvent was removed. The crude product was stirred with acetonitrile and filtered off with suction. This gave 1370 mg (67% of theory) of the title compound.

¹H NMR (400 MHz, DMSO-d₆): δ=ppm 0.75-0.91 (m, 2H), 1.04-1.29 (m, 3H), 1.37 (s, 9H), 1.53-1.72 (m, 4H), 2.04-2.17 (m, 1H), 2.75 (m, 3H), 2.93-3.03 (m, 1H), 3.80 (s, 3H), 4.50-4.67 (m, 1H), 6.73-6.84 (m, 1H), 7.03 (d, 1H), 7.27 (dd, 1H), 7.54 (d, 1H), 7.81 (d, 2H), 8.00 (d, 2H), 8.14 (d, 1H), 10.45 (s, 1H), 16.73 (br. s, 1H).

LC-MS (Method 1): Rₜ=1.05 min; MS (ESIneg): m/z=654 [M−H]⁻.

### Example 30A

### Methyl 5′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-carboxylate

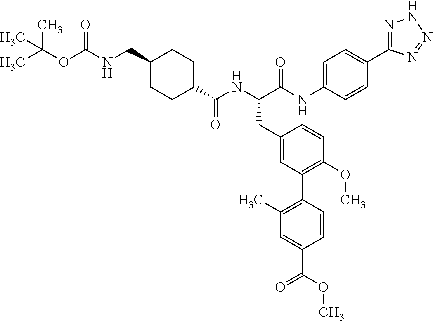

400 mg (0.61 mmol) of 3-bromo-N-alpha-[(trans-4-{[(tert-butoxycarbonyl)amino]methyl}cyclohexyl)carbonyl]-O-methyl-N-[4-(2H-tetrazol-5-yl)phenyl]-L-tyrosinamide, 70 mg (0.06 mmol) of tetrakis(triphenyl-phosphine)palladium(0) and 336 mg (1.22 mmol) of methyl 3-methyl-4-(4,4,5,5-tetramethyl-1,3,2-dioxaborolan-2-yl)benzoate were taken up in 6 ml of 1,2-dimethoxyethane and 2 ml of ethanol. After the addition of 2 ml of 2N aqueous sodium carbonate solution, the reaction mixture was stirred at reflux for 3 h, the salts were then filtered off and the filtrate was separated by preparative HPLC (mobile phase: acetonitrile/water gradient, 0.1% trifluoroacetic acid). The product-containing fractions were neutralized with N,N-diisopropylethylamine and the mixture was concentrated to dryness. This gave 1.06 g (quant., 50% pure) of the title compound.

LC-MS (Method 1): Rₜ=1.12 min; MS (ESIpos): m/z=726 [M+H]⁺.

### Example 31A

### 5′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-carboxylic acid

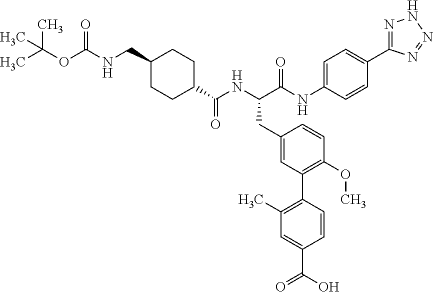

910 mg of the contaminated methyl 5′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2′-methoxy-2-methylbiphenyl-4-carboxylate were taken up in 15 ml of THF and 5 ml of water, 526 mg (12.54 mmol) of lithium hydroxide monohydrate were added and the mixture was stirred at RT for 16 h. 50 ml of water and 1N hydrochloric acid to pH 4 were added to the reaction mixture. The mixture was extracted twice with dichloromethane. The combined organic phases were washed with saturated aqueous sodium chloride solution, dried over sodium sulphate, filtered and concentrated to dryness. This gave 289 mg (76% of theory) of the title compound over two stages.

¹H NMR (400 MHz, DMSO-d₆): δ=ppm 0.71-0.93 (m, 2H), 1.05-1.31 (m, 5H), 1.37 (s, 9H), 1.46-1.57 (m, 1H), 1.58-1.72 (m, 3H), 1.94-2.16 (m, 4H), 2.69-2.78 (m, 2H), 2.80-2.90 (m, 1H), 2.96-3.07 (m, 1H), 3.67 (s, 3H), 4.59-4.73 (m, 1H), 6.72-6.83 (m, 1H), 6.96-7.23 (m, 3H), 7.26-7.37 (m, 1H), 7.67-7.86 (m, 4H), 7.98 (d, 2H), 8.11 (d, 1H), 10.42 (s, 1H), 12.9 (br. s, 1H), 16.7 (br. s, 1H).

LC-MS (Method 1): Rₜ=1.00 min; MS (ESIpos): m/z=712 [M+H]⁺.

### Example 32A

### 3-Bromo-N-alpha-[(trans-4-{[(tert-butoxycarbonyl)amino]methyl}cyclohexyl)carbonyl]-N-1H-indazol-6-yl-L-phenylalaninamide

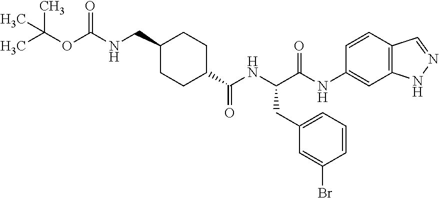

2.70 ml (15.52 mmol) of N,N-diisopropylethylamine and 7.1 ml (7.45 mmol) of 2,4,6-tripropyl-1,3,5,2,4,6-trioxatriphosphinane 2,4,6-trioxide solution (50% in DMF) were added to a solution of 3.00 g (6.21 mmol) of 3-bromo-N-[(trans-4-{[(tert-butoxycarbonyl)amino]methyl}-cyclohexyl)carbonyl]-L-phenylalanine and 0.99 g (7.45 mmol) of 6-aminoindazole in 75 ml of ethyl acetate. The suspension was stirred at RT for 16 h. 680 ml of ethyl acetate were added and the reaction mixture was washed three times with water. The organic phase was dried over sodium sulphate and filtered, and the solvent was removed. The residue was stirred with 10 ml of acetonitrile and 20 ml of diethyl ether. The solid was filtered off, washed with diethyl ether and then dried under high vacuum. This gave 2.51 g (66% of theory) of the title compound. On a rotary evaporator, the mother liquor was freed of the solvent, and the residue was separated by preparative HPLC (Method 16). This gave a further 0.25 g (6% of theory, 88% pure) of the title compound.

¹H NMR (400 MHz, DMSO-d₆): δ=ppm 0.74-0.91 (m, 2H), 1.04-1.31 (m, 3H), 1.37 (s, 9H), 1.51-1.61 (m, 1H), 1.61-1.73 (m, 3H), 2.04-2.16 (m, 1H), 2.75 (t, 2H), 2.81-2.91 (m, 1H), 3.00-3.10 (m, 1H), 3.29-3.41 (m, 2H), 4.63-4.73 (m, 1H), 6.74-6.83 (m, 1H), 7.10 (dd, 1H), 7.21-7.35 (m, 2H), 7.36-7.42 (m, 1H), 7.54 (s, 1H), 7.67 (d, 1H), 7.97 (s, 1H), 8.08-8.19 (m, 2H), 10.27 (s, 1H), 12.91 (s, 1H).

LC-MS (Method 1): Rₜ=1.09 min; MS (ESIpos): m/z=598 [M+H]⁺.

### Example 33A

### 3′-[(2S)-2-{[(trans-4-{[(tert-Butoxycarbonyl)amino]methyl}cyclohexyl)carbonyl]amino}-3-(1H-indazol-6-ylamino)-3-oxopropyl]biphenyl-4-carboxylic acid

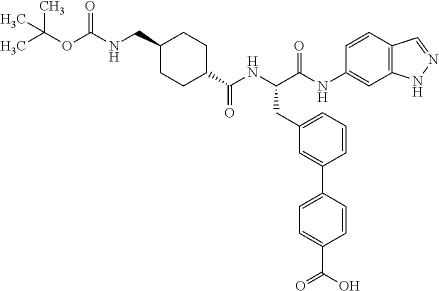

In each of two analogous reactions, 500 mg (0.84 mmol) of 3-bromo-N-alpha-[(trans-4-{[(tert-butoxycarbonyl)amino]methyl}cyclohexyl)carbonyl]-N-1H-indazol-6-yl-L-phenylalaninamide, 61 mg (0.08 mmol) of 1,1′-bis(diphenylphosphino)ferrocenepalladium(II) chloride and 376 mg (2.09 mmol) of 4-methoxycarbonylphenylboronic acid were taken up in 7.5 ml of 1,2-dimethoxyethane and 2.50 ml of ethanol. After the addition of 4 ml in each case of 2N aqueous sodium carbonate solution, the reaction mixtures were irradiated in a microwave at 120° C. for 1 h, then filtered through kieselguhr, and the filtrates were separated by preparative HPLC (mobile phase: acetonitrile/water gradient, 0.1% trifluoroacetic acid). The product-containing fractions were adjusted to pH 7 using 1 N aqueous sodium hydroxide solution, and the solvents were removed. This gave, in total, 705 mg of a mixture of the title compound and the methyl ester and 132 mg (23% of theory, 94% pure) of the title compound.

700 mg (1.07 mmol) of the mixture of the title compound and the methyl ester were taken up in 30 ml of THF and 10 ml of water, 449 mg (10.71 mmol) of lithium hydroxide monohydrate were added and the mixture was stirred at RT for 16 h and at 40° C. for 6 h. Dichloromethane and 1N hydrochloric acid to pH 4 were added to the reaction mixture, the phases were separated and the aqueous phase was extracted three times with dichloromethane. The precipitate that formed was re-dissolved with a little methanol and the aqueous phase was extracted four more times with dichloromethane. The combined organic phases were dried over sodium sulphate, filtered and concentrated. This gave 510 mg (48% of theory, 2 steps) of the title compound. The aqueous phase was extracted five more times with dichloromethane, and the combined organic phases were dried over sodium sulphate, filtered and concentrated. This gave a further 77 mg (5% of theory, 77% pure, 2 steps) of the title compound.

LC-MS (Method 1): Rₜ=1.01 min; MS (ESIneg): m/z=638 [M−H]⁻.

### Example 34A

### N-[(trans-4-{[(tert-Butoxycarbonyl)amino]methyl}cyclohexyl)carbonyl]-3-{6-[3-(dimethylamino)propoxy]pyridin-3-yl}-L-phenylalanine

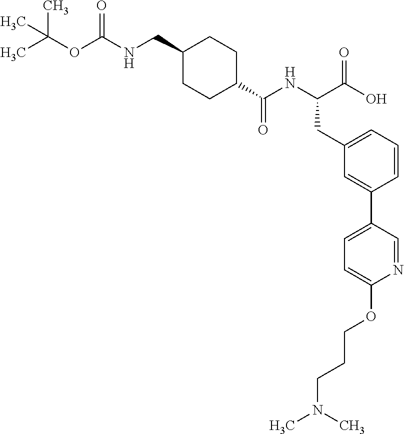

3-Bromo-N-[(trans-4-{[(tert-butoxycarbonyl)amino]methyl}cyclohexyl)carbonyl]-L-phenylalanine (500 mg, 1.0 mmol) and N,N-dimethyl-3-{[5-(4,4,5,5-tetramethyl-1,3,2-dioxaborolan-2-yl)pyridin-2-yl]oxy}propane-1-amine (475 mg, 1.5 mmol) were dissolved in dimethylformamide (8 ml), and tetrakis(triphenylphosphine)palladium(0) (120 mg, 103 μmol), sodium carbonate (329 mg, 3.1 mmol) and water (1.6 ml, 86 mmol) were added. The reaction mixture was stirred at 110° C. in a microwave (Biotage Initiator) for 120 min, cooled, filtered and, in three portions, purified chromatographically via HPLC (Method 8). This gave 381 mg (63% of theory) of the title compound.

¹H NMR (300 MHz, DMSO-d₆): δ=ppm 0.63-0.83 (m, 2H), 0.96-1.11 (m, 1H), 1.17 (m, 2H), 1.32 (s, 9H), 1.39-1.55 (m, 2H), 1.60 (m, 2H), 1.80-1.92 (m, 2H), 1.93-2.07 (m, 1H), 2.21 (s, 6H), 2.68 (t, 2H), 2.88 (dd, 1H), 3.08 (dd, 1H), 4.27 (t, 2H), 4.33-4.43 (m, 1H), 6.64-6.73 (m, 1H), 6.83 (d, 1H), 7.14 (d, 1H), 7.29 (t, 1H), 7.38-7.45 (m, 2H), 7.78 (d, 1H), 7.91 (dd, 1H), 8.37 (d, 1H).

LC-MS (Method 4): Rₜ=0.82 min; MS (ESIpos): m/z=584.4 [M+H]⁺.

### Example 35A

### tert-Butyl 4-{[(4-bromo-3-methylphenyl)sulphonyl]amino}piperidine-1-carboxylate

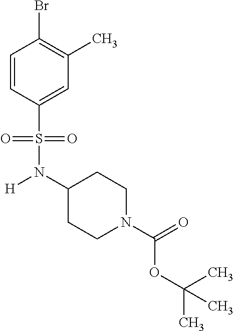 [SMILES: Cc1cc(S(=O)(=O)NC2CCN(C(=O)OC(C)(C)C)CC2)ccc1Br]

4-Bromo-3-methylbenzenesulphonyl chloride (1 g, 3.7 mmol) was dissolved in dichloromethane (20 ml), the mixture was cooled to 0° C. and tert-butyl 4-aminopiperidine-1-carboxylate (1.11 g, 5.6 mmol) and N,N-diisopropylethylamine (1.62 ml, 9.3 mmol) were added. The reaction mixture was stirred at RT overnight and concentrated, and the residue was purified chromatographically by HPLC (Method 9). This gave 1.2 g (74% of theory) of the title compound.

¹H NMR (300 MHz, DMSO-d₆): δ=ppm 1.09-1.23 (m, 2H), 1.32 (s, 9H), 1.50 (dd, 2H), 2.39 (s, 3H), 2.64-2.82 (m, 2H), 3.06-3.21 (m, 1H), 3.67 (d, 2H), 7.51 (dd, 1H), 7.72-7.84 (m, 3H).

LC-MS (Method 4): Rₜ=1.37 min; MS (ESIpos): m/z=435.2 [M+H]⁺.

### Example 36A

### tert-Butyl 4-{[(5-bromopyridin-3-yl) sulphonyl]amino}piperidine-1-carboxylate

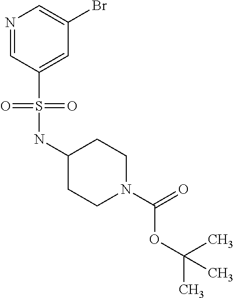 [SMILES: CC(C)(C)OC(=O)N1CCC(NS(=O)(=O)c2cncc(Br)c2)CC1]

5-Bromopyridine-3-sulphonyl chloride (1 g, 3.9 mmol) was dissolved in dichloromethane (20 ml), the mixture was cooled to 0° C. and tert-butyl 4-aminopiperidine-1-carboxylate (1.18 g, 5.8 mmol) and N,N-diisopropylethylamine (1.7 ml, 9.7 mmol) were added. The reaction mixture was stirred at RT overnight and concentrated, and the residue was purified chromatographically by HPLC (Method 9). This gave 1.0 g (62% of theory) of the title compound.

¹H NMR (300 MHz, DMSO-d₆): δ=ppm 1.09-1.24 (m, 2H), 1.33 (s, 9H), 1.47-1.59 (m, 2H), 2.66-2.83 (m, 2H), 3.19-3.25 (m, 1H), 3.63-3.75 (m, 2H), 8.09 (br. s, 1H), 8.36 (t, 1H), 8.91 (d, 1H), 8.95 (d, 1H).

LC-MS (Method 4): Rₜ=1.19 min; MS (ESIneg): m/z=420.3 [M−H]⁻.

### Example 37A

### tert-Butyl [(trans-4-{[(2S)-3-[5′-(benzylsulphamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate

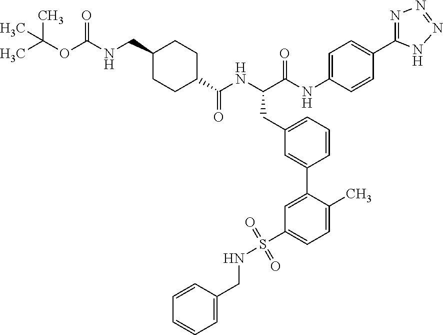

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [5-(benzylsulphamoyl)-2-methylphenyl]boronic acid (110 mg, 0.36 mmol) were dissolved in dimethyl sulphoxide (2 ml), and 1,1′-bis(diphenylphosphine)ferrocenedichloropalladium(II) (20 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 10). This gave 49 mg (25% of theory) of the title compound.

LC-MS (Method 4): Rt=1.31 min; MS (ESIpos): m/z=807.5 [M+H]⁺.

### Example 38A

### tert-Butyl [(trans-4-{[(2S)-3-[2′-methyl-5′-(pyrrolidin-1-ylsulphonyl)biphenyl-3-yl]-1-oxo-1-{[4-(1H-tetrazol-5-yl)phenyl]amino}propan-2-yl]carbamoyl}cyclohexyl)methyl]carbamate

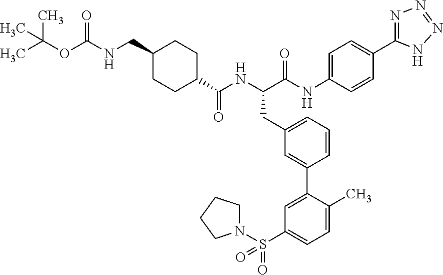

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 2-methyl-5-(pyrrolidin-1-ylsulphonyl)phenyl]boronic acid (99 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 10). This gave 92 mg (53% of theory) of the title compound.

LC-MS (Method 4): Rₜ=1.33 min; MS (ESIpos): m/z=771.5 [M+H]⁺.

### Example 39A

### tert-Butyl [(trans-4-{[(2S)-3-[2′-methyl-5′-(morpholin-4-ylsulphonyl)biphenyl-3-yl]-1-oxo-1-{[4-(1H-tetrazol-5-yl)phenyl]amino}propan-2-yl]carbamoyl}cyclohexyl)methyl]carbamate

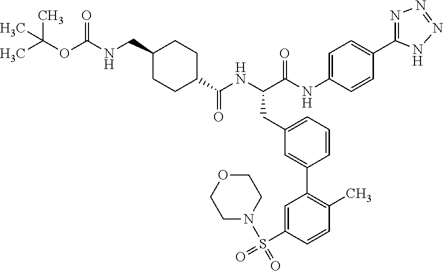

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [2-methyl-5-(morpholin-4-ylsulphonyl)phenyl]boronic acid (75 mg, 0.26 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 9). This gave 78 mg (42% of theory) of the title compound.

LC-MS (Method 4): Rₜ=1.26 min; MS (ESIpos): m/z=787.2 [M+H]⁺.

### Example 40A

### tert-Butyl [(trans-4-{[(2S)-3-[5′-(dimethylsulphamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate

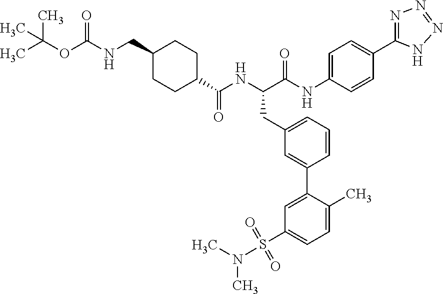

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [5-(dimethylsulphamoyl)-2-methylphenyl]boronic acid (87 mg, 0.36 mmol) were dissolved in dimethyl sulphoxide (2 ml), and 1,1′-bis(diphenylphosphine)ferrocenedichloropalladium(II) (19.5 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 53 mg (30% of theory) of the title compound.

LC-MS (Method 4): Rₜ=1.25 min; MS (ESIpos): m/z=745.5 [M+H]⁺.

### Example 41A

### tert-Butyl [(trans-4-{[(2S)-1-oxo-3-(3′-sulphamoylbiphenyl-3-yl)-1-{[4-(1H-tetrazol-5-yl)phenyl]-amino}propan-2-yl]carbamoyl}cyclohexyl)methyl]carbamate

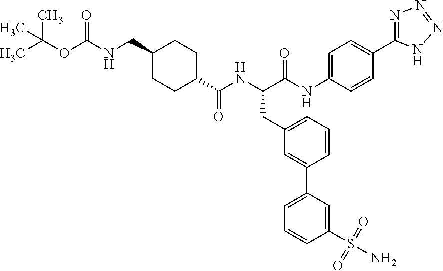

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 3-(4,4,5,5-tetramethyl-1,3,2-dioxaborolan-2-yl)benzenesulphonamide (104 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 33 mg (19% of theory) of the title compound.

LC-MS (Method 4): Rₜ=1.12 min; MS (ESIpos): m/z=703.3 [M+H]⁺.

### Example 42A

### tert-Butyl [(trans-4-{[(2S)-3-[3′-(cyclopropylsulphamoyl)biphenyl-3-yl]-1-oxo-1-{[4-(1H-tetrazol-5-yl)phenyl]amino}propan-2-yl]carbamoyl}cyclohexyl)methyl]carbamate

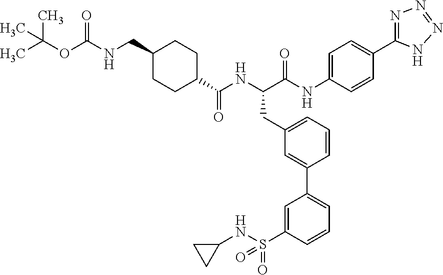

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [3-(cyclopropylsulphamoyl)phenyl]boronic acid (88 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 25 mg (18% of theory) of the title compound.

LC-MS (Method 4): Rₜ=1.23 min; MS (ESIpos): m/z=743.4 [M+H]⁺.

### Example 43A

### tert-Butyl [(trans-4-{[(2S)-3-[4′-(dimethylsulphamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate

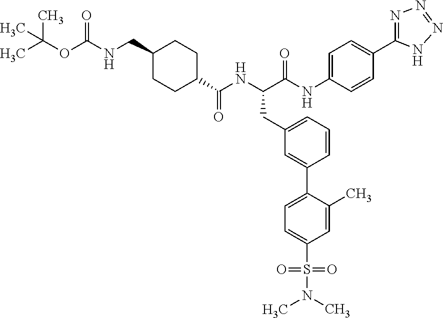

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [4-(dimethylsulphamoyl)-2-methylphenyl]boronic acid (87 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 67 mg (37% of theory) of the title compound.

LC-MS (Method 4): Rₜ=1.24 min; MS (ESIpos): m/z=745.0 [M+H]⁺.

### Example 44A

### tert-Butyl [(trans-4-{[(2S)-3-[4′-methoxy-3′-(morpholin-4-ylsulphonyl)biphenyl-3-yl]-1-oxo-1-{[4-(1H-tetrazol-5-yl)phenyl]amino}propan-2-yl]carbamoyl}cyclohexyl)methyl]carbamate

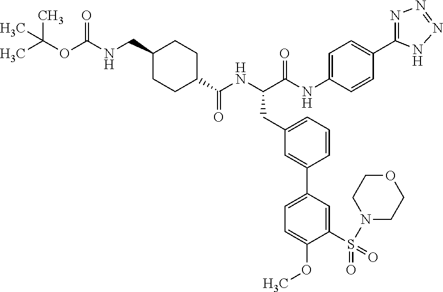

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [4-methoxy-3-(morpholin-4-ylsulphonyl)phenyl]boronic acid (110 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 80 mg (42% of theory) of the title compound.

LC-MS (Method 4): Rₜ=1.21 min; MS (ESIpos): m/z=803.5 [M+H]⁺.

### Example 45A

### tert-Butyl [(trans-4-{[(2S)-3-[2′-methyl-5′-(morpholin-4-ylsulphonyl)biphenyl-3-yl]-1-oxo-1-{[4-(5-oxo-4,5-dihydro-1H-1,2,4-triazol-3-yl)phenyl]amino}propan-2-yl]carbamoyl}cyclohexyl)methyl]carbamate

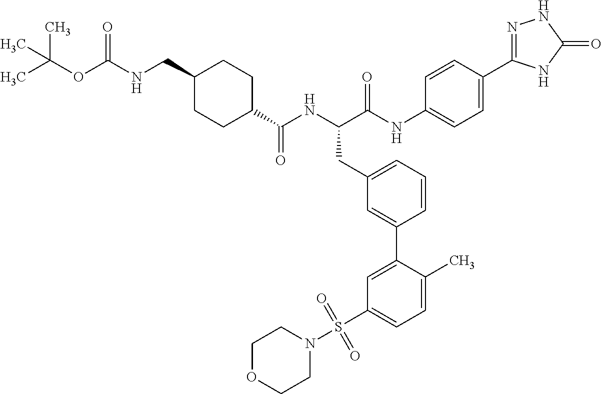

3-Bromo-N-alpha-[(trans-4-{[(tert-butoxycarbonyl)amino]methyl}cyclohexyl)carbonyl]-N-[4-(5-oxo-4,5-dihydro-1H-1,2,4-triazol-3-yl)phenyl]-L-phenylalaninamide (150 mg, 0.23 mmol) and [2-methyl-5-(morpholin-4-ylsulphonyl)phenyl]boronic acid (102 mg, 0.35 mmol) were dissolved in dimethyl sulphoxide (2 ml), and tetrakis(triphenylphosphine)palladium(0) (28 mg, 24 μmol), sodium carbonate (74 mg, 0.70 mmol) and water (0.35 ml, 19.5 mmol) were added. The reaction mixture was stirred at 110° C. in a microwave (Biotage Initiator) for 90 min, cooled, filtered and purified by chromatography via HPLC (Method 10). This gave 100 mg (53% of theory) of the title compound.

LC-MS (Method 4): Rₜ=1.22 min; MS (ESIpos): m/z=802.5 [M+H]⁺.

### Example 46A

### tert-Butyl {[trans-4-({(2S)-3-[2′-methyl-5′-(morpholin-4-ylsulphonyl)biphenyl-3-yl]-1-oxo-1-[(2-oxo-2,3-dihydro-1H-benzimidazol-5-yl)amino]propan-2-yl}carbamoyl)cyclohexyl]methyl}carbamate

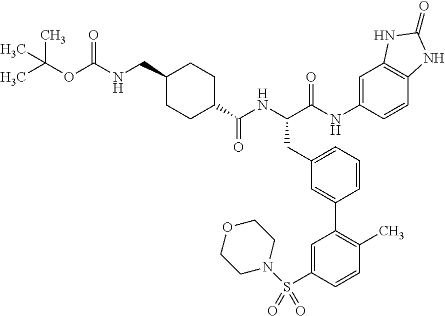

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and [2-methyl-5-(morpholin-4-ylsulphonyl)phenyl]boronic acid (104 mg, 0.36 mmol) were dissolved in dimethyl sulphoxide (2 ml), and tetrakis(triphenylphosphine)palladium(0) (28 mg, 24 μmol), sodium carbonate (77 mg, 0.73 mmol) and water (0.37 ml, 20 mmol) were added. The reaction mixture was stirred at 110° C. in a microwave (Biotage Initiator) for 90 min, cooled, filtered and purified by chromatography via HPLC (Method 10). This gave 108 mg (57% of theory) of the title compound.

LC-MS (Method 4): Rₜ=1.21 min; MS (ESIpos): m/z=775.4 [M+H]⁺.

### Example 47A

### tert-Butyl {[trans-4-({(2S)-3-[2′-methyl-5′-(pyrrolidin-1-ylsulphonyl)biphenyl-3-yl]-1-oxo-1-[(2-oxo-2,3-dihydro-1H-benzimidazol-5-yl)amino]propan-2-yl}carbamoyl)cyclohexyl]methyl}carbamate

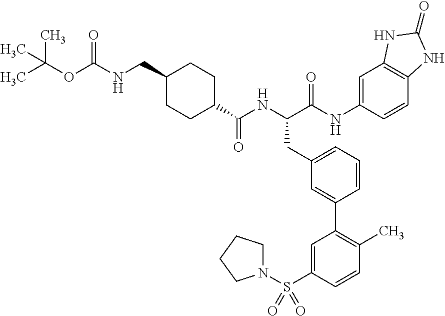

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and [2-methyl-5-(pyrrolidin-1-ylsulphonyl)phenyl]boronic acid (98 mg, 0.36 mmol) were dissolved in dimethyl sulphoxide (2 ml), and tetrakis(triphenylphosphine)palladium(0) (28 mg, 24 μmol), sodium carbonate (77 mg, 0.73 mmol) and water (0.37 ml, 20 mmol) were added. The reaction mixture was stirred at 110° C. in a microwave (Biotage Initiator) for 90 min, cooled, filtered and purified by chromatography via HPLC (Method 10). This gave 33 mg (18% of theory) of the title compound.

LC-MS (Method 4): Rₜ=1.23 min; MS (ESIpos): m/z=759.3 [M+H]⁺.

### Example 48A

### tert-Butyl {[trans-4-({(2S)-3-[5′-(benzylsulphamoyl)-2′-methylbiphenyl-3-yl]-1-oxo-1-[(2-oxo-2,3-dihydro-1H-benzimidazol-5-yl)amino]propan-2-yl}carbamoyl)cyclohexyl]methyl}carbamate

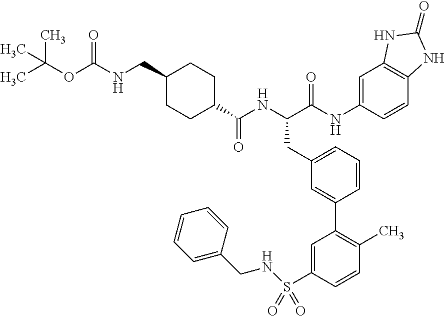

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and [5-(benzylsulphamoyl)-2-methylphenyl]boronic acid (112 mg, 0.36 mmol) were dissolved in dimethyl sulphoxide (2 ml), and tetrakis(triphenylphosphine)palladium(0) (28 mg, 24 mol), sodium carbonate (77 mg, 0.73 mmol) and water (0.37 ml, 20 mmol) were added. The reaction mixture was stirred at 110° C. in a microwave (Biotage Initiator) for 90 min, cooled, filtered and purified by chromatography via HPLC (Method 10). This gave 127 mg (65% of theory) of the title compound.

LC-MS (Method 4): Rₜ=1.27 min; MS (ESIpos): m/z=795.4 [M+H]⁺.

### Example 49A

### tert-Butyl [(trans-4-{[(2S)-3-[5′-(benzylsulphamoyl)-2′-methylbiphenyl-3-yl]-1-oxo-1-{[4-(5-oxo-4,5-dihydro-1H-1,2,4-triazol-3-yl)phenyl]amino}propan-2-yl]carbamoyl}cyclohexyl)methyl]carbamate

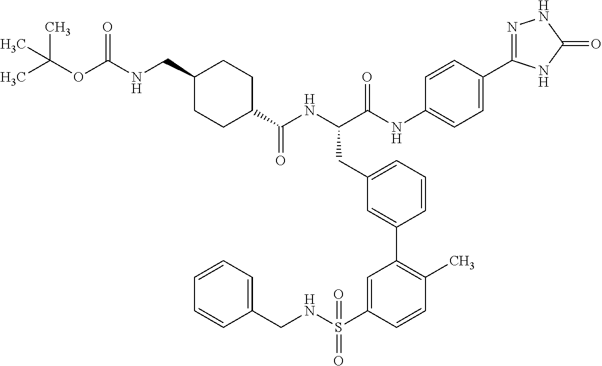

3-Bromo-N-alpha-[(trans-4-{[(tert-butoxycarbonyl)amino]methyl}cyclohexyl)carbonyl]-N-[4-(5-oxo-4,5-dihydro-1H-1,2,4-triazol-3-yl)phenyl]-L-phenylalaninamide (150 mg, 0.23 mmol) and 5-(N-benzylsulphamoyl)-2-methylphenylboronic acid (109 mg, 0.35 mmol) were dissolved in dimethyl sulphoxide (2 ml), and tetrakis(triphenylphosphine)palladium(0) (28 mg, 24 μmol), sodium carbonate (74 mg, 0.70 mmol) and water (0.35 ml, 19.5 mmol) were added. The reaction mixture was stirred at 110° C. in a microwave (Biotage Initiator) for 90 min, cooled, filtered and purified by chromatography via HPLC (Method 10). This gave 68 mg (35% of theory) of the title compound.

LC-MS (Method 4): Rₜ=1.35 min; MS (ESIpos): m/z=822.5 [M+H]⁺.

### Example 50A

### tert-Butyl 4-[({3′-[(2S)-2-{[(trans-4-{[(tert-butoxycarbonyl)amino]methyl}cyclohexyl)carbonyl]-amino}-3-oxo-3-{[4-(1H-tetrazol-5-yl)phenyl]amino}propyl]-6-methylbiphenyl-3-yl}carbonyl)amino]piperidine-1-carboxylate

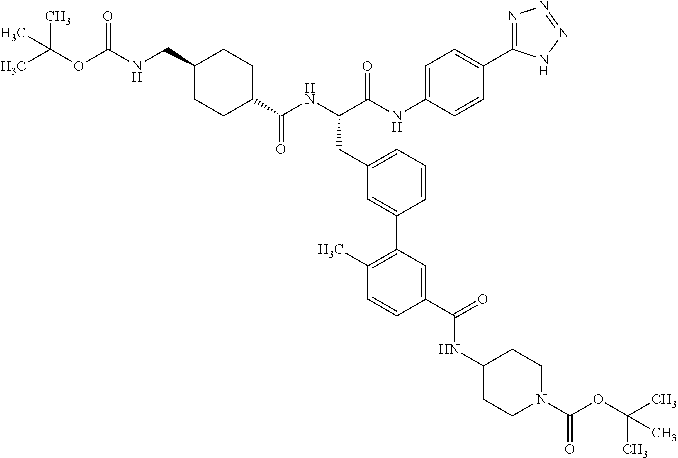

3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-6-methylbiphenyl-3-carboxylic acid (100 mg, 0.15 mmol) and tert-butyl 4-aminopiperidine-1-carboxylate (35 mg, 0.17 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.17 mmol) and N,N-diisopropylethylamine (23 mg, 0.17 mmol) were added and the mixture was stirred at RT overnight. Subsequently, the mixture was concentrated and the residue was purified chromatographically by HPLC (Method 10). This gave 23 mg (18% of theory) of the title compound.

LC-MS (Method 4): Rₜ=1.36 min; MS (ESIpos): m/z=864.7 [M+H]⁺.

### Example 51A

### tert-Butyl 4-[({3′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2-methylbiphenyl-4-yl}carbonyl)amino]piperidine-1-carboxylate

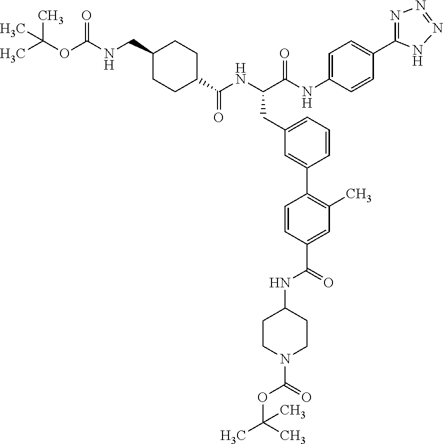

N-alpha-[(trans-4-{[(tert-Butoxycarbonyl)amino]methyl}cyclohexyl)carbonyl]-3-(4,4,5,5-tetramethyl-1,3,2-dioxaborolan-2-yl)-N-[4-(1H-tetrazol-5-yl)phenyl]-L-phenylalaninamide (179 mg, 0.26 mmol, crude product) and tert-butyl 4-[(4-bromo-3-methylbenzoyl)amino]piperidine-1-carboxylate (159 mg, 0.4 mmol) were dissolved in dimethyl sulphoxide (2 ml), and tetrakis(triphenylphosphine)palladium(0) (31 mg, 26 μmol), sodium carbonate (141 mg, 1.3 mmol) and water (0.4 ml, 22 mmol) were added. The reaction mixture was stirred at 110° C. in a microwave (Biotage Initiator) for 120 min, cooled, filtered and purified by chromatography via HPLC (Method 11). This gave 28 mg (12% of theory) of the title compound.

LC-MS (Method 5): Rₜ=0.94 min; MS (ESIpos): m/z=864.7 [M+H]⁺.

### Example 52A

### tert-Butyl 6-[({3′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-6-methylbiphenyl-3-yl}carbonyl)amino]-3-azabicyclo[3.1.0]hexane-3-carboxylate

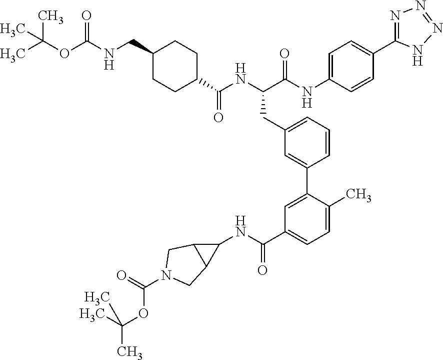

3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-6-methylbiphenyl-3-carboxylic acid (100 mg, 0.15 mmol) and tert-butyl 6-amino-3-azabicyclo[3.1.0]hexane-3-carboxylate (35 mg, 0.17 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.17 mmol) and N,N-diisopropylethylamine (23 mg, 0.17 mmol) were added and the mixture was stirred at RT for 16 h. Subsequently, the mixture was concentrated and the residue was purified chromatographically by HPLC (Method 10). This gave 18 mg (15% of theory) of the title compound.

LC-MS (Method 4): Rₜ=1.33 min; MS (ESIpos): m/z=862.7 [M+H]⁺.

### Example 53A

### tert-Butyl [(trans-4-{[(2S)-3-[6-methyl-3′-(morpholin-4-ylcarbonyl)biphenyl-3-yl]-1-oxo-1-{[4-(1H-tetrazol-5-yl)phenyl]amino}propan-2-yl]carbamoyl}cyclohexyl)methyl]carbamate

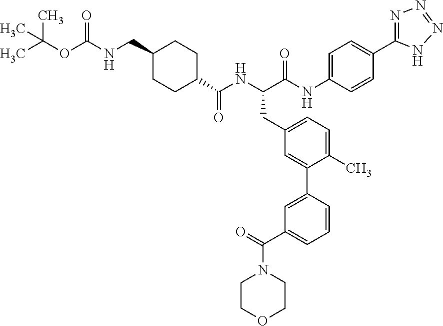

3-Bromo-N-alpha-[(trans-4-{[(tert-butoxycarbonyl)amino]methyl}cyclohexyl)carbonyl]-4-methyl-N-[4-(1H-tetrazol-5-yl)phenyl]-L-phenylalaninamide (150 mg, 0.23 mmol) and [3-(morpholin-4-ylcarbonyl)phenyl]boronic acid (82.5 mg, 0.35 mmol) were dissolved in dimethyl sulphoxide (2 ml), and tetrakis(triphenylphosphine)palladium(0) (27 mg, 23 μmol), sodium carbonate (74 mg, 0.7 mmol) and water (0.35 ml, 19.5 mmol) were added. The reaction mixture was stirred at 110° C. in a microwave (Biotage Initiator) for 90 min, cooled, filtered and purified by chromatography via HPLC (Method 11). This gave 119 mg (68% of theory) of the title compound.

LC-MS (Method 5): Rₜ=0.83 min; MS (ESIpos): m/z=751.5 [M+H]⁺.

### Example 54A

### tert-Butyl [(trans-4-{[(2S)-3-[3′-({2-[2-(2-methoxyethoxy)ethoxy]ethyl}carbamoyl)-6-methylbiphenyl-3-yl]-1-oxo-1-{[4-(1H-tetrazol-5-yl)phenyl]amino}propan-2-yl]carbamoyl}cyclohexyl)methyl]carbamate

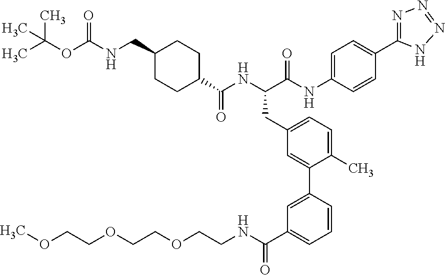

5′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2′-methylbiphenyl-3-carboxylic acid (90 mg, 0.13 mmol) and 2-[2-(2-methoxyethoxy)ethoxy]ethanamine (26 mg, 0.16 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (60 mg, 0.16 mmol) and N,N-diisopropylethylamine (20 mg, 0.16 mmol) were added and the mixture was stirred at RT overnight. Subsequently, the mixture was concentrated and the residue was purified chromatographically by HPLC (Method 10). This gave 42 mg (38% of theory) of the title compound.

LC-MS (Method 4): Rₜ=1.20 min; MS (ESIpos): m/z=827.6 [M+H]⁺.

### Example 55A

### tert-Butyl [(trans-4-{[(2S)-3-(6-methyl-3′-{[1-(propan-2-yl)piperidin-4-yl]carbamoyl}biphenyl-3-yl)-1-oxo-1-{[4-(1H-tetrazol-5-yl)phenyl]amino}propan-2-yl]carbamoyl}cyclohexyl)methyl]carbamate

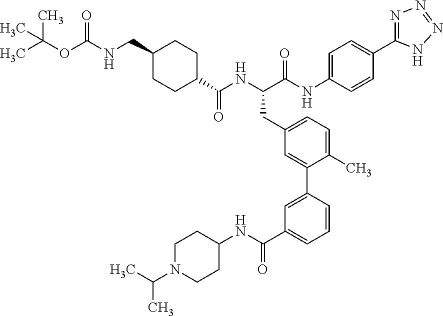

3-Bromo-N-alpha-[(trans-4-{[(tert-butoxycarbonyl)amino]methyl}cyclohexyl)carbonyl]-4-methyl-N-[4-(1H-tetrazol-5-yl)phenyl]-L-phenylalaninamide (150 mg, 0.23 mmol) and (3-{[1-(propan-2-yl)piperidin-4-yl]carbamoyl}phenyl)boronic acid (82.5 mg, 0.35 mmol) were dissolved in dimethyl sulphoxide (2 ml), and tetrakis(triphenylphosphine)palladium(0) (27 mg, 23 μmol), sodium carbonate (74 mg, 0.7 mmol) and water (0.35 ml, 19.5 mmol) were added. The reaction mixture was stirred at 110° C. in a microwave (Biotage Initiator) for 90 min, cooled, filtered and purified by chromatography via HPLC (Method 11). This gave 44 mg (23% of theory) of the title compound.

LC-MS (Method 5): Rₜ=0.91 min; MS (ESIpos): m/z=806.7 [M+H]⁺.

### Example 56A

### tert-Butyl [(trans-4-{[(2S)-3-[5′-({2-[2-(2-methoxyethoxy)ethoxy]ethyl}carbamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate

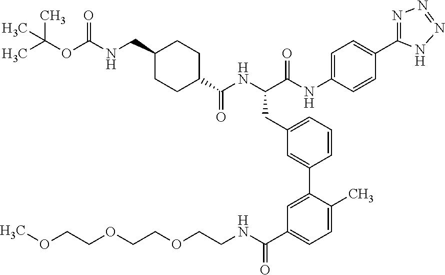

3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-6-methylbiphenyl-3-carboxylic acid (100 mg, 0.15 mmol) and 2-[2-(2-methoxyethoxy)ethoxy]ethanamine (29 mg, 0.17 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.17 mmol) and N,N-diisopropylethylamine (23 mg, 0.17 mmol) were added and the mixture was stirred at RT overnight. Subsequently, the mixture was concentrated and the residue was purified chromatographically by HPLC (Method 10). This gave 45 mg (37% of theory) of the title compound.

LC-MS (Method 4): Rₜ=1.20 min; MS (ESIpos): m/z=827.6 [M+H]⁺.

### Example 57A

### tert-Butyl [(trans-4-{[(2S)3-[5′-({2-[2-(2-hydroxyethoxy)ethoxy]ethyl}carbamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate

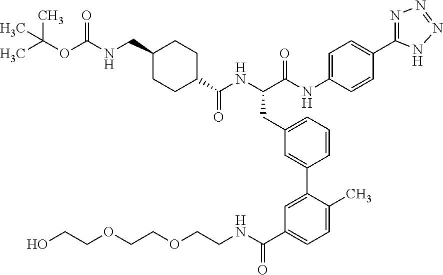

3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-6-methylbiphenyl-3-carboxylic acid (100 mg, 0.15 mmol) and 2-[2-(2-aminoethoxy)ethoxy]ethanol (26 mg, 0.17 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.17 mmol) and N,N-diisopropylethylamine (23 mg, 0.17 mmol) were added and the mixture was stirred at RT overnight. Subsequently, the mixture was concentrated and the residue was purified chromatographically by HPLC (Method 10). This gave 56 mg (47% of theory) of the title compound.

LC-MS (Method 4): Rₜ=1.12 min; MS (ESIpos): m/z=813.6 [M+H]⁺.

### Example 58A

### tert-Butyl [(trans-4-{[(2S)-3-[4′-({2-[2-(2-methoxyethoxy)ethoxy]ethyl}carbamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate

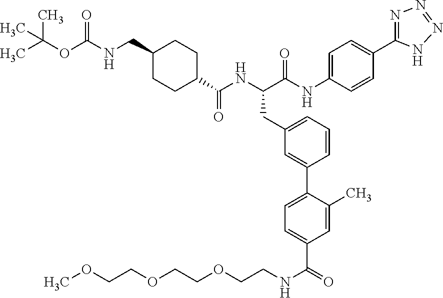

3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2-methylbiphenyl-4-carboxylic acid and 2-[2-(2-methoxyethoxy)ethoxy]ethanamine (29 mg, 0.17 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.17 mmol) and N,N-diisopropylethylamine (23 mg, 0.17 mmol) were added and the mixture was stirred at RT overnight. Subsequently, the mixture was concentrated and the residue was purified chromatographically by HPLC (Method 10). This gave 53 mg (44% of theory) of the title compound.

LC-MS (Method 4): Rₜ=1.17 min; MS (ESIpos): m/z=827.6 [M+H]⁺.

### Example 59A

### tert-Butyl [(trans-4-{[(2S)-3-[5′-(dimethylsulphamoyl)-2′-methylbiphenyl-3-yl]-1-oxo-1-{[4-(5-oxo-4,5-dihydro-1,2,4-oxadiazol-3-yl)phenyl]amino}propan-2-yl]carbamoyl}cyclohexyl)methyl]carbamate

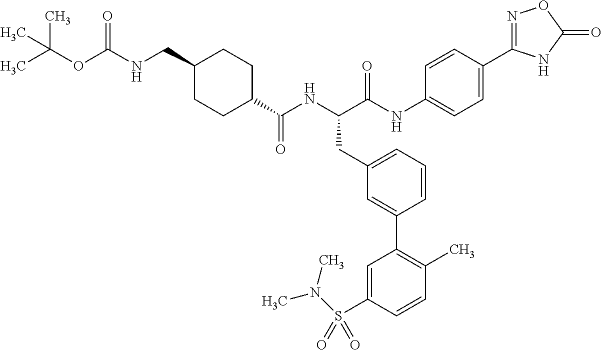

3-Bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide (150 mg, 0.24 mmol) and [5-(dimethylsulphamoyl)-2-methylphenyl]boronic acid (85 mg, 0.35 mmol) were dissolved in dimethyl sulphoxide (2 ml), and tetrakis(triphenylphosphine)palladium(0) (27 mg, 23 μmol), sodium carbonate (74 mg, 0.70 mmol) and water (0.35 ml, 19.5 mmol) were added. The reaction mixture was stirred at 110° C. in a microwave (Biotage Initiator) for 90 min, cooled, filtered and purified by chromatography via HPLC (Method 10). This gave 83 mg (47% of theory) of the title compound.

LC-MS (Method 4): Rₜ=1.31 min; MS (ESIpos): m/z=759.5 [M+H]⁺.

### Example 60A

### tert-Butyl [(trans-4-{[(2S)-1-{[4-(3-chloro-4H-1,2,4-triazol-5-yl)phenyl]amino}-3-{3′-[(4-methylpiperazin-1-yl)carbonyl]biphenyl-3-yl}-1-oxopropan-2-yl]carbamoyl}cyclohexyl)methyl]carbamate

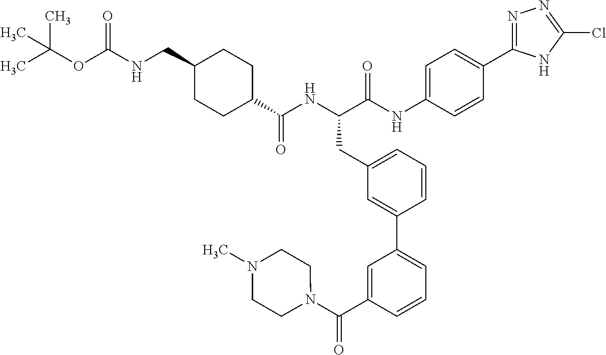

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and {3-[(4-methylpiperazin-1-yl)carbonyl]phenyl}boronic acid (97 mg, 0.34 mmol) were dissolved in dimethyl sulphoxide (2 ml), and tetrakis(triphenylphosphine)palladium(0) (26 mg, 23 μmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified by chromatography via HPLC (Method 11). This gave 13 mg (7% of theory) of the title compound.

LC-MS (Method 5): Rₜ=0.83 min; MS (ESIpos): m/z=783.5 [M+H]⁺.

### Example 61A

### tert-Butyl [(trans-4-{[(2S)-3-[4′-({2-[2-(2-hydroxyethoxy)ethoxy]ethyl}carbamnoyl)-2′-methylbiphenyl-3-yl]-1-oxo-1-{[4-(1H-tetrazol-5-yl)phenyl]amino}propan-2-yl]carbamoyl}-cyclohexyl)methyl]carbamate

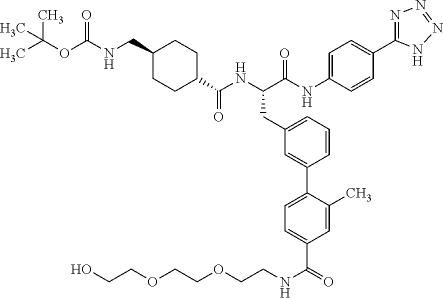

3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2-methylbiphenyl-4-carboxylic acid (100 mg, 0.15 mmol) and 2-[2-(2-aminoethoxy)ethoxy]ethanol (26 mg, 0.17 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.17 mmol) and N,N-diisopropylethylamine (23 mg, 0.17 mmol) were added and the mixture was stirred at RT overnight. Subsequently, the mixture was concentrated and the residue was purified chromatographically by HPLC (Method 10). This gave 55 mg (46% of theory) of the title compound.

LC-MS (Method 4): Rₜ=1.10 min; MS (ESIpos): m/z=813.6 [M+H]⁺.

### Example 62A

### tert-Butyl {[trans-4-({(2S)-1-{[4-(3-chloro-4H-1,2,4-triazol-5-yl)phenyl]amino}-3-[4′-(morpholin-4-ylcarbonyl)biphenyl-3-yl]-1-oxopropan-2-yl}carbamoyl)cyclohexyl]methyl}carbamate

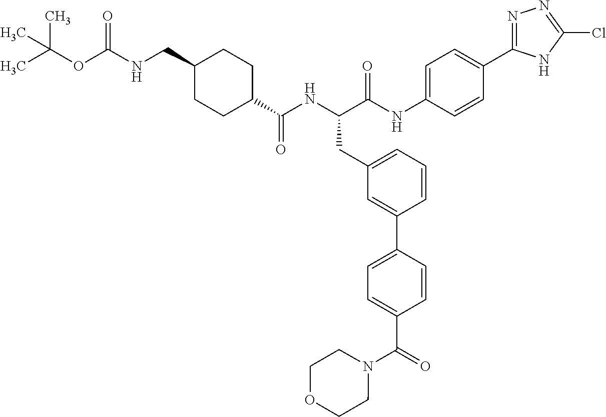

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and morpholin-4-yl[4-(4,4,5,5-tetramethyl-1,3,2-dioxaborolan-2-yl)phenyl]methanone (108 mg, 0.34 mmol) were dissolved in dimethyl sulphoxide (2 ml), and tetrakis(triphenylphosphine)palladium(0) (26 mg, 23 μmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified by chromatography via HPLC (Method 10). This gave 52 mg (30% of theory) of the title compound.

LC-MS (Method 4): Rₜ=1.21 min; MS (ESIpos): m/z=770.5 [M+H]⁺.

### Example 63A

### tert-Butyl [(trans-4-{[(2S)-1-{[4-(3-chloro-4H-1,2,4-triazol-5-yl)phenyl]amino}-3-{4′-[(4-methylpiperazin-1-yl)carbonyl]biphenyl-3-yl}-1-oxopropan-2-yl]carbamoyl}cyclohexyl)methyl]carbamate

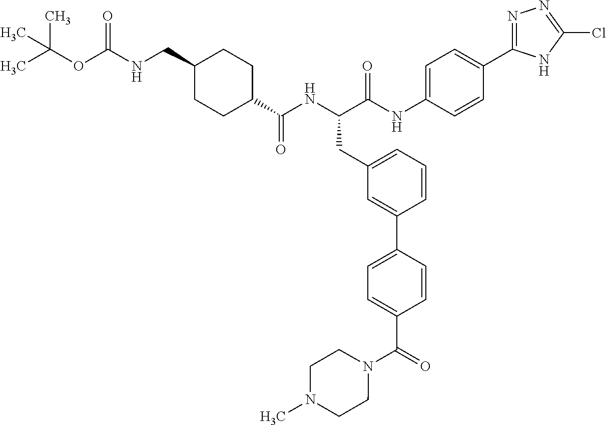

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and (4-methylpiperazin-1-yl)[4-(4,4,5,5-tetramethyl-1,3,2-dioxaborolan-2-yl)phenyl]methanone (125 mg, 0.34 mmol) were dissolved in dimethyl sulphoxide (2 ml), and tetrakis(triphenylphosphine)palladium(0) (26 mg, 23 μmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified by chromatography via HPLC (Method 11). This gave 69 mg (39% of theory) of the title compound.

LC-MS (Method 5): Rₜ=0.84 min; MS (ESIpos): m/z=783.5 [M+H]⁺.

### Example 64A

### tert-Butyl [(trans-4-{[(2S)-3-[3′-({2-[2-(2-hydroxyethoxy)ethoxy]ethyl}carbamoyl)-6-methylbiphenyl-3-yl]-1-oxo-1-{[4-(1H-tetrazol-5-yl)phenyl]amino}propan-2-yl]carbamoyl}-cyclohexyl)methyl]carbamate

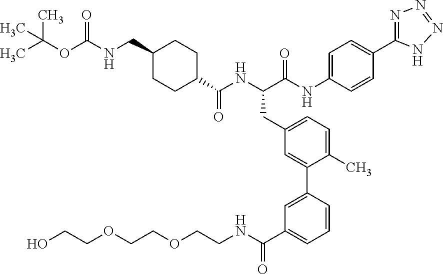

5′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2′-methylbiphenyl-3-carboxylic acid (100 mg, 0.15 mmol) and 2-[2-(2-aminoethoxy)ethoxy]ethanol (26 mg, 0.18 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.18 mmol) and N,N-diisopropylethylamine (23 mg, 0.18 mmol) were added and the mixture was stirred at RT overnight. Subsequently, the mixture was concentrated and the residue was purified chromatographically by HPLC (Method 10). This gave 48 mg (41% of theory) of the title compound.

LC-MS (Method 4): Rₜ=1.12 min; MS (ESIpos): m/z=813.6 [M+H]⁺.

### Example 65A

### tert-Butyl [(trans-4-{[(2S)-3-(3′-{[2-(morpholin-4-yl)ethyl]carbamoyl}biphenyl-3-yl)-1-oxo-1-{[4-(1H-tetrazol-5-yl)phenyl]amino}propan-2-yl]carbamoyl}cyclohexyl)methyl]carbamate

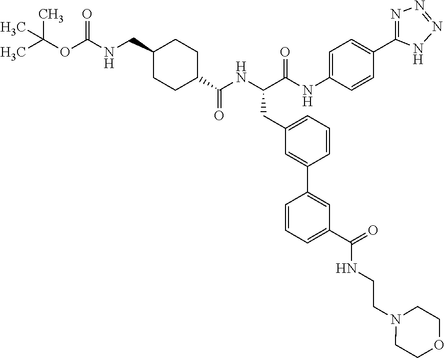

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (3-{[2-(morpholin-4-yl)ethyl]carbamoyl}phenyl)boronic acid (100 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 11). This gave 40 mg (21% of theory) of the title compound.

LC-MS (Method 5): Rₜ=0.8 min; MS (ESIpos): m/z=780.6 [M+H]⁺.

### Example 66A

### tert-Butyl [(trans-4-{[(2S)-1-{[4-(3-chloro-4H-1,2,4-triazol-5-yl)phenyl]amino}-1-oxo-3-(3′-{[1-(propan-2-yl)piperidin-4-yl]carbamoyl}biphenyl-3-yl)propan-2-yl]carbamoyl}cyclohexyl)methyl]carbamate

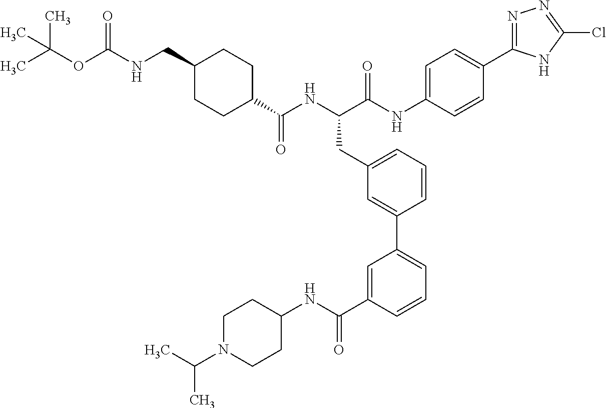

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and {3-[(1-isopropylpiperidin-4-yl)carbamoyl]phenyl}boronic acid (111 mg, 0.34 mmol) were dissolved in dimethyl sulphoxide (2 ml), and tetrakis(triphenylphosphine)palladium(0) (26 mg, 23 μmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified by chromatography via HPLC (Method 11). This gave 49 mg (26% of theory) of the title compound.

LC-MS (Method 5): Rₜ=0.93 min; MS (ESIpos): m/z=825.6 [M+H]⁺.

### Example 67A

### tert-Butyl [(trans-4-{[(2S)-3-{3′-[(4-methylpiperazin-1-yl)carbonyl]biphenyl-3-yl}-1-oxo-1-{[4-(1H-tetrazol-5-yl)phenyl]amino}propan-2-yl]carbamoyl}cyclohexyl)methyl]carbamate

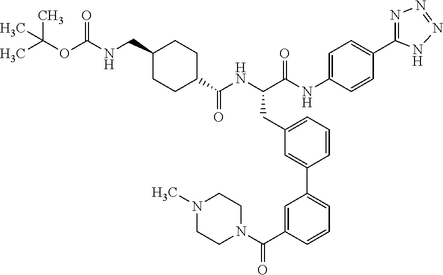

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and {3-[(4-methylpiperazin-1-yl)carbonyl]phenyl}boronic acid (102 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 135 min, cooled, filtered and purified by chromatography via HPLC (Method 11). This gave 47 mg (26% of theory) of the title compound.

LC-MS (Method 5): Rₜ=0.8 min; MS (ESIpos): m/z=750.6 [M+H]⁺.

### Example 68A

### tert-Butyl [(trans-4-{[(2S)-1-oxo-3-(3′-{[1-(propan-2-yl)piperidin-4-yl]carbamoyl}biphenyl-3-yl)-1-{[4-(1H-tetrazol-5-yl)phenyl]amino}propan-2-yl]carbamoyl}cyclohexyl)methyl]carbamate

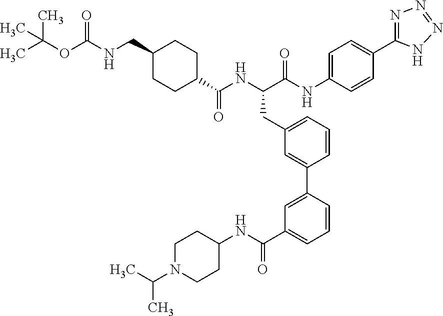

3-Bromo-N-alpha-[(trans-4-{[(tert-butoxycarbonyl)amino]methyl}cyclohexyl)carbonyl_{]}-N-[4-(1H-tetrazol-5-yl)phenyl]-L-phenylalaninamide (150 mg, 0.24 mmol) and (3-{[1-(propan-2-yl)piperidin-4-yl]carbamoyl}phenyl)boronic acid (104 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 47 mg (25% of theory) of the title compound.

LC-MS (Method 5): Rₜ=0.88 min; MS (ESIpos): m/z=792.6 [M+H]⁺.

### Example 69A

### tert-Butyl 6-[({3′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2-methylbiphenyl-4-yl}carbonyl)amino]-3-azabicyclo[3.1.0]hexane-3-carboxylate

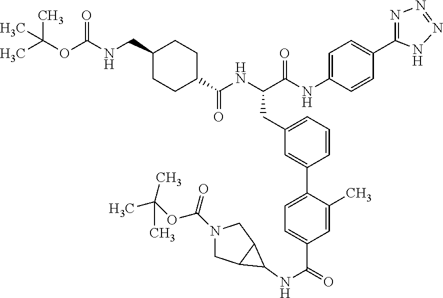

3′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2-methylbiphenyl-4-carboxylic acid (100 mg, 0.15 mmol) and tert-butyl 6-amino-3-azabicyclo[3.1.0]hexane-3-carboxylate (35 mg, 0.17 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.17 mmol) and N,N-diisopropylethylamine (23 mg, 0.17 mmol) were added and the mixture was stirred at RT overnight. Subsequently, the mixture was concentrated and the residue was purified chromatographically by HPLC (Method 10). This gave 51 mg (41% of theory) of the title compound.

LC-MS (Method 4): Rₜ=1.32 min; MS (ESIpos): m/z=862.7 [M+H]⁺.

### Example 70A

### tert-Butyl [(trans-4-{[(2S)-3-(3′-{[2-(diethylamino)ethyl]carbamoyl}biphenyl-3-yl)-1-oxo-1-{[4-(1H-tetrazol-5-yl)phenyl]amino}propan-2-yl]carbamoyl}cyclohexyl)methyl]carbamate

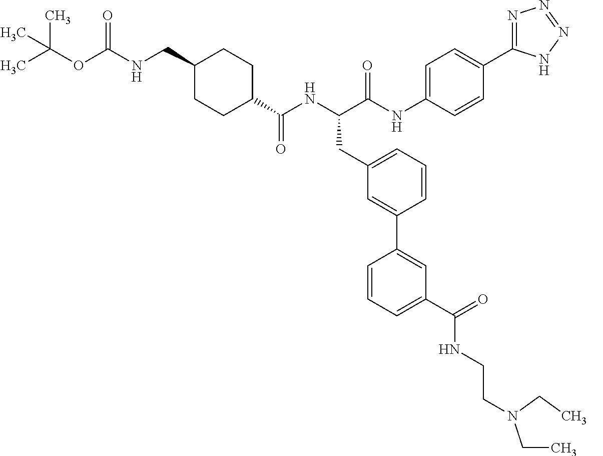

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (3-{[2-(diethylamino)ethyl]carbamoyl}phenyl)boronic acid (108 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 210 min, cooled, filtered and purified by chromatography via HPLC (Method 11). This gave 50 mg (27% of theory) of the title compound.

LC-MS (Method 5): Rₜ=0.89 min; MS (ESIpos): m/z=766.6 [M+H]⁺.

### Example 71A

### tert-Butyl 6-[({5′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2′-methylbiphenyl-3-yl}carbonyl)amino]-3-azabicyclo[3.1.0]hexane-3-carboxylate

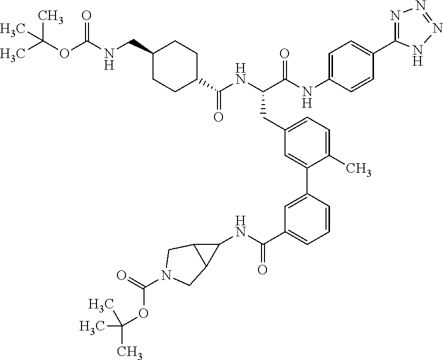

5′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2′-methylbiphenyl-3-carboxylic acid (100 mg, 0.15 mmol) and tert-butyl 6-amino-3-azabicyclo[3.1.0]hexane-3-carboxylate (35 mg, 0.18 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.18 mmol) and N,N-diisopropylethylamine (23 mg, 0.18 mmol) were added and the mixture was stirred at RT overnight. Subsequently, the mixture was concentrated and the residue was purified chromatographically by HPLC (Method 10). This gave 50 mg (40% of theory) of the title compound.

LC-MS (Method 4): Rₜ=1.33 min; MS (ESIpos): m/z=862.7 [M+H]⁺.

### Example 72A

### tert-Butyl [(trans-4-{[(2S)-3-[2′-methyl-5′-(pyrrolidin-1-ylsulphonyl)biphenyl-3-yol]-1-oxo-1-{[4-(5-oxo-4,5-dihydro-1,2,4-oxadiazol-3-yl)phenyl]amino}propan-2-yl]carbamoyl}cyclohexyl)methyl]carbamate

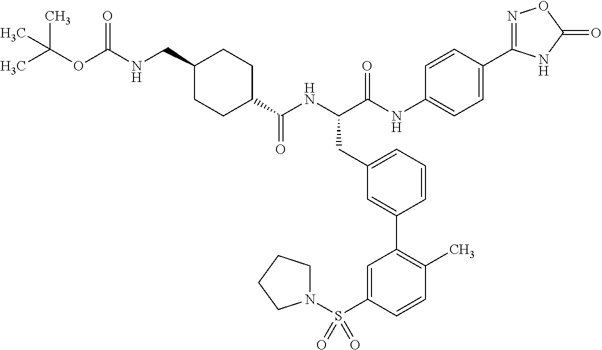

3-Bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide (150 mg, 0.24 mmol) and [2-methyl-5-(pyrrolidin-1-ylsulphonyl)phenyl]boronic acid (94 mg, 0.35 mmol) were dissolved in dimethyl sulphoxide (2 ml), and tetrakis(triphenylphosphine)palladium(0) (27 mg, 23 μmol), sodium carbonate (74 mg, 0.70 mmol) and water (0.35 ml, 19.5 mmol) were added. The reaction mixture was stirred at 110° C. in a microwave (Biotage Initiator) for 180 min, cooled, filtered and purified by chromatography via HPLC (Method 10). This gave 58 mg (32% of theory) of the title compound.

LC-MS (Method 4): Rₜ=1.34 min; MS (ESIpos): m/z=787.5 [M+H]⁺.

### Example 73A

### tert-Butyl [(trans-4-{[(2S)-1-oxo-3-(4′-{[2-(piperidin-1-yl)ethyl]carbamoyl}biphenyl-3-yl)-1-{[4-(1H-tetrazol-5-yl)phenyl]amino}propan-2-yl]carbamoyl}cyclohexyl)methyl]carbamate

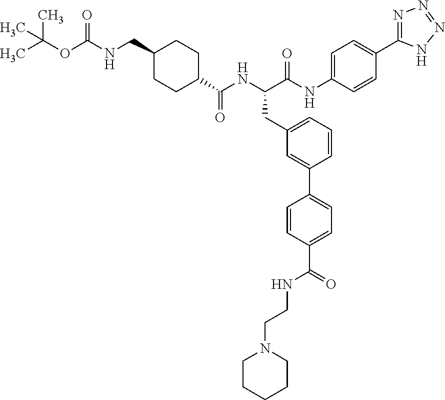

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (4-{[2-(piperidin-1-yl)ethyl]carbamoyl}phenyl)boronic acid (99 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 250 min, cooled, filtered and purified by chromatography via HPLC (Method 11). This gave 73 mg (39% of theory) of the title compound.

LC-MS (Method 5): Rₜ=0.90 min; MS (ESIpos): m/z=778.6 [M+H]⁺.

### Example 74A

### tert-Butyl [(trans-4-{[(2S)-3-(5′-carbamoyl-2′-chlorobiphenyl-3-yl)-1-oxo-1-{[4-(1H-tetrazol-5-yl)phenyl]amino}propan-2-yl]carbamoyl}cyclohexyl)methyl]carbamate

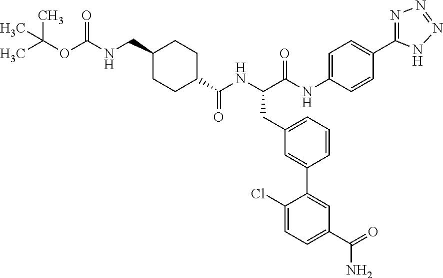

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (5-carbamoyl-2-chlorophenyl)boronic acid (72 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 55 mg (33% of theory) of the title compound.

LC-MS (Method 5): Rₜ=0.81 min; MS (ESIpos): m/z=701.5 [M+H]⁺.

### Example 75A

### tert-Butyl [(trans-4-{[(2S)-3-(3′-{[2-(dimethylamino)ethyl]carbamoyl}biphenyl-3-yl)-1-oxo-1-{[4-(1H-tetrazol-5-yl)phenyl]amino}propan-2-yl]carbamoyl}cyclohexyl)methyl]carbamate

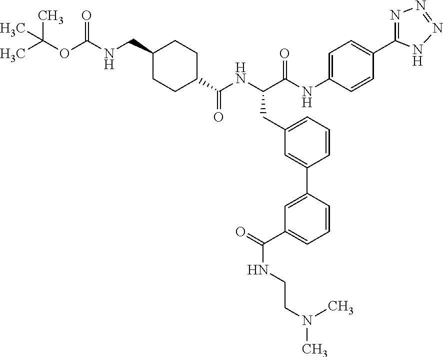

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and N-[2-(dimethylamino)ethyl]-3-(4,4,5,5-tetramethyl-1,3,2-dioxaborolan-2-yl)benzamide (114 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 81 mg (46% of theory) of the title compound.

LC-MS (Method 5): Rₜ=0.84 min; MS (ESIpos): m/z=738.6 [M+H]⁺.

### Example 76A

### tert-Butyl [(trans-4-{[(2S)-1-{[4-(3-chloro-4H-1,2,4-triazol-5-yl)phenyl]amino}-3-(3′-{[2-(diethylamino)ethyl]carbamoyl}biphenyl-3-yl)-1-oxopropan-2-yl]carbamoyl}cyclohexyl)methyl]carbamate

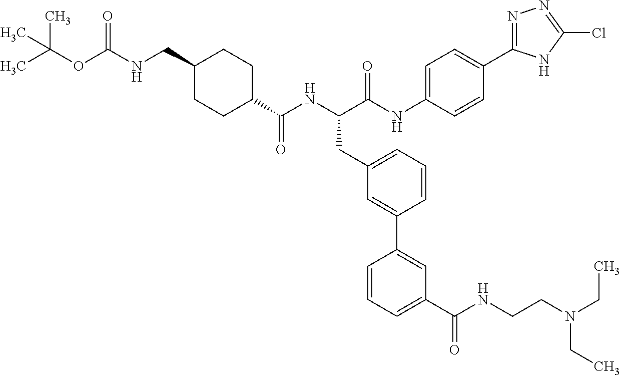

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and (3-{[2-(diethylamino)ethyl]carbamoyl}phenyl)boronic acid (102 mg, 0.34 mmol) were dissolved in dimethyl sulphoxide (2 ml), and tetrakis(triphenylphosphine)palladium(0) (26 mg, 23 μmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified chromatographically via HPLC (Method 11, 0-8 min 25-50% B). This gave 29 mg (16% of theory) of the title compound.

LC-MS (Method 5): Rₜ=0.86 min; MS (ESIpos): m/z=799.7 [M+H]⁺.

### Example 77A

### tert-Butyl [(trans-4-{[(2S)-1-oxo-3-(4′-{[2-(pyrrolidin-1-yl)ethyl]carbamoyl}biphenyl-3-yl)-1-{[4-(1H-tetrazol-5-yl)phenyl]amino}propan-2-yl]carbamoyl}cyclohexyl)methyl]carbamate

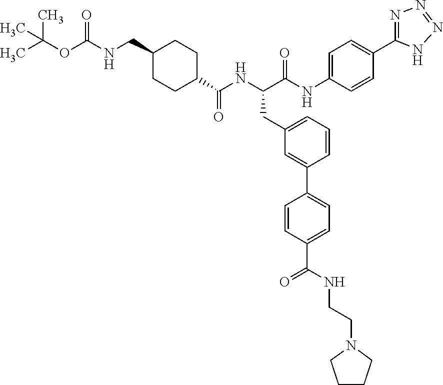

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (4-{[2-(pyrrolidin-1-yl)ethyl]carbamoyl}phenyl)boronic acid (107 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 51 mg (28% of theory) of the title compound.

LC-MS (Method 5): Rₜ=0.86 min; MS (ESIpos): m/z=764.6 [M+H]⁺.

### Example 78A

### tert-Butyl [(trans-4-{[(2S)-3-(4′-{[2-(diethylamino)ethyl]carbamoyl}biphenyl-3-yl)-1-oxo-1-{[4-(1H-tetrazol-5-yl)phenyl]amino}propan-2-yl]carbamoyl}cyclohexyl)methyl]carbamate

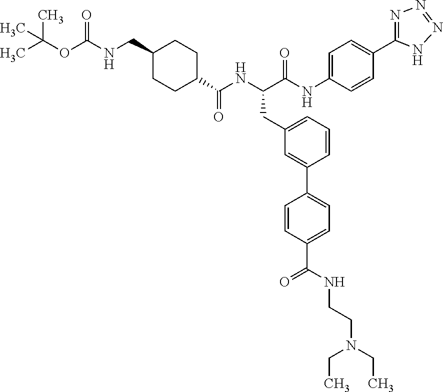

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (4-{[2-(diethylamino)ethyl]carbamoyl}phenyl)boronic acid (108 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 89 mg (49% of theory) of the title compound.

LC-MS (Method 5): Rₜ=0.87 min; MS (ESIpos): m/z=766.6 [M+H]⁺.

### Example 79A

### tert-Butyl [(trans-4-{[(2S)-3-[3′-(dimethylcarbamoyl)biphenyl-3-yl]-1-oxo-1-{[4-(1H-tetrazol-5-yl)phenyl]amino}propan-2-yl]carbamoyl}cyclohexyl)methyl]carbamate

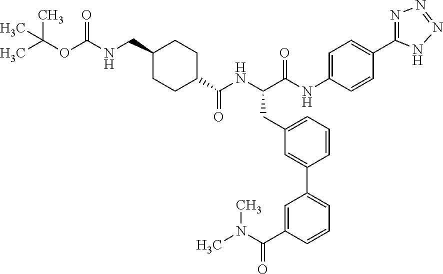

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [3-(dimethylcarbamoyl)phenyl]boronic acid (69 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 10). This gave 55 mg (33% of theory) of the title compound.

LC-MS (Method 5): Rₜ=1.13 min; MS (ESIpos): m/z=696.6 [M+H]⁺.

### Example 80A

### tert-Butyl {[trans-4-({(2S)-3-[5′-(dimethylsulphamoyl)-2′-methylbiphenyl-3-yl]-1-oxo-1-[(2-oxo-2,3-dihydro-1H-benzimidazol-5-yl)amino]propan-2-yl}carbamoyl)cyclohexyl]methyl}carbamate

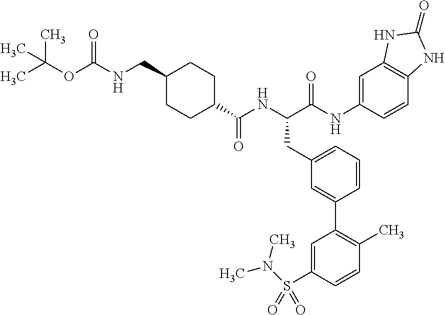

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and [5-(dimethylsulphamoyl)-2-methylphenyl]boronic acid (89 mg, 0.36 mmol) were dissolved in dimethyl sulphoxide (2 ml), and tetrakis(triphenylphosphine)palladium(0) (28 mg, 24 μmol), sodium carbonate (77 mg, 0.73 mmol) and water (0.37 ml, 20 mmol) were added. The reaction mixture was stirred at 110° C. in a microwave (Biotage Initiator) for 90 min, cooled, filtered and purified by chromatography via HPLC (Method 10). This gave 34 mg (19% of theory) of the title compound.

LC-MS (Method 4): Rₜ=1.22 min; MS (ESIpos): m/z=733.4 [M+H]⁺.

### Example 81A

### tert-Butyl [(trans-4-{[(2S)-3-{2′-fluoro-5′-[methoxy(methyl)carbamoyl]biphenyl-3-yl}-1-oxo-1-{[4-(1H-tetrazol-5-yl)phenyl]amino}propan-2-yl]carbamoyl}cyclohexyl)methyl]carbamate

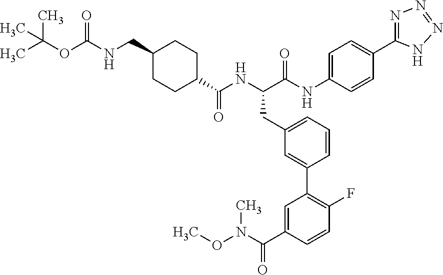

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and {2-fluoro-5-[methoxy-(methyl)carbamoyl]phenyl}boronic acid (82 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 105 mg (60% of theory) of the title compound.

LC-MS (Method 5): Rₜ=0.87 min; MS (ESIpos): m/z=729.5 [M+H]⁺.

### Example 82A

### tert-Butyl [(trans-4-{[(2S)-3-{4′-[(4-methylpiperazin-1-yl)carbonyl]biphenyl-3-yl}-1-oxo-1-{[4-(1H-tetrazol-5-yl)phenyl]amino}propan-2-yl]carbamoyl}cyclohexyl)methyl]carbamate

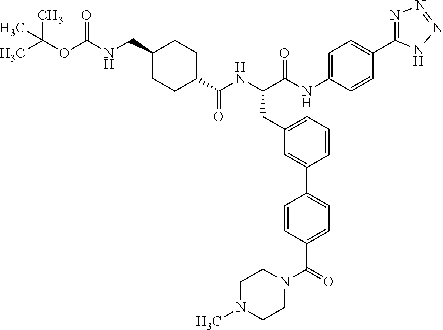

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (4-methylpiperazin-1-yl)[4-(4,4,5,5-tetramethyl-1,3,2-dioxaborolan-2-yl)phenyl]methanone (119 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 11). This gave 96 mg (54% of theory) of the title compound.

LC-MS (Method 5): Rₜ=0.82 min; MS (ESIpos): m/z=750.6 [M+H]⁺.

### Example 83A

### tert-Butyl [(trans-4-{[(2S)-3-[3′-(morpholin-4-ylcarbonyl)biphenyl-3-yl]-1-oxo-1-{[4-(1H-tetrazol-5-yl)phenyl]amino}propan-2-yl]carbamoyl}cyclohexyl)methyl]carbamate

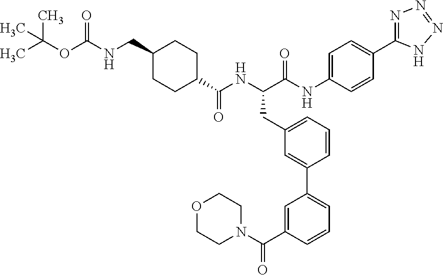

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [3-(morpholin-4-ylcarbonyl)phenyl]boronic acid (84 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 68 mg (39% of theory) of the title compound.

LC-MS (Method 4): Rₜ=1.11 min; MS (ESIpos): m/z=737.9 [M+H]⁺.

### Example 84A

### tert-Butyl [(trans-4-{[(2S)-3-(4′-{[4-(2-hydroxyethyl)piperazin-1-yl]carbonyl}biphenyl-3-yl)-1-oxo-1-{[4-(1H-tetrazol-5-yl)phenyl]amino}propan-2-yl]carbamoyl}cyclohexyl)methyl]carbamate

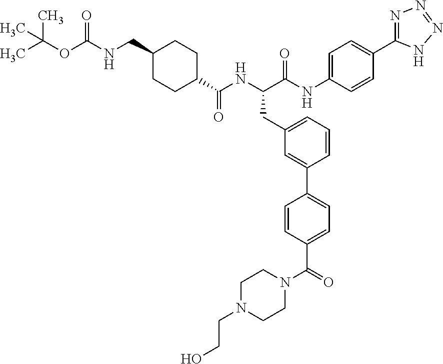

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (4-{[4-(2-hydroxyethyl)piperazin-1-yl]carbonyl}phenyl)boronic acid (113 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 11). This gave 72 mg (39% of theory) of the title compound.

LC-MS (Method 5): Rₜ=0.80 min; MS (ESIpos): m/z=780.6 [M+H]⁺.

### Example 85A

### tert-Butyl 4-[({5′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2′-methylbiphenyl-4-yl}carbonyl)amino]piperidine-1-carboxylate

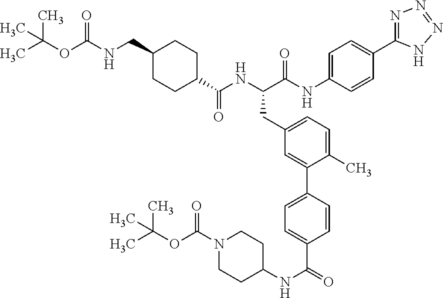

5′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-2′-methylbiphenyl-4-carboxylic acid (100 mg, 0.15 mmol) and tert-butyl 4-aminopiperidine-1-carboxylate (35 mg, 0.18 mmol) were dissolved in tetrahydrofuran (4 ml), N-[(dimethylamino)(3H-[1,2,3]triazolo[4,5-b]pyridin-3-yloxy)methylidene]-N-methylmethanaminium hexafluorophosphate (67 mg, 0.18 mmol) and N,N-diisopropylethylamine (23 mg, 0.18 mmol) were added and the mixture was stirred at RT overnight. Subsequently, the mixture was concentrated and the residue was purified chromatographically by HPLC (Method 9). This gave 72 mg (57% of theory) of the title compound.

LC-MS (Method 4): Rₜ=1.35 min; MS (ESIpos): m/z=864.9 [M+H]⁺

### Example 86A

### tert-Butyl [(trans-4-{[(2S)-3-[4′-({2-[(tert-butoxycarbonyl)amino]ethyl}carbamoyl)biphenyl-3-yl]-1-(1H-indazol-6-ylamino)-1-oxopropan-2-yl]carbamoyl}cyclohexyl)methyl]carbamate trifluoroacetate

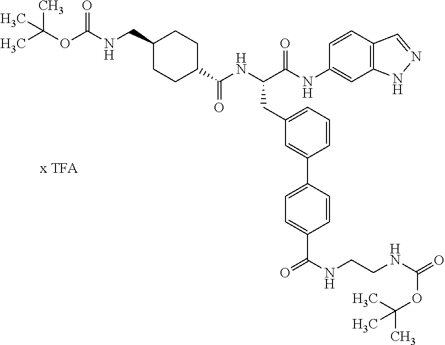

100 mg (0.16 mmol) of 3′-[(2S)-2-{[(trans-4-{[(tert-butoxycarbonyl)amino]methyl}-cyclohexyl)-carbonyl]amino}-3-(1H-indazol-6-ylamino)-3-oxopropyl]biphenyl-4-carboxylic acid and 30 mg (0.19 mmol) of tert-butyl (2-aminoethyl)carbamate were dissolved in 5 ml of THF, 71 mg (0.19 mmol) of N-[(dimethylamino)(3H-[1,2,3]triazolo[4,5-b]pyridin-3-yloxy)methylidene]-N-methylmethanaminium hexafluorophosphate and 0.03 ml (0.19 mmol) of N,N-diisopropylethylamine were added and the solution was stirred at RT for 20 h. The reaction mixture was separated directly by preparative HPLC (mobile phase: acetonitrile/water gradient, 0.1% trifluoroacetic acid). This gave 66 mg (47% of theory) of the title compound.

LC-MS (Method 1): Rₜ=1.07 min; MS (ESIneg): m/z=780 [M−H−TFA]⁻.

### Example 87A

### tert-Butyl 4-[({3′-[(2S)-2-{[(trans-4-{[(tert-butoxycarbonyl)amino]methyl}cyclohexyl)carbonyl]amino}-3-(1H-indazol-6-ylamino)-3-oxopropyl]biphenyl-4-yl}carbonyl)amino]piperidine-1-carboxylate trifluoroacetate

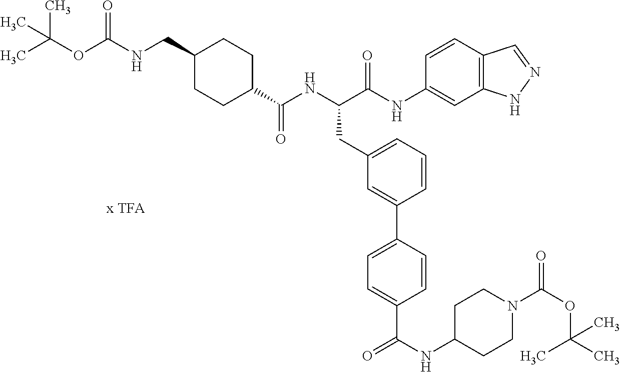

100 mg (0.16 mmol) of 3′-[(2S)-2-{[(trans-4-{[(tert-butoxycarbonyl)amino]methyl}cyclohexyl)-carbonyl]amino}-3-(1H-indazol-6-ylamino)-3-oxopropyl]biphenyl-4-carboxylic acid and 38 mg (0.19 mmol) of tert-butyl 4-aminopiperidine-1-carboxylate were dissolved in 5 ml of THF, 71 mg (0.19 mmol) of N-[(dimethylamino)(3H-[1,2,3]triazolo[4,5-b]pyridin-3-yloxy)methylidene]-N-methylmethanaminium hexafluorophosphate and 0.03 ml (0.19 mmol) of N,N-diisopropylethylamine were added and the solution was stirred at RT for 4 h. The reaction mixture was separated directly by preparative HPLC (mobile phase: acetonitrile/water gradient, 0.1% trifluoroacetic acid). This gave 85 mg (55% of theory, 94% pure) of the title compound.

LC-MS (Method 1): Rₜ=1.16 min; MS (ESIneg): m/z=820 [M−H−TFA]⁻.

### Example 88A

### tert-Butyl [(trans-4-{[(2S)-3-[2′-methyl-5′-(morpholin-4-ylsulphonyl)biphenyl-3-yl]-1-oxo-1-{[4-(5-oxo-4,5-dihydro-1,2,4-oxadiazol-3-yl)phenyl]amino}propan-2-yl]carbamoyl}cyclohexyl)methyl]carbamate

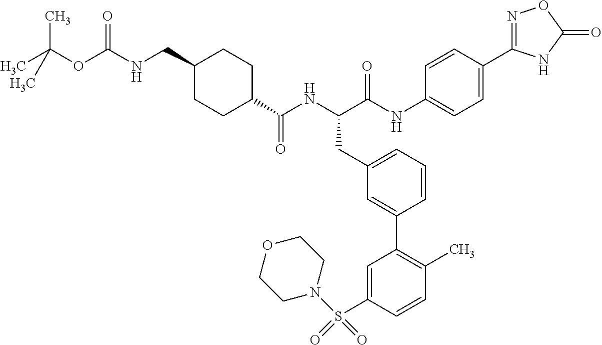

3-Bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide (150 mg, 0.23 mmol) and [2-methyl-5-(morpholin-4-ylsulphonyl)phenyl]boronic acid (100 mg, 0.35 mmol) were dissolved in dimethyl sulphoxide (2 ml), and tetrakis(triphenylphosphine)palladium(0) (27 mg, 23.3 μmol), sodium carbonate (74 mg, 0.7 mmol) and water (0.35 ml, 19.5 mmol) were added. The reaction mixture was stirred at 110° C. in a microwave (Biotage Initiator) for 90 min, cooled, filtered and purified by chromatography via HPLC (Method 10). This gave 21 mg (11% of theory) of the title compound.

LC-MS (Method 4): Rₜ=1.23 min; MS (ESIpos): m/z=803.4 [M+H]⁺.

### Example 89A

### tert-Butyl [(trans-4-{[(2S)-3-(4′-{[2-(diethylamino)ethyl]carbamoyl}biphenyl-3-yl)-1-(1H-indazol-6-ylamino)-1-oxopropan-2-yl]carbamoyl}cyclohexyl)methyl]carbamate trifluoroacetate

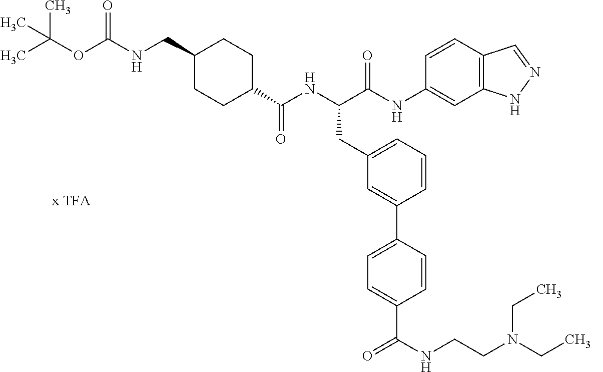

100 mg (0.16 mmol) of 3′-[(2S)-2-{[(trans-4-{[(tert-butoxycarbonyl)amino]methyl}cyclohexyl)carbonyl]amino}-3-(1H-indazol-6-ylamino)-3-oxopropyl]biphenyl-4-carboxylic acid and 22 mg (0.19 mmol) of diethylaminoethylamine were dissolved in 5 ml of THF, 71 mg (0.19 mmol) of N-[(dimethylamino)(3H-[1,2,3]triazolo[4,5-b]pyridin-3-yloxy)methylidene]-N-methylmethanaminium hexafluorophosphate and 0.03 ml (0.19 mmol) of N,N-diisopropylethylamine were added and the solution was stirred at RT for 64 h. The reaction mixture was separated directly by preparative HPLC (mobile phase: acetonitrile/water gradient, 0.1% trifluoroacetic acid). This gave 101 mg of a mixture of the title compound and the corresponding deprotected amine, which was used directly in the next stage.

LC-MS (Method 1): Rₜ=0.82 min; MS (ESIneg): m/z=736 [M−H−TFA]⁻.

### Example 90A

### tert-Butyl [(trans-4-{[(2S)-3-[2′-(morpholin-4-ylsulphonyl)biphenyl-3-yl]-1-oxo-1-{[4-(2H-tetrazol-5-yl)phenyl]amino}propan-2-yl]carbamoyl}cyclohexyl)methyl]carbamate

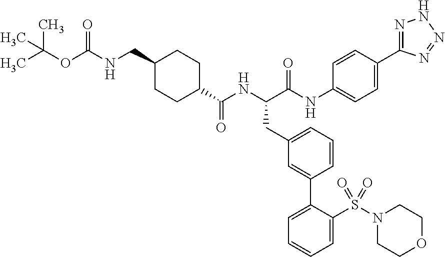

100 mg (0.16 mmol) of 3-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide, 18.4 mg (0.02 mmol) of tetrakis(triphenylphosphine)palladium(0) and 87 mg (0.32 mmol) of [3-(morpholin-4-ylsulphonyl)pyridin-4-yl]boronic acid were taken up in 1.5 ml of 1,2-dimethoxyethane and 0.50 ml of ethanol. After the addition of 1.2 ml of 2N aqueous sodium carbonate solution, the mixture was stirred under reflux for 5 h. The salts were removed by filtration from the reaction mixture and the filtrate was separated by preparative HPLC (mobile phase: acetonitrile/water gradient, 0.1% trifluoroacetic acid). This gave 40 mg of a mixture of the title compound and the corresponding deprotected amine, which was used directly in the next stage.

LC-MS (Method 1): Rₜ=1.06 min; MS (ESIneg): m/z=771 [M−H]⁻.

### Example 91A

### tert-Butyl 4-[({3′-[(2S)-2-{[(trans-4-{[(tert-butoxycarbonyl)amino]methyl}cyclohexyl)carbonyl]amino}-3-{[4-(3-chloro-4H-1,2,4-triazol-5-yl)phenyl]amino}-3-oxopropyl]-2-methylbiphenyl-4-yl}sulphonyl)amino]piperidine-1-carboxylate

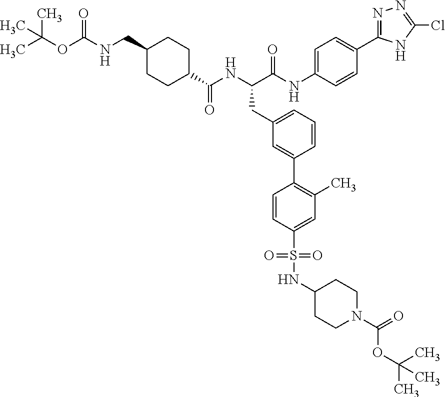

N-alpha-[(trans-4-{[(tert-Butoxycarbonyl)amino]methyl}cyclohexyl)carbonyl]-N-[4-(3-chloro-4H-1,2,4-triazol-5-yl)phenyl]-3-(4,4,5,5-tetramethyl-1,3,2-dioxaborolan-2-yl)-L-phenylalaninamide (200 mg, 0.28 mmol) and tert-butyl 4-{[(4-bromo-3-methylphenyl)sulphonyl]amino}piperidine-1-carboxylate (147 mg, 0.34 mmol) were dissolved in 1.9 ml of dimethyl sulphoxide, the solution was flushed with argon and tetrakis(triphenylphosphine)palladium(0) (32.6 mg, 28.3 μM), sodium carbonate (150 mg, 1.4 mmol) and water (0.43 ml, 23.6 mmol) were added. The reaction mixture was stirred at 110° C. in a microwave (Biotage Initiator) for 150 min, cooled, filtered and purified by chromatography via HPLC (Method 11). This gave 9.3 mg (3.4% of theory) of the title compound.

LC-MS (Method 5): Rₜ=0.99 min; MS (ESIpos): m/z=933.6 [M+H]⁺.

### Example 92A

### tert-Butyl 4-[({5′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-yl}carbonyl)amino]piperidine-1-carboxylate

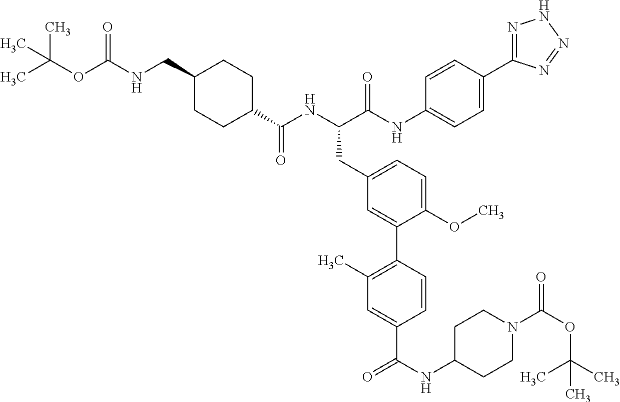

64 mg (0.17 mmol) of N-[(dimethylamino)(3H-[1,2,3]triazolo[4,5-b]pyridin-3-yloxy)methylidene]-N-methylmethanaminium hexafluorophosphate and 0.03 ml (0.17 mmol) of N,N-diisopropylethylamine were added to a solution of 100 mg (0.14 mmol) of 5′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-carboxylic acid and 34 mg (0.17 mmol) of tert-butyl 4-aminopiperidine-1-carboxylate in 5 ml of THF, and the mixture was stirred at RT for 16 h. The reaction mixture was separated directly by preparative HPLC (mobile phase: acetonitrile/water gradient, 0.1% trifluoroacetic acid). This gave 82 mg of a mixture of the title compound and the corresponding deprotected amine, which was used directly in the next stage.

LC-MS (Method 1): Rₜ=1.16 min; MS (ESIneg): m/z=893 [M−H]⁻.

### Example 93A

### tert-Butyl (3R)-3-[({5′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-yl}carbonyl)amino]pyrrolidine-1-carboxylate

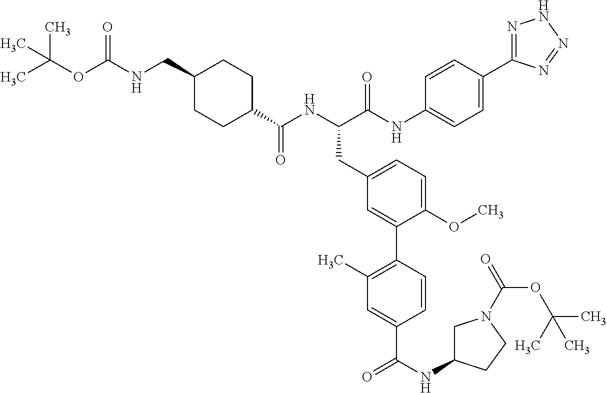

45 mg (0.12 mmol) of N-[(dimethylamino)(3H-[1,2,3]triazolo[4,5-b]pyridin-3-yloxy)methylidene]-N-methylmethanaminium hexafluorophosphate and 0.02 ml (0.12 mmol) of N,N-diisopropylethylamine were added to a solution of 70 mg (0.10 mmol) of 5′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-carboxylic acid and 22 mg (0.12 mmol) of tert-butyl (3R)-3-aminopyrrolidine-1-carboxylate in 3.5 ml of THF, and the mixture was stirred at RT for 16 h. The reaction mixture was separated directly by preparative HPLC (mobile phase: acetonitrile/water gradient, 0.1% trifluoroacetic acid). This gave 54 mg of a mixture of the title compound and the corresponding deprotected amine, which was used directly in the next stage.

LC-MS (Method 1): Rₜ=1.12 min; MS (ESIneg): m/z=878 [M−H]⁻.

### Example 94A

### tert-Butyl [(trans-4-{[(2S)-3-[4′-({2-[(tert-butoxycarbonyl)amino]ethyl}carbamoyl)-6-methoxy-2′-methylbiphenyl-3-yl]-1-oxo-1-{[4-(2H-tetrazol-5-yl)phenyl]amino}propan-2-yl]carbamoyl}cyclohexyl)methyl]carbamate

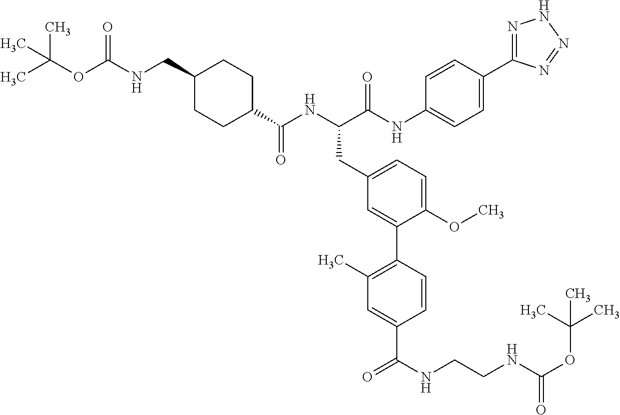

45 mg (0.12 mmol) of N-[(dimethylamino)(3H-[1,2,3]triazolo[4,5-b]pyridin-3-yloxy)methylidene]-N-methylmethanaminium hexafluorophosphate and 0.02 ml (0.12 mmol) of N,N-diisopropylethylamine were added to a solution of 70 mg (0.1 mmol) of 5′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-carboxylic acid and 19 mg (0.12 mmol) of tert-butyl (2-aminoethyl)carbamate in 3.5 ml of THF, and the mixture was stirred at RT for 16 h. The reaction mixture was separated directly by preparative HPLC (mobile phase: acetonitrile/water gradient, 0.1% trifluoroacetic acid)). This gave 45 mg of a mixture of the title compound and the corresponding deprotected amine, which was used directly in the next stage.

LC-MS (Method 1): Rₜ=1.07 min; MS (ESIneg): m/z=852 [M−H]⁻.

### Example 95A

### N-alpha-[(trans-4-{[(tert-Butoxycarbonyl)amino]methyl}cyclohexyl)carbonyl]-3-{6-[3-(dimethylamino)propoxy]pyridin-3-yl}-N-[4-(1H-tetrazol-5-yl)phenyl]-L-phenylalaninamide

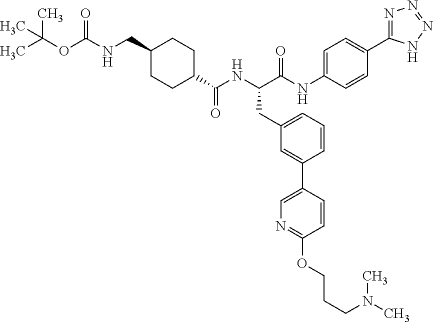

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and N,N-dimethyl-3-{[5-(4,4,5,5-tetramethyl-1,3,2-dioxaborolan-2-yl)pyridin-2-yl]oxy}propane-1-amine (110 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 10). This gave 92 mg (53% of theory) of the title compound.

LC-MS (Method 4): Rₜ=0.94 min; MS (ESIpos): m/z=726.5 [M+H]⁺.

### Example 96A

### tert-Butyl [(trans-4-{[(2S)-3-[5′-(benzylsulphamoyl)-2′-methylbiphenyl-3-yl]-1-oxo-1-{[4-(5-oxo-4,5-dihydro-1,2,4-oxadiazol-3-yl)phenyl]amino}propan-2-yl]carbamoyl}cyclohexyl)methyl]carbamate

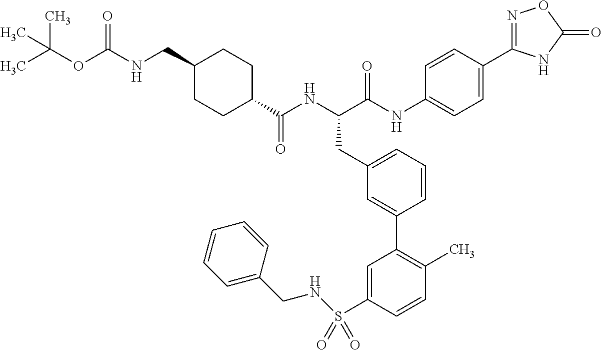 [SMILES: Cc1ccc(S(=O)(=O)NCc2ccccc2)cc1-c1cccc(C[C@H](NC(=O)[C@H]2CC[C@H](CNC(=O)OC(C)(C)C)CC2)C(=O)Nc2ccc(-c3noc(=O)[nH]3)cc2)c1]

Argon was passed through a solution of 150 mg (0.23 mmol) of 3-bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide and 106.9 mg (0.35 mmol) of [5-(benzylsulphamoyl)-2-methylphenyl]boronic acid in 2 ml of DMSO. Under argon, 27.0 mg (0.02 mmol) of tetrakis(triphenylphosphine)palladium(0), 74.2 mg (0.7 mmol) of sodium carbonate and 0.35 ml (19.5 mmol) of water were added. The mixture was treated in a microwave at 110° C. for 90 min. The mixture was filtered and purified by preparative HPLC (Method 10). This gave 36 mg (19% of theory) of the title compound.

¹H NMR (300 MHz, DMSO-d₆): δ=ppm 0.64-0.87 (m, 2H), 1.00-1.26 (m, 3H), 1.33 (s, 9H), 1.39-1.49 (m, 1H), 1.52-1.69 (m, 3H), 2.00-2.10 (m, 1H), 2.18 (s, 3H), 2.65-2.73 (m, 2H), 2.90 (dd, 1H), 3.07 (dd, 1H), 3.90-4.00 (m, 3H), 4.61-4.76 (m, 1H), 6.62-6.76 (m, 1H), 7.21 (m, 11H), 7.29-7.38 (m, 3H), 7.38-7.49 (m, 3H), 7.49-7.54 (m, 2H), 7.60-7.77 (m, 7H), 7.99-8.15 (m, 3H), 10.38 (s, 1H).

HPLC (Method 4): Rt=1.34 min; MS (ESIpos): m/z=823.4 [M+H]⁺.

### Example 97A

### tert-Butyl [(trans-4-{[(2S)-1-oxo-3-(3′-{[2-(pyrrolidin-1-yl)ethyl]carbamoyl}biphenyl-3-yl)-1-{[4-(1H-tetrazol-5-yl)phenyl]amino}propan-2-yl]carbamoyl}cyclohexyl)methyl]carbamate

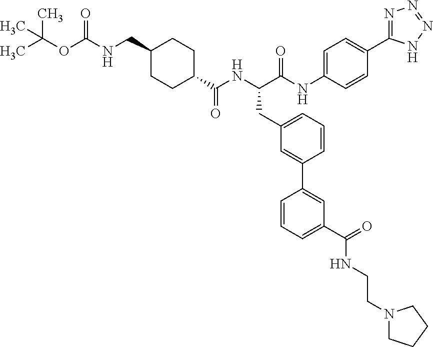 [SMILES: CC(C)(C)OC(=O)NC[C@H]1CC[C@H](C(=O)N[C@@H](Cc2cccc(-c3cccc(C(=O)NCCN4CCCC4)c3)c2)C(=O)Nc2ccc(-c3nnn[nH]3)cc2)CC1]

Argon was passed through a solution of 150 mg (0.24 mmol) of 3-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide and 107.2 mg (0.35 mmol) of (3-{[2-(pyrrolidin-1-yl)ethyl]carbamoyl}phenyl)boronic acid in 1.8 ml of DMSO. Under argon, 27.7 mg (0.02 mmol) of tetrakis(triphenylphosphine)palladium(0), 76.1 mg (0.72 mmol) of sodium carbonate and 0.36 ml (20.0 mmol) of water were added. The mixture was treated in a microwave at 110° C. for 2 h. The mixture was filtered and purified by preparative HPLC. 28 mg (15% of theory) of the title compound were obtained.

¹H NMR (400 MHz, DMSO-d₆): δ=ppm 0.69-0.84 (m, 2H), 1.04-1.13 (m, 1H), 1.14-1.25 (m, 2H), 1.33 (s, 9H), 1.47-1.59 (m, 2H), 1.60-1.69 (m, 2H), 1.82 (br. s., 4H), 2.05-2.14 (m, 1H), 2.67-2.73 (m, 2H), 2.89-2.97 (m, 2H), 2.99-3.11 (m, 6H), 3.49-3.55 (m, 2H), 4.66-4.73 (m, 1H), 6.71 (t, 1H), 7.26-7.32 (m, 1H), 7.37 (t, 1H), 7.47-7.54 (m, 2H), 7.59 (m, 3H), 7.67-7.81 (m, 3H), 7.85 (d, 2H), 8.05-8.11 (m, 2H), 8.66 (t, 1H), 10.13 (s, 1H).

HPLC (Method 4): Rₜ=0.94 min; MS (ESIpos): m/z=764.6 [M+H]⁺.

### Example 98A

### tert-Butyl [(trans-4-{[(2S)-3-[2′-fluoro-5′-(methylcarbamoyl)biphenyl-3-yl]-1-oxo-1-{[4-(1H-tetrazol-5-yl)phenyl]amino}propan-2-yl]carbamoyl}cyclohexyl)methyl]carbamate

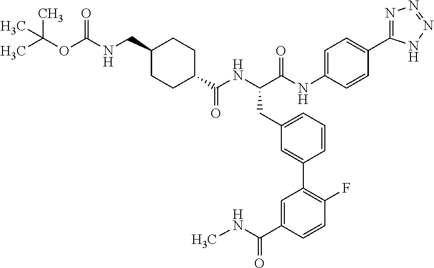 [SMILES: CNC(=O)c1ccc(F)c(-c2cccc(C[C@H](NC(=O)[C@H]3CC[C@H](CNC(=O)OC(C)(C)C)CC3)C(=O)Nc3ccc(-c4nnn[nH]4)cc3)c2)c1]

Argon was passed through a solution of 150 mg (0.24 mmol) of 3-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide and 70.7 mg (0.36 mmol) of [2-fluoro-5-(methylcarbamoyl)phenyl]boronic acid in 1.8 ml of DMSO. Under argon, 27.7 mg (0.02 mmol) of tetrakis(triphenylphosphine)palladium(0), 76.1 mg (0.72 mmol) of sodium carbonate and 0.36 ml (20.0 mmol) of water were added. The mixture was treated in a microwave at 110° C. for 90 min. The mixture was filtered and purified by preparative HPLC. 103 mg (62% of theory) of the title compound were obtained.

HPLC (Method 5): Rₜ=1.10 min; MS (ESIneg): m/z=697 [M−H]⁻.

### Example 99A

### tert-Butyl [(trans-4-{[(2S)-3-(2′-methyl-4′-{[(3R)-2-oxopiperidin-3-yl]carbamoyl}biphenyl-3-yl)-1-oxo-1-{[2-(pentafluoroethyl)-1H-benzimidazol-6-yl]amino}propan-2-yl]carbamoyl}cyclohexyl)methyl]carbamate

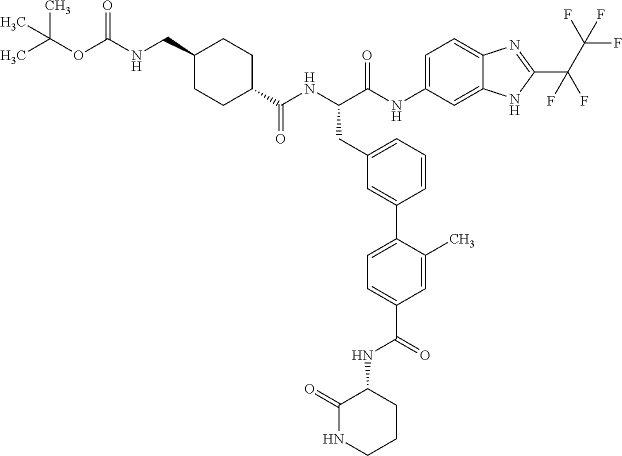 [SMILES: Cc1cc(C(=O)N[C@@H]2CCCNC2=O)ccc1-c1cccc(C[C@H](NC(=O)[C@H]2CC[C@H](CNC(=O)OC(C)(C)C)CC2)C(=O)Nc2ccc3nc(C(F)(F)C(F)(F)F)[nH]c3c2)c1]

75 μl (0.4 mmol) of N,N-diisopropylethylamine and 81.3 mg (0.21 mmol) of N-[(dimethylamino)(3H-[1,2,3]triazolo[4,5-b]pyridin-3-yloxy)methylidene]-N-methylmethanaminium hexafluorophosphate were added to a solution of 110 mg (0.14 mmol) of 3′-[(2S)-2-{[(trans-4-{[(tert-butoxycarbonyl)amino]methyl}cyclohexyl)carbonyl]amino}-3-oxo-3-{[2-(pentafluoroethyl)-1H-benzimidazol-6-yl]amino}propyl]-2-methylbiphenyl-4-carboxylic acid and 42.9 mg (0.29 mmol) of (3R)-3-aminopiperidin-2-one hydrochloride in 2.25 ml of DMF, and the mixture was stirred at RT for 16 h. The reaction mixture was filtered and separated by means of preparative HPLC (Method 11). 24 mg (19% of theory) of the title compound were obtained.

HPLC (Method 5): Rₜ=0.92 min; MS (ESIpos): m/z=868.5 [M+H]⁺.

### Example 100A

### tert-Butyl [(trans-4-{[(2S)-3-(4′-{[4-(dimethylamino)cyclohexy]carbamoyl}-2′-methylbiphenyl-3-yl)-1-{[3-fluoro-4-(1H-tetrazol-5-yl)phenyl]amino}-1-oxopropan-2-yl]carbamoyl}cyclohexyl)-methyl]carbamate

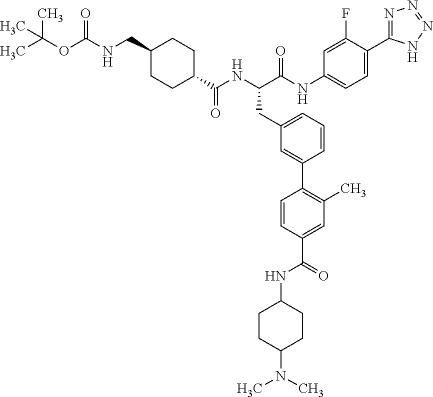 [SMILES: Cc1cc(C(=O)NC2CCC(N(C)C)CC2)ccc1-c1cccc(C[C@H](NC(=O)[C@H]2CC[C@H](CNC(=O)OC(C)(C)C)CC2)C(=O)Nc2ccc(-c3nnn[nH]3)c(F)c2)c1]

26.9 mg (0.02 mmol) of tetrakis(triphenylphosphine)palladium(0), 74.0 mg (0.70 mmol) of sodium carbonate and 0.35 ml (19.4 mmol) of water were added to a solution of 150 mg (0.23 mmol) of 3-bromo-N-alpha-[(trans-4-{[(tert-butoxycarbonyl)amino]methyl}cyclohexyl)carbonyl]-N-[3-fluoro-4-(1H-tetrazol-5-yl)phenyl]-L-phenylalaninamide and 354 mg (0.35 mmol) of (4-{[4-(dimethylamino)cyclohexyl]carbamoyl}-2-methylphenyl)boronic acid in 2 ml of DMSO. The mixture was treated in a microwave at 110° C. for 90 min. Another 0.1 eq. of (4-{[4-(dimethylamino)cyclohexyl]carbamoyl}-2-methylphenyl)boronic acid was added and the mixture was irradiated in a microwave at 110° C. for 90 min. The mixture was filtered and purified by preparative HPLC (Method 11). 9 mg (5% of theory) of the title compound were obtained.

HPLC (Method 5): Rₜ=0.85 min; MS (ESIpos): m/z=824.5 [M+H]⁺.

### Example 101A

### N-alpha-[(trans-4-{[(tert-Butoxycarbonyl)amino]methyl}cyclohexyl)carbonyl]-3-[2-(4-methylpiperazin-1-yl)pyridin-4-yl]-N-[4-(1H-tetrazol-5-yl)phenyl]-L-phenylalaninamide

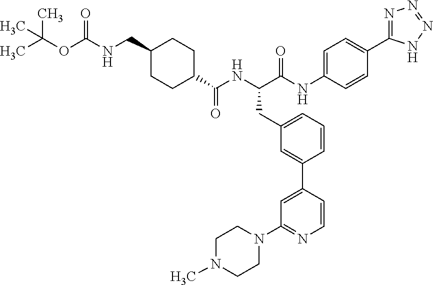

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 2-(4-methylpiperazin-1-yl)pyridine-4-boronic acid (109 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 10). This gave 52 mg (30% of theory) of the title compound.

LC-MS (Method 4): Rₜ=0.90 min; MS (ESIpos): m/z=723.2 [M+H]⁺.

### Example 102A

### 3-(6-Amino-2-methylpyridin-3-yl)-N-alpha-[(trans-4-{[(tert-butoxycarbonyl)amino]methyl}cyclohexyl)carbonyl]-N-[4-(1H-tetrazol-5-yl)phenyl]-L-phenylalaninamide

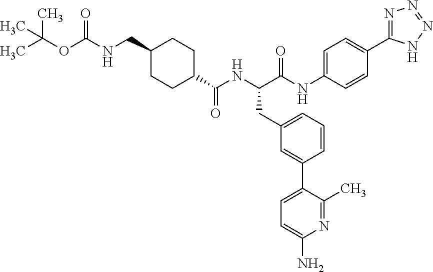

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 6-methyl-5-(4,4,5,5-tetramethyl-1,3,2-dioxaborolan-2-yl)pyridine-2-amine (62 mg, 0.26 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 60 min, cooled, filtered and purified by chromatography via HPLC (Method 10). This gave 58 mg (37% of theory) of the title compound.

LC-MS (Method 4): Rₜ=0.90 min; MS (ESIpos): m/z=654.2 [M+H]⁺.

### Example 103A

### tert-Butyl 4-[5-(3-{(2S)-2-{[(trans-4-{[(tert-butoxycarbonyl)amino]methyl}cyclohexyl)carbonyl]amino}-3-oxo-3-[(2-oxo-2,3-dihydro-1H-benzimidazol-5-yl)amino]propyl}phenyl)pyrimidin-2-yl]piperazine-1-carboxylate

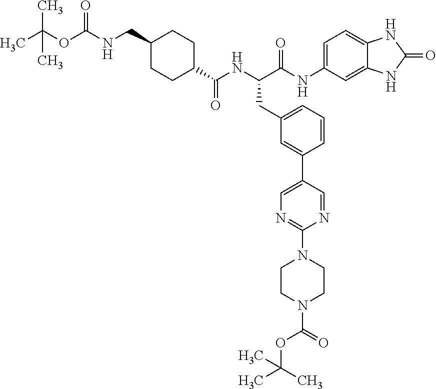

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and tert-butyl 4-[5-(4,4,5,5-tetramethyl-1,3,2-dioxaborolan-2-yl)pyrimidin-2-yl]piperazine-1-carboxylate (143 mg, 0.37 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 70 mg (36% of theory) of the title compound.

LC-MS (Method 4): Rₜ=1.29 min; MS (ESIpos): m/z=798.5 [M+H]⁺.

### Example 104A

### tert-Butyl 4-(5-{3-[(2S)-2-{[(trans-4-{[(tert-butoxycarbonyl)amino]methyl}cyclohexyl)carbonyl]-amino}-3-oxo-3-{[4-(1H-tetrazol-5-yl)phenyl]amino}propyl]phenyl}pyrimidin-2-yl)piperazine-1-carboxylate

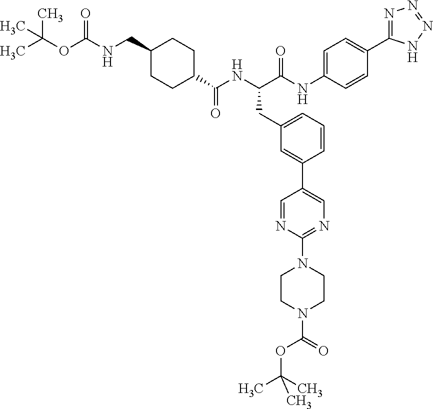

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and tert-butyl 4-[5-(4,4,5,5-tetramethyl-1,3,2-dioxaborolan-2-yl)pyrimidin-2-yl]piperazine-1-carboxylate (140 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 10). This gave 70 mg (36% of theory) of the title compound.

LC-MS (Method 4): Rₜ=1.39 min; MS (ESIpos): m/z=810.5 [M+H]⁺.

### Example 105A

### N-alpha-[(trans-4-{[(tert-Butoxycarbonyl)amino]methyl}cyclohexyl)carbonyl]-3-[2-(4-methylpiperazin-1-yl)pyrimidin-5-yl]-N-[4-(1H-tetrazol-5-yl)phenyl]-L-phenylalaninamide

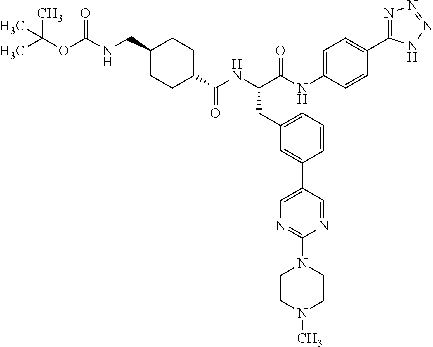

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (184 mg, 0.29 mmol) and 2-(4-methylpiperazin-1-yl)-5-(4,4,5,5-tetramethyl-1,3,2-dioxaborolan-2-yl)pyrimidine (98 mg, 0.32 mmol) were dissolved in dimethyl sulphoxide (2 ml), and tetrakis(triphenylphosphine)palladium(0) (34 mg, 29 μmol), sodium carbonate (93 mg, 0.88 mmol) and water (0.44 ml, 25 mmol) were added. The reaction mixture was stirred at 110° C. in a microwave (Biotage Initiator) for 120 min, cooled, filtered and purified by chromatography via HPLC (Method 10). This gave 86 mg (40% of theory) of the title compound.

LC-MS (Method 4): Rₜ=0.92 min; MS (ESIpos): m/z=724.3 [M+H]⁺.

### Example 106A

### N-alpha-[(trans-4-{[(tert-Butoxycarbonyl)amino]methyl}cyclohexyl)carbonyl]-3-[2-(dimethylamino)pyrimidin-5-yl]-N-[4-(1H-tetrazol-5-yl)phenyl]-L-phenylalaninamide

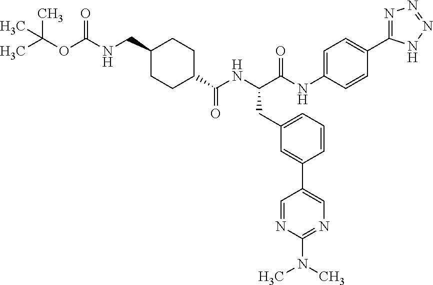

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (200 mg, 0.32 mmol) and N,N-dimethyl-5-(4,4,5,5-tetramethyl-1,3,2-dioxaborolan-2-yl)pyrimidine-2-amine (87 mg, 0.35 mmol) were dissolved in dimethylformamide (3 ml), and 1,1′-bis(diphenylphosphine)ferrocenedichloropalladium(II) (26 mg, 32 μmol), sodium carbonate (101 mg, 0.96 mmol) and water (0.48 ml, 27 mmol) were added. The reaction mixture was stirred at 110° C. in a microwave (Biotage Initiator) for 60 min. The reaction mixture was concentrated and converted further as the crude product.

LC-MS (Method 4): Rₜ=1.18 min; MS (ESIpos): m/z=669.4 [M+H]⁺.

### Example 107A

### N-alpha-[(trans-4-{[(tert-Butoxycarbonyl)amino]methyl}cyclohexyl)carbonyl]-3-[6-(dimethylamino)pyridin-3-yl]-N-[4-(1H-tetrazol-5-yl)phenyl]-L-phenylalaninamide

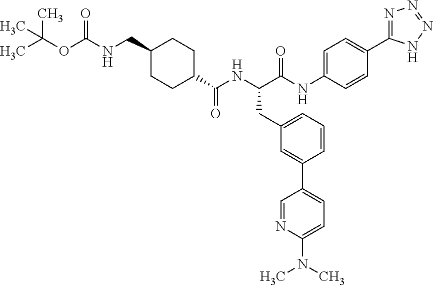

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [6-(dimethylamino)pyridin-3-yl]boronic acid (44 mg, 0.26 mmol) were dissolved in dimethylformamide (2 ml), and 1,1′-bis(diphenylphosphine)ferrocenedichloropalladium(II) (19.5 mg, 24 μmol), sodium carbonate (76 mg, 0.72 mmol) and water (0.36 ml, 20 mmol) were added. The reaction mixture was stirred at 110° C. in a microwave (Biotage Initiator) for 60 min and filtered through Celite, washing with methanol. This gave 168 mg of crude product. This was reacted further without further purification.

LC-MS (Method 4): Rₜ=0.92 min; MS (ESIpos): m/z=668.4 [M+H]⁺.

### Example 108A

### 3-(5-Aminopyridin-3-yl)-N-alpha-[(trans-4-{[(tert-butoxycarbonyl)amino]methyl}cyclohexyl)carbonyl]-N-[4-(1H-tetrazol-5-yl)phenyl]-L-phenylalaninamide

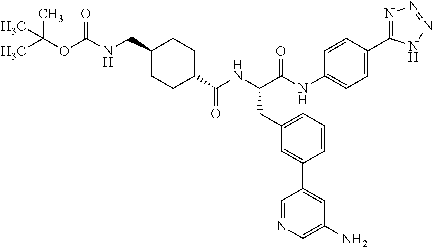

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 5-(4,4,5,5-tetramethyl-1,3,2-dioxaborolan-2-yl)pyridine-3-amine (63 mg, 0.29 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 10). This gave 29 mg (19% of theory) of the title compound.

LC-MS (Method 4): Rₜ=0.88 min; MS (ESIpos): m/z=640.2 [M+H]⁺.

### Example 109A

### N-alpha-[(trans-4-{[(tert-Butoxycarbonyl)amino]methyl}cyclohexyl)carbonyl]-3-[2-(dimethylamino)pyrimidin-5-yl]-N-(2-oxo-2,3-dihydro-1H-benzimidazol-5-yl)-L-phenylalaninamide

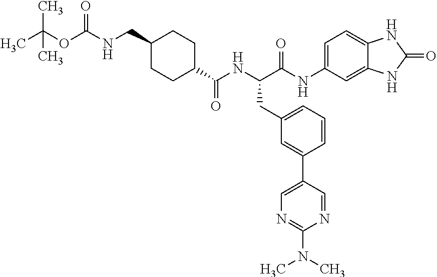

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and N,N-dimethyl-5-(4,4,5,5-tetramethyl-1,3,2-dioxaborolan-2-yl)pyrimidine-2-amine (91 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 47 mg (29% of theory) of the title compound.

LC-MS (Method 4): Rₜ=1.11 min; MS (ESIpos): m/z=657.4 [M+H]⁺.

### Example 110A

### N-alpha-[(trans-4-{[(tert-Butoxycarbonyl)amino]methyl}cyclohexyl)carbonyl]-3-[2-(morpholin-4-yl)pyrimidin-5-yl]-N-[4-(1H-tetrazol-5-yl)phenyl]-L-phenylalaninamide

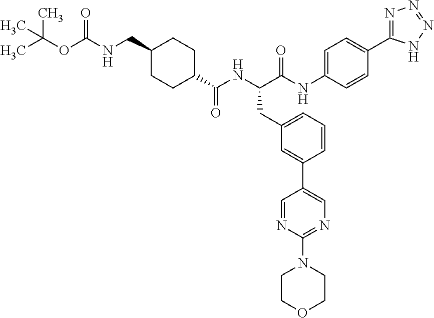

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 4-[5-(4,4,5,5-tetramethyl-1,3,2-dioxaborolan-2-yl)pyrimidin-2-yl]morpholine (111 mg, 0.38 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 150 min and reacted further as crude product.

LC-MS (Method 4): Rₜ=1.15 min; MS (ESIpos): m/z=711.4 [M+H]⁺.

### Example 111A

### N-alpha-[(trans-4-{[(tert-Butoxycarbonyl)amino]methyl}cyclohexyl)carbonyl]-3-(3-methylpyridin-4-yl)-N-(2-oxo-2,3-dihydro-1H-benzimidazol-5-yl)-L-phenylalaninamide

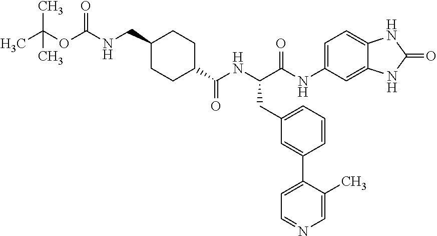

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and 3-methyl-4-(4,4,5,5-tetramethyl-1,3,2-dioxaborolan-2-yl)pyridine (80 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 51 mg (33% of theory) of the title compound.

LC-MS (Method 4): Rₜ=0.84 min; MS (ESIpos): m/z=627.3 [M+H]⁺.

### Example 112A

### N-alpha-[(trans-4-{[(tert-Butoxycarbonyl)amino]methyl}cyclohexyl)carbonyl]-3-[2-(4-methylpiperazin-1-yl)pyridin-4-yl]-N-(2-oxo-2,3-dihydro-1H-benzimidazol-5-yl)-L-phenylalaninamide

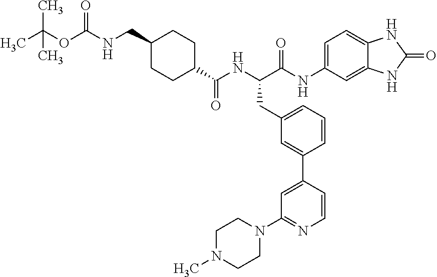

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and 1-methyl-4-[4-(4,4,5,5-tetramethyl-1,3,2-dioxaborolan-2-yl)pyridin-2-yl]piperazine (111 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 98 mg (57% of theory) of the title compound.

LC-MS (Method 4): Rₜ=0.82 min; MS (ESIpos): m/z=711.4 [M+H]⁺.

### Example 113A

### N-alpha-[(trans-4-{[(tert-Butoxycarbonyl)amino]methyl}cyclohexyl)carbonyl]-3-[6-(4-methylpiperazin-1-yl)pyridin-3-yl]-N-[4-(1H-tetrazol-5-yl)phenyl]-L-phenylalaninamide

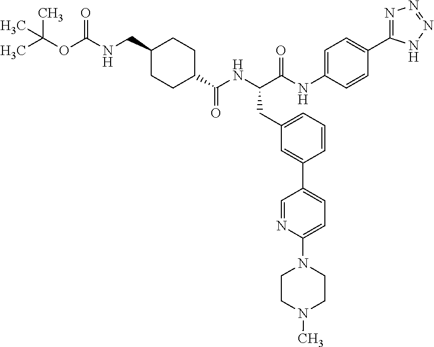

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 1-methyl-4-[5-(4,4,5,5-tetramethyl-1,3,2-dioxaborolan-2-yl)pyridin-2-yl]piperazine (109 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 70 mg (40% of theory) of the title compound.

LC-MS (Method 4): Rₜ=0.89 min; MS (ESIpos): m/z=723.2 [M+H]⁺.

### Example 114A

### 3-[4-(Benzyloxy)-2-(dimethylamino)pyrimidin-5-yl]-N-alpha-[(trans-4-{[(tert-butoxycarbonyl)amino]methyl}cyclohexyl)carbonyl]-N-[4-(1H-tetrazol-5-yl)phenyl]-L-phenylalaninamide

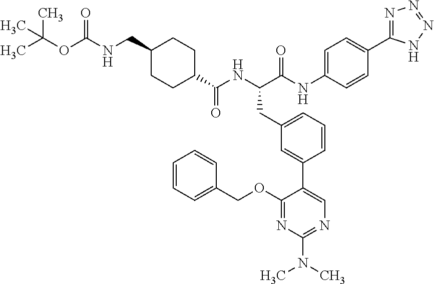

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [4-(benzyloxy)-2-(dimethylamino)pyrimidin-5-yl]boronic acid (98 mg, 0.36 mmol) were dissolved in dimethyl sulphoxide (2 ml), and 1,1′-bis(diphenylphosphine)ferrocenedichloropalladium(II) (19.5 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 73 mg (39% of theory) of the title compound.

LC-MS (Method 4): Rₜ=1.24 min; MS (ESIpos): m/z=775.5 [M+H]⁺.

### Example 115A

### 3-(2-Aminopyrimidin-5-yl)-N-alpha-[(trans-4-{[(tert-butoxycarbonyl)amino]methyl}cyclohexyl)carbonyl]-N-[4-(1H-tetrazol-5-yl)phenyl]-L-phenylalaninamide

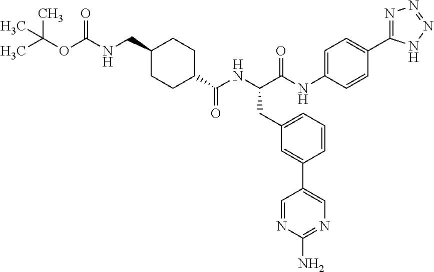

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (200 mg, 0.32 mmol) and 5-(4,4,5,5-tetramethyl-1,3,2-dioxaborolan-2-yl)pyrimidine-2-amine (78 mg, 0.35 mmol) were dissolved in dimethylformamide (3 ml), and 1,1′-bis(diphenylphosphine)ferrocenedichloropalladium(II) (26 mg, 32 μmol), sodium carbonate (101 mg, 0.96 mmol) and water (0.48 ml, 27 mmol) were added. The reaction mixture was stirred at 110° C. in a microwave (Biotage Initiator) for 240 min. 0.5 ml of 1M hydrochloric acid was added and the reaction mixture was filtered through a cartridge, concentrated and converted further as the crude product.

LC-MS (Method 4): Rₜ=0.98 min; MS (ESIpos): m/z=641.3 [M+H]⁺.

### Example 116A

### N-alpha-[(trans-4-{[(tert-Butoxycarbonyl)amino]methyl}cyclohexyl)carbonyl]-3-(3-methylpyridin-4-yl)-N-[4-(1H-tetrazol-5-yl)phenyl]-L-phenylalaninamide

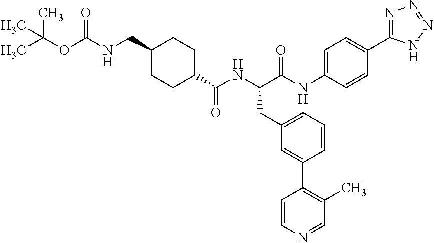

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 3-methyl-4-(4,4,5,5-tetramethyl-1,3,2-dioxaborolan-2-yl)pyridine (84 mg, 0.54 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 150 min and reacted further as crude product.

LC-MS (Method 4): Rₜ=0.89 min; MS (ESIpos): m/z=639.3 [M+H]⁺.

### Example 117A

### N-alpha-[(trans-4-{[(tert-Butoxycarbonyl)amino]methyl}cyclohexyl)carbonyl]-N-[4-(1H-tetrazol-5-yl)phenyl]-3-[2-(trifluoromethyl)pyridin-3-yl]-L-phenylalaninamide

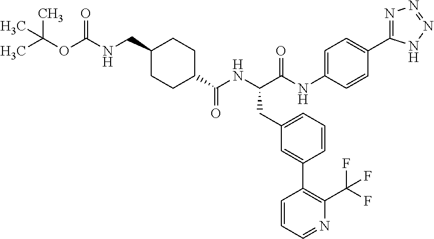

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 3-(4,4,5,5-tetramethyl-1,3,2-dioxaborolan-2-yl)-2-(trifluoromethyl)pyridine (98 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 24 mg (15% of theory) of the title compound.

LC-MS (Method 4): Rₜ=1.25 min; MS (ESIpos): m/z=693.3 [M+H]⁺.

### Example 118A

### N-alpha-[(trans-4-{[(tert-Butoxycarbonyl)amino]methyl}cyclohexyl)carbonyl]-3-[2-(cyclopropylamino)pyrimidin-5-yl]-N-[4-(1H-tetrazol-5-yl)phenyl]-L-phenylalaninamide

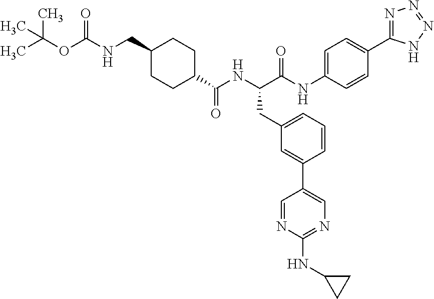

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and N-cyclopropyl-5-(4,4,5,5-tetramethyl-1,3,2-dioxaborolan-2-yl)pyrimidine-2-amine (94 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 44 mg (27% of theory) of the title compound.

LC-MS (Method 4): Rₜ=1.11 min; MS (ESIpos): m/z=681.4 [M+H]⁺.

### Example 119A

### N-alpha-[(trans-4-{[(tert-Butoxycarbonyl)amino]methyhyl}cyclohexyl)carbonyl]-3-(5-methyl-1H-indazol-4-yl)-N-[4-(1H-tetrazol-5-yl)phenyl]-L-phenylalaninamide

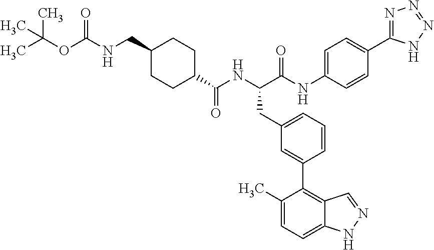

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (5-methyl-1H-indazol-4-yl)boronic acid (63 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 9). This gave 39 mg (24% of theory) of the title compound.

LC-MS (Method 4): Rₜ=1.18 min; MS (ESIpos): m/z=678.4 [M+H]⁺.

### Example 120A

### tert-Butyl {[trans-4-({(2S)-1-oxo-1-{[4-(1H-tetrazol-5-yl)phenyl]amino}-3-[2′-(trifluoromethyl)biphenyl-3-yl]propan-2-yl}carbamoyl)cyclohexyl]methyl}carbamate

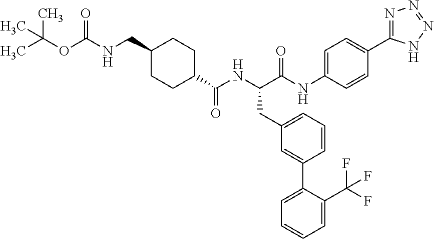

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (200 mg, 0.32 mmol) und 4,4,5,5-tetramethyl-2-[2-(trifluoromethyl)phenyl]-1,3,2-dioxaborolane (138 mg, 0.51 mmol) were dissolved in dimethylformamide (3 ml), and 1,1′-bis(diphenylphosphine)ferrocenedichloropalladium(II) (26 mg, 32 μmol), sodium carbonate (101 mg, 0.96 mmol) and water (0.48 ml, 27 mmol) were added. The reaction mixture was stirred at 110° C. in a microwave (Biotage Initiator) for 120 min. 0.5 ml of 1N hydrochloric acid was added and the reaction mixture was concentrated and converted further as the crude product.

LC-MS (Method 4): Rₜ=1.22 min; MS (ESIpos): m/z=693.3 [M+H]⁺.

### Example 121A

### 3-Bromo-N-alpha-[(trans-4-{[(tert-butoxycarbonyl)amino]methyl}cyclohexyl)carbonyl]-N-{4-[3-(methoxymethyl)-4H-1,2,4-triazol-5-yl]phenyl}-L-phenylalaninamide

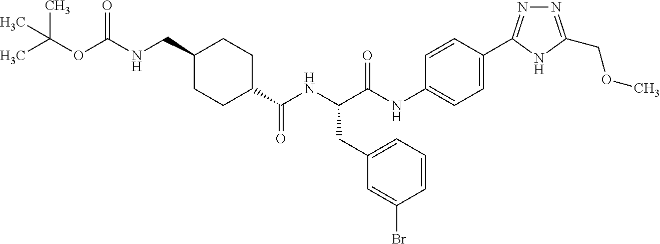

3-Bromo-N-[(trans-4-{[(tert-butoxycarbonyl)amino]methyl}cyclohexyl)carbonyl]-L-phenylalanine (10 g, 20.7 mmol), 4-[3-(methoxymethyl)-4H-1,2,4-triazol-5-yl]aniline hydrochloride (5.0 g, 20.7 mmol) and N,N-diisopropylethylamine (14.4 ml, 7.6 mmol) were suspended in 100 ml of ethyl acetate, and 2,4,6-tripropyl-1,3,5,2,4,6-trioxatriphosphinane 2,4,6-trioxide (50% in ethyl acetate, 31 ml, 33 g, 52 mmol) was added. The reaction mixture was heated under reflux for 3 h, water was added and the phases were separated. The aqueous phase was extracted twice with ethyl acetate. The combined organic phases were washed with saturated aqueous ammonium chloride solution and saturated aqueous sodium chloride solution. This resulted in the precipitation of a solid. The solid was filtered off with suction and dried under high vacuum. This gave 2.86 g (21% of theory) of the title compound.

¹H NMR (400 MHz, DMSO-d₆): δ=ppm 0.83 (m, 2H), 1.05-1.31 (m, 3H), 1.37 (s, 9H), 1.53-1.62 (m, 1H), 1.68 (m, 3H), 2.04-2.17 (m, 1H), 2.75 (t, 2H), 2.81-2.91 (m, 1H), 3.04 (m, 1H), 3.35 (s, 3H), 4.51 (s, 2H), 4.62-4.72 (m, 1H), 6.69-6.81 (m, 1H), 7.20-7.27 (m, 1H), 7.29-7.33 (m, 1H), 7.39 (d, 1H), 7.53 (s, 1H), 7.70 (d, 2H), 7.94 (d, 2H), 8.11 (d, 1H), 10.28 (s, 1H), 14.10 (br. s, 1H).

LC-MS (Method 4): Rₜ=1.23 min; MS (ESIpos): m/z=671.4 [M+H]⁺.

### Example 122A

### tert-Butyl [(trans-4-{[(2S)-3-[5′-(acetylamino)-2′-chlorobiphenyl-3-yl]-1-oxo-1-{[4-(1H-tetrazol-5-yl)phenyl]amino}propan-2-yl]carbamoyl}cyclohexyl)methyl]carbamate

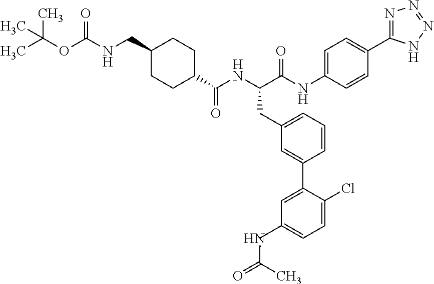

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and N-[4-chloro-3-(4,4,5,5-tetramethyl-1,3,2-dioxaborolan-2-yl)phenyl]acetamide (106 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 90 min, cooled, filtered and purified by chromatography via HPLC (Method 10). This gave 73 mg (42% of theory) of the title compound.

LC-MS (Method 4): Rₜ=1.21 min; MS (ESIpos): m/z=715.3 [M+H]⁺.

### Example 123A

### N-alpha-[(trans-4-{[(tert-Butoxycarbonyl)amino]methyl}cyclohexyl)carbonyl]-3-(5-methyl-1H-indazol-4-yl)-N-[4-(5-oxo-4, 5-dihydro-1,2,4-oxadiazol-3-yl)phenyl]-L-phenylalaninamide

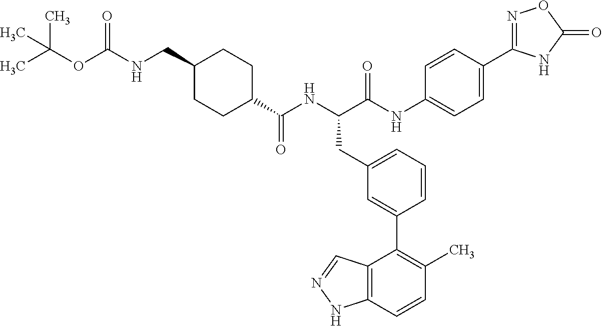

3-Bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide (150 mg, 0.24 mmol) and (5-methyl-1H-indazol-4-yl)boronic acid (62 mg, 0.35 mmol) were dissolved in dimethyl sulphoxide (2 ml), and tetrakis(triphenylphosphine)palladium(0) (27 mg, 23 μmol), sodium carbonate (74 mg, 0.70 mmol) and water (0.35 ml, 19.5 mmol) were added. The reaction mixture was stirred at 110° C. in a microwave (Biotage Initiator) for 150 min, cooled, filtered and purified by chromatography via HPLC (Method 10). This gave 47 mg (29% of theory) of the title compound.

LC-MS (Method 4): Rₜ=1.19 min; MS (ESIpos): m/z=694.4 [M+H]⁺.

### Example 124A

### N-alpha-[(trans-4-{[(tert-Butoxycarbonyl)amino]methyl}cyclohexyl)carbonyl]-3-[2-(morpholin-4-yl)pyrimidin-5-yl]-N-[4-(5-oxo-4,5-dihydro-1H-1,2,4-triazol-3-yl)phenyl]-L-phenylalaninamide

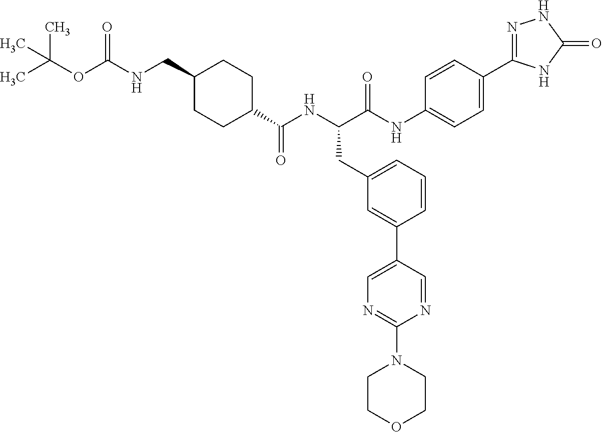

3-Bromo-N-alpha-[(trans-4-{[(tert-butoxycarbonyl)amino]methyl}cyclohexyl)carbonyl]-N-[4-(5-oxo-4,5-dihydro-1H-1,2,4-triazol-3-yl)phenyl]-L-phenylalaninamide (150 mg, 0.23 mmol) and 4-[5-(4,4,5,5-tetramethyl-1,3,2-dioxaborolan-2-yl)pyrimidin-2-yl]morpholine (107 mg, 0.35 mmol) were dissolved in dimethyl sulphoxide (2 ml), and tetrakis(triphenylphosphine)palladium(0) (27 mg, 23 gmol), sodium carbonate (74 mg, 0.70 mmol) and water (0.35 ml, 19.5 mmol) were added. The reaction mixture was stirred at 110° C. in a microwave (Biotage Initiator) for 90 min, cooled, filtered and purified by chromatography via HPLC (Method 9). This gave 15 mg (9% of theory) of the title compound.

LC-MS (Method 4): Rₜ=1.19 min; MS (ESIpos): m/z=726.4 [M+H]⁺.

### Example 125A

### N-alpha-[(trans-4-{[(tert-Butoxycarbonyl)amino]methyl}cyclohexyl)carbonyl]-3-(3-methylpyridin-4-yl)-N-[4-(5-oxo-4,5-dihydro-1H-1,2,4-triazol-3-yl)phenyl]-L-phenylalaninamide

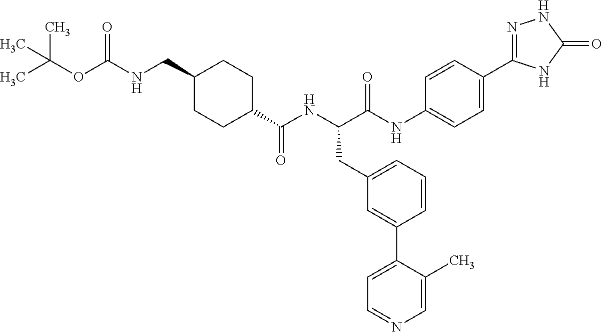

3-Brom-N-alpha-[(trans-4-{[(tert-butoxycarbonyl)amino]methyl}cyclohexyl)carbonyl]-N-[4-(5-oxo-4,5-dihydro-1H-1,2,4-triazol-3-yl)phenyl]-L-phenylalaninamid (150 mg, 0.23 mmol) und 3-methyl-4-(4,4,5,5-tetramethyl-1,3,2-dioxaborolan-2-yl)pyridine (76 mg, 0.35 mmol) were dissolved in dimethyl sulphoxide (2 ml), and tetrakis(triphenylphosphine)palladium(0) (27 mg, 23 μmol), sodium carbonate (74 mg, 0.70 mmol) and water (0.35 ml, 19.5 mmol) were added. The reaction mixture was stirred at 110° C. in a microwave (Biotage Initiator) for 90 min, cooled, filtered and purified by chromatography via HPLC (Method 9). This gave 59 mg (39% of theory) of the title compound.

LC-MS (Method 4): Rₜ=0.99 min; MS (ESIpos): m/z=654.4 [M+H]⁺.

### Example 126A

### N-alpha-[(trans-4-{[(tert-Butoxycarbonyl)amino]methyl}cyclohexyl)carbonyl]-N-[4-(3-chloro-4H-1,2,4-triazol-5-yl)phenyl]-3-{6-[3-(dimethylamino)propoxy]pyridin-3-yl}-L-phenylalaninamide

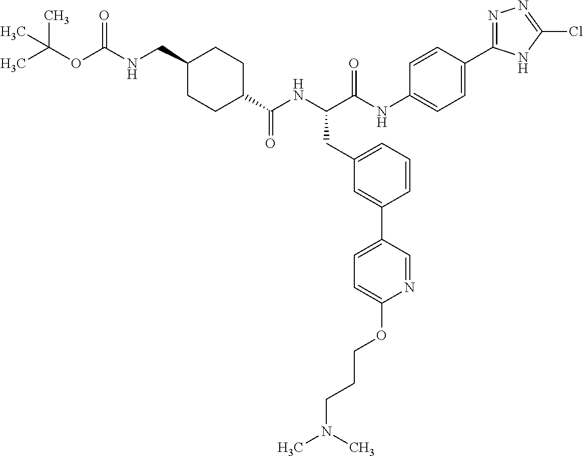

N-[(trans-4-{[(tert-Butoxycarbonyl)amino]methyl}cyclohexyl)carbonyl]-3-{6-[3-(dimethylamino)propoxy]pyridin-3-yl}-L-phenylalanine (150 mg, 0.26 mmol), 4-(3-chloro-4H-1,2,4-triazol-5-yl)aniline (110 mg, 50%, 0.28 mmol) and triethylamine (0.09 ml, 0.64 mmol) were suspended in 2.5 ml of ethyl acetate, and 2,4,6-tripropyl-1,3,5,2,4,6-trioxatriphosphinane 2,4,6-trioxide (50% in ethyl acetate, 0.3 ml, 0.5 mmol) was added. The reaction mixture was then heated under reflux for 2 h and stirred at RT overnight. Water was added to the reaction mixture, and the precipitated solid was filtered off with suction and purified chromatographically by HPLC (Method 8). This gave 81 mg (41% of theory) of the title compound.

LC-MS (Method 4): Rₜ=1.04 min; MS (ESIpos): m/z=759.5 [M+H]⁺.

### Example 127A

### N-alpha-[(trans-4-{[(tert-Butoxycarbonyl)amino]methyl}cyclohexyl)carbonyl]-N-[4-(3-chloro-4H-1,2,4-triazol-5-yl)phenyl]-3-[2-(4-methylpiperazin-1-yl)pyrimidin-5-yl]-L-phenylalaninamide

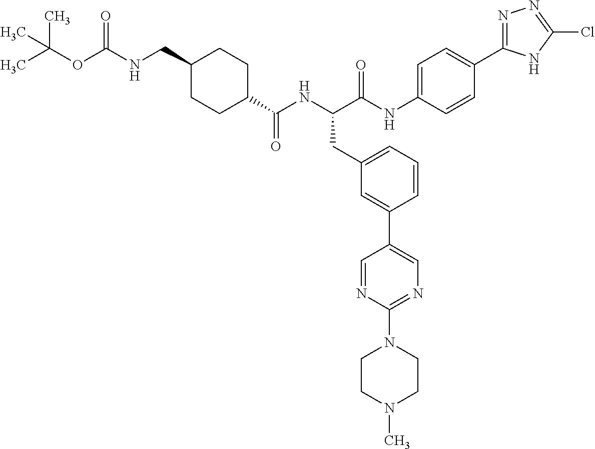

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and 2-(4-methylpiperazin-1-yl)-5-(4,4,5,5-tetramethyl-1,3,2-dioxaborolan-2-yl)pyrimidine (104 mg, 0.34 mmol) were dissolved in dimethyl sulphoxide (2 ml), and tetrakis(triphenylphosphine)palladium(0) (26 mg, 23 μmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified by chromatography via HPLC (Method 11). This gave 52 mg (30% of theory) of the title compound.

LC-MS (Method 5): Rₜ=0.85 min; MS (ESIpos): m/z=757.6 [M+H]⁺.

### Example 128A

### N-alpha-[(trans-4-{[(tert-Butoxycarbonyl)amino]methyl}cyclohexyl)carbonyl]-N-[4-(3-chloro-4H-1,2,4-triazol-5-yl)phenyl]-3-[2-(morpholin-4-yl)pyrimidin-5-yl]-L-phenylalaninamide

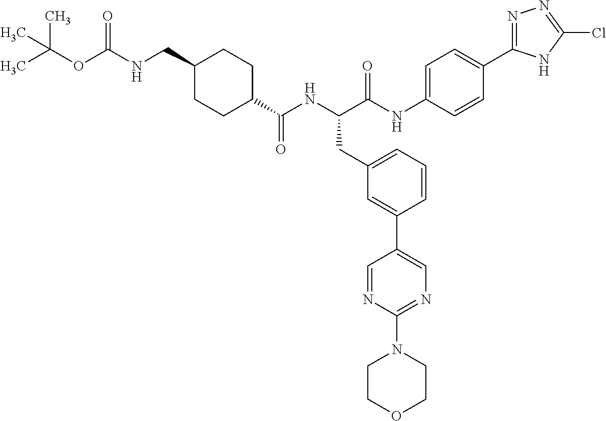

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and 4-[5-(4,4,5,5-tetramethyl-1,3,2-dioxaborolan-2-yl)pyrimidin-2-yl]morpholine (99 mg, 0.34 mmol) were dissolved in dimethyl sulphoxide (2 ml), and tetrakis(triphenylphosphine)palladium(0) (26 mg, 23 μmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified by chromatography via HPLC (Method 10). This gave 43 mg (25% of theory) of the title compound.

LC-MS (Method 4): Rₜ=1.26 min; MS (ESIpos): m/z=744.5 [M+H]⁺.

### Example 129A

### N-alpha-[(trans-4-{[(tert-Butoxycarbonyl)amino]methyl}cyclohexyl)carbonyl]-N-[4-(3-chloro-4H-1,2,4-triazol-5-yl)phenyl]-3-[2-(dimethylamino)pyrimidin-5-yl]-L-phenylalaninamide

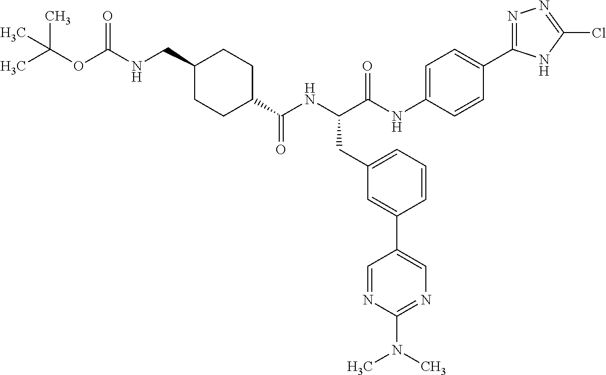

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and N,N-dimethyl-5-(4,4,5,5-tetramethyl-1,3,2-dioxaborolan-2-yl)pyrimidine-2-amine (85 mg, 0.34 mmol) were dissolved in dimethyl sulphoxide (2 ml), and tetrakis(triphenylphosphine)palladium(0) (26 mg, 23 gmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified by chromatography via HPLC (Method 10). This gave 74 mg (47% of theory) of the title compound.

LC-MS (Method 4): Rₜ=1.27 min; MS (ESIpos): m/z=702.5 [M+H]⁺.

### Example 130A

### 3-[2-(Acetylamino)-4-methylpyrimidin-5-yl]-N-alpha-[(trans-4-{[(tert-butoxycarbonyl)amino]methyl}cyclohexyl)carbonyl]-N-[4-(1H-tetrazol-5-yl)phenyl]-L-phenylalaninamide

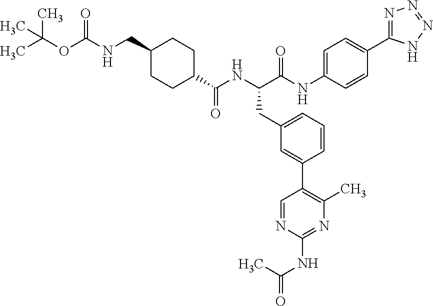

N-alpha-[(trans-4-{[(tert-Butoxycarbonyl)amino]methyl}cyclohexyl)carbonyl]-3-(4,4,5,5-tetramethyl-1,3,2-dioxaborolan-2-yl)-N-[4-(1H-tetrazol-5-yl)phenyl]-L-phenylalaninamide (179 mg, 0.26 mmol, crude product) and N-(5-bromo-4-methylpyrimidin-2-yl)acetamide (92 mg, 0.4 mmol) were dissolved in dimethyl sulphoxide (2 ml), and tetrakis(triphenylphosphine)palladium(0) (31 mg, 26 μmol), sodium carbonate (141 mg, 1.3 mmol) and water (0.4 ml, 22 mmol) were added. The reaction mixture was stirred at 110° C. in a microwave (Biotage Initiator) for 120 min, cooled, filtered and purified by chromatography via HPLC (Method 11). This gave 25 mg (14% of theory) of the title compound.

LC-MS (Method 5): Rₜ=0.74 min; MS (ESIpos): m/z=697.5 [M+H]⁺.

### Example 131A

### 3-(2-Amino-4-methylpyrimidin-5-yl)-N-alpha-[(trans-4-{[(tert-butoxycarbonyl)amino]methyl}-cyclohexyl)carbonyl]-N-[4-(1H-tetrazol-5-yl)phenyl]-L-phenylalaninamide

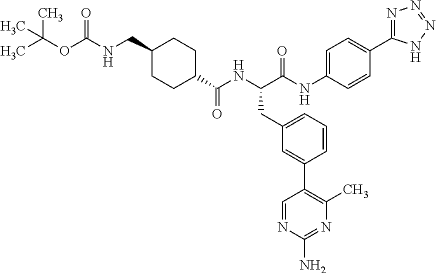

N-alpha-[(trans-4-{[(tert-Butoxycarbonyl)amino]methyl}cyclohexyl)carbonyl]-3-(4,4,5,5-tetramethyl-1,3,2-dioxaborolan-2-yl)-N-[4-(1H-tetrazol-5-yl)phenyl]-L-phenylalaninamide (179 mg, 0.26 mmol, crude product) and 5-bromo-4-methylpyrimidine-2-amine (75 mg, 0.4 mmol) were dissolved in dimethyl sulphoxide (2 ml), and tetrakis(triphenylphosphine)palladium(0) (31 mg, 26 μmol), sodium carbonate (141 mg, 1.3 mmol) and water (0.4 ml, 22 mmol) were added. The reaction mixture was stirred at 110° C. in a microwave (Biotage Initiator) for 120 min, cooled, filtered and purified by chromatography via HPLC (Method 11). This gave 17 mg (10% of theory) of the title compound.

LC-MS (Method 5): Rₜ=0.74 min; MS (ESIpos): m/z=655.5 [M+H]⁺.

### Example 132A

### 3-{3-[4-({N-[(trans-4-{[(tert-Butoxycarbonyl)amino]methyl}cyclohexyl)carbonyl]-3-[2-(morpholin-4-yl)pyrimidin-5-yl]-L-phenylalanyl}amino)phenyl]-1H-1,2,4-triazol-5-yl}-2,2,3,3-tetrafluoropropanoic acid

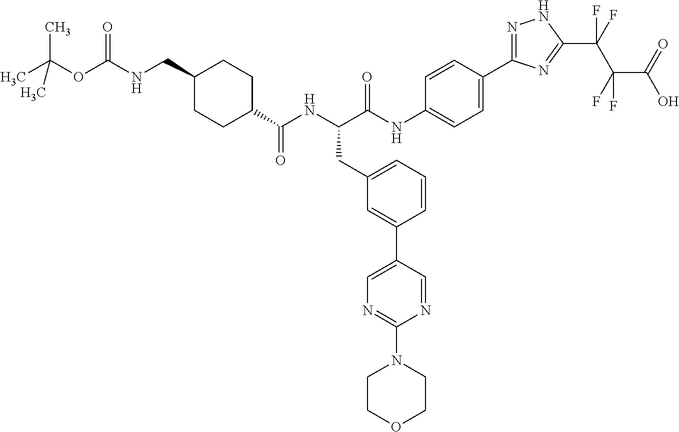

N-[(trans-4-{[(tert-Butoxycarbonyl)amino]methyl}cyclohexyl)carbonyl]-3-[2-(morpholin-4-yl)-pyrimidin-5-yl]-L-phenylalanine (58 mg, 0.1 mmol), 3-[3-(4-aminophenyl)-1H-1,2,4-triazol-5-yl]-2,2,3,3-tetrafluoropropanoic acid (35 mg, 0.11 mmol) and N,N-diisopropylethylamine (54 μl, 0.31 mmol) were dissolved in 1.5 ml of ethyl acetate, and 2,4,6-tripropyl-1,3,5,2,4,6-trioxatriphosphinane 2,4,6-trioxide (50% in ethyl acetate, 165 mg, 0.26 mmol) was added. The reaction mixture was heated under reflux for 3 h. After addition of 3-[3-(4-aminophenyl)-1H-1,2,4-triazol-5-yl]-2,2,3,3-tetrafluoropropanoic acid (35 mg, 0.11 mmol), N,N-diisopropylethylamine (54 μl, 0.31 mmol) and 2,4,6-tripropyl-1,3,5,2,4,6-trioxatriphosphinane 2,4,6-trioxide (50% in ethyl acetate, 165 mg, 0.26 mmol), the mixture was heated under reflux for another 17 h. Water was added to the reaction mixture, and the phases were separated. The aqueous phase was extracted twice with ethyl acetate. The combined organic phases were washed with saturated aqueous ammonium chloride solution and saturated aqueous sodium chloride solution, dried over sodium sulphate, filtered off and concentrated. This gave 78 mg (88% of theory) of the title compound.

LC-MS (Method 4): Rₜ=1.10 min; MS (ESIpos): m/z=854.4 [M+H]⁺.

### Example 133A

### tert-Butyl [(trans-4-{[(2S)-3-(4′-amino-2′-methylbiphenyl-3-yl)-1-oxo-1-{[4-(1H-tetrazol-5-yl)phenyl]amino}propan-2-yl]carbamoyl}cyclohexyl)methyl]carbamate

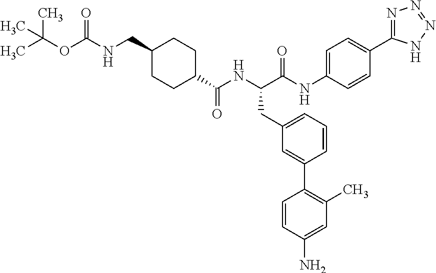

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and 4-amino-2-methylphenylboronic acid (61 mg, 0.26 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 150 min and reacted further as crude product.

LC-MS (Method 4): Rₜ=1.07 min; MS (ESIpos): m/z=653.4 [M+H]⁺.

### Example 134A

### N-alpha-[(trans-4-{[(tert-Butoxycarbonyl)amino]methyl}cyclohexyl)carbonyl]-3-(6-hydroxy-2-methylpyridin-3-yl)-N-[4-(1H-tetrazol-5-yl)phenyl]-L-phenylalaninamide

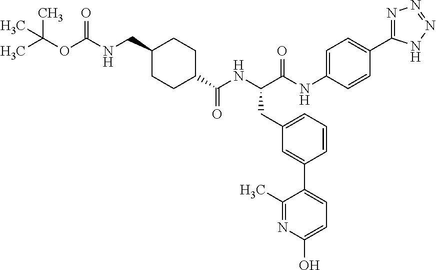

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (1.0 g, 1.6 mmol) and 6-methyl-5-(4,4,5,5-tetramethyl-1,3,2-dioxaborolan-2-yl)pyridin-2-ol (563 mg, 2.4 mmol) were dissolved in dimethyl sulphoxide (12 ml), and tetrakis(triphenylphosphine)palladium(0) (184 mg, 0.16 mmol), sodium carbonate (507 mg, 4.8 mmol) and water (2.4 ml, 133 mmol) were added. The reaction mixture was stirred at 110° C. in a microwave (Biotage Initiator) for 90 min, cooled, filtered and purified by chromatography via HPLC (Method 8). This gave 687 mg (66% of theory) of the title compound.

LC-MS (Method 5): Rₜ=1.03 min; MS (ESIpos): m/z=655.6 [M+H]⁺.

### Example 135A

### N-alpha-[(trans-4-{[(tert-Butoxycarbonyl)amino]methyl}cyclohexyl)carbonyl]-3-(2-hydroxypyrimidin-5-yl)-N-[4-(1H-tetrazol-5-yl)phenyl]-L-phenylalaninamide

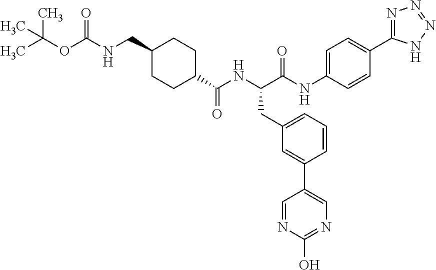

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (1.0 g, 1.6 mmol) and (2-hydroxypyrimidin-5-yl)boronic acid (335 mg, 2.4 mmol) were dissolved in dimethyl sulphoxide (12 ml), and tetrakis(triphenylphosphine)palladium(0) (184 mg, 0.16 mmol), sodium carbonate (507 mg, 4.8 mmol) and water (2.4 ml, 133 mmol) were added. The reaction mixture was stirred at 110° C. in a microwave (Biotage Initiator) for 120 min, cooled, filtered and purified by chromatography via HPLC (Method 17). This gave 315 mg (31% of theory) of the title compound.

LC-MS (Method 4): Rₜ=0.92 min; MS (ESIpos): m/z=642.4 [M+H]⁺.

### Example 136A

### N-alpha-[(trans-4-{[(tert-Butoxycarbonyl)amino]methyl}cyclohexyl)carbonyl]-3-{6-[3-(dimethylamino)propoxy]pyridin-3-yl}-N-(2-oxo-2,3-dihydro-1H-benzimidazol-5-yl)-L-phenylalaninamide

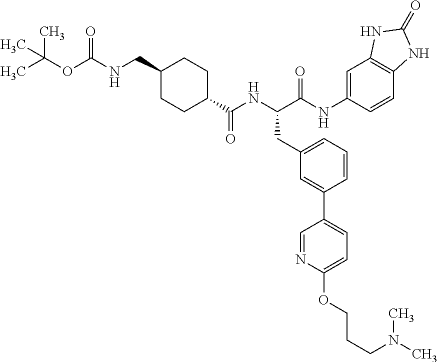 [SMILES: CN(C)CCCOc1ccc(-c2cccc(C[C@H](NC(=O)[C@H]3CC[C@H](CNC(=O)OC(C)(C)C)CC3)C(=O)Nc3ccc4[nH]c(=O)[nH]c4c3)c2)cn1]

3-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and N,N-dimethyl-3-{[5-(4,4,5,5-tetramethyl-1,3,2-dioxaborolan-2-yl)pyridin-2-yl]oxy}propane-1-amine (112 mg, 0.36 mmol) were dissolved in dimethyl sulphoxide (2 ml), and tetrakis(triphenylphosphine)palladium(0) (28 mg, 24 μmol), sodium carbonate (77 mg, 0.73 mmol) and water (0.37 ml, 20 mmol) were added. The reaction mixture was stirred at 110° C. in a microwave (Biotage Initiator) for 90 min, cooled, filtered and purified by chromatography via HPLC (Method 9). This gave 92 mg (53% of theory) of the title compound.

LC-MS (Method 1): Rₜ=0.9 min; MS (ESIpos): m/z=714.5 [M+H]⁺.

### Example 137A

### N-alpha-[(trans-4-{[(tert-Butoxycarbonyl)amino]methyl}cyclohexyl)carbonyl]-3-{6-[3-(dimethylamino)propoxy]pyridin-3-yl}-N-{4-[3-(trifluoromethyl)-4H-1,2,4-triazol-5-yl]phenyl}-L-phenylalaninamide

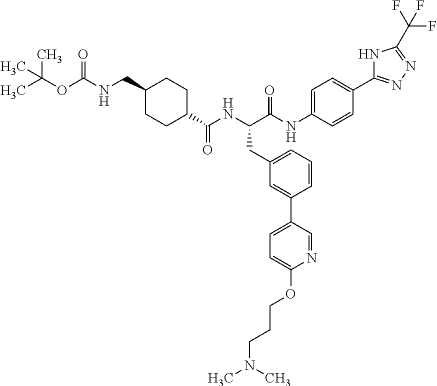

N-[(trans-4-{[(tert-Butoxycarbonyl)amino]methyl}cyclohexyl)carbonyl]-3-{6-[3-(dimethylamino)propoxy]pyridin-3-yl}-L-phenylalanine (150 mg, 0.26 mmol), 2-(4-aminophenyl)-5-trifluoromethyl-1,3,4-triazole (69 mg, 0.26 mmol) and N,N-diisopropylethylamine (0.18 ml, 1.0 mmol) were suspended in 2.5 ml of ethyl acetate, and 2,4,6-tripropyl-1,3,5,2,4,6-trioxatriphosphinane 2,4,6-trioxide (50% in ethyl acetate, 0.38 ml, 0.64 mmol) was added. The reaction mixture was subsequently heated under reflux for 3 h, and water and plenty of dichloromethane were then added. The organic phase was washed with saturated aqueous ammonium chloride solution and saturated aqueous sodium chloride solution, dried over sodium sulphate and concentrated. This gave 204 mg (100% of theory) of the title compound.

LC-MS (Method 4): Rₜ=1.18 min; MS (ESIpos): m/z=793.2 [M+H]⁺.

### Example 138A

### N-alpha-[(trans-4-{[(tert-Butoxycarbonyl)amino]methyl}cyclohexyl)carbonyl]-N-[4-(3-chloro-4H-1,2,4-triazol-5-yl)phenyl]-3-[4-methyl-6-(morpholin-4-yl)pyridin-3-yl]-L-phenylalaninamide

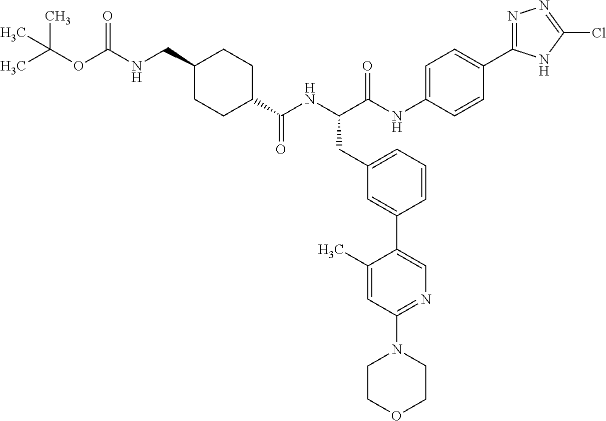

3-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide (150 mg, 0.23 mmol) and 4-[4-methyl-5-(4,4,5,5-tetramethyl-1,3,2-dioxaborolan-2-yl)pyridin-2-yl]morpholine (104 mg, 0.34 mmol) were dissolved in dimethyl sulphoxide (2 ml), and tetrakis(triphenylphosphine)palladium(0) (26 mg, 23 μmol), sodium carbonate (72 mg, 0.68 mmol) and water (0.34 ml, 19 mmol) were added. The reaction mixture was stirred at 110° C. in a microwave (Biotage Initiator) for 120 min, cooled, filtered and purified by chromatography via HPLC (Method 11). This gave 17 mg (10% of theory) of the title compound.

LC-MS (Method 5): Rₜ=0.87 min; MS (ESIpos): m/z=757.4 [M+H]⁺.

### Example 139A

### tert-Butyl [(trans-4-{[(2S)-3-(5′-amino-2′-methylbiphenyl-3-yl)-1-oxo-1-{[4-(1H-tetrazol-5-yl)phenyl]amino}propan-2-yl]carbamoyl}cyclohexyl)methyl]carbamate

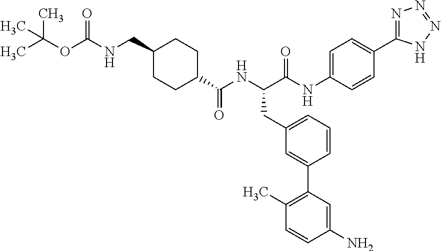

3-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (200 mg, 0.32 mmol) and 4-methyl-3-(4,4,5,5-tetramethyl-1,3,2-dioxaborolan-2-yl)aniline (164 mg, 0.70 mmol) were dissolved in dimethyl sulphoxide (2 ml), and 1,1′-bis(diphenylphosphine)ferrocenedichloropalladium(II) (26 mg, 32 μmol), sodium carbonate (101 mg, 0.96 mmol) and water (0.48 ml, 27 mmol) were added. The reaction mixture was stirred at 110° C. in a microwave (Biotage Initiator) for 240 min, 0.5 ml of 1M hydrochloric acid was added and the mixture was filtered through a cartridge and concentrated. The residue was converted further as the crude product.

LC-MS (Method 4): Rₜ=1.04 min; MS (ESIpos): m/z=653.4 [M+H]⁺.

### Example 140A

### tert-Butyl [(trans-4-{[(2S)-3-(2′-isopropylbiphenyl-3-yl)-1-({4-[3-(methoxymethyl)-4H-1,2,4-triazol-5-yl]phenyl}amino)-1-oxopropan-2-yl]carbamoyl}cyclohexyl)methyl]carbamate

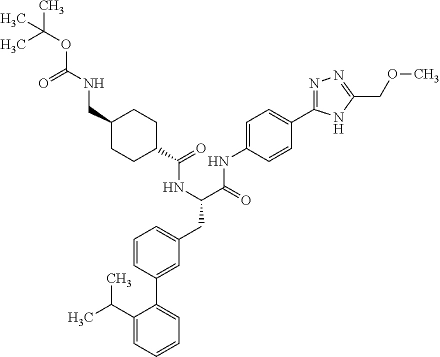

0.19 ml (0.37 mmol) of a 2M sodium carbonate solution in water was added to a solution of 125 mg (0.19 mmol) of 3-bromo-N-alpha-[(trans-4-{[(tert-butoxycarbonyl)amino]methyl}cyclohexyl)carbonyl]-N-{4-[3-(methoxymethyl)-4H-1,2,4-triazol-5-yl]phenyl}-L-phenylalaninamide and 77 mg (0.47 mmol) of (2-isopropylphenyl)boronic acid in 2 ml of N,N-dimethylformamide, and the mixture was degassed with argon for 5 min. 13.7 mg (0.02 mmol) of 1,1′-bis(diphenylphosphine)ferrocenepalladium(II) chloride were added and the mixture was stirred at 120° C. in a preheated oil bath for 30 min. The reaction solution was partitioned between water and ethyl acetate, and the organic phase was washed with water and aqueous saturated sodium chloride solution and dried over sodium sulphate. The solvent was removed and the residue was dissolved in acetonitrile and separated by preparative HPLC (mobile phase: acetonitrile/water gradient, 0.01% trifluoroacetic acid). The product-containing fractions were combined and concentrated on a rotary evaporator. The residue was dried under high vacuum. 57 mg (43% of theory) of the title compound were obtained.

¹H NMR (400 MHz, DMSO-d₆): δ=ppm 0.67-0.93 (m, 2H), 0.96-1.42 (m, 17H), 1.47-1.74 (m, 4H), 2.00-2.13 (m, 1H), 2.74 (t, 2H), 2.84-3.01 (m, 2H), 3.03-3.13 (m, 1H), 3.26-3.41 (m, 4H), 4.51 (m, 2H), 4.66-4.78 (m, 1H), 6.74-6.86 (m, 1H), 7.03 (d, 1H), 7.10 (d, 1H), 7.14-7.24 (m, 2H), 7.26-7.45 (m, 4H), 7.70 (d, 2H), 7.93 (d, 2H), 8.12 (d, 1H), 10.30 (s, 1H).

LC-MS (Method 1): Rₜ=1.24 min; MS (ESIpos): m/z=709 [M+H]⁺.

### Example 141A

### tert-Butyl [(trans-4-{[(2S)-3-[3-(quinolin-5-yl)phenyl]-1-({4-[3-(methoxymethyl)-4H-1,2,4-triazol-5-yl]phenyl}amino)-1-oxopropan-2-yl]carbamoyl}cyclohexyl)methyl]carbamate

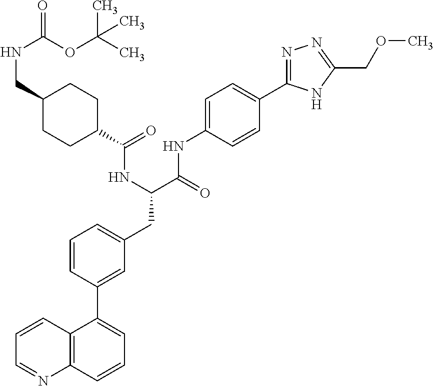

0.19 ml (0.37 mmol) of a 2M sodium carbonate solution in water was added to a solution of 125 mg (0.19 mmol) of 3-bromo-N-alpha-[(trans-4-{[(tert-butoxycarbonyl)amino]methyl}cyclohexyl)carbonyl]-N-{4-[3-(methoxymethyl)-4H-1,2,4-triazol-5-yl]phenyl}-L-phenylalaninamide and 81 mg (0.47 mmol) of quinolin-5-ylboronic acid in 2 ml of N,N-dimethylformamide, and the mixture was degassed with argon for 5 min. 13.7 mg (0.02 mmol) of 1,1′-bis(diphenylphosphine)ferrocenepalladium(II) chloride were added and the mixture was stirred at 120° C. in a preheated oil bath for 30 min. The reaction solution was partitioned between water and ethyl acetate, and the organic phase was washed with water and aqueous saturated sodium chloride solution and dried over sodium sulphate. The solvent was removed and the residue was dissolved in acetonitrile and separated by preparative HPLC (mobile phase: acetonitrile/water gradient, 0.01% trifluoroacetic acid). The product-containing fractions were combined and concentrated on a rotary evaporator. The residue was dried under high vacuum. 97 mg (71% of theory) of the title compound were obtained.

¹H NMR (400 MHz, DMSO-d₆): δ=ppm 0.63-0.89 (m, 2H), 1.04 (m, 4H), 1.37 (s, 9H), 1.42-1.59 (m, 2H), 1.60-1.71 (m, 2H), 2.01-2.18 (m, 1H), 2.72 (m, 2H), 2.92-3.05 (m, 1H), 3.09-3.24 (m, 1H), 4.52 (s, 2H), 4.73-4.84 (m, 1H), 6.71-6.85 (m, 1H), 7.35 (d, 1H), 7.41-7.56 (m, 3H), 7.60-7.79 (m, 4H), 7.85-8.01 (m, 3H), 8.17 (dd, 2H), 8.42 (d, 1H), 9.08 (d, 1H), 10.31 (s, 1H).

LC-MS (Method 1): Rₜ=0.94 min; MS (ESIpos): m/z=716 [M+H]⁺.

### Example 142A

### tert-Butyl 4-{[(5-{3-[(2S)-2-{[(trans-4-{[(tert-butoxycarbonyl)amino]methyl}cyclohexyl)carbonyl]amino}-3-{[4-(3-chloro-4H-1,2,4-triazol-5-yl)phenyl]amino}-3-oxopropyl]phenyl}pyridin-3-yl)sulphonyl]amino}piperidine-1-carboxylate

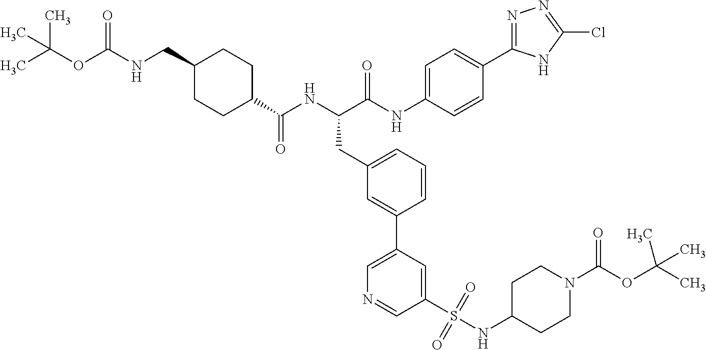

N-alpha-[(trans-4-{[(tert-Butoxycarbonyl)amino]methyl}cyclohexyl)carbonyl]-N-[4-(3-chloro-4H-1,2,4-triazol-5-yl)phenyl]-3-(4,4,5,5-tetramethyl-1,3,2-dioxaborolan-2-yl)-L-phenylalaninamide (195 mg, 0.28 mmol) and tert-butyl 4-{[(5-bromopyridin-3-yl)sulphonyl]amino}piperidine-1-carboxylate (139 mg, 0.33 mmol) were dissolved in 2 ml of dimethyl sulphoxide, the solution was flushed with argon and tetrakis(triphenylphosphine)palladium(0) (32 mg, 27.6 μmol), sodium carbonate (146 mg, 1.4 mmol) and water (0.42 ml) were added under argon. The reaction mixture was stirred at 110° C. in a microwave (Biotage Initiator) for 150 min, filtered and purified chromatographically by HPLC (Method 10). This gave 29.7 mg (12% of theory) of the title compound.

LC-MS (Method 1): Rₜ=1.34 min; MS (ESIpos): m/z=920.6 [M+H]⁺.

### Example 143A

### N-alpha-[(trans-4-{[(tert-Butoxycarbonyl)amino]methyl}cyclohexyl)carbonyl]-3-[2-(morpholin-4-yl)pyridin-4-yl]-N-[4-(2H-tetrazol-5-yl)phenyl]-L-phenylalaninamide

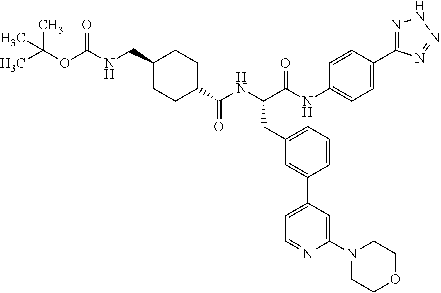

100 mg (0.16 mmol) of 3-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide, 18.4 mg (0.02 mmol) of tetrakis(triphenylphosphine)palladium(0) and 93 mg (0.32 mmol) of 4-[4-(4,4,5,5-tetramethyl-1,3,2-dioxaborolan-2-yl)pyridin-2-yl]morpholine were taken up in 1.5 ml of 1,2-dimethoxyethane and 0.50 ml of ethanol. After the addition of 1.2 ml of 2N aqueous sodium carbonate solution, the mixture was stirred under reflux for 5 h. The salts were removed by filtration from the reaction mixture and the filtrate was separated by preparative HPLC (mobile phase: acetonitrile/water gradient, 0.1% trifluoroacetic acid). This gave 66 mg of a mixture of the title compound and the corresponding deprotected amine, which was used directly in the next stage.

LC-MS (Method 1): Rₜ=0.88 min; MS (ESIneg): m/z=708 [M−H]⁻.

### Example 144A

### N-[(trans-4-{[(tert-Butoxycarbonyl)amino]methyl}cyclohexyl)carbonyl]-3-[2-(morpholin-4-yl)pyrimidin-5-yl]-L-phenylalanine

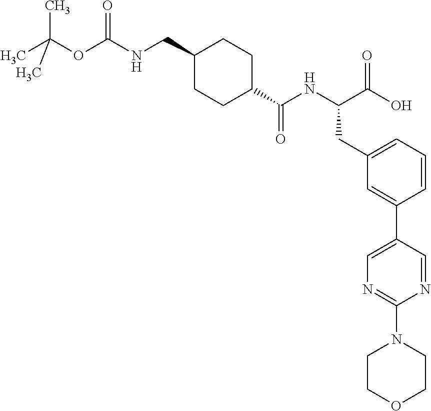

3-Bromo-N-[(trans-4-{[(tert-butoxycarbonyl)amino]methyl}cyclohexyl)carbonyl]-L-phenylalanine (368 mg, 0.76 mmol) and 4-[5-(4,4,5,5-tetramethyl-1,3,2-dioxaborolan-2-yl)pyrimidin-2-yl]morpholine were dissolved in dimethyl sulphoxide (6 ml), and tetrakis(triphenylphosphine)palladium(0) (89 mg, 0.076 mmol), sodium carbonate (242 mg, 2.3 mmol) and water (1.15 ml, 63.6 mmol) were added. The reaction mixture was stirred at 110° C. in a microwave (Biotage Initiator) for 90 min and then purified chromatographically by HPLC (Method 8). This gave 61 mg (14% of theory) of the title compound.

¹H NMR (300 MHz, DMSO-d₆): δ=ppm 0.66-0.86 (m, 2H), 1.03-1.27 (m, 3H), 1.35 (s, 9H), 1.44-1.69 (m, 5H), 1.87-2.02 (m, 1H), 2.65-2.76 (m, 2H), 2.94 (dd, 1H), 3.09 (dd, 1H), 3.60-3.76 (m, 8H), 3.87-3.98 (m, 1H), 6.65-6.81 (m, 1H), 6.94 (d, 1H), 7.03 (d, 1H), 7.23 (t, 1H), 7.27-7.41 (m, 2H), 8.62 (s, 2H).

LC-MS (Method 4): Rₜ=1.12 min; MS (ESIpos): m/z=568.3 [M+H]⁺.

### Example 145A

### 2,2,3,3-Tetrafluoro-3-[3-(4-nitrophenyl)-1H-1,2,4-triazol-5-yl]propanoic acid

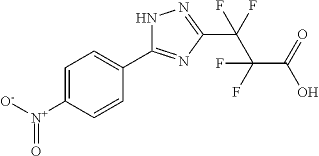 [SMILES: O=C(O)C(F)(F)C(F)(F)c1n[nH]c(-c2ccc([N+](=O)[O-])cc2)n1]

A solution of 1.22 g (6.8 mmol) of 4-nitrobenzenecarboximidohydrazide (described in: J. Liebigs Ann. Chem. 1897, 298, 51-52) in 50 ml of dichloromethane was stirred with 3.5 g (20 mmol) of 3,3,4,4-tetrafluorodihydrofuran-2,5-dione at RT for 2 min. 50 ml of acetonitrile were then added, and the mixture was stirred at RT overnight and then concentrated. This gave 2.3 g (quant.) of the title compound, which was reacted further without purification.

LC-MS (Method 4): Rₜ=0.72 min; MS (ESIneg): m/z=333.1 [M−H]⁻.

### Example 146A

### 3-[3-(4-Aminophenyl)-1H-1,2,4-triazol-5-yl]-2,2,3,3-tetrafluoropropanoic acid

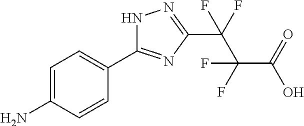 [SMILES: Nc1ccc(-c2nc(C(F)(F)C(F)(F)C(=O)O)n[nH]2)cc1]

Ammonium formate (1.7 g, 27 mmol) and palladium/carbon (10%, 732 mg, 0.69 mmol) were added to a solution of 2.3 g (6.9 mmol) of 2,2,3,3-tetrafluoro-3-[3-(4-nitrophenyl)-1H-1,2,4-triazol-5-yl]propanoic acid in methanol (115 ml), and the mixture was stirred at RT for 30 min. The reaction mixture was filtered and concentrated. This gave 4.1 g (quant., 50% pure) of the title compound, which was reacted further without purification.

LC-MS (Method 4): Rₜ=0.45 min; MS (ESIpos): m/z=305.0 [M+H]⁺.

### Example 147A

### N-alpha-[(trans-4-{[(tert-Butoxycarbonyl)amino]methyl}cyclohexyl)carbonyl]-3-(4-chloropyridin-3-yl)-N-[4-(5-oxo-4,5-dihydro-1,2,4-oxadiazol-3-yl)phenyl]-L-phenylalaninamide

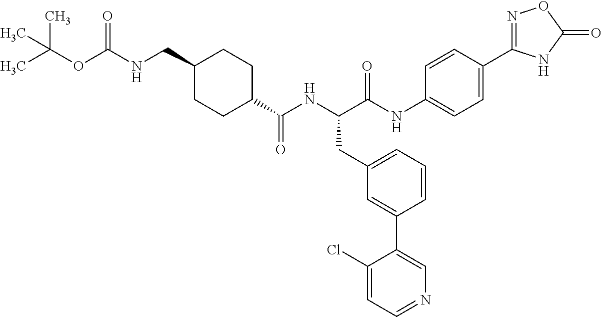

3-Bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide (150 mg, 0.24 mmol) and 4-chloro-3-(4,4,5,5-tetramethyl-1,3,2-dioxaborolan-2-yl)pyridine (84 mg, 0.35 mmol) were dissolved in dimethyl sulphoxide (2 ml), and tetrakis(triphenylphosphine)palladium(0) (27 mg, 23 μmol), sodium carbonate (74 mg, 0.70 mmol) and water (0.35 ml, 19.5 mmol) were added. The reaction mixture was stirred at 110° C. in a microwave (Biotage Initiator) for 210 min, cooled, filtered and purified by chromatography via HPLC (Method 10). This gave 17 mg (11% of theory) of the title compound.

LC-MS (Method 4): Rₜ=1.22 min; MS (ESIpos): m/z=675.4 [M+H]⁺.

### Example 148A

### 4-(5-{4-[(N-[(trans-4-{[(tert-Butoxycarbonyl)amino]methyl}cyclohexyl)carbonyl]-3-{6-[3-(dimethylamino)propoxy]pyridin-3-yl}-L-phenylalanyl)amino]phenyl}-1H-1,2,4-triazol-3-yl)-2,2,3,3,4,4-hexafluorobutanoic acid

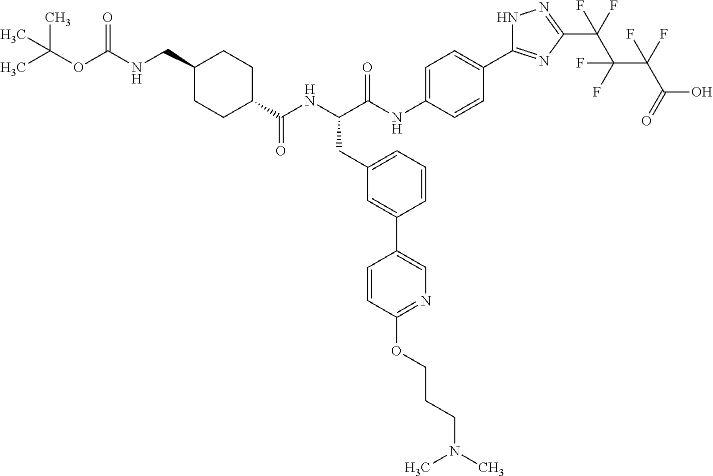 [SMILES: CN(C)CCCOc1ccc(-c2cccc(C[C@H](NC(=O)[C@H]3CC[C@H](CNC(=O)OC(C)(C)C)CC3)C(=O)Nc3ccc(-c4nc(C(F)(F)C(F)(F)C(F)(F)C(=O)O)n[nH]4)cc3)c2)cn1]

N,N-Diisopropylamine (0.1 ml, 0.55 mmol) was added to a solution of N-[(trans-4-{[(tert-butoxycarbonyl)amino]methyl}cyclohexyl)carbonyl]-3-{6-[3-(dimethylamino)propoxy]pyridin-3-yl}-L-phenylalanine (80 mg, 0.14 mmol) and 4-[5-(4-aminophenyl)-1H-1,2,4-triazol-3-yl]-2,2,3,3,4,4-hexafluorobutanoic acid/trifluoroacetic acid (1:1) (96 mg, 0.21 mmol) in DMF (2.5 ml), and HATU (63 mg, 0.17 mmol) was added thereto. The reaction mixture was stirred at RT overnight (about 16 h). Additional 4-[5-(4-aminophenyl)-1H-1,2,4-triazol-3-yl]-2,2,3,3,4,4-hexafluorobutanoic acid/trifluoroacetic acid (1:1) (64 mg, 0.14 mmol) and HATU (52 mg, 0.14 mmol) were then added, and stirring of the mixture was continued at RT overnight. The residue was diluted with water/acetonitrile and filtered through a Millipore filter, then purified by preparative HPLC (mobile phase: gradient of acetonitrile/water with 0.1% trifluoroacetic acid). This gave 32 mg (25% of theory) of the title compound.

LC-MS (Method 1): Rₜ=0.85 min; MS (ESIpos): m/z=919 [M+H]⁺.

### Example 149A

### 3-(5-{4-[(N-[(trans-4-{[(tert-Butoxycarbonyl)amino]methyl}cyclohexyl)carbonyl]-3-{6-[3-(dimethylamino)propoxy]pyridin-3-yl}-L-phenylalanyl)amino]phenyl}-1H-1,2,4-triazol-3-yl)-2,2,3,3-tetrafluoropropanoic acid

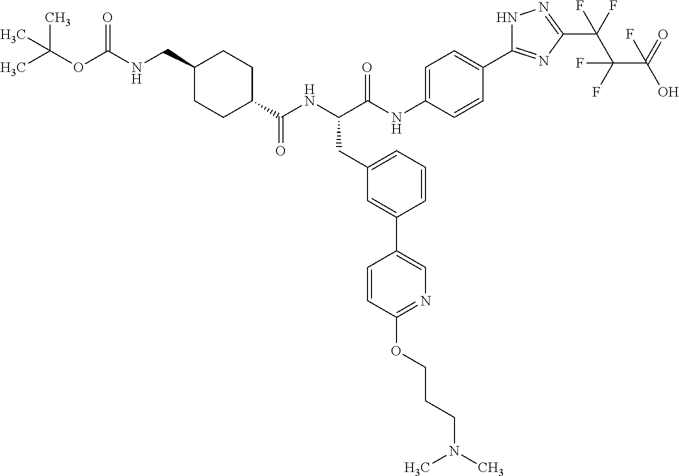

N,N-Diisopropylamine (0.1 ml, 0.55 mmol) was added to a solution of N-[(trans-4-{[(tert-butoxycarbonyl)amino]methyl}cyclohexyl)carbonyl]-3-{6-[3-(dimethylamino)propoxy]pyridin-3-yl}-L-phenylalanine (80 mg, 0.14 mmol) and 3-[5-(4-aminophenyl)-1H-1,2,4-triazol-3-yl]-2,2,3,3-tetrafluoropropanoic acid/trifluoroacetic acid (1:1) (78 mg, 0.21 mmol) in DMF (2.5 ml), and HATU (63 mg, 0.17 mmol) was added thereto. The reaction mixture was stirred at RT overnight (about 16 h). The residue was diluted with water/acetonitrile and filtered through a Millipore filter, then purified by preparative HPLC (mobile phase: gradient of acetonitrile/water with 0.1% trifluoroacetic acid). This gave 52 mg (39% of theory) of the title compound.

LC-MS (Method 1): Rₜ=0.85 min; MS (ESIpos): m/z=869 [M+H]⁺.

### Example 150A

### 3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide

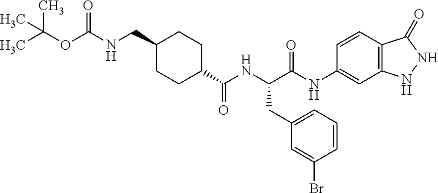

3-Bromo-N-[(trans-4-{[(tert-butoxycarbonyl)amino]methyl}cyclohexyl)carbonyl]-L-phenylalanine, 6-amino-1H-indazol-3(2H)-one and N,N-diisopropylethylamine are suspended in ethyl acetate, and 2,4,6-tripropyl-1,3,5,2,4,6-trioxatriphosphinane 2,4,6-trioxide (50% in ethyl acetate) is added. The reaction mixture is then heated under reflux for 2 h and stirred at RT for a further 48 h. The reaction mixture is worked up by methods known to those skilled in the art and the residue is separated by means of preparative HPLC. This gives the title compound.

### Example 151A

### tert-Butyl {[trans-4-({(2S)-3-[2′-methyl-5′-(morpholin-4-ylsulphonyl)biphenyl-3-yl]-1-oxo-1-[(3-oxo-2,3-dihydro-1H-indazol-6-yl)amino]propan-2-yl}carbamoyl)cyclohexyl]methyl}carbamate

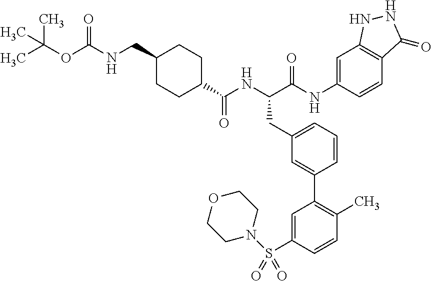

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [2-methyl-5-(morpholin-4-ylsulphonyl)phenyl]boronic acid are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 152A

### tert-Butyl {[trans-4-({(2S)-3-[2′-methyl-5′-(pyrrolidin-1-ylsulphonyl)biphenyl-3-yl]-1-oxo-1-[(3-oxo-2,3-dihydro-1H-indazol-6-yl)amino]propan-2-yl}carbamoyl)cyclohexyl]methyl}carbamate

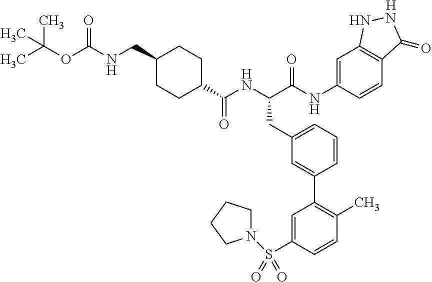

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [2-methyl-5-(pyrrolidin-1-ylsulphonyl)phenyl]boronic acid are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 153A

### tert-Butyl {[trans-4-({(2S)-3-[5′-(benzylsulphamoyl)-2′-methylbiphenyl-3-yl]-1-oxo-1-[(3-oxo-2,3-dihydro-1H-indazol-6-yl)amino]propan-2-yl}carbamoyl)cyclohexyl]methyl}carbamate

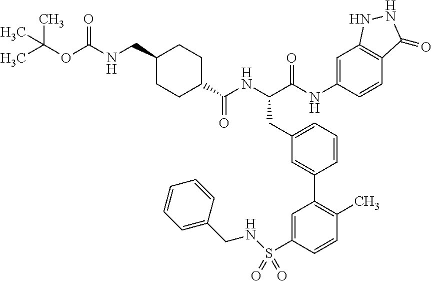

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [5-(benzylsulphamoyl)-2-methylphenyl]boronic acid are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 154A

### tert-Butyl {[trans-4-({(2S)-3-[5′-(dimethylsulphamoyl)-2′-methylbiphenyl-3-yl]-1-oxo-1-[(3-oxo-2,3-dihydro-1H-indazol-6-yl)amino]propan-2-yl}carbamoyl)cyclohexyl]methyl}carbamate

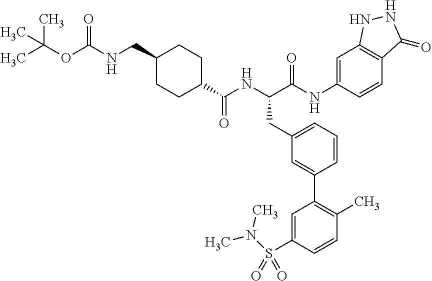

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [5-(dimethylsulphamoyl)-2-methylphenyl]boronic acid are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 155A

### tert-Butyl 4-[5-(3-{(2S)-2-{[(trans-4-{[(tert-butoxycarbonyl)amino]methyl}cyclohexyl)carbonyl]amino}-3-oxo-3-[(3-oxo-2,3-dihydro-1H-indazol-6-yl)amino]propyl}phenyl)pyrimidin-2-yl]piperazine-1-carboxylate

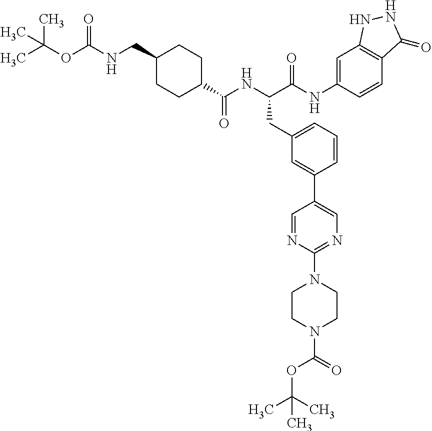

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and tert-butyl 4-[5-(4,4,5,5-tetramethyl-1,3,2-dioxaborolan-2-yl)pyrimidin-2-yl]piperazine-1-carboxylate are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 156A

### N-alpha-[(trans-4-{[(tert-Butoxycarbonyl)amino]methyl}cyclohexyl)carbonyl]-3-[2-(dimethylamino)pyrimidin-5-yl]-N-(3-oxo-2,3-dihydro-1H-indazol-6-yl)-L-phenylalaninamide

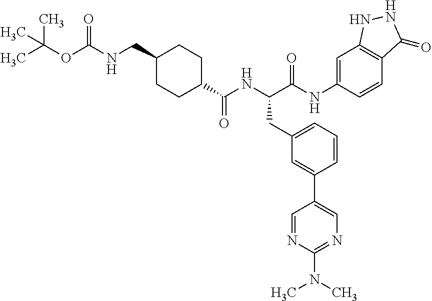

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and N,N-dimethyl-5-(4,4,5,5-tetramethyl-1,3,2-dioxaborolan-2-yl)pyrimidine-2-amine are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 157A

### N-alpha-[(trans-4-{[(tert-Butoxycarbonyl)amino]methyl}cyclohexyl)carbonyl]-3-(3-methylpyridin-4-yl)-N-(3-oxo-2,3-dihydro-1H-indazol-6-yl)-L-phenylalaninamide

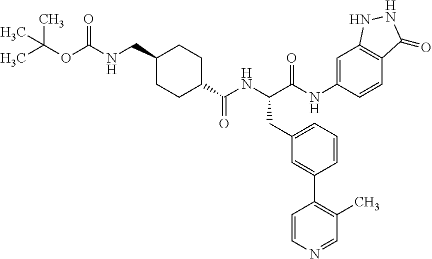

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and 3-methyl-4-(4,4,5,5-tetramethyl-1,3,2-dioxaborolan-2-yl)pyridine are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 158A

### N-alpha-[(trans-4-{[(tert-Butoxycarbonyl)amino]methyl}cyclohexyl)carbonyl]-3-[2-(4-methylpiperazin-1-yl)pyridin-4-yl]-N-(3-oxo-2,3-dihydro-1H-indazol-6-yl)-L-phenylalaninamide

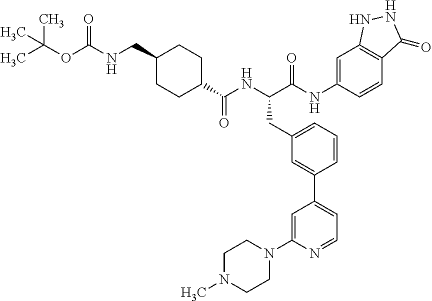

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and 1-methyl-4-[4-(4,4,5,5-tetramethyl-1,3,2-dioxaborolan-2-yl)pyridin-2-yl]piperazine are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 159A

### N-alpha-[(trans-4-{[(tert-Butoxycarbonyl)amino]methyl}cyclohexyl)carbonyl]-3-{6-[3-(dimethylamino)propoxy]pyridin-3-yl}-N-(3-oxo-2,3-dihydro-1H-indazol-6-yl)-L-phenylalaninamide

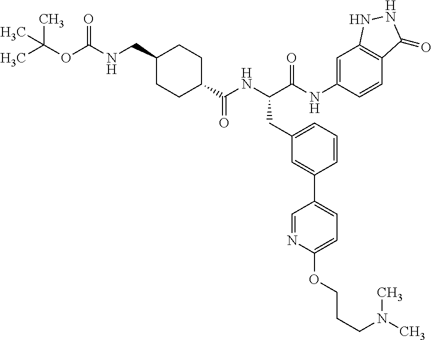 [SMILES: CN(C)CCCOc1ccc(-c2cccc(C[C@H](NC(=O)[C@H]3CC[C@H](CNC(=O)OC(C)(C)C)CC3)C(=O)Nc3ccc4c(=O)[nH][nH]c4c3)c2)cn1]

3-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and N,N-dimethyl-3-{[5-(4,4,5,5-tetramethyl-1,3,2-dioxaborolan-2-yl)pyridin-2-yl]oxy}propane-1-amine are dissolved in dimethyl sulphoxide, and tetrakis(triphenylphosphine)palladium(0), sodium carbonate and water are added. The reaction mixture is stirred at 110° C. in a microwave (Biotage Initiator) for 90 min, cooled, filtered and purified chromatographically by HPLC. This gives the title compound.

### Example 160A

### 2,2,3,3,4,4-Hexafluoro-4-[5-(4-nitrophenyl)-4H-1,2,4-triazol-3-yl]butanoic acid

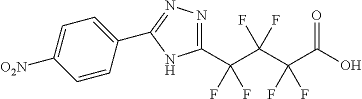 [SMILES: O=C(O)C(F)(F)C(F)(F)C(F)(F)c1nnc(-c2ccc([N+](=O)[O-])cc2)[nH]1]

3,3,4,4,5,5-Hexafluorodihydro-2H-pyran-2,6(3H)-dione (2.0 ml, 15.0 mmol) was added to a solution of 4-nitrobenzenecarboximidohydrazide (900 mg, 5.0 mmol) in dichloromethane (20 ml), followed by addition of acetonitrile (20 ml) and stirring at 50° C. for 3 h, then at RT overnight. The reaction mixture was stirred at 90° C. for another 4 h and, after addition of 4A molecular sieve, stirred at RT for a further 4 days. The solvent was removed on a rotary evaporator and the residue was diluted with water and acetonitrile, filtered through a Millipore filter and purified by preparative HPLC (eluent: gradient of acetonitrile/water with 0.1% trifluoroacetic acid). This gave 1.49 g (77% of theory) of the title compound.

LC-MS (Method 1): Rₜ=0.69 min; MS (ESIneg): m/z=383 [M−H]⁻.

### Example 161A

### 4-[5-(4-Aminophenyl)-4H-1,2,4-triazol-3-yl]-2,2,3,3,4,4-hexafluorobutanoic acid hydrochloride

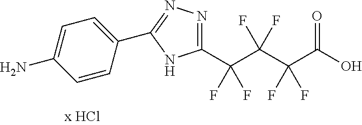 [SMILES: Nc1ccc(-c2nnc(C(F)(F)C(F)(F)C(F)(F)C(=O)O)[nH]2)cc1]

2,2,3,3,4,4-Hexafluoro-4-[5-(4-nitrophenyl)-4H-1,2,4-triazol-3-yl]butanoic acid (4.21 g, 10.96 mmol) and tin(II) chloride hydrate (9.89 g, 43.8 mmol) were stirred in ethanol (70 ml) at 70° C. for 1 h. The reaction mixture was poured onto ice-water and adjusted to pH 8 with solid sodium carbonate. The salts were filtered out of the mixture and the mixture was washed with ethyl acetate. The aqueous phase was acidified with 1N hydrochloric acid and the solvent was removed on a rotary evaporator. The residue was stirred with acetone and a little methanol and filtered off with suction. The residue was subsequently dried under high vacuum. This gave 3.59 g (76% of theory) of the title compound.

LC-MS (Method 1): Rₜ=0.44 min; MS (ESIneg): m/z=353 [M−H−HCl]⁻.

### WORKING EXAMPLES

### Example 1

### trans-4-(Aminomethyl)-N-[(2S)-3-[5′-(benzylsulphamoyl)-2′-methylbiphenyl-3-yl]-1-oxo-1-{[4-(1H-tetrazol-5-yl)phenyl]amino}propan-2-yl]cyclohexanecarboxamide hydrochloride

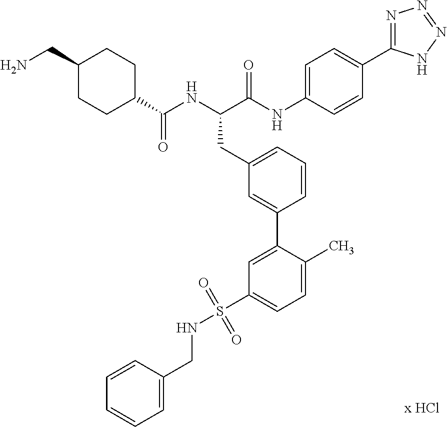

0.1 ml (0.40 mmol) of 4M hydrogen chloride in 1,4-dioxane was added to a suspension of 47 mg (0.07 mmol) of tert-butyl [(trans-4-{[(2S)-3-[5′-(benzylsulphamoyl)-2′-methylbiphenyl-3-yl]-1-oxo-1-{[4-(1H-tetrazol-5-yl)phenyl]amino)}propan-2-yl]carbamoyl}cyclohexy)methyl)]carbamate in 2 ml of 1,4-dioxane, and the mixture was stirred at 40° C. for 5 h. After the addition of a further 0.015 ml (0.058 mmol) of 4M hydrogen chloride in 1,4-dioxane, the reaction mixture was stirred at RT overnight. 5 ml of acetonitrile were added to the reaction mixture and the resulting precipitate was filtered off with suction, washed with acetonitrile and then dried under high vacuum. This gave 21 mg (49% of theory) of the title compound.

¹H NMR (300 MHz, DMSO-d₆): δ=ppm 0.75-0.95 (m, 2H), 1.03-1.28 (m, 2H), 1.34-1.53 (m, 2H), 1.60-1.76 (m, 3H), 2.04-2.14 (m, 1H), 2.19 (s, 3H), 2.55-2.62 (m, 2H), 2.85-2.97 (m, 1H), 3.06-3.15 (m, 1H), 3.96 (d, 2H), 4.66-4.76 (m, 1H), 7.12-7.39 (m, 9H), 7.44 (d, 1H), 7.52 (d, 1H), 7.65 (d, 1H), 7.71 (br. s, 3H), 7.78 (d, 2H), 7.96 (d, 2H) 8.10 (t, 1H), 8.22 (d, 1H) 10.48 (s, 1H).

LC-MS (Method 4) Rₜ=0.97 min MS (ESIpos): m/z=707 [M+H−HCl]⁺.

### Example 2

trans-4-(Aminomethyl)-N-[(2S)-3-[2′-methyl-5′-(pyrrolidin-1-ylsulphonyl)biphenyl-3-yl]-1-oxo-1-{[4-(1H-tetrazol-5-yl)phenyl]amino}propan-2-yl]cyclohexanecarboxamide hydrochloride

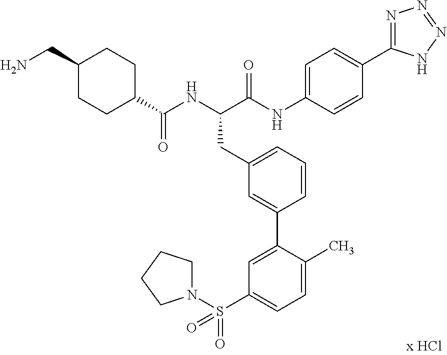

0.1 ml (0.38 mmol) of 4M hydrogen chloride in 1,4-dioxane was added to a suspension of 58 mg (0.076 mmol) of tert-butyl [(trans-4-{[(2S)-3-[2′-methyl-5′-(pyrrolidin-1-ylsulphonyl)biphenyl-3-yl]-1-oxo-1-{[4-(1H-tetrazol-5-yl)phenyl]amino}propan-2-yl]carbamoyl}cyclohexyl)methyl]carbamate in 1.5 ml of 1,4-dioxane, and the mixture was stirred at RT for 4 h and at 40° C. for 2 h. After the addition of a further 0.047 ml (0.19 mmol) of 4M hydrogen chloride in 1,4-dioxane, the reaction mixture was stirred at 40° C. for a further 2 h. 5 ml of acetonitrile were added to the reaction mixture and the resulting precipitate was filtered off with suction, washed with acetonitrile and then dried under high vacuum. This gave 37 mg (53% of theory) of the title compound.

¹H NMR (300 MHz, DMSO-d₆): δ=ppm 0.76-0.95 (m, 2H), 1.02-1.26 (m, 2H), 1.34-1.52 (m, 2H), 1.58-1.77 (m, 8H), 2.02-2.14 (m, 1H), 2.23 (s, 3H), 2.56-2.64 (m, 2H), 2.86-2.98 (m, 1H), 3.05-3.17 (m, 6H), 4.66-4.78 (m, 1H), 7.19 (br. d, 1H), 7.27 (br. s, 1H), 7.30-7.40 (m, 2H), 7.47 (d, 1H), 7.51 (d, 1H), 7.63-7.72 (m, 3H), 7.76 (d, 2H), 7.95 (d, 2H), 8.21 (d, 1H), 10.45 (s, 1H).

LC-MS (Method 4): Rₜ=0.92 min; MS (ESIpos): m/z=671 [M+H−HCl]⁺.

### Example 3

### trans-4-(Aminomethyl)-N-[(2S)-3-[2′-methyl-5′-(morpholin-4-ylsulphonyl)biphenyl-3-yl]-1-oxo-1-{[4-(1H-tetrazol-5-yl)phenyl]amino}propan-2-yl]cyclohexanecarboxamide hydrochloride

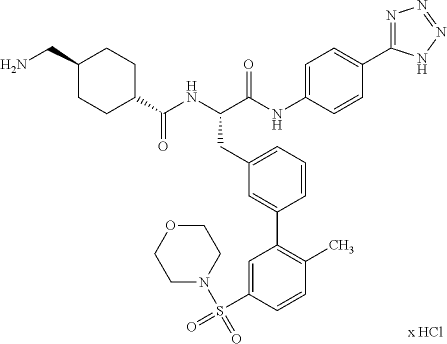

0.17 ml (0.7 mmol) of 4M hydrogen chloride in 1,4-dioxane was added to a suspension of 78 mg (0.099 mmol) of tert-butyl [(trans-4-{[(2S)-3-[2′-methyl-5′-(morpholin-4-ylsulphonyl)biphenyl-3-yl]-1-oxo-1-{[4-(1H-tetrazol-5-yl)phenyl]amino}propan-2-yl]carbamoyl}cyclohexyl)methyl]carbamate in 5 ml of 1,4-dioxane, and the mixture was stirred at RT overnight and at 40° C. for 2 h. After the addition of a further 0.025 ml (0.1 mmol) of 4M hydrogen chloride in 1,4-dioxane, the reaction mixture was stirred at 40° C. for a further 2 h. 5 ml of acetonitrile were added to the reaction mixture and the resulting precipitate was filtered off with suction, washed with acetonitrile and then dried under high vacuum. This gave 46 mg (64% of theory) of the title compound.

¹H NMR (300 MHz, DMSO-d₆): δ=ppm 0.75-0.95 (m, 2H), 1.01-1.29 (m, 2H), 1.34-1.51 (m, 2H), 1.58-1.78 (m, 3H), 2.06-2.14 (m, 1H), 2.24 (s, 3H), 2.53-2.62 (m, 2H), 2.84 (m, 4H), 2.87-2.97 (m, 1H), 3.07-3.17 (m, 1H), 3.56-3.65 (m, 4H), 4.67-4.78 (m, 1H), 7.18-7.24 (m, 1H), 7.27-7.42 (m, 4H), 7.53-7.62 (m, 2H), 7.67-7.75 (m, 3H), 7.78 (d, 2H) 7.96 (d, 2H) 8.23 (d, 1H) 10.50 (s, 1H)

LC-MS (Method 4): Rₜ=0.87 min; MS (ESIpos): m/z=686 [M+H−HCl]⁺.

### Example 4

### trans-4-(Aminomethyl)-N-[(2S)-3-[5′-(dimethylsulphamoyl)-2′-methylbiphenyl-3-yl]-1-oxo-1-{[4-(1H-tetrazol-5-yl)phenyl]amino}propan-2-yl]cyclohexanecarboxamide hydrochloride

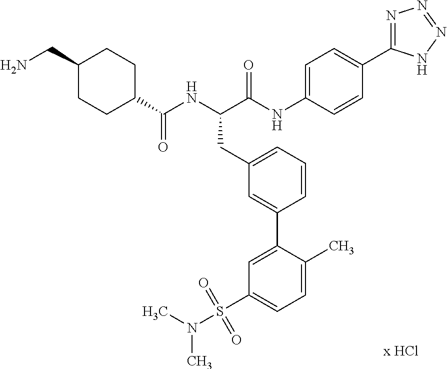

0.125 ml (0.5 mmol) of 4M hydrogen chloride in 1,4-dioxane was added to a suspension of 53 mg (0.071 mmol) of tert-butyl [(trans-4-{[(2S)-3-[5′-(dimethylsulphamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate in 3.5 ml of 1,4-dioxane, and the mixture was stirred at 40° C. for 5 h. 5 ml of acetonitrile were added to the reaction mixture and the resulting precipitate was filtered off with suction, washed with acetonitrile and then dried under high vacuum. This gave 47 mg (97% of theory) of the title compound.

¹H NMR (300 MHz, DMSO-d₆): δ=ppm 0.76-0.96 (m, 2H), 1.01-1.30 (m, 2H), 1.32-1.51 (m, 2H), 1.57-1.79 (m, 3H), 2.04-2.13 (m, 1H), 2.23 (s, 3H), 2.58 (s, 6H), 2.86-2.98 (m, 1H), 3.07-3.16 (m, 1H), 4.65-4.80 (m, 1H), 7.16-7.23 (m, 1H), 7.25-7.30 (m, 1H), 7.30-7.39 (m, 2H), 7.40-7.44 (m, 1H), 7.53 (dd, 1H), 7.60 (dd, 1H), 7.64-7.72 (br. s, 3H), 7.77 (d, 2H), 7.96 (d, 2H), 8.22 (d, 1H), 10.46 (s, 1H).

LC-MS (Method 4): Rₜ=0.89 min; MS (ESIpos): m/z=645 [M+H−HCl]⁺.

### Example 5

### trans-4-(Aminomethyl)-N-[(2S)-1-oxo-3-(3′-sulphamoylbiphenyl-3-yl)-1-{[4-(1H-tetrazol-5-yl)phenyl]amino}propan-2-yl]cyclohexanecarboxamide hydrochloride

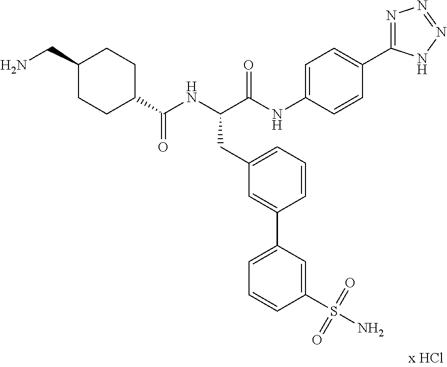

0.18 ml (0.8 mmol) of 4M hydrogen chloride in 1,4-dioxane was added to a suspension of 37 mg (0.053 mmol) of tert-butyl [(trans-4-{[(2S)-1-oxo-3-(3′-sulphamoylbiphenyl-3-yl)-1-{[4-(1H-tetrazol-5-yl)phenyl]amino}propan-2-yl]carbamoyl}cyclohexyl)methyl]carbamate in 1.5 ml of 1,4-dioxane, and the mixture was stirred at 40° C. for 4 h and at RT for 72 h. 5 ml of acetonitrile were added to the reaction mixture and the resulting precipitate was filtered off with suction, washed with acetonitrile, dried under high vacuum and then chromatographed by HPLC (Method 8). This gave 14 mg (39% of theory) of the title compound.

¹H NMR (400 MHz, MeOH-d₄): δ=ppm 0.93-1.07 (m, 2H), 1.24-1.48 (m, 3H), 1.48-1.59 (m, 1H), 1.61-1.78 (m, 2H), 1.78-1.91 (m, 2H), 2.16-2.26 (m, 1H), 2.73 (d, 2H), 3.06 (dd, 1H), 7.33 (d, 1H), 7.40 (t, 1H), 7.48-7.52 (m, 1H), 7.53-7.56 (m, 2H), 7.57-7.62 (m, 2H), 7.71-7.75 (m, 1H), 7.81-7.85 (m, 1H), 7.91-7.96 (m, 2H), 8.09-8.12 (m, 1H).

LC-MS (Method 4): Rₜ=0.74 min; MS (ESIpos): m/z=603 [M+H−HCl]⁺.

### Example 6

### trans-4-(Aminomethyl)-N-[(2S)-3-[3′-(cyclopropylsulphamoyl)biphenyl-3-yl]-1-oxo-1-{[4-(1H-tetrazol-5-yl)phenyl]amino}propan-2-yl]cyclohexanecarboxamide hydrochloride

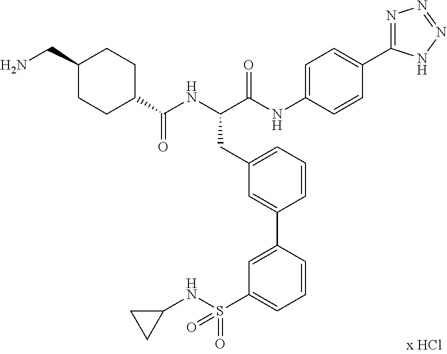

0.04 ml (0.16 mmol) of 4M hydrogen chloride in 1,4-dioxane was added to a suspension of 24 mg (0.032 mmol) of tert-butyl [(trans-4-{[(2S)-3-[3′-(cyclopropylsulphamoyl)biphenyl-3-yl]-1-oxo-1-{[4-(1H-tetrazol-5-yl)phenyl]amino}propan-2-yl]carbamoyl}cyclohexyl)methyl]carbamate in 1.5 ml of 1,4-dioxane, and the mixture was stirred at RT overnight and then at 40° C. for 2 h. After addition of a further 0.04 ml (0.16 mmol) of 4M hydrogen chloride in 1,4-dioxane and 2 h at 40° C., 5 ml of acetonitrile were added to the reaction mixture and the resulting precipitate was filtered off with suction, washed with acetonitrile, dried under high vacuum and then chromatographed by HPLC (Method 8). This gave 6 mg (27% of theory) of the title compound.

¹H NMR (400 MHz, MeOH-d₄): δ=ppm 0.43-0.53 (m, 4H), 0.93-1.07 (m, 3H), 1.25-1.47 (m, 3H), 1.48-1.60 (m, 1H), 1.62-1.78 (m, 2H), 1.79-1.91 (m, 2H), 2.12-2.27 (m, 2H), 2.73 (d, 2H), 3.03-3.12 (m, 1H), 7.31-7.36 (m, 1H), 7.40 (t, 1H), 7.50 (d, 1H), 7.53-7.61 (m, 4H), 7.76-7.82 (m, 2H), 7.90-7.96 (m, 2H), 8.04-8.07 (m, 1H).

LC-MS (Method 4): Rₜ=0.86 min; MS (ESIpos): m/z=643 [M+H−HCl]⁺.

### Example 7

### trans-4-(Aminomethyl)-N-[(2S)-3-[4′-(dimethylsulphamoyl)-2′-methylbiphenyl-3-yl]-1-oxo-1-{[4-(1H-tetrazol-5-yl)phenyl]amino}propan-2-yl]cyclohexanecarboxamide hydrochloride

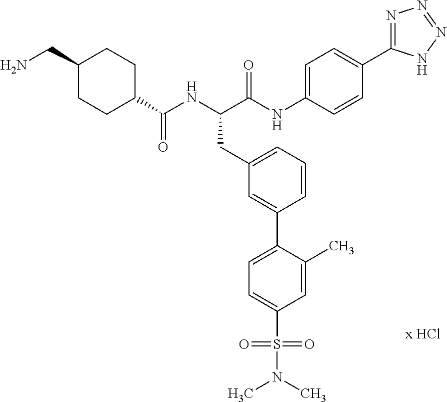

0.157 ml (0.6 mmol) of 4M hydrogen chloride in 1,4-dioxane was added to a suspension of 67 mg (0.09 mmol) of tert-butyl [(trans-4-{[(2S)-3-[4′-(dimethylsulphamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate in 3 ml of 1,4-dioxane, and the mixture was stirred at 40° C. for 5 h. After addition of a further 0.07 ml (0.27 mmol) of 4M hydrogen chloride in 1,4-dioxane and stirring overnight, 5 ml of acetonitrile were added to the reaction mixture and the resulting precipitate was filtered off with suction, washed with acetonitrile and dried under high vacuum. This gave 28 mg (45% of theory) of the title compound.

¹H NMR (300 MHz, DMSO-d₆): δ=ppm 0.77-0.97 (m, 2H), 1.04-1.30 (m, 2H), 1.34-1.54 (m, 2H), 1.61-1.78 (m, 3H), 2.04-2.15 (m, 1H), 2.24 (s, 3H), 2.60 (s, m, 7H), 2.92 (dd, 1H), 3.10 (dd, 1H), 4.65-4.77 (m, 1H), 7.16-7.21 (m, 1H), 7.27 (s, 1H), 7.34 (m, 3H), 7.52-7.58 (m, 1H), 7.60-7.64 (m, 1H), 7.65-7.73 (m, 3H), 7.78 (d, 2H), 7.95 (d, 2H), 8.18 (d, 1H), 10.44 (s, 1H).

LC-MS (Method 4): Rₜ=0.89 min; MS (ESIpos): m/z=645 [M+H−HCl]⁺.

### Example 8

### trans-4-(Aminomethyl)-N-[(2S)-3-[4′-methoxy-3′-(morpholin-4-ylsulphonyl)biphenyl-3-yl]-1-oxo-1-{[4-(1H-tetrazol-5-yl)phenyl]amino}propan-2-yl]cyclohexanecarboxamide hydrochloride

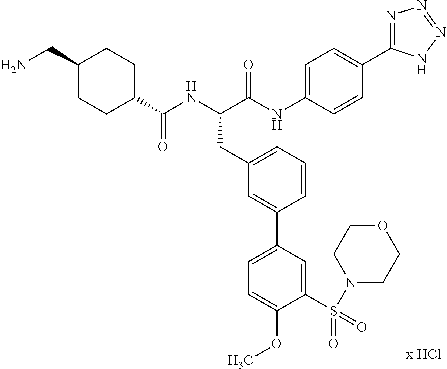

0.12 ml (0.48 mmol) of 4M hydrogen chloride in 1,4-dioxane was added to a suspension of 78 mg (0.1 mmol) of tert-butyl [(trans-4-{[(2S)-3-[4′-methoxy-3′-(morpholin-4-ylsulphonyl)biphenyl-3-yl]-1-oxo-1-{[4-(1H-tetrazol-5-yl)phenyl]amino}propan-2-yl]carbamoyl}cyclohexyl)methyl]carbamate in 2 ml of 1,4-dioxane, and the mixture was stirred at RT overnight and at 40° C. for 2 h. 5 ml of acetonitrile were added to the reaction mixture and the resulting precipitate was filtered off with suction and dried under high vacuum. This gave 57 mg (78% of theory) of the title compound.

¹H NMR (400 MHz, MeOH-d₄): δ=ppm 0.96-1.11 (m, 2H), 1.28-1.49 (m, 2H), 1.50-1.61 (m, 1H), 1.65-1.91 (m, 4H), 2.20-2.28 (m, 1H), 2.74 (d, 2H), 3.07 (dd, 1H), 3.14-3.18 (m, 4H), 3.23 (dd, 1H), 3.64 (s, 3H), 3.64-3.67 (m, 3H), 3.92 (s, 3H), 4.77 (dd, 1H), 7.22 (d, 1H), 7.28 (d, 1H), 7.37 (t, 1H), 7.41-7.45 (m, 2H), 7.69-7.73 (m, 2H), 7.75 (dd, 1H), 7.89-7.93 (m, 2H), 7.97 (d, 1H).

LC-MS (Method 4): Rₜ=0.83 min; MS (ESIpos): m/z=703 [M+H−HCl]⁺.

### Example 9

### trans-4-(Aminomethyl)-N-[(2S)-3-[2′-methyl-5′-(morpholin-4-ylsulphonyl)biphenyl-3-yl]-1-oxo-1-{[4-(5-oxo-4,5-dihydro-1H-1,2,4-triazol-3-yl)phenyl]amino}propan-2-yl]cyclohexanecarboxamide hydrochloride

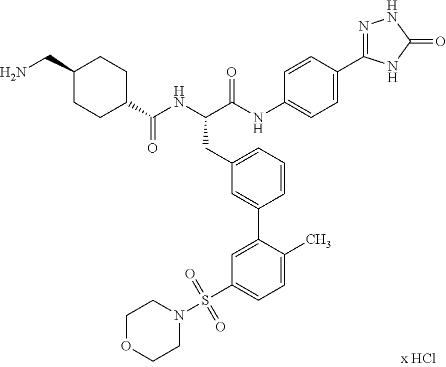

0.22 ml (0.87 mmol) of 4M hydrogen chloride in 1,4-dioxane was added to a suspension of 100 mg (0.12 mmol) of tert-butyl [(trans-4-{[(2S)-3-[2′-methyl-5′-(morpholin-4-ylsulphonyl)biphenyl-3-yl]-1-oxo-1-{[4-(5-oxo-4,5-dihydro-1H-1,2,4-triazol-3-yl)phenyl]amino}propan-2-yl]carbamoyl}cyclohexyl)methyl]carbamate in 4 ml of 1,4-dioxane, and the mixture was stirred at RT overnight. 5 ml of acetonitrile were added to the reaction mixture and the resulting precipitate was filtered off with suction and dried under high vacuum. This gave 74 mg (80% of theory) of the title compound.

¹H NMR (300 MHz, DMSO-d₆): δ=ppm 0.74-0.96 (m, 2H), 1.01-1.29 (m, 2H), 1.35-1.50 (m, 2H), 1.57-1.77 (m, 3H), 2.01-2.13 (m, 1H), 2.24 (s, 3H), 2.55-2.64 (m, 2H), 2.80-2.88 (m, 4H), 2.92 (dd, 1H), 3.10 (dd, 1H), 3.60 (d, 4H), 4.65-4.78 (m, 1H), 7.17-7.23 (m, 1H), 7.25-7.39 (m, 3H), 7.41 (d, 1H), 7.53-7.75 (m, 10H), 8.17 (d, 1H), 10.31-10.36 (m, 1H), 11.50-11.55 (m, 1H), 11.83-11.89 (m, 1H).

LC-MS (Method 4): Rₜ=0.83 min; MS (ESIpos): m/z=702 [M+H−HCl]⁺.

### Example 10

### trans-4-(Aminomethyl)-N-{(2S)-3-[2′-methyl-5′-(morpholin-4-ylsulphonyl)biphenyl-3-yl]-1-oxo-1-[(2-oxo-2,3-dihydro-1H-benzimidazol-5-yl)amino]propan-2-yl}cyclohexanecarboxamide hydrochloride

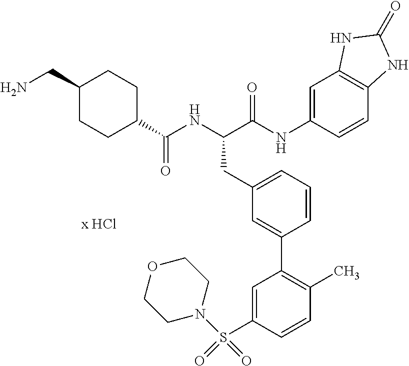

0.24 ml (0.97 mmol) of 4M hydrogen chloride in 1,4-dioxane was added to a suspension of 108 mg (0.14 mmol) of tert-butyl {[trans-4-({(2S)-3-[2′-methyl-5′-(morpholin-4-ylsulfonyl)biphenyl-3-yl]-1-oxo-1-[(2-oxo-2,3-dihydro-1H-benzimidazol-5-yl)amino]propan-2-yl}carbamoyl)cyclohexyl]methyl}carbamate in 7 ml of 1,4-dioxane, and the mixture was stirred at RT overnight. After addition of a further 0.24 ml (0.97 mmol) of 4M hydrogen chloride in 1,4-dioxane and 4 h at 50° C., 5 ml of acetonitrile were added to the reaction mixture and the precipitate formed was filtered off with suction and dried under high vacuum. This gave 77 mg (78% of theory) of the title compound.

¹H NMR (300 MHz, DMSO-d₆): δ=ppm 0.71-0.94 (m, 2H), 0.97-1.29 (m, 2H), 1.31-1.49 (m, 2H), 1.57-1.77 (m, 3H), 1.98-2.12 (m, 1H), 2.24 (s, 3H), 2.54-2.62 (m, 2H), 2.76-2.85 (m, 4H), 2.90 (dd, 1H), 3.07 (dd, 1H), 3.56-3.66 (m, 4H), 4.61-4.73 (m, 1H), 6.79 (dd, 1H), 6.98 (dd, 1H), 7.17-7.23 (m, 1H), 7.25-7.37 (m, 3H), 7.40 (m, 2H), 7.58 (m, 2H), 7.72 (br. s, 3H), 8.16 (d, 1H), 9.99 (s, 1H), 10.47 (s, 1H), 10.53 (s, 1H).

LC-MS (Method 4): Rₜ=0.84 min; MS (ESIpos): m/z=675 [M+H−HCl]⁺.

### Example 11

### trans-4-(Aminomethyl)-N-{(2S)-3-[2′-methyl-5′-(pyrrolidin-1-ylsulphonyl)biphenyl-3-yl]-1-oxo-1-[(2-oxo-2,3-dihydro-1H-benzimidazol-5-yl)amino]propan-2-yl}cyclohexanecarboxamide hydrochloride

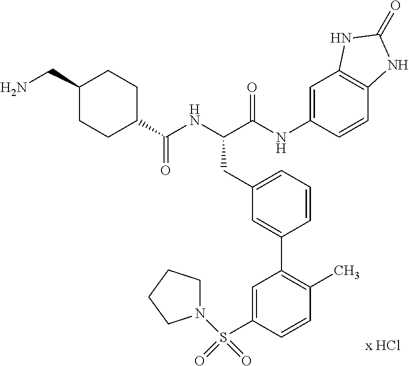

0.076 ml (0.3 mmol) of 4M hydrogen chloride in 1,4-dioxane was added to a suspension of 33 mg (0.043 mmol) of tert-butyl {[trans-4-({(2S)-3-[2′-methyl-5′-(pyrrolidin-1-ylsulfonyl)biphenyl-3-yl]-1-oxo-1-[(2-oxo-2,3-dihydro-1H-benzimidazol-5-yl)amino]propan-2-yl}carbamoyl)cyclohexyl]methyl}carbamate in 1.5 ml of 1,4-dioxane, and the mixture was stirred at RT overnight. After two further additions of 0.03 ml (0.12 mmol) of 4M hydrogen chloride in 1,4-dioxane and stirring overnight at RT or at 40° C., 5 ml of acetonitrile were added to the reaction mixture and the precipitate formed was filtered off with suction and dried under high vacuum. This gave 26 mg (83% of theory) of the title compound.

¹H NMR (300 MHz, DMSO-d₆): δ=ppm 0.72-0.96 (m, 2H), 0.99-1.29 (m, 2H), 1.32-1.48 (m, 2H), 1.64 (m, 7H), 1.98-2.12 (m, 1H), 2.23 (s, 3H), (2.55-2.63 (m, 2H), 2.81-2.94 (m, 1H), 3.02-3.15 (m, 5H), 3.53 (s, 1H), 4.60-4.73 (m, 1H), 6.78 (d, 1H), 6.97 (dd, 1H), 7.14-7.20 (m, 1H), 7.23-7.28 (m, 1H), 7.28-7.36 (m, 2H), 7.36-7.40 (m, 1H), 7.44-7.48 (m, 1H), 7.52 (d, 1H), 7.62-7.74 (m, 3H), 8.13 (d, 1H), 9.97 (s, 1H), 10.46 (s, 1H), 10.52 (s, 1H).

LC-MS (Method 4): Rₜ=0.80 min; MS (ESIpos): m/z=659 [M+H−HCl]⁺.

### Example 12

trans-4-(Aminomethyl)-N-{(2S)-3-[5′-(benzylsulphamoyl)-2′-methylbiphenyl-3-yl]-1-oxo-1-[(2-oxo-2,3-dihydro-1H-benzimidazol-5-yl)amino]propan-2-yl}cyclohexanecarboxamide hydrochloride

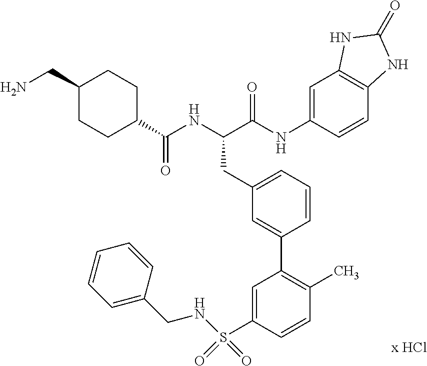

0.28 ml (1.1 mmol) of 4M hydrogen chloride in 1,4-dioxane was added to a suspension of 127 mg (0.16 mmol) of tert-butyl {[trans-4-({(2S)-3-[5′-(benzylsulphamoyl)-2′-methylbiphenyl-3-yl]-1-oxo-1-[(2-oxo-2,3-dihydro-1H-benzimidazol-5-yl)amino]propan-2-yl}carbamoyl)cyclohexyl]methyl}carbamate in 5.6 ml of 1,4-dioxane, and the mixture was stirred at RT overnight. After further addition of 0.2 ml (0.8 mmol) of 4M hydrogen chloride in 1,4-dioxane and stirring at 40° C. for 4 h, 5 ml of acetonitrile were added to the reaction mixture and the precipitate formed was filtered off with suction, suspended in water and lyophilized. This gave 86 mg (72% of theory) of the title compound.

¹H NMR (300 MHz, MeOH-d₄): δ=ppm 0.90-1.12 (m, 2H), 1.21-1.45 (m, 2H), 1.45-1.59 (m, 1H), 1.60-1.69 (m, 1H), 1.73-1.90 (m, 3H), 2.13-2.23 (m, 4H), 2.72 (d, 2H), 3.03 (dd, 1H), 3.22 (dd, 1H), 4.05 (s, 2H), 4.77 (dd, 1H), 6.91 (d, 1H), 6.98 (dd, 1H), 7.18 (d, 7H), 7.30-7.43 (m, 4H), 7.51-7.56 (m, 1H), 7.66 (dd, 1H).

LC-MS (Method 4): Rₜ=0.89 min; MS (ESIpos): m/z=695 [M+H−HCl]⁺.

### Example 13

### trans-4-(Aminomethyl)-N-[(2S)-3-[5′-(benzylsulphamoyl)-2′-methylbiphenyl-3-yl]-1-oxo-1-{[4-(5-oxo-4,5-dihydro-1H-1,2,4-triazol-3-yl)phenyl]amino}propan-2-yl]cyclohexanecarboxamide hydrochloride

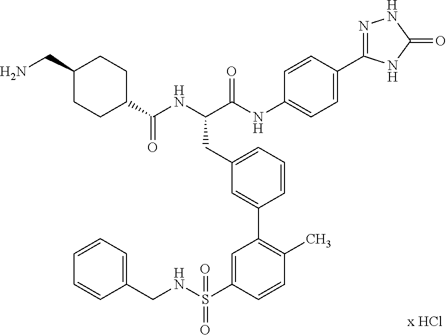

0.1 ml (0.4 mmol) of 4M hydrogen chloride in 1,4-dioxane was added to a suspension of 66 mg (0.08 mmol) of tert-butyl [(trans-4-{[(2S)-3-[5′-(benzylsulphamoyl)-2′-methylbiphenyl-3-yl]-1-oxo-1-{[4-(5-oxo-4,5-dihydro-1H-1,2,4-triazol-3-yl)phenyl]amino}propan-2-yl]carbamoyl}cyclohexyl)methyl]carbamate in 2 ml of 1,4-dioxane, and the mixture was stirred at RT overnight. After further addition of 0.1 ml (0.4 mmol) of 4M hydrogen chloride in 1,4-dioxane and stirring at 45-50° C. for 3 h, 5 ml of acetonitrile were added to the reaction mixture and the precipitate formed was filtered off with suction and dried under high vacuum. This gave 52 mg (84% of theory) of the title compound.

¹H NMR (300 MHz, MeOH-d₄): δ=ppm 0.92-1.12 (m, 2H), 1.24-1.47 (m, 2H), 1.48-1.61 (m, 1H), 1.62-1.72 (m, 1H), 1.74-1.91 (m, 3H), 2.20 (m, 4H), 2.70-2.77 (m, 2H), 3.00-3.10 (m, 1H), 3.20-3.26 (m, 1H), 3.65 (s, 2H), 4.04-4.08 (m, 2H), 7.10-7.25 (m, 7H), 7.31-7.42 (m, 3H), 7.52-7.55 (m, 1H), 7.60-7.74 (m, 1H).

LC-MS (Method 4): Rₜ=0.91 min; MS (ESIpos): m/z=722 [M+H−HCl]⁺.

### Example 14

### 3′-[(2S)-2-({[trans-4-(Aminomethyl)cyclohexyl]carbonyl}amino)-3-oxo-3-{[4-(1H-tetrazol-5-yl)phenyl]amino}propyl]-6-methyl-N-(piperidin-4-yl)biphenyl-3-carboxamide hydrochloride

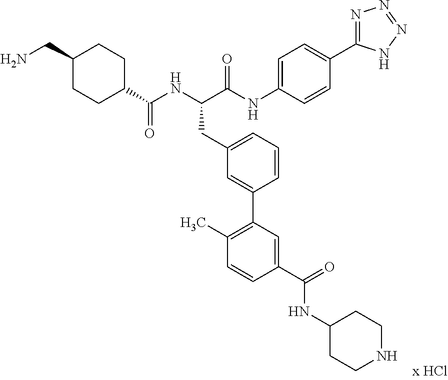

0.05 ml (0.18 mmol) of 4M hydrogen chloride in 1,4-dioxane was added to a suspension of 23 mg (0.026 mmol) of tert-butyl 4-[({3′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-6-methylbiphenyl-3-yl}carbonyl)amino]piperidine-1-carboxylate in 1.6 ml of 1,4-dioxane, and the mixture was stirred at RT overnight. After further addition of 0.03 ml (0.13 mmol) of 4M hydrogen chloride in 1,4-dioxane and stirring at RT for 48 h, 5 ml of acetonitrile were added to the reaction mixture and the precipitate formed was filtered off with suction, taken up in water and lyophilized. This gave 19 mg (95% of theory) of the title compound.

¹H NMR (300 MHz, DMSO-d₆): δ=ppm 0.75-0.97 (m, 2H), 1.03-1.30 (m, 2H), 1.34-1.53 (m, 2H), 1.70 (m, 5H), 1.85-1.97 (m, 2H), 2.03-2.13 (m, 1H), 2.16 (s, 3H), 2.56-2.63 (m, 2H), 2.82-3.04 (m, 3H), 3.10 (dd, 1H), 3.93-4.10 (m, 1H), 4.65-4.77 (m, 1H), 7.12-7.19 (m, 1H), 7.33 (m, 4H), 7.78 (d, 7H), 7.95 (d, 2H), 8.23 (d, 1H), 8.44 (d, 1H), 8.57-8.82 (m, 2H), 10.54 (s, 1H).

LC-MS (Method 4): Rₜ=0.65 min; MS (ESIpos): m/z=664 [M+H−HCl]⁺.

### Example 15

### 3′-[(2S)-2-({[trans-4-(Aminomethyl)cyclohexyl]carbonyl}amino)-3-oxo-3-{[4-(1H-tetrazol-5-yl)phenyl]amino}propyl]-2-methyl-N-(piperidin-4-yl)biphenyl-4-carboxamide hydrochloride

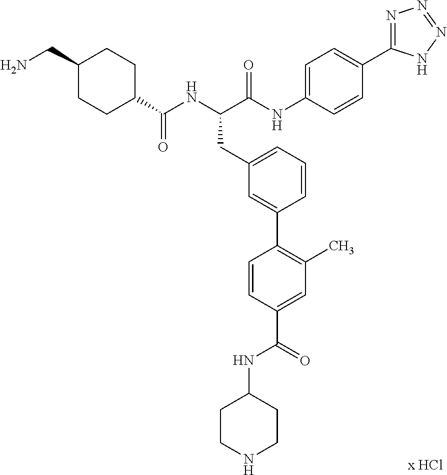

0.08 ml (0.33 mmol) of 4M hydrogen chloride in 1,4-dioxane was added to a suspension of 28 mg (0.033 mmol) of tert-butyl 4-[({3′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2-methylbiphenyl-4-yl}carbonyl)amino]piperidine-1-carboxylate in 1.2 ml of 1,4-dioxane, and the mixture was stirred at RT overnight. 5 ml of acetonitrile were added to the reaction mixture and the resulting precipitate was filtered off with suction and dried under high vacuum. This gave 22 mg (88% of theory) of the title compound.

¹H NMR (400 MHz, DMSO-d₆): δ=ppm 0.78-0.95 (m, 2H), 1.05-1.17 (m, 1H), 1.17-1.29 (m, 1H), 1.37-1.47 (m, 1H), 1.47-1.55 (m, 1H), 1.64-1.82 (m, 5H), 1.89-1.98 (m, 2H), 2.06-2.14 (m, 1H), 2.18 (s, 3H), 2.55-2.62 (m, 2H), 2.87-3.04 (m, 3H), 3.10 (dd, 1H), 3.98-4.09 (m, 1H), 4.66-4.76 (m, 1H), 7.10-7.15 (m, 1H), 7.17 (d, 1H), 7.24 (s, 1H), 7.28-7.37 (m, 2H), 7.69 (d, 1H), 7.78 (m, 6H), 7.96 (d, 2H), 8.18-8.24 (m, 1H), 8.44 (d, 1H), 8.62-8.81 (m, 2H), 10.49 (s, 1H).

LC-MS (Method 4): Rₜ=0.65 min; MS (ESIpos): m/z=630 [M+H−HCl]⁺.

### Example 16

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-(3-azabicyclo[3.1.0]hex-6-yl)-6-methylbiphenyl-3-carboxamide hydrochloride

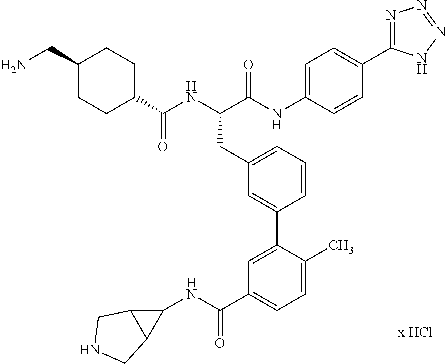

0.05 ml (0.21 mmol) of 4M hydrogen chloride in 1,4-dioxane was added to a suspension of 19 mg (0.021 mmol) of tert-butyl 6-[({3′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-6-methylbiphenyl-3-yl}carbonyl)amino]-3-azabicyclo[3.1.0]hexane-3-carboxylate in 0.6 ml of 1,4-dioxane, and the mixture was stirred at RT overnight. After further addition of 0.03 ml (0.11 mmol) of 4M hydrogen chloride in 1,4-dioxane and stirring at RT for 48 h, 5 ml of acetonitrile were added to the reaction mixture and the precipitate formed was filtered off with suction, taken up in water and lyophilized. This gave 13 mg (79% of theory) of the title compound.

¹H NMR (300 MHz, DMSO-d₆): δ=ppm 0.74-0.97 (m, 3H), 1.01-1.29 (m, 3H), 1.34-1.53 (m, 3H), 1.70 (s, 4H), 1.96 (br. s., 2H), 2.03-2.14 (m, 1H), 2.17 (s, 3H), 2.55-2.64 (m, 2H), 2.91 (dd, 1H), 2.98-3.03 (m, 1H), 3.10 (dd, 1H), 4.64-4.78 (m, 1H), 7.11-7.21 (m, 1H), 7.26-7.38 (m, 5H), 7.64 (s, 1H), 7.70 (d, 1H), 7.80 (m, 5H), 7.98 (d, 2H), 8.24 (d, 1H), 8.57 (d, 1H), 8.87-9.05 (m, 1H), 9.39-9.56 (m, 1H), 10.50-10.62 (m, 1H).

LC-MS (Method 4): Rₜ=0.65 min; MS (ESIpos): m/z=662 [M+H−HCl]⁺.

### Example 17

### trans-4-(Aminomethyl)-N-[(2S)-3-[6-methyl-3′-(morpholin-4-ylcarbonyl)biphenyl-3-yl]-1-oxo-1-{[4-(1H-tetrazol-5-yl)phenyl]amino}propan-2-yl]cyclohexanecarboxamide hydrochloride

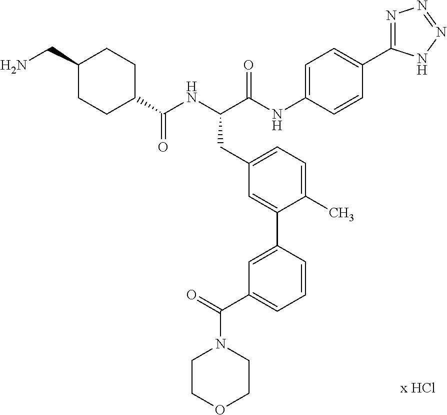

0.27 ml (1.1 mmol) of 4M hydrogen chloride in 1,4-dioxane was added to a suspension of 119 mg (0.16 mmol) of tert-butyl [(trans-4-{[(2S)-3-[6-methyl-3′-(morpholin-4-ylcarbonyl)biphenyl-3-yl]-1-oxo-1-{[4-(1H-tetrazol-5-yl)phenyl]amino}propan-2-yl]carbamoyl}cyclohexyl)methyl]carbamate in 5.6 ml of 1,4-dioxane, and the mixture was stirred at RT overnight. 5 ml of acetonitrile were then added to the reaction mixture and the resulting precipitate was filtered off with suction and dried under high vacuum. This gave 71 mg (65% of theory) of the title compound.

¹H NMR (300 MHz, DMSO-d₆): δ=ppm 0.76-0.95 (m, 2H), 1.05-1.31 (m, 2H), 1.35-1.47 (m, 1H), 1.49-1.58 (m, 1H), 1.63-1.78 (m, 3H), 2.02-2.12 (m, 1H), 2.16 (s, 3H), 2.56-2.65 (m, 2H), 2.86 (dd, 1H), 3.03 (dd, 1H), 3.51-3.66 (m, 4H), 4.59-4.72 (m, 1H), 7.10-7.21 (m, 3H), 7.24-7.28 (m, 1H), 7.29-7.38 (m, 2H), 7.42-7.50 (m, 1H), 7.77 (m, 6H), 7.95 (d, 2H), 8.15 (d, 1H), 10.44 (s, 1H).

LC-MS (Method 4): Rₜ=0.86 min; MS (ESIpos): m/z=651 [M+H−HCl]⁺.

### Example 18

### 5′-[(2S)-2-({[trans-4-(Aminomethyl)cyclohexyl]carbonyl}amino)-3-oxo-3-{[4-(1H-tetrazol-5-yl)phenyl]amino}propyl]-N-{2-[2-(2-methoxyethoxy)ethoxy]ethyl}-2′-methylbiphenyl-3-carboxamide hydrochloride

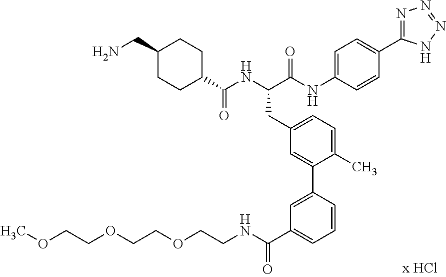

0.09 ml (0.35 mmol) of 4M hydrogen chloride in 1,4-dioxane was added to a suspension of 42 mg (0.05 mmol) of tert-butyl [(trans-4-{[(2S)-3-[3′-({2-[2-(2-methoxyethoxy)ethoxy]ethyl}carbamoyl)-6-methylbiphenyl-3-yl]-1-oxo-1-{[4-(1H-tetrazol-5-yl)phenyl]amino}propan-2-yl]carbamoyl}cyclohexyl)methyl]carbamate in 1.5 ml of 1,4-dioxane, and the mixture was stirred at RT overnight. After addition of a further 0.04 ml (0.15 mmol) of 4M hydrogen chloride in 1,4-dioxane and 48 h at RT, 5 ml of acetonitrile were added to the reaction mixture and the precipitate formed was filtered off with suction and dried under high vacuum. This gave 30 mg (76% of theory) of the title compound.

¹H NMR (300 MHz, DMSO-d₆): δ=ppm 0.76-0.96 (m, 1H), 1.03-1.30 (m, 2H), 1.33-1.47 (m, 1H), 1.47-1.58 (m, 1H), 1.61-1.78 (m, 4H), 2.01-2.11 (m, 1H), 2.14 (s, 3H), 2.55-2.64 (m, 2H), 2.85 (dd, 1H), 3.02 (dd, 1H), 3.16 (s, 3H), 3.38-3.42 (m, 2H), 3.43-3.53 (m, 9H), 4.59-4.70 (m, 1H), 7.10-7.21 (m, 3H), 7.35-7.42 (m, 1H), 7.43-7.52 (m, 1H), 7.67 (br. s, 3H), 7.77 (m, 5H), 7.94 (d, 2H), 8.16 (d, 1H), 8.51-8.59 (m, 1H), 10.46 (s, 1H).

LC-MS (Method 4): Rₜ=0.82 min; MS (ESIpos): m/z=727 [M+H−HCl]⁺.

### Example 19

### 5′-[(2S)-2-({[trans-4-(Aminomethyl)cyclohexyl]carbonyl}amino)-3-oxo-3-{[4-(1H-tetrazol-5-yl)phenyl]amino}propyl]-2′-methyl-N-[1-(propan-2-yl)piperidin-4-yl]biphenyl-3-carboxamide hydrochloride

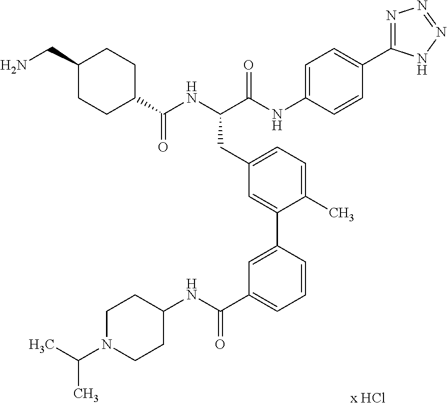

0.1 ml (0.4 mmol) of 4M hydrogen chloride in 1,4-dioxane was added to a suspension of 44 mg (0.05 mmol) of tert-butyl [(trans-4-{[(2S)-3-(6-methyl-3′-{[1-(propan-2-yl)piperidin-4-yl]carbamoyl}biphenyl-3-yl)-1-oxo-1-{[4-(1H-tetrazol-5-yl)phenyl]amino}propan-2-yl]carbamoyl}cyclohexyl)methyl]carbamate in 1.8 ml of 1,4-dioxane, and the mixture was stirred at RT overnight. 5 ml of acetonitrile were then added to the reaction mixture and the resulting precipitate was filtered off with suction and dried under high vacuum. This gave 42 mg (96% of theory) of the title compound.

¹H NMR (300 MHz, DMSO-d₆): δ=ppm 0.75-0.96 (m, 2H), 1.02-1.21 (m, 2H), 1.24 (d, 6H), 1.36-1.48 (m, 1H), 1.49-1.60 (m, 1H), 1.63-1.80 (m, 3H), 1.89-2.04 (m, 4H), 2.09-2.16 (m, 4H), 2.55-2.64 (m, 2H), 2.86 (dd, 1H), 2.96-3.12 (m, 3H), 3.39-3.45 (m, 1H), 3.93-4.12 (m, 1H), 4.59-4.70 (m, 1H), 7.12-7.24 (m, 3H), 7.34-7.42 (m, 1H), 7.47 (t, 1H), 7.70-7.87 (m, 7H), 7.96 (d, 2H), 8.19 (d, 1H), 8.52-8.62 (m, 1H), 10.48 (s, 1H).

LC-MS (Method 4): Rₜ=0.70 min; MS (ESIpos): m/z=706 [M+H−HCl]⁺.

### Example 20

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-{2-[2-(2-methoxyethoxy)ethoxy]ethyl}-6-methylbiphenyl-3-carboxamide hydrochloride

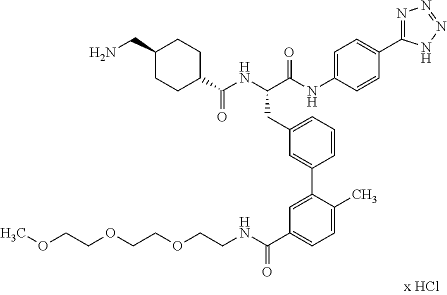

0.1 ml (0.38 mmol) of 4M hydrogen chloride in 1,4-dioxane was added to a suspension of 45 mg (0.05 mmol) of tert-butyl [(trans-4-{[(2S)-3-[5′-({2-[2-(2-methoxyethoxy)ethoxy]ethyl}carbamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate in 1.5 ml of 1,4-dioxane, and the mixture was stirred at RT overnight. 5 ml of acetonitrile were then added to the reaction mixture and the resulting precipitate was filtered off with suction and purified chromatographically by HPLC (Method 8). This gave 19 mg (47% of theory) of the title compound.

¹H NMR (300 MHz, DMSO-d₆): δ=ppm 0.74-0.95 (m, 2H), 1.01-1.30 (m, 2H), 1.32-1.52 (m, 2H), 1.57-1.78 (m, 3H), 2.02-2.14 (m, 1H), 2.18 (s, 3H), 2.59 (d, 2H), 2.89 (dd, 1H), 3.10 (dd, 1H), 3.17 (s, 3H), 3.34-3.41 (m, 7H), 3.43-3.53 (m, 9H), 4.65-4.76 (m, 1H), 7.12-7.19 (m, 1H), 7.26-7.35 (m, 4H), 7.55 (d, 2H), 7.67 (s, 1H), 7.69-7.74 (m, 1H), 7.85 (d, 2H), 8.09 (d, 1H), 8.42-8.49 (m, 1H), 10.08 (s, 1H).

LC-MS (Method 4): Rₜ=0.80 min; MS (ESIpos): m/z=727 [M+H−HCl]⁺.

### Example 21

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-{2-[2-(2-hydroxy ethoxy)ethoxy]ethyl}-6-methylbiphenyl-3-carboxamide hydrochloride

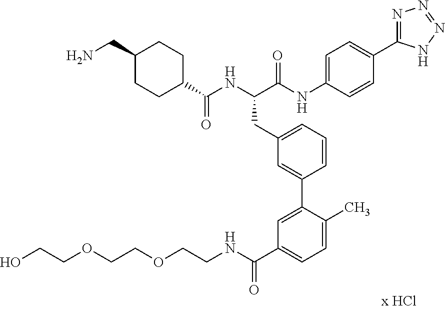

0.12 ml (0.48 mmol) of 4M hydrogen chloride in 1,4-dioxane was added to a suspension of 56 mg (0.07 mmol) of tert-butyl [(trans-4-{[(2S)-3-[5′-({2-[2-(2-hydroxyethoxy)ethoxy]ethyl}carbamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate in 2.1 ml of 1,4-dioxane, and the mixture was stirred at RT overnight. 5 ml of acetonitrile were then added to the reaction mixture and the resulting precipitate was filtered off with suction and dried under high vacuum. This gave 40 mg (73% of theory) of the title compound.

¹H NMR (400 MHz, DMSO-d₆): δ=ppm 0.77-0.94 (m, 2H), 1.05-1.16 (m, 1H), 1.16-1.28 (m, 1H), 1.36-1.44 (m, 1H), 1.45-1.52 (m, 1H), 1.61-1.77 (m, 3H), 2.04-2.14 (m, 1H), 2.18 (s, 3H), 2.55-2.62 (m, 2H), 2.91 (dd, 1H), 3.11 (dd, 1H), 3.37 (m, 4H), 3.42 (m, 3H), 3.46-3.52 (m, 7H), 4.67-4.76 (m, 1H), 7.13-7.18 (m, 1H), 7.27-7.37 (m, 4H), 7.64-7.75 (m, 5H), 7.78 (d, 2H), 7.95 (d, 2H), 8.19 (d, 1H), 8.43-8.51 (m, 1H), 10.48 (s, 1H).

LC-MS (Method 4): Rₜ=0.77 min; MS (ESIpos): m/z=713.6 [M+H−HCl]⁺.

### Example 22

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-{2-[2-(2-methoxyethoxy)ethoxy]ethyl}-2-methylbiphenyl-4-carboxamide hydrochloride

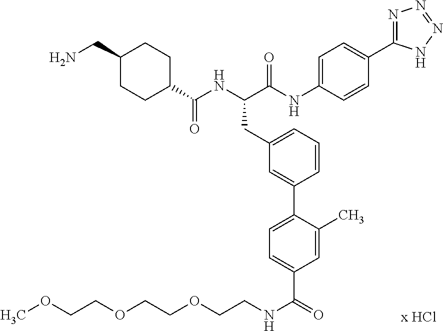

0.11 ml (0.45 mmol) of 4M hydrogen chloride in 1,4-dioxane was added to a suspension of 53 mg (0.065 mmol) of tert-butyl [(trans-4-{[(2S)-3-[4′-({2-[2-(2-methoxyethoxy)ethoxy]ethyl}carbamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate in 2.0 ml of 1,4-dioxane, and the mixture was stirred at RT overnight. 5 ml of acetonitrile were then added to the reaction mixture and the resulting precipitate was filtered off with suction and dried under high vacuum. This gave 39 mg (76% of theory) of the title compound.

¹H NMR (300 MHz, DMSO-d₆): δ=ppm 0.76-0.97 (m, 2H), 1.01-1.30 (m, 2H), 1.34-1.54 (m, 2H), 1.60-1.77 (m, 3H), 1.99-2.15 (m, 1H), 2.18 (s, 3H), 2.56-2.64 (m, 2H), 2.90 (dd, 1H), 3.10 (dd, 1H), 3.18 (s, 3H), 3.34-3.43 (m, 4H), 3.45-3.55 (m, 8H), 4.65-4.76 (m, 1H), 7.09-7.37 (m, 5H), 7.64-7.85 (m, 8H), 7.97 (d, 2H), 8.22 (d, 1H), 8.44-8.53 (m, 1H), 10.49 (s, 1H).

LC-MS (Method 4): Rₜ=0.80 min; MS (ESIpos): m/z=727.6 [M+H−HCl]⁺.

### Example 23

### trans-4-(Aminomethyl)-N-[(2S)-3-[5′-(dimethylsulphamoyl)-2′-methylbiphenyl-3-yl]-1-oxo-1-{[4-(5-oxo-4,5-dihydro-1,2,4-oxadiazol-3-yl)phenyl]amino}propan-2-yl]cyclohexanecarboxamide hydrochloride

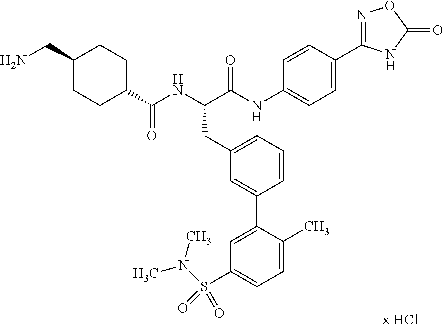

0.19 ml (0.76 mmol) of 4M hydrogen chloride in 1,4-dioxane was added to a suspension of 83 mg (0.11 mmol) of tert-butyl [(trans-4-{[(2S)-3-[5′-(dimethylsulphamoyl)-2′-methylbiphenyl-3-yl]-1-oxo-1-{[4-(5-oxo-4,5-dihydro-1,2,4-oxadiazol-3-yl)phenyl]amino}propan-2-yl]carbamoyl}cyclohexyl)methyl]carbamate in 4.0 ml of 1,4-dioxane, and the mixture was stirred at RT overnight. 5 ml of acetonitrile were then added to the reaction mixture and the resulting precipitate was filtered off with suction and purified chromatographically by HPLC (Method 10). This gave 45 mg (59% of theory) of the title compound.

¹H NMR (300 MHz, DMSO-d₆): δ=ppm 0.75-0.95 (m, 2H), 1.01-1.30 (m, 2H), 1.33-1.52 (m, 2H), 1.58-1.78 (m, 3H), 2.00-2.15 (m, 1H), 2.23 (s, 3H), 2.59 (s, 6H), 2.91 (dd, 1H), 3.10 (dd, 1H), 4.67-4.77 (m, 1H), 7.19 (m, 1H), 7.27 (s, 1H), 7.29-7.39 (m, 2H), 7.42 (d, 1H), 7.55 (d, 1H), 7.61 (dd, 1H), 7.65-7.77 (m, 6H), 8.19 (d, 1H), 10.47 (s, 1H).

LC-MS (Method 4): Rₜ=0.93 min; MS (ESIpos): m/z=661.5 [M+H−HCl]⁺.

### Example 24

### trans-4-(Aminomethyl)-N-[(2S)-1-{[4-(3-chloro-4H-1,2,4-triazol-5-yl)phenyl]amino}-3-{3′-[(4-methylpiperazin-1-yl)carbonyl]biphenyl-3-yl}-1-oxopropan-2-yl]cyclohexanecarboxamide hydrochloride

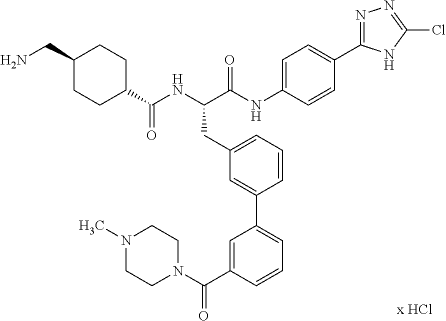

0.03 ml (0.13 mmol) of 4M hydrogen chloride in 1,4-dioxane was added to a suspension of 14 mg (0.02 mmol) of tert-butyl [(trans-4-{[(2S)-1-{[4-(3-chloro-4H-1,2,4-triazol-5-yl)phenyl]amino}-3-{3′-[(4-methylpiperazin-1-yl)carbonyl]biphenyl-3-yl}-1-oxopropan-2-yl]carbamoyl}cyclohexyl)methyl]carbamate in 0.5 ml of 1,4-dioxane, and the mixture was stirred at RT overnight. 5 ml of acetonitrile were then added to the reaction mixture and the resulting precipitate was filtered off with suction and dried under high vacuum. This gave 11 mg (75% of theory) of the title compound.

¹H NMR (400 MHz, DMSO-d₆): δ=ppm 0.75-0.95 (m, 2H), 1.02-1.16 (m, 1H), 1.16-1.28 (m, 1H), 1.36-1.52 (m, 2H), 1.60-1.76 (m, 3H), 2.05-2.16 (m, 1H), 2.54-2.61 (m, 2H), 2.72-2.79 (m, 3H), 2.93 (dd, 1H), 3.04-3.17 (m, 3H), 4.68-4.78 (m, 1H), 7.27-7.43 (m, 3H), 7.48-7.55 (m, 2H), 7.64-7.82 (m, 8H), 7.89 (d, 2H), 8.25 (d, 1H), 10.56 (s, 1H), 10.87-11.02 (m, 1H), 14.72 (s, 1H).

LC-MS (Method 4): Rₜ=0.66 min; MS (ESIpos): m/z=683.5 [M+H−HCl]⁺.

### Example 25

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-{2-[2-(2-hydroxy ethoxy)ethoxy]ethyl}-2-methylbiphenyl-4-carboxamide hydrochloride

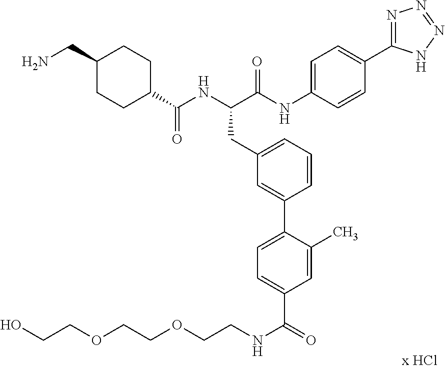

0.12 ml (0.5 mmol) of 4M hydrogen chloride in 1,4-dioxane was added to a suspension of 55 mg (0.07 mmol) of tert-butyl [(trans-4-{[(2S)-3-[4′-({2-[2-(2-hydroxyethoxy)ethoxy]ethyl}carbamoyl)-2′-methylbiphenyl-3-yl]-1-oxo-1-{[4-(1H-tetrazol-5-yl)phenyl]amino}propan-2-yl]carbamoyl}cyclohexyl)methyl]carbamate in 2.0 ml of 1,4-dioxane, and the mixture was stirred at RT overnight. After addition of a further 0.05 ml (0.2 mmol) of 4M hydrogen chloride in 1,4-dioxane and 48 h at RT, 5 ml of acetonitrile were added to the reaction mixture and the precipitate formed was filtered off with suction, suspended in water and lyophilized. This gave 27 mg (51% of theory) of the title compound.

¹H NMR (300 MHz, DMSO-d₆): δ=ppm 0.74-0.96 (m, 2H), 1.02-1.30 (m, 2H), 1.46 (br. s., 2H), 1.61-1.77 (m, 3H), 2.01-2.15 (m, 1H), 2.18 (s, 3H), 2.54-2.63 (m, 2H), 2.90 (dd, 1H), 3.10 (dd, 1H), 3.33-3.55 (m, 16H), 4.64-4.76 (m, 1H), 7.09-7.38 (m, 5H), 7.68 (d, 1H), 7.74 (s, 1H), 7.79 (m, 4H), 7.98 (d, 2H), 8.23 (d, 1H), 8.51 (t, 1H), 10.54 (s, 1H).

LC-MS (Method 4): Rt=0.75 min; MS (ESIpos): m/z=713.6 [M+H−HCl]⁺.

### Example 26

### trans-4-(Aminomethyl)-N-{(2S)-1-{[4-(3-chloro-4H-1,2,4-triazol-5-yl)phenyl]amino}-3-[4′-(morpholin-4-ylcarbonyl)biphenyl-3-yl]-1-oxopropan-2-yl}cyclohexanecarboxamide hydrochloride

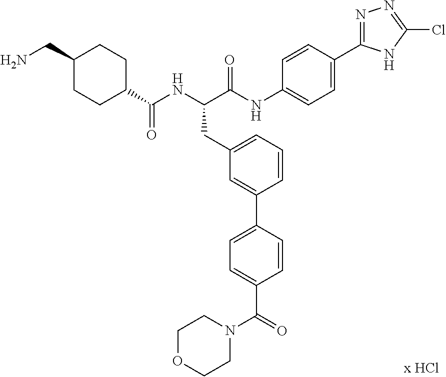

0.12 ml (0.5 mmol) of 4M hydrogen chloride in 1,4-dioxane was added to a suspension of 52 mg (0.07 mmol) of tert-butyl {[trans-4-({(2)-1-{[4-(3-chloro-4H-1,2,4-triazol-5-yl)phenyl]amino}-3-[4′-(morpholin-4-ylcarbonyl)biphenyl-3-yl]-1-oxopropan-2-yl}carbamoyl)cyclohexyl]methyl}carbamate in 2.0 ml of 1,4-dioxane, and the mixture was stirred at RT overnight. 5 ml of acetonitrile were then added to the reaction mixture and the resulting precipitate was filtered off with suction and dried under high vacuum. This gave 42 mg (87% of theory) of the title compound.

¹H NMR (300 MHz, DMSO-d₆): δ=ppm 0.73-0.95 (m, 2H), 0.97-1.28 (m, 2H), 1.31-1.52 (m, 2H), 1.57-1.76 (m, 3H), 2.04-2.17 (m, 1H), 2.55-2.64 (m, 2H), 2.92 (dd, 1H), 3.11 (dd, 1H), 3.49-3.69 (m, 8H), 4.65-4.76 (m, 1H), 7.25-7.33 (m, 1H), 7.34-7.38 (m, 1H), 7.45 (m, 3H), 7.60 (s, 1H), 7.62-7.78 (m, 7H), 7.88 (d, 2H), 8.22 (d, 1H), 10.46 (s, 1H), 14.67 (s, 1H).

LC-MS (Method 4): Rₜ=0.83 min; MS (ESIpos): m/z=670.5 [M+H−HCl]⁺.

### Example 27

### trans-4-(Aminomethyl)-N-[(2S)-1-{[4-(3-chloro-4H-1,2,4-triazol-5-yl)phenyl]amino}-3-{4′-[(4-methylpiperazin-1-yl)carbonyl]biphenyl-3-yl}-1-oxopropan-2-yl]cyclohexanecarboxamide hydrochloride

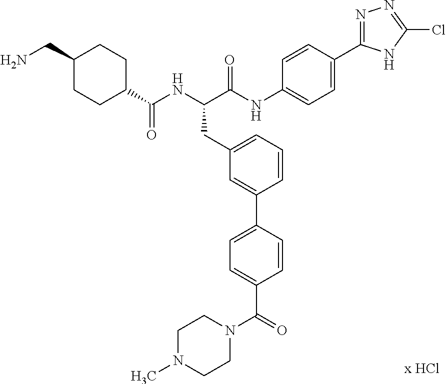

0.15 ml (0.6 mmol) of 4M hydrogen chloride in 1,4-dioxane was added to a suspension of 69 mg (0.07 mmol) of tert-butyl [(trans-4-{[(2S)-1-{[4-(3-chloro-4H-1,2,4-triazol-5-yl)phenyl]amino}-3-{4′-[(4-methylpiperazin-1-yl)carbonyl]biphenyl-3-yl}-1-oxopropan-2-yl]carbamoyl}cyclohexyl)methyl]carbamate in 2.6 ml of 1,4-dioxane, and the mixture was stirred at RT overnight. 5 ml of acetonitrile were then added to the reaction mixture and the resulting precipitate was filtered off with suction and dried under high vacuum. This gave 61 mg (95% of theory) of the title compound.

¹H NMR (300 MHz, DMSO-d₆): δ=ppm 0.75-0.95 (m, 2H), 1.00-1.30 (m, 2H), 1.33-1.54 (m, 2H), 1.59-1.77 (m, 3H), 2.04-2.16 (m, 1H), 2.54-2.63 (m, 2H), 2.75 (m, 3H), 2.93 (dd, 1H), 3.00-3.17 (m, 4H), 3.31-3.43 (m, 4H), 4.67-4.77 (m, 1H), 7.30 (d, 1H), 7.36 (t, 1H), 7.51 (m, 3H), 7.64 (s, 1H), 7.70 (d, 2H), 7.76 (d, 2H), 7.79-7.86 (m, 3H), 7.89 (d, 2H), 8.28 (d, 1H), 10.55 (s, 1H), 10.97-11.15 (m, 1H), 14.61-14.90 (m, 1H).

LC-MS (Method 4): Rₜ=0.65 min; MS (ESIpos): m/z=683.5 [M+H−HCl]⁺.

### Example 28

### 5′-[(2S)-2-({[trans-4-(Aminomethyl)cyclohexyl]carbonyl}amino)-3-oxo-3-{[4-(1H-tetrazol-5-yl)phenyl]amino}propyl]-N-{2-[2-(2-hydroxyethoxy)ethoxy]ethyl}-2′-methylbiphenyl-3-carboxamide hydrochloride

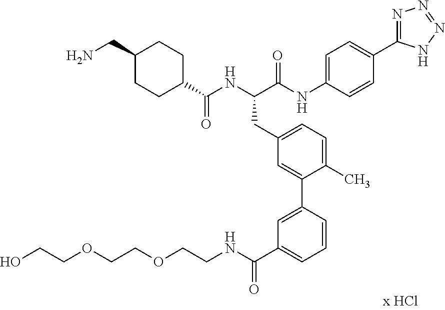

0.12 ml (0.5 mmol) of 4M hydrogen chloride in 1,4-dioxane was added to a suspension of 55 mg (0.07 mmol) of tert-butyl [(trans-4-{[(2S)-3-[3′-({2-[2-(2-hydroxyethoxy)ethoxy]ethyl}carbamoyl)-6-methylbiphenyl-3-yl]-1-oxo-1-{[4-(1H-tetrazol-5-yl)phenyl]amino}propan-2-yl]carbamoyl}cyclohexyl)methyl]carbamate in 2.0 ml of 1,4-dioxane, and the mixture was stirred at RT overnight. After addition of a further 0.07 ml (0.27 mmol) of 4M hydrogen chloride in 1,4-dioxane and 48 h at RT, 5 ml of acetonitrile were added to the reaction mixture and the precipitate formed was filtered off with suction, suspended in water and lyophilized. This gave 31 mg (60% of theory) of the title compound.

¹H NMR (400 MHz, DMSO-d₆): δ=ppm 0.78-0.94 (m, 2H), 1.06-1.28 (m, 2H), 1.35-1.48 (m, 1H), 1.49-1.56 (m, 1H), 1.64-1.77 (m, 3H), 2.05-2.12 (m, 1H), 2.14 (s, 3H), 2.55-2.61 (m, 2H), 2.86 (dd, 1H), 3.04 (dd, 1H), 3.40 (m, 7H), 3.46-3.53 (m, 8H), 4.61-4.69 (m, 1H), 7.15-7.22 (m, 3H), 7.38 (d, 1H), 7.47 (t, 1H), 7.67-7.75 (m, 2H), 7.78 (d, 3H), 7.79-7.83 (m, 1H), 7.97 (d, 2H), 8.15 (d, 1H), 8.54 (t, 1H), 10.46 (s, 1H).

LC-MS (Method 4): Rₜ=0.77 min; MS (ESIpos): m/z=713.6 [M+H−HCl]⁺.

### Example 29

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(morpholin-4-yl)ethyl]biphenyl-3-carboxamide hydrochloride

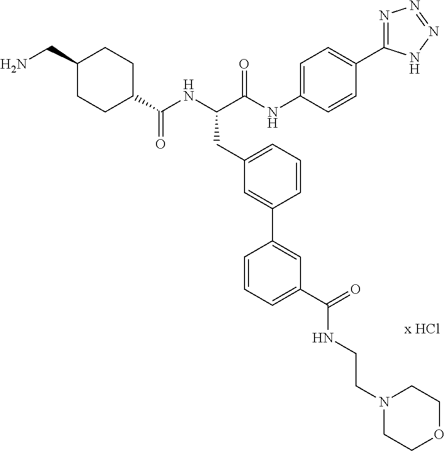

0.09 ml (0.36 mmol) of 4M hydrogen chloride in 1,4-dioxane was added to a suspension of 40 mg (0.05 mmol) of tert-butyl [(trans-4-{[(2S)-3-(3′-{[2-(morpholin-4-yl)ethyl]carbamoyl}biphenyl-3-yl)-1-oxo-1-{[4-(1H-tetrazol-5-yl)phenyl]amino}propan-2-yl]carbamoyl}cyclohexyl)methyl]carbamate in 1.5 ml of 1,4-dioxane, and the mixture was stirred at RT overnight. After addition of a further 0.09 ml (0.36 mmol) of 4M hydrogen chloride in 1,4-dioxane and 24 h at RT, 5 ml of acetonitrile were added to the reaction mixture and the precipitate formed was filtered off with suction, suspended in water and lyophilized. This gave 37 mg (92% of theory) of the title compound.

¹H NMR (300 MHz, DMSO-d₆): δ=ppm 0.72-0.97 (m, 2H), 0.99-1.28 (m, 2H), 1.30-1.56 (m, 2H), 1.59-1.76 (m, 3H), 2.07-2.19 (m, 1H), 2.54-2.62 (m, 2H), 2.94 (dd, 1H), 3.05-3.18 (m, 3H), 3.30-3.35 (m, 4H), 3.65-3.74 (m, 3H), 3.76-3.86 (m, 2H), 3.89-4.00 (m, 2H), 4.69-4.79 (m, 1H), 7.30 (d, 1H), 7.37 (t, 1H), 7.51 (d, 1H), 7.57 (d, 1H), 7.69-7.90 (m, 8H), 7.96 (d, 2H), 8.18-8.28 (m, 2H), 8.98-9.06 (m, 1H), 10.57 (s, 1H).

LC-MS (Method 4): Rₜ=0.62 min; MS (ESIpos): m/z=680.5 [M+H−HCl]⁺.

### Example 30

### 3′-[(2S)-2-({[trans-4-(Aminomethyl)cyclohexyl]carbonyl}amino)-3-{[4-(3-chloro-4H-1,2,4-triazol-5-yl)phenyl]amino}-3-oxopropyl]-N-[1-(propan-2-yl)piperidin-4-yl]biphenyl-3-carboxamide hydrochloride

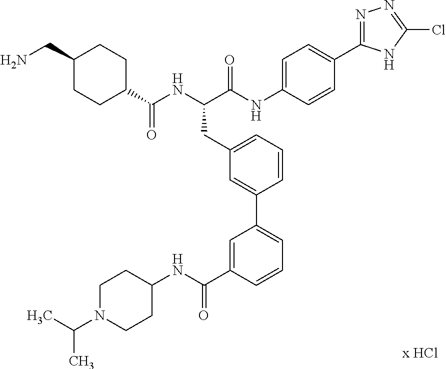

0.1 ml (0.42 mmol) of 4M hydrogen chloride in 1,4-dioxane was added to a suspension of 49 mg (0.06 mmol) of tert-butyl [(trans-4-{[(2S)-1-{[4-(3-chloro-4H-1,2,4-triazol-5-yl)phenyl]amino}-1-oxo-3-(3′-{[1-(propan-2-yl)piperidin-4-yl]carbamoyl}biphenyl-3-yl)propan-2-yl]carbamoyl}cyclohexyl)methyl]carbamate in 1.8 ml of 1,4-dioxane, and the mixture was stirred at RT overnight. 5 ml of acetonitrile were then added to the reaction mixture and the resulting precipitate was filtered off with suction and dried under high vacuum. This gave 45 mg (94% of theory) of the title compound.

¹H NMR (300 MHz, DMSO-d₆): δ=ppm 0.75-0.95 (m, 2H), 1.01-1.20 (m, 2H), 1.25 (d, 6H), 1.35-1.53 (m, 3H), 1.59-1.77 (m, 4H), 1.94-2.05 (m, 4H), 2.07-2.17 (m, 1H), 2.54-2.61 (m, 2H), 2.93 (dd, 1H), 3.01-3.17 (m, 3H), 3.31-3.41 (m, 3H), 3.99-4.11 (m, 1H), 4.67-4.78 (m, 1H), 7.30 (d, 1H), 7.36 (t, 1H), 7.47-7.56 (m, 2H), 7.68 (s, 1H), 7.72-7.85 (m, 9H), 7.87 (d, 2H), 8.12 (s, 1H), 8.26 (d, 1H), 8.67 (d, 1H), 10.02-10.13 (m, 1H), 10.54 (s, 1H).

LC-MS (Method 4): Rₜ=0.70 min; MS (ESIpos): m/z=725.6 [M+H−HCl]⁺.

### Example 31

### trans-4-(Aminomethyl)-N-[(2S)-3-{3′-[(4-methylpiperazin-1-yl)carbonyl]biphenyl-3-yl}-1-oxo-1-{[4-(1H-tetrazol-5-yl)phenyl]amino}propan-2-yl]cyclohexanecarboxamide hydrochloride

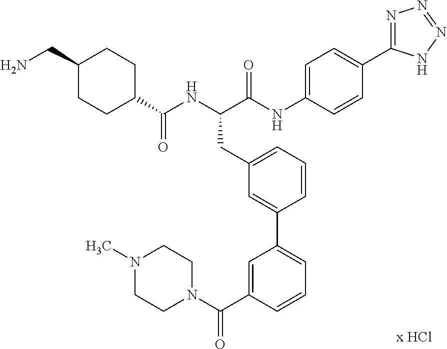

0.11 ml (0.4 mmol) of 4M hydrogen chloride in 1,4-dioxane was added to a suspension of 47 mg (0.06 mmol) of tert-butyl [(trans-4-{[(2S)-3-{3′-[(4-methylpiperazin-1-yl)carbonyl]biphenyl-3-yl}-1-oxo-1-{[4-(1H-tetrazol-5-yl)phenyl]amino}propan-2-yl]carbamoyl}cyclohexyl)methyl]carbamate in 2.0 ml of 1,4-dioxane, and the mixture was stirred at RT overnight. After addition of a further 0.03 ml (0.13 mmol) of 4M hydrogen chloride in 1,4-dioxane and 24 h at RT, 5 ml of acetonitrile were added to the reaction mixture and the precipitate formed was filtered off with suction and dried under high vacuum. This gave 43 mg (88% of theory) of the title compound.

¹H NMR (300 MHz, DMSO-d₆): δ=ppm 0.68-0.96 (m, 2H), 1.02-1.26 (m, 2H), 1.34-1.53 (m, 2H), 1.59-1.77 (m, 3H), 2.05-2.16 (m, 1H), 2.55-2.63 (m, 2H), 2.76 (s, 3H), 2.93 (dd, 1H), 3.03-3.19 (m, 4H), 4.68-4.79 (m, 1H), 7.27-7.42 (m, 3H), 7.47-7.56 (m, 2H), 7.63-7.84 (m, 9H), 7.96 (d, 2H), 8.25 (d, 1H), 10.60 (s, 1H), 10.89 (br. s, 1H).

LC-MS (Method 4): Rₜ=0.61 min; MS (ESIpos): m/z=650.5 [M+H−HCl]⁺.

### Example 32

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-[1-(propan-2-yl)piperidin-4-yl]biphenyl-3-carboxamide hydrochloride

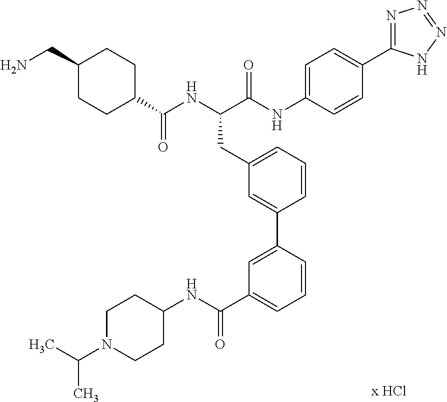

0.1 ml (0.4 mmol) of 4M hydrogen chloride in 1,4-dioxane was added to a suspension of 47 mg (0.06 mmol) of tert-butyl [(trans-4-{[(2S)-1-oxo-3-(3′-{[1-(propan-2-yl)piperidin-4-yl]carbamoyl}biphenyl-3-yl)-1-{[4-(1H-tetrazol-5-yl)phenyl]amino}propan-2-yl]carbamoyl}cyclohexyl)methyl]carbamate in 2.0 ml of 1,4-dioxane, and the mixture was stirred at RT for 48 h. 5 ml of acetonitrile were then added to the reaction mixture and the resulting precipitate was filtered off with suction and dried under high vacuum. This gave 36 mg (77% of theory) of the title compound.

¹H NMR (300 MHz, DMSO-d₆): δ=ppm 0.75-0.95 (m, 2H), 1.01-1.20 (m, 2H), 1.25 (m, 8H), 1.34-1.54 (m, 2H), 1.58-1.77 (m, 3H), 1.90-2.18 (m, 6H), 2.54-2.63 (m, 2H), 2.93 (dd, 1H), 2.99-3.18 (m, 3H), 3.97-4.13 (m, 1H), 4.66-4.79 (m, 1H), 7.30 (d, 1H), 7.36 (t, 1H), 7.47-7.57 (m, 3H), 7.67 (s, 1H), 7.71-7.86 (m, 7H), 7.96 (d, 2H), 8.11 (s, 1H), 8.26 (d, 1H), 8.66 (d, 1H), 9.84-9.97 (m, 1H), 10.56 (s, 1H).

LC-MS (Method 4): Rₜ=0.65 min; MS (ESIpos): m/z=692.6 [M+H−HCl]⁺.

### Example 33

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-(3-azabicyclo[3.1.0]hex-6-yl)-2-methylbiphenyl-4-carboxamide hydrochloride

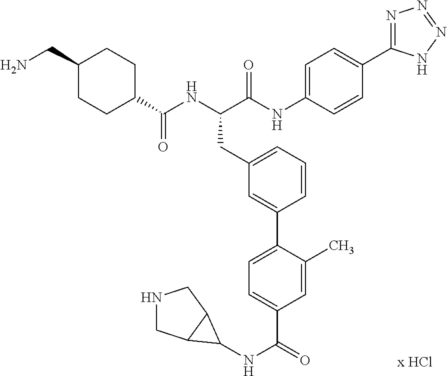

0.15 ml (0.6 mmol) of 4M hydrogen chloride in 1,4-dioxane was added to a suspension of 51 mg (0.06 mmol) of tert-butyl 6-[({3′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2-methylbiphenyl-4-yl}carbonyl)amino]-3-azabicyclo[3.1.0]hexane-3-carboxylate in 2.0 ml of 1,4-dioxane, and the mixture was stirred at RT overnight. After addition of a further 0.07 ml (0.3 mmol) of 4M hydrogen chloride in 1,4-dioxane and 48 h at RT, 5 ml of acetonitrile were added to the reaction mixture and the precipitate formed was filtered off with suction, suspended in water and lyophilized. This gave 37 mg (83% of theory) of the title compound.

¹H NMR (300 MHz, DMSO-d₆): δ=ppm 0.74-0.97 (m, 2H), 1.02-1.28 (m, 2H), 1.34-1.53 (m, 2H), 1.62-1.77 (m, 3H), 1.95-2.02 (m, 2H), 2.03-2.14 (m, 1H), 2.17 (s, 3H), 2.56-2.65 (m, 2H), 2.91 (dd, 1H), 2.98-3.04 (m, 1H), 3.13 (dd, 1H), 3.33-3.36 (m, 5H), 4.64-4.76 (m, 1H), 7.16 (m, 2H), 7.24 (s, 1H), 7.28-7.37 (m, 2H), 7.65 (d, 1H), 7.71 (s, 1H), 7.79 (d, 2H), 7.83 (br. s., 3H), 7.98 (d, 2H), 8.23 (d, 1H), 8.59 (d, 1H), 8.90-9.06 (m, 1H), 9.44-9.59 (m, 1H), 10.54 (s, 1H).

LC-MS (Method 4): Rt=0.63 min; MS (ESIpos): m/z=662.6 [M+H−HCl]⁺.

### Example 34

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(diethylamino)ethyl]biphenyl-3-carboxamide hydrochloride

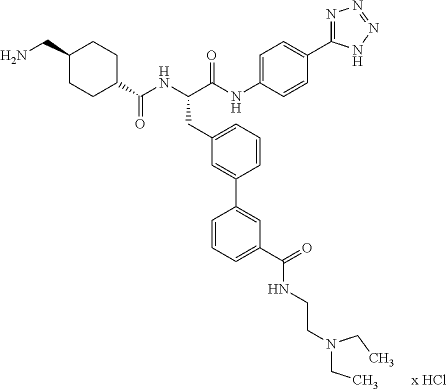

0.11 ml (0.45 mmol) of 4M hydrogen chloride in 1,4-dioxane was added to a suspension of 50 mg (0.06 mmol) of tert-butyl [(trans-4-{[(2S)-3-(3′-{[2-(diethylamino)ethyl]carbamoyl}biphenyl-3-yl)-1-oxo-1-{[4-(1H-tetrazol-5-yl)phenyl]amino}propan-2-yl]carbamoyl}cyclohexyl)methyl]carbamate in 2.0 ml of 1,4-dioxane, and the mixture was stirred at RT overnight. 5 ml of acetonitrile were then added to the reaction mixture and the resulting precipitate was filtered off with suction and dried under high vacuum. This gave 38 mg (77% of theory) of the title compound.

¹H NMR (400 MHz, DMSO-d₆): δ=ppm 0.77-0.94 (m, 2H), 1.02-1.17 (m, 2H), 1.21 (t, 6H), 1.35-1.44 (m, 1H), 1.46-1.53 (m, 1H), 1.60-1.76 (m, 3H), 2.07-2.17 (m, 1H), 2.54-2.62 (m, 2H), 2.93 (dd, 1H), 3.12 (dd, 1H), 3.19 (q, 4H), 3.23-3.27 (m, 3H), 3.62-3.69 (m, 2H), 4.69-4.77 (m, 1H), 7.31 (d, 1H), 7.36 (t, 1H), 7.52 (d, 1H), 7.57 (dd, 1H), 7.72 (br. s, 3H), 7.76-7.88 (m, 6H), 7.96 (d, 2H), 8.22-8.29 (m, 2H), 9.04 (t, 1H), 10.62 (s, 1H).

LC-MS (Method 4): Rₜ=0.64 min; MS (ESIpos): m/z=666.8 [M+H−HCl]⁺.

### Example 35

### 5′-[(2S)-2-({[trans-4-(Aminomethyl)cyclohexyl]carbonyl}amino)-3-oxo-3-{[4-(1H-tetrazol-5-yl)phenyl]amino}propyl]-N-(3-azabicyclo[3.1.0]hex-6-yl)-2′-methylbiphenyl-3-carboxamide hydrochloride

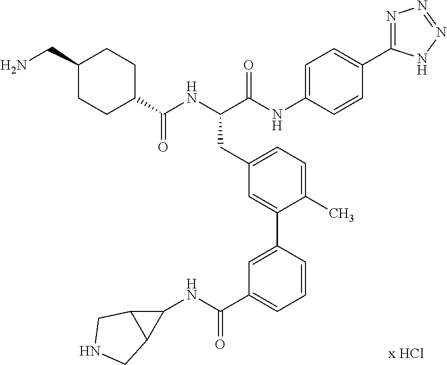

0.15 ml (0.6 mmol) of 4M hydrogen chloride in 1,4-dioxane was added to a suspension of 50 mg (0.06 mmol) of tert-butyl 6-[({5′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2′-methylbiphenyl-3-yl}carbonyl)amino]-3-azabicyclo[3.1.0]hexane-3-carboxylate in 2.0 ml of 1,4-dioxane, and the mixture was stirred at RT overnight. After adding a further 0.07 ml (0.3 mmol) of 4M hydrogen chloride in 1,4-dioxane and 48 h of stirring at RT and 24 h of stirring at 40° C., 5 ml of acetonitrile were added to the reaction mixture and the precipitate formed was filtered off with suction, suspended in water and lyophilized. This gave 28 mg (63% of theory) of the title compound.

¹H NMR (300 MHz, DMSO-d₆): δ=ppm 0.78-0.96 (m, 2H), 1.02-1.30 (m, 2H), 1.35-1.59 (m, 2H), 1.61-1.79 (m, 3H), 1.98 (br. s., 2H), 2.09-2.16 (m, 4H), 2.57-2.65 (m, 2H), 2.86 (dd, 1H), 2.97-3.08 (m, 2H), 4.58-4.70 (m, 1H), 7.13-7.23 (m, 3H), 7.39 (d, 1H), 7.47 (t, 1H), 7.78 (m, 8H), 7.96 (d, 2H), 8.19 (d, 1H), 8.61 (d, 1H), 8.83-9.01 (m, 1H), 9.35-9.50 (m, 1H), 10.50 (s, 1H).

LC-MS (Method 4): Rₜ=0.64 min; MS (ESIpos): m/z=666.8 [M+H−HCl]⁺.

### Example 36

### trans-4-(Aminomethyl)-N-[(2S)-3-[2′-methyl-5′-(pyrrolidin-1-ylsulphonyl)biphenyl-3-yl]-1-oxo-1-{[4-(5-oxo-4,5-dihydro-1,2,4-oxadiazol-3-yl)phenyl]amino}propan-2-yl]cyclohexanecarboxamide hydrochloride

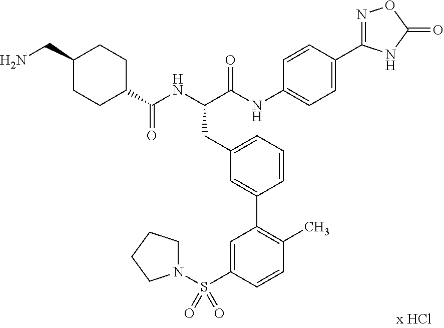

0.13 ml (0.52 mmol) of 4M hydrogen chloride in 1,4-dioxane was added to a suspension of 58 mg (0.07 mmol) of tert-butyl [(trans-4-{[(2S)-3-[2′-methyl-5′-(pyrrolidin-1-ylsulphonyl)biphenyl-3-yl]-1-oxo-1-{[4-(5-oxo-4,5-dihydro-1,2,4-oxadiazol-3-yl)phenyl]amino}propan-2-yl]carbamoyl}cyclohexyl)methyl]carbamate in 2.6 ml of 1,4-dioxane, and the mixture was stirred at RT overnight. After adding a further 0.06 ml (0.22 mmol) of 4M hydrogen chloride in 1,4-dioxane and 48 h of stirring at RT and 48 h of stirring at 40° C., 5 ml of acetonitrile were added to the reaction mixture and the precipitate formed was filtered off with suction and dried under high vacuum. This gave 41 mg (76% of theory) of the title compound.

¹H NMR (300 MHz, DMSO-d₆): δ=ppm 0.76-0.94 (m, 2H), 1.01-1.30 (m, 2H), 1.33-1.51 (m, 2H), 1.58-1.77 (m, 7H), 2.01-2.13 (m, 1H), 2.22 (s, 3H), 2.56-2.63 (m, 2H), 2.91 (dd, 1H), 3.05-3.15 (m, 5H), 4.65-4.77 (m, 1H), 7.15-7.22 (m, 1H), 7.24-7.28 (m, 1H), 7.29-7.39 (m, 2H), 7.46 (d, 1H), 7.52 (d, 1H), 7.61-7.79 (m, 8H), 8.21 (d, 1H), 10.49 (s, 1H).

LC-MS (Method 4): Rₜ=0.92 min; MS (ESIpos): m/z=687.4 [M+H−HCl]⁺

### Example 37

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(piperidin-1-yl)ethyl]biphenyl-4-carboxamide hydrochloride

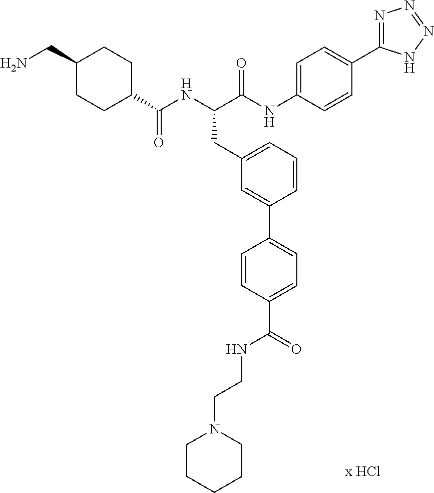

0.17 ml (0.67 mmol) of 4M hydrogen chloride in 1,4-dioxane was added to a suspension of 74 mg (0.09 mmol) of tert-butyl [(trans-4-{[(2S)-1-oxo-3-(4′-{[2-(piperidin-1-yl)ethyl]carbamoyl}biphenyl-3-yl)-1-{[4-(1H-tetrazol-5-yl)phenyl]amino}propan-2-yl]carbamoyl}cyclohexyl)methyl]carbamate in 3.5 ml of 1,4-dioxane, and the mixture was stirred at RT overnight. 5 ml of acetonitrile were then added to the reaction mixture and the resulting precipitate was filtered off with suction and dried under high vacuum. This gave 68 mg (91% of theory) of the title compound.

¹H NMR (300 MHz, DMSO-d₆): δ=ppm 0.74-0.95 (m, 2H), 1.00-1.27 (m, 2H), 1.30-1.54 (m, 2H), 1.73 (m, 7H), 2.07-2.19 (m, 1H), 2.55-2.63 (m, 2H), 2.84-2.98 (m, 3H), 3.14 (dd, 1H), 3.20-3.26 (m, 3H), 3.49-3.59 (m, 2H), 3.63-3.73 (m, 2H), 4.68-4.79 (m, 1H), 7.27-7.39 (m, 2H), 7.48-7.59 (m, 2H), 7.83 (m, 9H), 7.96 (d, 2H), 8.20-8.31 (m, 2H), 9.00-9.08 (m, 1H), 9.80-9.99 (m, 1H), 10.62 (s, 1H).

LC-MS (Method 4): Rₜ=0.79 min; MS (ESIpos): m/z=678.6 [M+H−HCl]⁺

### Example 38

### 3′-[(2S)-2-({[trans-4-(Aminomethyl)cyclohexyl]carbonyl}amino)-3-oxo-3-{[4-(1H-tetrazol-5-yl)phenyl]amino}propyl]-6-chlorobiphenyl-3-carboxamide hydrochloride

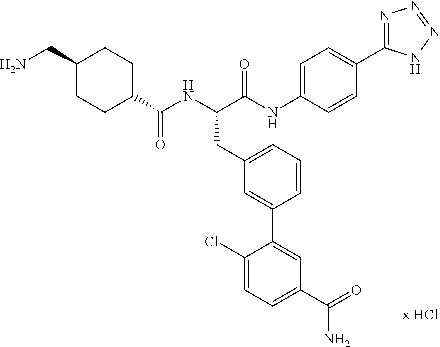

0.14 ml (0.55 mmol) of 4M hydrogen chloride in 1,4-dioxane was added to a suspension of 55 mg (0.08 mmol) of tert-butyl [(trans-4-{[(2S)-3-(5′-carbamoyl-2′-chlorobiphenyl-3-yl)-1-oxo-1-{[4-(1H-tetrazol-5-yl)phenyl]amino}propan-2-yl]carbamoyl}cyclohexyl)methyl]carbamate in 2.5 ml of dichloromethane, and the mixture was stirred at RT overnight. After adding a further 0.14 ml (0.55 mmol) of 4M hydrogen chloride in 1,4-dioxane and 24 h of stirring at RT, 5 ml of acetonitrile were added to the reaction mixture and the precipitate formed was filtered off with suction. The residue was purified by chromatography via HPLC (Method 7). This gave 19 mg (35% of theory) of the title compound.

¹H NMR (300 MHz, DMSO-d₆): δ=ppm 0.74-0.93 (m, 2H), 1.02-1.30 (m, 2H), 1.30-1.42 (m, 1H), 1.43-1.53 (m, 1H), 1.57-1.77 (m, 3H), 2.01-2.15 (m, 1H), 2.56-2.60 (m, 2H), 2.90 (dd, 1H), 3.12 (dd, 2H), 4.65-4.77 (m, 1H), 7.25-7.31 (m, 1H), 7.33-7.40 (m, 3H), 7.43-7.48 (m, 1H), 7.55 (d, 2H), 7.61 (d, 1H), 7.79-7.90 (m, 4H), 8.02-8.12 (m, 2H), 10.09 (s, 1H).

LC-MS (Method 4): Rₜ=0.77 min; MS (ESIpos): m/z=601.4 [M+H−HCl]⁺.

### Example 39

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(dimethylamino)ethyl]biphenyl-3-carboxamide hydrochloride

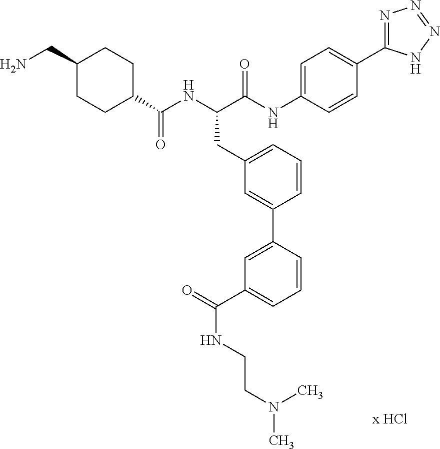

0.19 ml (0.77 mmol) of 4M hydrogen chloride in 1,4-dioxane was added to a suspension of 81 mg (0.11 mmol) of tert-butyl [(trans-4-{[(2S)-3-(3′-{[2-(dimethylamino)ethyl]carbamoyl}biphenyl-3-yl)-1-oxo-1-{[4-(1H-tetrazol-5-yl)phenyl]amino}propan-2-yl]carbamoyl}cyclohexyl)methyl]carbamate in 3.9 ml of 1,4-dioxane, and the mixture was stirred at RT overnight. 5 ml of acetonitrile were then added to the reaction mixture and the resulting precipitate was filtered off with suction and dried under high vacuum. This gave 57 mg (78% of theory) of the title compound.

¹H NMR (400 MHz, DMSO-d₆): δ=ppm 0.77-0.94 (m, 2H), 1.03-1.15 (m, 1H), 1.15-1.28 (m, 1H), 1.34-1.46 (m, 1H), 1.47-1.54 (m, 1H), 1.60-1.77 (m, 3H), 2.08-2.18 (m, 1H), 2.54-2.61 (m, 2H), 2.82 (s, 6H), 2.94 (dd, 1H), 3.15 (dd, 1H), 3.29 (dd, 2H), 3.65 (dd, 2H), 4.70-4.78 (m, 1H), 7.32 (d, 1H), 7.36 (t, 1H), 7.53 (t, 1H), 7.55-7.59 (m, 1H), 7.76-7.89 (m, 9H), 7.99 (d, 2H), 8.27 (s, 2H), 8.96-9.04 (m, 1H), 10.08-10.20 (m, 1H), 10.64 (s, 1H).

LC-MS (Method 4): Rₜ=0.61 min; MS (ESIpos): m/z=638.7 [M+H−HCl]⁺

### Example 40

### 3′-[(2S)-2-({[trans-4-(Aminomethyl)cyclohexyl]carbonyl}amino)-3-{[4-(3-chloro-4H-1,2,4-triazol-5-yl)phenyl]amino}-3-oxopropyl]-N-[2-(diethylamino)ethyl]biphenyl-3-carboxamide hydrochloride

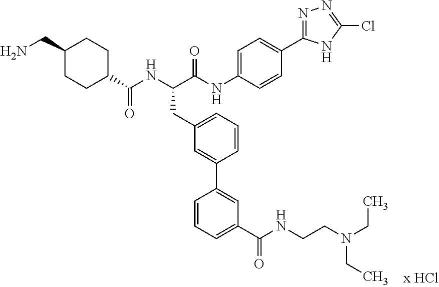

0.06 ml (0.24 mmol) of 4M hydrogen chloride in 1,4-dioxane was added to a suspension of 27 mg (0.03 mmol) of tert-butyl [(trans-4-{[(2S)-1-{[4-(3-chloro-4H-1,2,4-triazol-5-yl)phenyl]amino}-3-(3′-{[2-(diethylamino)ethyl]carbamoyl}biphenyl-3-yl)-1-oxopropan-2-yl]carbamoyl}cyclohexyl)methyl]carbamate in 1.0 ml of dichloromethane, and the mixture was stirred at RT overnight. After adding a further 0.06 ml (0.24 mmol) of 4M hydrogen chloride in 1,4-dioxane and 24 h of stirring at RT, 5 ml of acetonitrile were added to the reaction mixture and the precipitate formed was filtered off with suction. The residue was purified by chromatography via HPLC (Method 7). This gave 26 mg (94% of theory) of the title compound.

¹H NMR (300 MHz, DMSO-d₆): δ=ppm 0.73-0.95 (m, 2H), 0.98-1.17 (m, 2H), 1.22 (t, 6H), 1.34-1.55 (m, 3H), 1.59-1.78 (m, 3H), 2.06-2.18 (m, 1H), 2.53-2.63 (m, 2H), 2.93 (dd, 1H), 3.08-3.31 (m, 8H), 4.67-4.78 (m, 1H), 7.27-7.39 (m, 2H), 7.48-7.60 (m, 3H), 7.69-7.92 (m, 10H), 8.22 (d, 1H), 8.27 (s, 1H), 9.01-9.09 (m, 1H), 9.99-10.12 (m, 1H), 10.57 (s, 1H).

LC-MS (Method 4): Rₜ=0.69 min; MS (ESIpos): m/z=699.5 [M+H−HCl]⁺

### Example 41

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(pyrrolidin-1-yl)ethyl]biphenyl-4-carboxamide hydrochloride

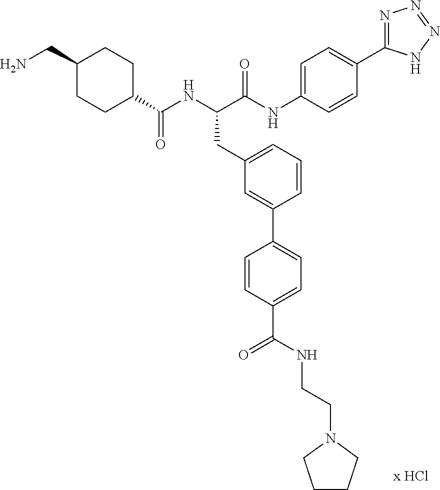

0.12 ml (0.47 mmol) of 4M hydrogen chloride in 1,4-dioxane was added to a suspension of 51 mg (0.07 mmol) of tert-butyl [(trans-4-{[(2S)-1-oxo-3-(4′-{[2-(pyrrolidin-1-yl)ethyl]carbamoyl}biphenyl-3-yl)-1-{[4-(1H-tetrazol-5-yl)phenyl]amino}propan-2-yl]carbamoyl}cyclohexyl)methyl]carbamate in 2.0 ml of 1,4-dioxane, and the mixture was stirred at RT for 48 h. 5 ml of acetonitrile were then added to the reaction mixture and the resulting precipitate was filtered off with suction and dried under high vacuum. This gave 49 mg (97% of theory) of the title compound.

¹H NMR (300 MHz, DMSO-d₆): δ=ppm 0.77-0.96 (m, 2H), 1.01-1.30 (m, 2H), 1.35-1.45 (m, 1H), 1.46-1.55 (m, 1H), 1.61-1.76 (m, 3H), 1.80-1.90 (m, 2H), 1.92-2.03 (m, 2H), 2.06-2.18 (m, 1H), 2.55-2.63 (m, 2H), 2.94 (dd, 1H), 2.99-3.07 (m, 2H), 3.13 (dd, 1H), 3.30-3.35 (m, 3H), 3.61 (m, 4H), 4.67-4.78 (m, 1H), 7.31 (d, 1H), 7.36 (t, 1H), 7.51-7.56 (m, 1H), 7.66 (s, 1H), 7.72 (d, 2H), 7.81 (m, 5H), 7.99 (d, 4H), 8.26 (d, 1H), 8.84-8.94 (m, 1H), 10.21-10.35 (m, 1H), 10.59 (s, 1H).

LC-MS (Method 4): Rₜ=0.63 min; MS (ESIpos): m/z=664.6 [M+H−HCl]⁺

### Example 42

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(diethylamino)ethyl]biphenyl-4-carboxamide hydrochloride

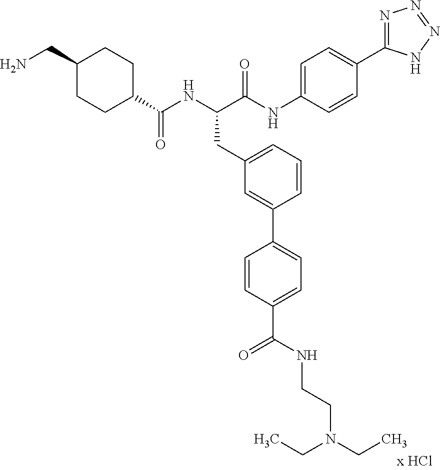

0.2 ml (0.82 mmol) of 4M hydrogen chloride in 1,4-dioxane was added to a suspension of 89 mg (0.12 mmol) of tert-butyl [(trans-4-{[(2S)-3-(4′-{[2-(diethylamino)ethyl]carbamoyl}biphenyl-3-yl)-1-oxo-1-{[4-(1H-tetrazol-5-yl)phenyl]amino}propan-2-yl]carbamoyl}cyclohexyl)methyl]carbamate in 3.5 ml of 1,4-dioxane, and the mixture was stirred at RT for 48 h. 5 ml of acetonitrile were then added to the reaction mixture and the resulting precipitate was filtered off with suction and dried under high vacuum. This gave 92 mg (99% of theory) of the title compound.

¹H NMR (300 MHz, DMSO-d₆): δ=ppm 0.76-0.95 (m, 2H), 1.00-1.17 (m, 2H), 1.20 (t, 6H), 1.35-1.45 (m, 1H), 1.46-1.55 (m, 1H), 1.60-1.77 (m, 3H), 2.06-2.18 (m, 1H), 2.55-2.63 (m, 2H), 2.93 (dd, 1H), 3.08-3.26 (m, 7H), 3.58-3.67 (m, 2H), 4.67-4.78 (m, 1H), 7.29-7.33 (m, 1H), 7.37 (t, 1H), 7.51-7.56 (m, 1H), 7.66 (s, 1H), 7.70-7.83 (m, 7H), 7.98 (m, 4H), 8.26 (d, 1H), 8.91-8.98 (m, 1H), 9.96-10.06 (m, 1H), 10.56 (s, 1H).

LC-MS (Method 4): Rₜ=0.64 min; MS (ESIpos): m/z=666.6 [M+H−HCl]⁺

### Example 43

### 3′-[(2S)-2-({[trans-4-(Aminomethyl)cyclohexyl]carbonyl}amino)-3-oxo-3-{[4-(1H-tetrazol-5-yl)phenyl]amino}propyl]-N,N-dimethylbiphenyl-3-carboxamide hydrochloride

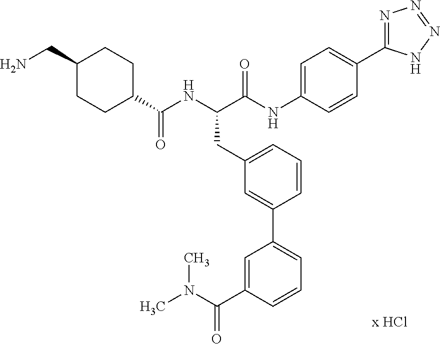

0.14 ml (0.55 mmol) of 4M hydrogen chloride in 1,4-dioxane was added to a suspension of 55 mg (0.08 mmol) of tert-butyl [(trans-4-{[(2S)-3-[3′-(dimethylcarbamoyl)biphenyl-3-yl]-1-oxo-1-{[4-(1H-tetrazol-5-yl)phenyl]amino}propan-2-yl]carbamoyl}cyclohexyl)methyl]carbamate in 3.0 ml of 1,4-dioxane, and the mixture was stirred at RT overnight. After addition of a further 0.14 ml (0.55 mmol) of 4M hydrogen chloride in 1,4-dioxane and 4 h at 40° C. and 24 h of stirring at RT, 5 ml of acetonitrile were added to the reaction mixture and the precipitate formed was filtered off with suction and dried under high vacuum. This gave 46 mg (88% of theory) of the title compound.

¹H NMR (400 MHz, DMSO-d₆): δ=ppm 0.79-0.98 (m, 2H), 1.05-1.19 (m, 1H), 1.20-1.33 (m, 1H), 1.39-1.48 (m, 1H), 1.50-1.56 (m, 1H), 1.62-1.80 (m, 3H), 2.10-2.19 (m, 1H), 2.58-2.66 (m, 2H), 2.91-2.99 (m, 4H), 3.02 (br. s., 3H), 3.17 (dd, 1H), 4.73-4.82 (m, 1H), 7.38 (m, 3H), 7.49-7.57 (m, 2H), 7.65 (d, 2H), 7.71 (d, 1H), 7.76-7.88 (m, 5H), 8.02 (d, 2H), 8.26 (d, 1H), 10.57 (s, 1H).

LC-MS (Method 4): Rₜ=0.76 min; MS (ESIpos): m/z=595.6 [M+H−HCl]⁺

### Example 44

### trans-4-(Aminomethyl)-N-{(2S)-3-[5′-(dimethylsulphamoyl)-2′-methylbiphenyl-3-yl]-1-oxo-1-[(2-oxo-2,3-dihydro-1H-benzimidazol-5-yl)amino]propan-2-yl}cyclohexanecarboxamide hydrochloride

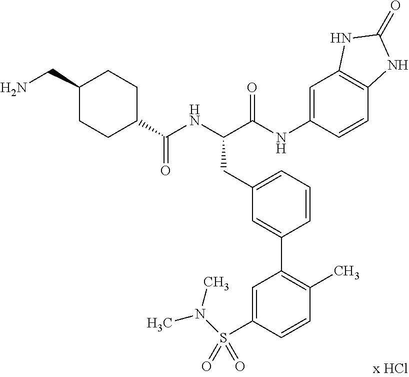

0.08 ml (0.32 mmol) of 4M hydrogen chloride in 1,4-dioxane was added to a suspension of 34 mg (0.05 mmol) of tert-butyl {[trans-4-({(2S)-3-[5′-(dimethylsulphamoyl)-2′-methylbiphenyl-3-yl]-1-oxo-1-[(2-oxo-2,3-dihydro-1H-benzimidazol-5-yl)amino]propan-2-yl}carbamoyl)cyclohexyl]methyl}carbamate in 1.6 ml of 1,4-dioxane, and the mixture was stirred at RT overnight. After adding a further 0.06 ml (0.23 mmol) of 4M hydrogen chloride in 1,4-dioxane and 4 h of stirring at 40° C., 5 ml of acetonitrile were added to the reaction mixture and the precipitate formed was filtered off with suction and dried under high vacuum. This gave 30 mg (93% of theory) of the title compound.

¹H NMR (300 MHz, MeOH-d₄): δ=ppm 0.90-1.11 (m, 2H), 1.24-1.50 (m, 2H), 1.50-1.60 (m, 1H), 1.61-1.71 (m, 1H), 1.74-1.90 (m, 3H), 2.19-2.25 (m, 1H), 2.23 (s, 3H), 2.64 (s, 6H), 2.75 (d, 2H), 3.04 (dd, 1H), 3.21 (dd, 1H), 4.77 (dd, 1H), 6.91 (d, 1H), 6.94-6.99 (m, 1H), 7.15-7.20 (m, 1H), 7.21 (s, 1H), 7.32-7.43 (m, 3H), 7.45-7.50 (m, 2H), 7.62 (dd, 1H).

LC-MS (Method 4): Rₜ=0.81 min; MS (ESIpos): m/z=633.3 [M+H−HCl]⁺

### Example 45

### 3′-[(2S)-2-({[trans-4-(Aminomethyl)cyclohexyl]carbonyl}amino)-3-oxo-3-{[4-(1H-tetrazol-5-yl)phenyl]amino}propyl]-6-fluoro-N-methoxy-N-methylbiphenyl-3-carboxamide hydrochloride

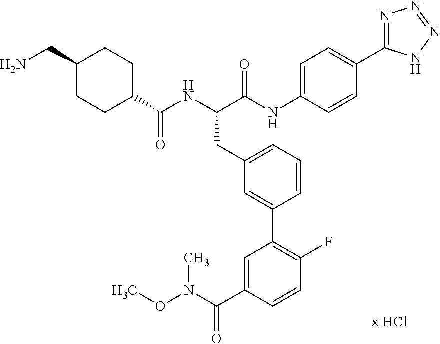

0.25 ml (1.00 mmol) of 4M hydrogen chloride in 1,4-dioxane was added to a suspension of 105 mg (0.14 mmol) of tert-butyl [(trans-4-{[(2S)-3-{2′-fluoro-5′-[methoxy(methyl)carbamoyl]biphenyl-3-yl}-1-oxo-1-{[4-(1H-tetrazol-5-yl)phenyl]amino}propan-2-yl]carbamoyl}cyclohexyl)methyl]carbamate in 5.0 ml of 1,4-dioxane, and the mixture was stirred at RT overnight. After adding a further 0.04 ml (0.14 mmol) of 4M hydrogen chloride in 1,4-dioxane and 4 h of stirring at 40° C., 5 ml of acetonitrile were added to the reaction mixture and the precipitate formed was filtered off with suction. The precipitate was purified chromatographically by HPLC (Method 11). This gave 20 mg (20% of theory) of the title compound.

LC-MS (Method 5): Rₜ=0.69 min; MS (ESIpos): m/z=629.5 [M+H−HCl]⁺

### Example 46

### trans-4-(Aminomethyl)-N-[(2S)-3-{4′-[(4-methylpiperazin-1-yl)carbonyl]biphenyl-3-yl}-1-oxo-1-{[4-(1H-tetrazol-5-yl)phenyl]amino}propan-2-yl]cyclohexanecarboxamide hydrochloride

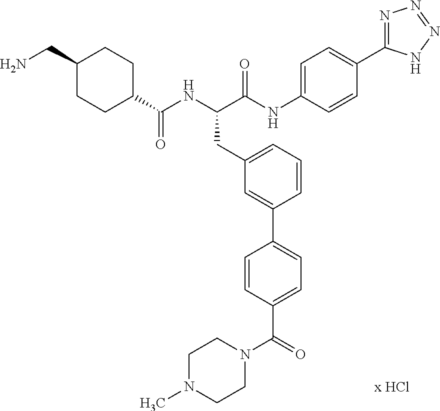

0.23 ml (0.90 mmol) of 4M hydrogen chloride in 1,4-dioxane was added to a suspension of 96 mg (0.13 mmol) of tert-butyl [(trans-4-{[(2S)-3-{4′-[(4-methylpiperazin-1-yl)carbonyl]biphenyl-3-yl}-1-oxo-1-{[4-(1H-tetrazol-5-yl)phenyl]amino}propan-2-yl]carbamoyl}cyclohexyl)methyl]carbamate in 4.5 ml of 1,4-dioxane, and the mixture was stirred at 40° C. for 4 h and at RT for 24 h. 5 ml of acetonitrile were then added to the reaction mixture and the resulting precipitate was filtered off with suction and dried under high vacuum. This gave 85 mg (90% of theory) of the title compound.

¹H NMR (300 MHz, DMSO-d₆): δ=ppm 0.76-0.96 (m, 2H), 1.00-1.30 (m, 2H), 1.34-1.54 (m, 2H), 1.59-1.78 (m, 3H), 2.06-2.17 (m, 1H), 2.54-2.62 (m, 2H), 2.74 (br. s., 3H), 2.94 (dd, 1H), 3.12 (m, 4H), 3.28-3.45 (m, 4H), 4.68-4.79 (m, 1H), 7.26-7.40 (m, 2H), 7.51 (m, 3H), 7.65 (s, 1H), 7.69 (d, 2H), 7.82 (d, 2H), 7.88 (br. s., 3H), 8.00 (d, 2H), 8.29 (d, 1H), 10.64 (s, 1H), 11.23 (br. s, 1H).

LC-MS (Method 4): Rₜ=0.70 min; MS (ESIpos): m/z=650.5 [M+H−HCl]⁺

### Example 47

### trans-4-(Aminomethyl)-N-[(2S)-3-[3′-(morpholin-4-ylcarbonyl)biphenyl-3-yl]-1-oxo-1-{[4-(1H-tetrazol-5-yl)phenyl]amino}propan-2-yl]cyclohexanecarboxamide hydrochloride

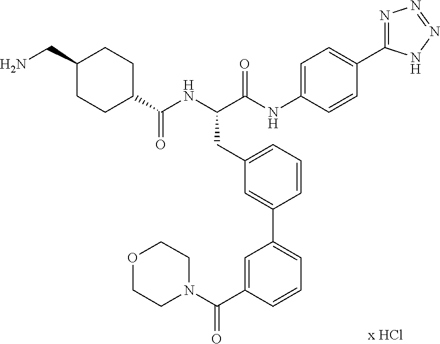

0.16 ml (0.65 mmol) of 4M hydrogen chloride in 1,4-dioxane was added to a suspension of 68 mg (0.09 mmol) of tert-butyl [(trans-4-{[(2S)-3-[3′-(morpholin-4-ylcarbonyl)biphenyl-3-yl]-1-oxo-1-{[4-(1H-tetrazol-5-yl)phenyl]amino}propan-2-yl]carbamoyl}cyclohexyl)methyl]carbamate in 3.2 ml of 1,4-dioxane, and the mixture was stirred at 40° C. for 4 h and at RT for 24 h. 5 ml of acetonitrile were then added to the reaction mixture and the resulting precipitate was filtered off with suction and dried under high vacuum. This gave 48 mg (75% of theory) of the title compound.

¹H NMR (300 MHz, DMSO-d₆): δ=ppm 0.74-0.95 (m, 2H), 0.99-1.28 (m, 2H), 1.33-1.52 (m, 2H), 1.58-1.77 (m, 3H), 2.05-2.16 (m, 1H), 2.55-2.62 (m, 2H), 2.92 (dd, 1H), 3.12 (dd, 1H), 3.55-3.75 (m, 8H), 4.67-4.78 (m, 1H), 7.25-7.39 (m, 3H), 7.45-7.53 (m, 2H), 7.62 (s, 2H), 7.66-7.77 (m, 4H), 7.79 (d, 2H), 7.97 (d, 2H), 8.24 (d, 1H), 10.54 (s, 1H).

LC-MS (Method 4): Rₜ=0.84 min; MS (ESIpos): m/z=637.5 [M+H−HCl]⁺

### Example 48

### trans-4-(Aminomethyl)-N-[(2S)-3-(4′-{[4-(2-hydroxyethyl)piperazin-1-yl]carbonyl}biphenyl-3-yl)-1-oxo-1-{[4-(1H-tetrazol-5-yl)phenyl]amino}propan-2-yl]cyclohexanecarboxamide hydrochloride

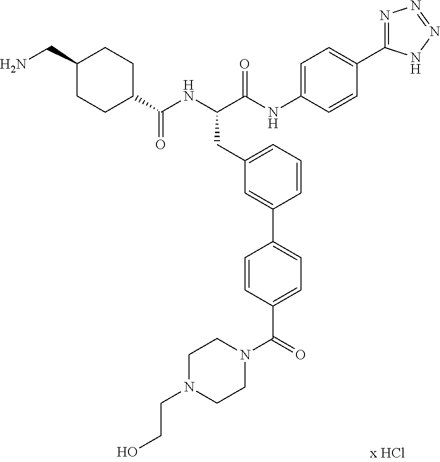

0.16 ml (0.65 mmol) of 4M hydrogen chloride in 1,4-dioxane was added to a suspension of 72 mg (0.09 mmol) of tert-butyl [(trans-4-{[(2S)-3-(4′-{[4-(2-hydroxyethyl)piperazin-1-yl]carbonyl}biphenyl-3-yl)-1-oxo-1-{[4-(1H-tetrazol-5-yl)phenyl]amino}propan-2-yl]carbamoyl}cyclohexyl)methyl]carbamate in 3.3 ml of 1,4-dioxane, and the mixture was stirred at RT overnight. 5 ml of acetonitrile were then added to the reaction mixture and the resulting precipitate was filtered off with suction and dried under high vacuum. This gave 66 mg (100% of theory) of the title compound.

¹H NMR (400 MHz, DMSO-d₆): δ=ppm 0.78-0.94 (m, 2H), 1.03-1.16 (m, 1H), 1.16-1.28 (m, 1H), 1.35-1.46 (m, 1H), 1.48-1.54 (m, 1H), 1.62-1.77 (m, 3H), 2.07-2.16 (m, 1H), 2.54-2.61 (m, 2H), 2.95 (dd, 1H), 3.10-3.19 (m, 4H), 3.72-3.79 (m, 2H), 4.68-4.77 (m, 1H), 5.25-5.39 (m, 1H), 7.31 (d, 1H), 7.36 (t, 1H), 7.51 (m, 3H), 7.63 (s, 1H), 7.69 (d, 2H), 7.80 (m, 5H), 7.98 (d, 2H), 8.24 (d, 1H), 10.48-10.58 (m, 1H).

LC-MS (Method 4): Rₜ=0.58 min; MS (ESIpos): m/z=680.4 [M+H−HCl]⁺

### Example 49

### 5′-[(2S)-2-({[trans-4-(Aminomethyl)cyclohexyl]carbonyl}amino)-3-oxo-3-{[4-(1H-tetrazol-5-yl)phenyl]amino}propyl]-2′-methyl-N-(piperidin-4-yl)biphenyl-4-carboxamide hydrochloride

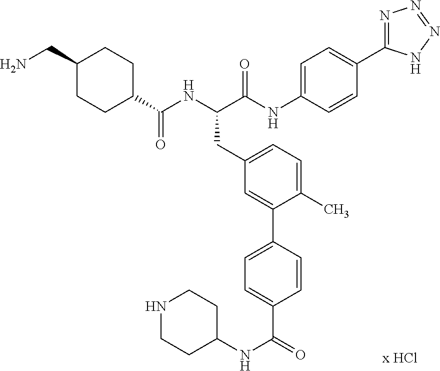

0.15 ml (0.65 mmol) of 4M hydrogen chloride in 1,4-dioxane was added to a suspension of 72 mg (0.084 mmol) of tert-butyl 4-[({5′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2′-methylbiphenyl-4-yl}carbonyl)amino]piperidine-1-carboxylate in 3.0 ml of 1,4-dioxane, and the mixture was stirred at RT overnight. 5 ml of acetonitrile were then added to the reaction mixture and the resulting precipitate was filtered off with suction and dried under high vacuum. This gave 66 mg (90% of theory) of the title compound.

¹H NMR (300 MHz, DMSO-d₆): δ=ppm 0.77-0.96 (m, 2H), 1.03-1.32 (m, 2H), 1.35-1.49 (m, 1H), 1.49-1.59 (m, 1H), 1.63-1.84 (m, 6H), 1.88-1.99 (m, 2H), 2.03-2.11 (m, 1H), 2.13 (s, 3H), 2.56-2.64 (m, 2H), 2.86 (dd, 1H), 2.93-3.08 (m, 3H), 3.97-4.12 (m, 1H), 4.59-4.70 (m, 1H), 7.12 (s, 1H), 7.19 (s, 2H), 7.32 (d, 2H), 7.78 (m, 5H), 7.89 (d, 2H), 7.98 (d, 2H), 8.21 (d, 1H), 8.54 (d, 1H), 8.62-8.86 (m, 2H), 10.50 (s, 1H).

LC-MS (Method 4): Rₜ=0.73 min; MS (ESIpos): m/z=664.5 [M+H−HCl]⁺

### Example 50

### N-(2-Aminoethyl)-3′-[(2S)-2-({[trans-4-(aminomethyl)cyclohexyl]carbonyl}amino)-3-(1H-indazol-6-ylamino)-3-oxopropyl]biphenyl-4-carboxamide hydrochloride

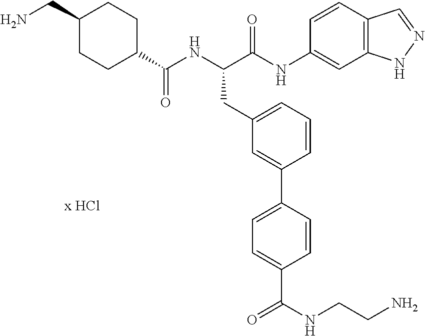

0.18 ml (0.70 mmol) of 4M hydrogen chloride in 1,4-dioxane was added to a suspension of 63 mg (0.07 mmol) of tert-butyl [(trans-4-{[(2S)-3-[4′-({2-[(tert-butoxycarbonyl)amino]ethyl}carbamoyl)biphenyl-3-yl]-1-(1H-indazol-6-ylamino)-1-oxopropan-2-yl]carbamoyl}cyclohexyl)methyl]carbamate trifluoroacetate in 3.0 ml of 1,4-dioxane, and the mixture was stirred at RT for 4 h. 5 ml of acetonitrile were then added to the reaction mixture and the resulting precipitate was filtered off with suction and dried under high vacuum. This gave 51 mg (quant.) of the title compound.

¹H NMR (400 MHz, DMSO-d₆): δ=ppm 0.83-0.98 (m, 2H), 1.06-1.35 (m, 2H), 1.40-1.58 (m, 2H), 1.65-1.80 (m, 3H), 2.11-2.22 (m, 1H), 2.57-2.69 (m, 2H), 2.93-3.06 (m, 4H), 3.17 (dd, 1H), 3.50-3.58 (m, 2H), 4.75-4.73 (m, 1H), 7.14 (d, 1H), 7.32-7.42 (m, 2H), 7.56 (d, 1H), 7.62-7.89 (m, 8H), 7.95-8.10 (m, 5H), 8.13 (s, 1H), 8.29 (d, 1H), 8.81 (t, 1H), 10.39 (s, 1H), 12.9 (bs, 1H).

LC-MS (Method 1): Rₜ=0.50 min; MS (ESIneg): m/z=580 [M−H−HCl]⁻.

### Example 51

### 3′-[(2S)-2-({[trans-4-(Aminomethyl)cyclohexyl]carbonyl}amino)-3-(1H-indazol-6-ylamino)-3-oxopropyl]-N-(piperidin-4-yl)biphenyl-4-carboxamide hydrochloride

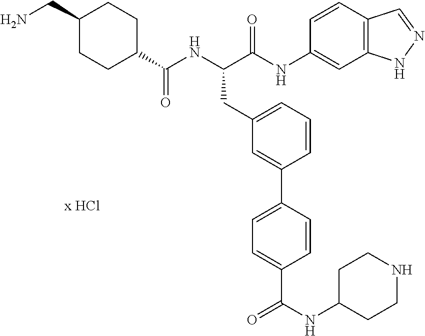

0.22 ml (0.86 mmol) of 4M hydrogen chloride in 1,4-dioxane was added to a suspension of 81 mg (0.087 mmol) of tert-butyl 4-[({3′-[(2S)-2-{[(trans-4-{[(tert-butoxycarbonyl)amino]methyl}cyclohexyl)carbonyl]amino}-3-(1H-indazol-6-ylamino)-3-oxopropyl]biphenyl-4-yl}carbonyl)amino]piperidine-1-carboxylate trifluoroacetate in 3.0 ml of 1,4-dioxane, and the mixture was stirred at RT for 4 h. 5 ml of acetonitrile were then added to the reaction mixture and the resulting precipitate was filtered off with suction and dried under high vacuum. This gave 72 mg (quant.) of the title compound.

¹H NMR (400 MHz, DMSO-d₆): δ=ppm 0.82-0.99 (m, 2H), 1.06-1.34 (m, 2H), 1.40-1.58 (m, 2H), 1.65-1.89 (m, 5H), 1.93-2.03 (m, 2H), 2.10-2.20 (m, 1H), 2.56-2.68 (m, 2H), 2.94-3.07 (m, 3H), 3.16 (dd, 1H), 3.26-3.36 (m, 2H), 4.02-4.14 (m, 1H), 4.75-4.82 (m, 1H), 7.15 (d, 1H), 7.31-7.41 (m, 2H), 7.54 (d, 1H), 7.62-7.75 (m, 4H), 7.78-7.92 (m, 3H), 7.94 (d, 2H), 8.14 (s, 1H), 8.29 (d, 1H), 8.58 (d, 1H), 8.80-8.97 (m, 2H), 10.43 (s, 1H), 12.9 (bs, 1H).

LC-MS (Method 1): Rₜ=0.58 min; MS (ESIneg): m/z=620 [M−H−HCl]⁻.

### Example 52

### trans-4-(Aminomethyl)-N-[(2S)-3-[2′-methyl-5′-(morpholin-4-ylsulphonyl)biphenyl-3-yl]-1-oxo-1-{[4-(5-oxo-4,5-dihydro-1,2,4-oxadiazol-3-yl)phenyl]amino}propan-2-yl]cyclohexanecarboxamide hydrochloride

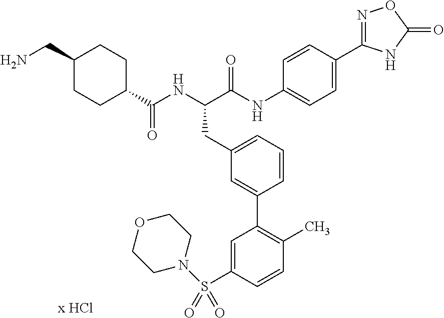 [SMILES: Cc1ccc(S(=O)(=O)N2CCOCC2)cc1-c1cccc(C[C@H](NC(=O)[C@H]2CC[C@H](CN)CC2)C(=O)Nc2ccc(-c3noc(=O)[nH]3)cc2)c1]

0.05 ml (0.19 mmol) of 4M hydrogen chloride in 1,4-dioxane was added to a suspension of 22 mg (0.027 mmol) of tert-butyl [(trans-4-{[(2S)-3-[2′-methyl-5′-(morpholin-4-ylsulphonyl)biphenyl-3-yl]-1-oxo-1-{[4-(5-oxo-4,5-dihydro-1,2,4-oxadiazol-3-yl)phenyl]amino}propan-2-yl]carbamoyl}cyclohexyl)methyl]carbamate in 1 ml of 1,4-dioxane, and the mixture was stirred at RT for 72 h. 5 ml of acetonitrile were then added to the reaction mixture and the resulting precipitate was filtered off with suction and dried under high vacuum. The residue was purified by chromatography via HPLC (Method 8). This gave 5 mg (32% of theory) of the title compound.

¹H NMR (300 MHz, MeOH-d₄): δ=ppm 0.97-1.13 (m, 4H), 1.26-1.50 (m, 5H), 1.51-1.66 (m, 2H), 1.69-1.79 (m, 2H), 1.80-1.91 (m, 5H), 2.17-2.29 (m, 2H), 2.74-2.80 (m, 3H), 2.96 (dd, 2H), 3.16 (dd, 2H), 4.69-4.73 (m, 1H), 7.14-7.21 (m, 2H), 7.22-7.27 (m, 2H), 7.32-7.38 (m, 2H), 7.43-7.48 (m, 2H), 7.67-7.77 (m, 7H), 8.19 (d, 1H).

LC-MS (Method 4): Rₜ=0.86 min; MS (ESIpos): m/z=703.3 [M+H−HCl]⁺

### Example 53

### 3′-[(2S)-2-({[trans-4-(Aminomethyl)cyclohexyl]carbonyl}amino)-3-(1H-indazol-6-ylamino)-3-oxopropyl]-N-[2-(diethylamino)ethyl]biphenyl-4-carboxamide hydrochloride

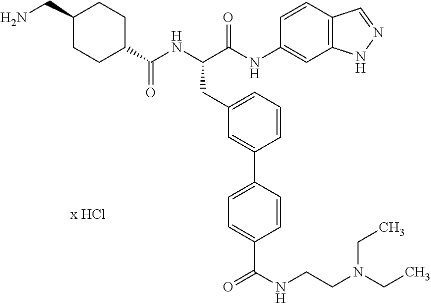

0.43 ml (1.72 mmol) of 4M hydrogen chloride in 1,4-dioxane was added to a suspension of 98 mg (0.115 mmol) of tert-butyl [(trans-4-{[(2S)-3-(4′-{[2-(diethylamino)ethyl]carbamoyl}biphenyl-3-yl)-1-(1H-indazol-6-ylamino)-1-oxopropan-2-yl]carbamoyl}cyclohexyl)methyl]carbamate trifluoroacetate in 3.0 ml of 1,4-dioxane, and the mixture was stirred at RT overnight. 5 ml of acetonitrile were then added to the reaction mixture and the resulting precipitate was filtered off with suction and dried under high vacuum. This gave 70 mg (86% of theory) of the title compound.

¹H NMR (400 MHz, DMSO-d₆): δ=ppm 0.81-0.97 (m, 2H), 1.04-1.18 (m, 1H), 1.20-1.33 (m, 7H), 1.38-1.58 (m, 2H), 1.64-1.80 (m, 3H), 2.11-2.21 (m, 1H), 2.55-2.66 (m, 2H), 2.97 (dd, 1H), 3.11-3.30 (m, 7H), 3.61-3.71 (m, 2H), 4.74-4.82 (m, 1H), 7.16 (d, 1H), 7.32-7.42 (m, 2H), 7.57 (d, 1H), 7.67 (d, 1H), 7.70 (s, 1H), 7.75 (d, 2H), 7.81-7.96 (m, 3H), 7.98 (s, 1H), 8.01 (d, 2H), 8.14 (s, 1H), 8.29 (d, 1H), 9.04 (t, 1H), 10.3 (bs, 1H), 10.42 (s, 1H), 12.9 (bs, 1H).

LC-MS (Method 1): Rₜ=0.63 min; MS (ESIneg): m/z=636 [M−H−HCl]⁻.

### Example 54

trans-4-(Aminomethyl)-N-[(2S)-3-[2′-(morpholin-4-ylsulphonyl)biphenyl-3-yl]-1-oxo-1-{[4-(2H-tetrazol-5-yl)phenyl]amino}propan-2-yl]cyclohexanecarboxamide hydrochloride

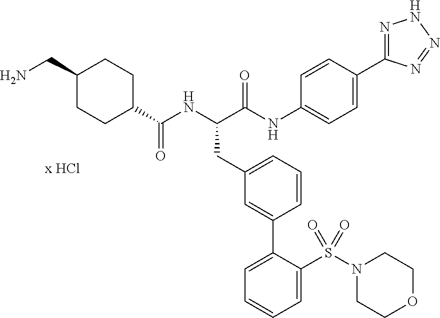

0.16 ml (0.64 mmol) of 4M hydrogen chloride in 1,4-dioxane was added to a suspension of 38 mg (0.043 mmol) of tert-butyl [(trans-4-{[(2S)-3-[2′-(morpholin-4-ylsulphonyl)biphenyl-3-yl]-1-oxo-1-{[4-(2H-tetrazol-5-yl)phenyl]amino}propan-2-yl]carbamoyl}cyclohexyl)methyl]carbamate in 2.0 ml of 1,4-dioxane, and the mixture was stirred at RT for 48 h. 5 ml of acetonitrile were then added to the reaction mixture and the resulting precipitate was filtered off with suction and dried under high vacuum. This gave 25 mg (75% of theory) of the title compound.

¹H NMR (400 MHz, DMSO-d₆): δ=ppm 0.84-1.00 (m, 2H), 1.10-1.33 (m, 2H), 1.40-1.65 (m, 2H), 1.66-1.80 (m, 3H), 2.05-2.19 (m, 1H), 2.57-2.73 (m, 4H), 2.93 (dd, 1H), 3.11 (dd, 1H), 3.26-3.40 (m, 4H), 3.4-3.8 (m, 2H), 4.66-4.75 (m, 1H), 7.19-7.37 (m, 5H), 7.58-7.90 (m, 7H), 7.93-8.05 (m, 3H), 8.17 (d, 1H), 10.48 (s, 1H).

LC-MS (Method 1): Rₜ=0.76 min; MS (ESIneg): m/z=671 [M−H−HCl]⁻.

### Example 55

### trans-4-(Aminomethyl)-N-{(2S)-1-{[4-(3-chloro-4H-1,2,4-triazol-5-yl)phenyl]amino}-3-[2′-methyl-4′-(piperidin-4-ylsulphamoyl)biphenyl-3-yl]-1-oxopropan-2-yl}cyclohexanecarboxamide hydrochloride

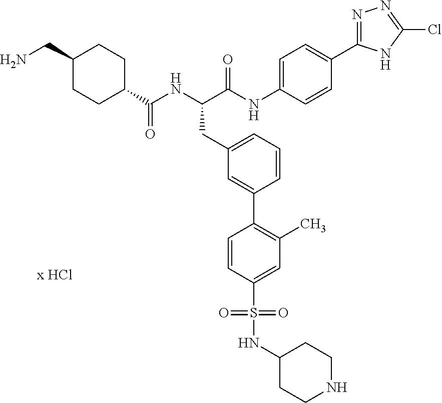 [SMILES: Cc1cc(S(=O)(=O)NC2CCNCC2)ccc1-c1cccc(C[C@H](NC(=O)[C@H]2CC[C@H](CN)CC2)C(=O)Nc2ccc(-c3nnc(Cl)[nH]3)cc2)c1]

0.025 ml (0.1 mmol) of 4M hydrogen chloride in 1,4-dioxane was added to a suspension of 9.3 mg (0.01 mmol) of tert-butyl 4-[({3′-[(2S)-2-{[(trans-4-{[(tert-butoxycarbonyl)amino]methyl}cyclohexyl)carbonyl]amino}-3-{[4-(3-chloro-4H-1,2,4-triazol-5-yl)phenyl]amino}-3-oxopropyl]-2-methylbiphenyl-4-yl}sulphonyl)amino]piperidine-1-carboxylate in 0.5 ml of 1,4-dioxane, and the mixture was stirred at 40° C. for 4 h and then at RT overnight. 5 ml of acetonitrile were then added to the reaction mixture and the resulting precipitate was filtered off with suction and dried under high vacuum. This gave 4.6 mg (54% of theory) of the title compound.

¹H NMR (300 MHz, DMSO-d₆): δ=ppm 0.76-0.96 (m, 2H), 1.04-1.30 (m, 2H), 1.35-1.61 (m, 4H), 1.64-1.81 (m, 5H), 2.03-2.16 (m, 1H), 2.21 (s, 3H), 2.54-2.65 (m, 2H), 2.81-2.97 (m, 3H), 3.06-3.20 (m, 3H), 4.61-4.75 (m, 1H), 7.14-7.19 (m, 1H), 7.24-7.38 (m, 4H), 7.60-7.79 (m, 7H), 7.87 (d, 2H), 7.96 (d, 1H), 8.20 (d, 1H), 8.39-8.54 (m, 1H), 8.58-8.72 (m, 1H), 10.41 (s, 1H), 14.70 (s, 1H).

LC-MS (Method 4): Rₜ=0.70 min; MS (ESIpos): m/z=733.4 [M+H−HCl]⁺

### Example 56

### 5′-[(2S)-2-({[trans-4-(Aminomethyl)cyclohexyl]carbonyl}amino)-3-oxo-3-{[4-(2H-tetrazol-5-yl)phenyl]amino}propyl]-2′-methoxy-2-methyl-N-(piperidin-4-yl)biphenyl-4-carboxamide hydrochloride

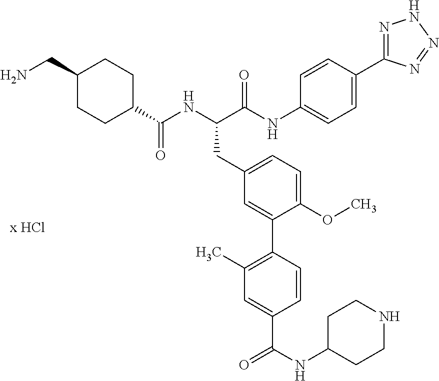

0.30 ml (1.19 mmol) of 4M hydrogen chloride in 1,4-dioxane was added to a solution of 80 mg (0.08 mmol) of tert-butyl 4-[({5′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-yl}carbonyl)amino]piperidine-1-carboxylate in 3 ml of 1,4-dioxane, and the mixture was stirred at RT overnight. The precipitate was filtered off, washed with a little acetonitrile and dried under high vacuum. This gave 65 mg (quantitative yield) of the title compound.

¹H NMR (400 MHz, DMSO-d₆): δ=ppm 0.82-1.01 (m, 2H), 1.09-1.34 (m, 2H), 1.40-1.64 (m, 2H), 1.66-1.86 (m, 5H), 1.91-2.20 (m, 5H), 2.59-2.69 (m, 2H), 2.81-2.92 (m, 1H), 2.94-3.09 (m, 3H), 3.25-3.48 (m, 3H), 3.66 (s, 3H), 3.99-4.14 (m, 1H), 4.62-4.73 (m, 1H), 6.95-7.17 (m, 3H), 7.28-7.37 (m, 1H), 7.58-7.74 (m, 2H), 7.75-7.90 (m, 4H), 8.01 (d, 2H), 8.21 (d, 1H), 8.46 (d, 1H), 8.62-8.84 (m, 2H), 10.50 (s, 1H), 16.8 (bs, 1H).

LC-MS (Method 1): Rₜ=0.62 min; MS (ESIneg): m/z=692 [M−H−HCl]⁻.

### Example 57

### 5′-[(2S)-2-({[trans-4-(Aminomethyl)cyclohexyl]carbonyl}amino)-3-oxo-3-{[4-(2H-tetrazol-5-yl)phenyl]amino}propyl]-2′-methoxy-2-methyl-N-[(3R)-pyrrolidin-3-yl]biphenyl-4-carboxamide hydrochloride

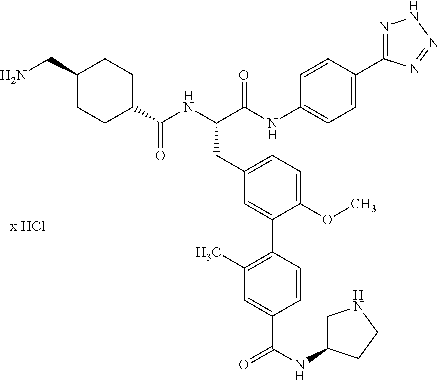

0.19 ml (0.77 mmol) of 4M hydrogen chloride in 1,4-dioxane was added to a solution of 51 mg (0.05 mmol) of tert-butyl (3R)-3-[({5′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-methoxy-2-methylbiphenyl-4-yl}carbonyl)amino]pyrrolidine-1-carboxylate in 2 ml of 1,4-dioxane, and the mixture was stirred at RT overnight. The precipitate was filtered off, washed with a little acetonitrile and dried under high vacuum. This gave 41 mg (quant., 94% pure) of the title compound.

¹H NMR (400 MHz, DMSO-d₆): δ=ppm 0.81-1.01 (m, 2H), 1.08-1.36 (m, 2H), 1.40-1.65 (m, 2H), 1.66-1.83 (m, 3H), 1.92-2.27 (m, 4H), 2.58-2.69 (m, 2H), 2.80-2.92 (m, 1H), 2.98-3.09 (m, 1H), 3.14-3.32 (m, 2H), 3.32-3.61 (m, 3H), 3.67 (s, 3H), 4.50-4.61 (m, 1H), 4.63-4.73 (m, 1H), 6.95-7.18 (m, 3H), 7.32 (d, 1H), 7.61-7.90 (m, 7H), 8.01 (d, 2H), 8.20 (d, 1H), 8.71 (d, 1H), 9.02-9.17 (m, 1H), 9.21-9.35 (m, 1H), 10.51 (s, 1H).

LC-MS (Method 1): Rₜ=0.61 min; MS (ESIneg): m/z=678 [M−H−HCl]⁻.

### Example 58

### N-(2-Aminoethyl)-5′-[(2S)-2-({[trans-4-(aminomethyl)cyclohexyl]carbonyl}amino)-3-oxo-3-{[4-(2H-tetrazol-5-yl)phenyl]amino}propyl]-2′-methoxy-2-methylbiphenyl-4-carboxamide hydrochloride

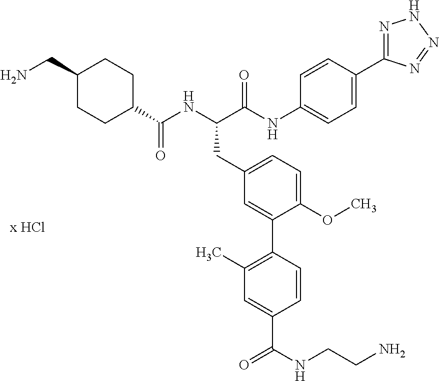

0.16 ml (0.65 mmol) of 4M hydrogen chloride in 1,4-dioxane was added to a solution of 42 mg (0.04 mmol) of tert-butyl [(trans-4-{[(2S)-3-[4′-({2-[(tert-butoxycarbonyl)amino]ethyl}carbamoyl)-6-methoxy-2′-methylbiphenyl-3-yl]-1-oxo-1-{[4-(2H-tetrazol-5-yl)-phenyl]amino}propan-2-yl]carbamoyl}cyclohexyl)methyl]carbamate in 2 ml of 1,4-dioxane, and the mixture was stirred at RT overnight. The precipitate was filtered off, washed with a little acetonitrile and dried under high vacuum. This gave 31 mg (91% of theory, 93% purity) of the title compound.

¹H NMR (400 MHz, DMSO-d₆): δ=ppm 0.80-1.01 (m, 2H), 1.08-1.34 (m, 2H), 1.41-1.64 (m, 2H), 1.66-1.83 (m, 3H), 1.92-2.20 (m, 4H), 2.60-2.69 (m, 2H), 2.80-2.92 (m, 1H), 2.94-3.09 (m, 3H), 3.27-3.61 (m, 3H), 3.67 (s, 3H), 4.62-4.73 (m, 1H), 6.96-7.19 (m, 3H), 7.29-7.36 (m, 1H), 7.64-7.92 (m, 7H), 7.93-8.07 (m, 4H), 8.22 (d, 1H), 8.66-8.75 (m, 1H), 10.52 (s, 1H).

LC-MS (Method 1): Rₜ=0.60 min; MS (ESIneg): m/z=652 [M−H−HCl]⁻.

### Example 59

### N-alpha-{[trans-4-(Aminomethyl)cyclohexyl]carbonyl}-3-{6-[3-(dimethylamino)propoxy]pyridin-3-yl}-N-[4-(1H-tetrazol-5-yl)phenyl]-L-phenylalaninamide hydrochloride

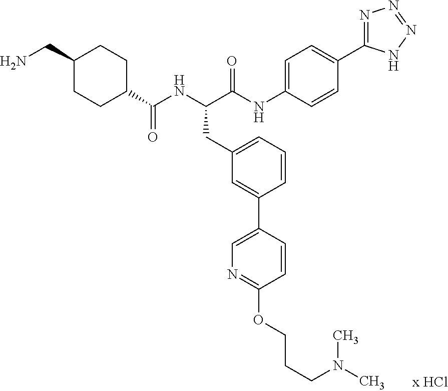

0.22 ml (0.88 mmol) of 4M hydrogen chloride in 1,4-dioxane was added to a suspension of 92 mg (0.127 mmol) of N-alpha-[(trans-4-{[(tert-butoxycarbonyl)amino]methyl}cyclohexyl)carbonyl]-3-{6-[3-(dimethylamino)propoxy]pyridin-3-yl}-N-[4-(1H-tetrazol-5-yl)phenyl]-L-phenylalaninamide in 5.6 ml of 1,4-dioxane, and the mixture was stirred at 40° C. overnight. After the addition of a further 0.095 ml (0.38 mmol) of 4M hydrogen chloride in 1,4-dioxane, the reaction mixture was stirred at RT for another night. 5 ml of acetonitrile were added to the reaction mixture and the resulting precipitate was filtered off with suction, washed with acetonitrile and then dried under high vacuum. This gave 90.5 mg (99% of theory) of the title compound.

¹H NMR (300 MHz, DMSO-d₆): δ=ppm 0.76-0.96 (m, 2H), 1.01-1.30 (m, 2H), 1.35-1.54 (m, 2H), 1.70 (m, 3H), 2.12 (m, 3H), 2.53-2.62 (m, 2H), 2.74 (s, 3H), 2.75 (s, 3H), 2.88-2.99 (m, 1H), 3.08-3.22 (m, 3H), 4.32 (t, 2H), 4.68-4.78 (m, 1H), 6.87 (d, 1H), 7.27 (br. d, 1H), 7.33 (t, 1H), 7.44 (br. d, 1H), 7.58 (s, 1H), 7.80 (d, 2H), 7.86 (br. s., 3H), 7.94 (dd, 1H), 7.99 (d, 2H), 8.24 (d, 1H), 8.39 (d, 1H), 10.40 (br. s, 1H), 10.59 (s, 1H).

LC-MS (Method 4): Rₜ=0.62 min; MS (ESIpos): m/z=626 [M+H−HCl]⁺.

### Example 60

### trans-4-(Aminomethyl)-N-[(2S)-3-[5′-(benzylsulphamoyl)-2′-methylbiphenyl-3-yl]-1-oxo-1-{[4-(5-oxo-4,5-dihydro-1,2,4-oxadiazol-3-yl)phenyl]amino}propan-2-yl]cyclohexanecarboxamide hydrochloride

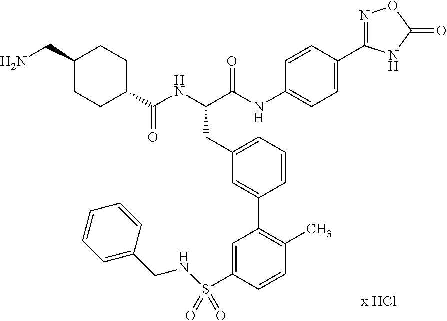 [SMILES: Cc1ccc(S(=O)(=O)NCc2ccccc2)cc1-c1cccc(C[C@H](NC(=O)[C@H]2CC[C@H](CN)CC2)C(=O)Nc2ccc(-c3noc(=O)[nH]3)cc2)c1]

0.07 ml (0.31 mmol) of 4 M hydrogen chloride in dioxane were added to a solution of 36.4 mg (0.04 mmol) of tert-butyl [(trans-4-{[(2S)-3-[5′-(benzylsulphamoyl)-2′-methylbiphenyl-3-yl]-1-oxo-1-{[4-(5-oxo-4,5-dihydro-1,2,4-oxadiazol-3-yl)phenyl]amino}propan-2-yl]carbamoyl}cyclohexyl)methyl]carbamate in 1.6 ml of dioxane. The mixture was stirred at RT for 72 h. Acetonitrile was added to the contents of the flask. The precipitated product was filtered off with suction, washed with acetonitrile and dried under high vacuum. 11 mg (91% of theory) of the title compound were obtained.

¹H NMR (400 MHz, DMSO-d₆): δ=ppm 0.80-0.89 (m, 2H), 1.07 (s, 3H), 1.11-1.26 (m, 2H), 1.35-1.42 (m, 1H), 1.44-1.53 (m, 1H), 1.60-1.76 (m, 3H), 2.02-2.13 (m, 1H), 2.19 (s, 3H), 2.58 (d, 2H), 2.85-2.93 (m, 1H), 3.06-3.15 (m, 2H), 3.97 (s, 2H), 4.65-4.74 (m, 1H), 7.13 (d, 1H), 7.17-7.27 (m, 6H), 7.30-7.38 (m, 2H), 7.44 (d, 1H), 7.50-7.58 (m, 3H), 7.63-7.69 (m, 3H), 8.12 (d, 1H), 10.16 (s, 1H).

LC-MS (Method 4): Rt=0.95 min; MS (ESIpos): m/z=723.4 [M+H−HCl]⁺

### Example 61

### 3′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(pyrrolidin-1-yl)ethyl]biphenyl-3-carboxamide hydrochloride

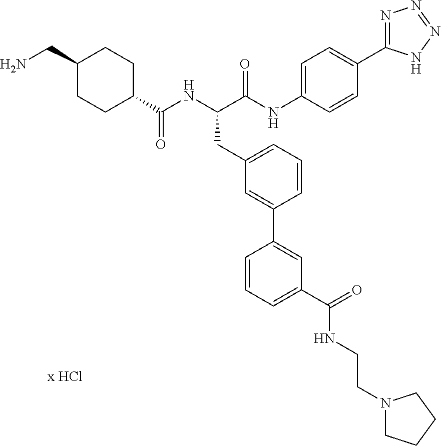 [SMILES: NC[C@H]1CC[C@H](C(=O)N[C@@H](Cc2cccc(-c3cccc(C(=O)NCCN4CCCC4)c3)c2)C(=O)Nc2ccc(-c3nnn[nH]3)cc2)CC1 ; "x HCl"]

0.06 ml (0.26 mmol) of 4 M hydrogen chloride in dioxane was added to a solution of 28.0 mg (0.04 mmol) of tert-butyl [(trans-4-{[(2S)-1-oxo-3-(3′-{[2-(pyrrolidin-1-yl)ethyl]carbamoyl}biphenyl-3-yl)-1-{[4-(1H-tetrazol-5-yl)phenyl]amino}propan-2-yl]carbamoyl}cyclohexyl)methyl]carbamate in 1.1 ml of dioxane. The mixture was stirred at RT for 24 h. Acetonitrile was added to the contents of the flask. The precipitated product was filtered off with suction, washed with acetonitrile and dried under high vacuum. 24 mg (84% of theory) of the title compound were obtained.

¹H NMR (300 MHz, DMSO-d₆): δ=ppm 0.76-0.97 (m, 2H), 1.01-1.30 (m, 2H), 1.32-1.55 (m, 2H), 1.57-1.79 (m, 4H), 1.82-1.92 (m, 2H), 1.95-2.03 (m, 2H), 2.07-2.18 (m, 1H), 2.20-2.25 (m, 1H), 2.55-2.61 (m, 2H), 2.67-2.71 (m, 1H), 2.87-2.98 (m, 1H), 2.98-3.07 (m, 2H), 3.09-3.18 (m, 1H), 3.59-3.68 (m, 3H), 4.67-4.80 (m, 1H), 7.27-7.39 (m, 2H), 7.48-7.59 (m, 2H), 7.83 (m, 9H), 7.97 (d, 2H), 8.21-8.30 (m, 2H), 8.93-9.02 (m, 1H), 10.13-10.26 (m, 1H), 10.59-10.65 (m, 1H).

LC-MS (Method 4): Rₜ=0.63 min; MS (ESIpos): m/z=664.6 [M+H−HCl]⁺

### Example 62

### 3′-[(2S)-2-({[trans-4-(Aminomethyl)cyclohexyl]carbonyl}amino)-3-oxo-3-{[4-(1H-tetrazol-5-yl)phenyl]amino}propyl]-6-fluoro-N-methylbiphenyl-3-carboxamide hydrochloride

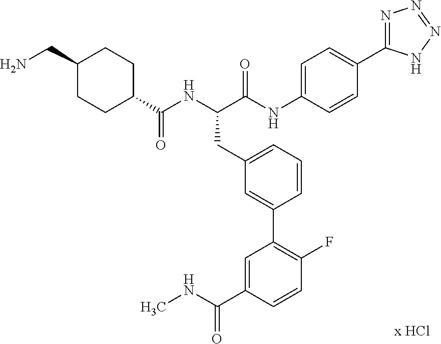 [SMILES: CNC(=O)c1ccc(F)c(-c2cccc(C[C@H](NC(=O)[C@H]3CC[C@H](CN)CC3)C(=O)Nc3ccc(-c4nnn[nH]4)cc3)c2)c1]

0.26 ml (1.03 mmol) of 4 M hydrogen chloride in dioxane was added to a solution of 103.0 mg (0.15 mmol) of tert-butyl [(trans-4-{[(2S)-3-[2′-fluoro-5′-(methylcarbamoyl)biphenyl-3-yl]-1-oxo-1-{[4-(1H-tetrazol-5-yl)phenyl]amino}propan-2-yl]carbamoyl}cyclohexyl)methyl]carbamate in 5.2 ml of dioxane. The mixture was stirred at 40° C. for 4 h. Acetonitrile was added to the contents of the flask. The precipitated product was filtered off with suction, washed with acetonitrile and dried under high vacuum. 73 mg (77% of theory) of the title compound were obtained.

¹H NMR (400 MHz, DMSO-d₆): δ=ppm 0.77-0.95 (m, 2H), 1.04-1.15 (m, 1H), 1.16-1.27 (m, 1H), 1.36-1.52 (m, 2H), 1.60-1.79 (m, 4H), 2.12 (t, 1H), 2.57 (br. t, 2H), 2.77 (d, 3H), 2.89-3.01 (m, 1H), 3.14 (dd, 1H), 4.64-4.76 (m, 1H), 7.31-7.44 (m, 4H), 7.58 (s, 1H), 7.79-7.89 (m, 6H), 7.96-8.03 (m, 3H), 8.23 (d, 1H), 8.56-8.63 (m, 1H), 10.59 (s, 1H).

LC-MS (Method 4): Rₜ=0.83 min; MS (ESIpos): m/z=599.5 [M+H−HCl]⁺

### Example 63

### 3′-[(2S)-2-({[trans-4-(Aminomethyl)cyclohexyl]carbonyl}amino)-3-oxo-3-{[2-(pentafluoroethyl)-1H-benzimidazol-6-yl]amino}propyl]-2-methyl-N-[(3R)-2-oxopiperidin-3-yl]biphenyl-4-carboxamide hydrochloride

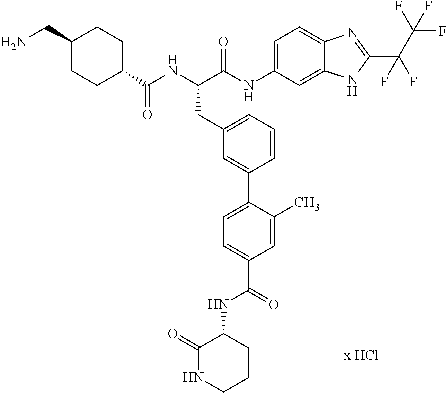 [SMILES: Cc1cc(C(=O)N[C@@H]2CCCNC2=O)ccc1-c1cccc(C[C@H](NC(=O)[C@H]2CC[C@H](CN)CC2)C(=O)Nc2ccc3nc(C(F)(F)C(F)(F)F)[nH]c3c2)c1]

0.04 ml (0.14 mmol) of 4 M hydrogen chloride in dioxane was added to a solution of 25.2 mg (0.03 mmol) of tert-butyl [(trans-4-{[(2S)-3-(2′-methyl-4′-{[(3R)-2-oxopiperidin-3-yl]carbamoyl}biphenyl-3-yl)-1-oxo-1-{[2-(pentafluoroethyl)-1H-benzimidazol-6-yl]amino}propan-2-yl]carbamoyl}cyclohexyl)methyl]carbamate in 0.4 ml of dichloromethane. The mixture was stirred at 30° C. for 48 h. Acetonitrile was added to the contents of the flask. The precipitated product was filtered off with suction, washed with acetonitrile and dried under high vacuum. 15 mg (67% of theory) of the title compound were obtained.

¹H NMR (300 MHz, DMSO-d₆): δ=ppm 0.77-0.95 (m, 2H), 1.03-1.34 (m, 2H), 1.39-1.57 (m, 2H), 1.64-1.86 (m, 6H), 2.07-2.18 (m, 1H), 2.22 (s, 3H), 2.62 (t, 2H), 2.89-3.01 (m, 1H), 3.15-3.22 (m, 2H), 4.32-4.44 (m, 1H), 4.72-4.82 (m, 1H), 7.14-7.25 (m, 2H), 7.26-7.37 (m, 3H), 7.37-7.47 (m, 1H), 7.59-7.80 (m, 7H), 8.19 (s, 2H), 8.55 (d, 1H), 10.34 (s, 1H).

LC-MS (Method 4): Rt=0.90 min; MS (ESIpos): m/z=768.4 [M+H−HCl]⁺

### Example 64

### 3′-[(2S)-2-({[trans-4-(Aminomethyl)cyclohexyl]carbonyl}amino)-3-{[3-fluoro-4-(1H-tetrazol-5-yl)phenyl]amino}-3-oxopropyl]-N-[4-(dimethylamino)cyclohexyl]-2-methylbiphenyl-4-carboxamide hydrochloride

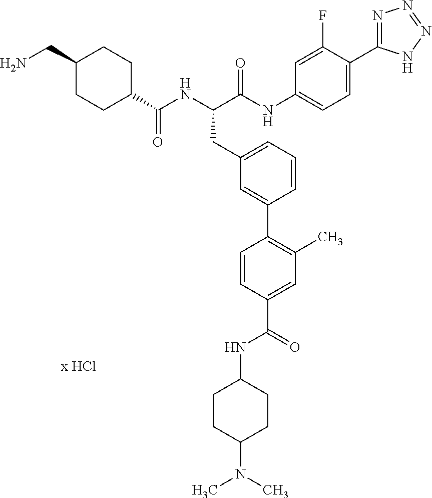

0.012 ml (0.05 mmol) of 4 M hydrogen chloride in dioxane was added to a solution of 7.8 mg (0.009 mmol) of tert-butyl [(trans-4-{[(2S)-3-(4′-{[4-(dimethylamino)cyclohexyl]carbamoyl}-2′-methylbiphenyl-3-yl)-1-{[3-fluoro-4-(1H-tetrazol-5-yl)phenyl]amino}-1-oxopropan-2-yl]carbamoyl}cyclohexyl)methyl]carbamate in 0.5 ml of dichloromethane. The mixture was stirred at RT for 2 h, concentrated and dried under high vacuum. This gave 5.8 mg (75% of theory) of the title compound.

¹H NMR (600 MHz, DMSO-d₆): δ=ppm 0.83-0.96 (m, 2H), 1.11-1.19 (m, 1H), 1.22-1.28 (m, 2H), 1.37-1.49 (m, 3H), 1.51-1.65 (m, 3H), 1.67-1.73 (m, 2H), 1.81-1.89 (m, 2H), 1.93-2.01 (m, 3H), 2.03-2.09 (m, 2H), 2.09-2.15 (m, 1H), 2.21 (s, 3H), 2.59-2.64 (m, 2H), 2.69-2.74 (m, 6H), 2.92-2.98 (m, 1H), 3.10-3.18 (m, 2H), 3.74-3.83 (m, 1H), 4.68-4.76 (m, 1H), 7.13-7.16 (m, 1H), 7.19 (d, 1H), 7.26 (s, 1H), 7.31-7.39 (m, 2H), 7.50-7.54 (m, 1H), 7.65-7.70 (m, 1H), 7.73 (d, 1H), 7.79 (br. s, 3H), 7.83-7.88 (m, 1H), 7.98-8.03 (m, 1H), 8.24-8.31 (m, 2H), 10.11-10.23 (m, 1H), 10.73-10.82 (m, 1H).

LC-MS (Method 4): Rₜ=0.84 min; MS (ESIpos): m/z=724.4 [M+H−HCl]⁺

### Example 65

### N-alpha-{[trans-4-(Aminomethyl)cyclohexyl]carbonyl}-3-[2-(4-methylpiperazin-1-yl)pyridin-4-yl]-N-[4-(1H-tetrazol-5-yl)phenyl]-L-phenylalaninamide hydrochloride

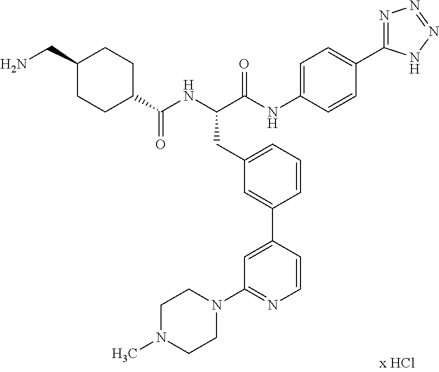

0.125 ml (0.5 mmol) of 4M hydrogen chloride in 1,4-dioxane was added to a suspension of 52 mg (0.072 mmol) of N-alpha-[(trans-4-{[(tert-butoxycarbonyl)amino]methyl}cyclohexyl)carbonyl]-3-[2-(4-methylpiperazin-1-yl)pyridin-4-yl]-N-[4-(1H-tetrazol-5-yl)phenyl]-L-phenylalaninamide in 3.2 ml of 1,4-dioxane, and the mixture was stirred at 40° C. overnight. After the addition of a further 0.05 ml (0.2 mmol) of 4M hydrogen chloride in 1,4-dioxane, the reaction mixture was stirred at 40° C. for a further 2 h. 5 ml of acetonitrile were added to the reaction mixture and the resulting precipitate was filtered off with suction, washed with acetonitrile and then dried under high vacuum. This gave 48 mg (96% of theory) of the title compound.

¹H NMR (400 MHz, DMSO-d₆): δ=ppm 0.75-0.94 (m, 2H), 1.00-1.13 (m, 1H), 1.16-1.29 (m, 1H), 1.36-1.50 (m, 2H), 1.60-1.77 (m, 3H), 2.05-2.16 (m, 1H), 2.54-2.62 (m, 2H), 2.79 (br. s., 3H), 2.94 (dd, 1H), 3.03-3.13 (m, 2H), 3.17 (dd, 1H), 3.30-3.43 (m, 3H), 3.45-3.53 (m, 2H), 4.50-4.62 (m, 2H), 4.72-4.80 (m, 1H), 7.08-7.15 (m, 1H), 7.30-7.36 (m, 1H), 7.37-7.42 (m, 2H), 7.61-7.67 (m, 1H), 7.78 (br. s, 3H), 7.84 (d, 2H), 7.88 (br. s, 1H), 7.99 (br. d, 2H), 8.17 (d, 1H), 8.27 (br. d, 1H), 10.70 (s, 1H), 10.80-10.98 (m, 1H).

LC-MS (Method 4): Rₜ=0.62 min; MS (ESIpos): m/z=622 [M+H−HCl]⁺.

### Example 66

### N-alpha-{[trans-4-(Aminomethyl)cyclohexyl]carbonyl}-3-(6-amino-2-methylpyridin-3-yl)-N-[4-(1H-tetrazol-5-yl)phenyl]-L-phenylalaninamide hydrochloride

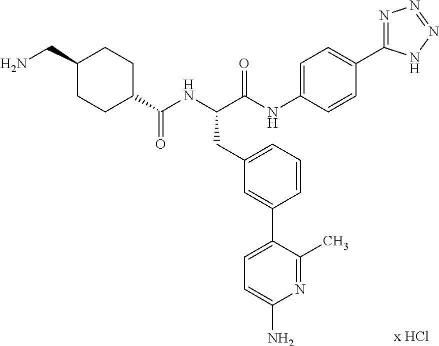

0.14 ml (0.55 mmol) of 4M hydrogen chloride in 1,4-dioxane was added to a suspension of 52 mg (0.079 mmol) of 3-(6-amino-2-methylpyridin-3-yl)-N-alpha-[(trans-4-{[(tert-butoxycarbonyl)amino]methyl}cyclohexyl)carbonyl]-N-[4-(1H-tetrazol-5-yl)phenyl]-L-phenylalaninamide in 3.5 ml of 1,4-dioxane, and the mixture was stirred at RT for 72 h. 5 ml of acetonitrile were added to the reaction mixture and the resulting precipitate was filtered off with suction, washed with acetonitrile and then dried under high vacuum. This gave 43 mg (86% of theory) of the title compound.

¹H NMR (300 MHz, DMSO-d₆): δ=ppm 0.78-0.97 (m, 2H), 1.03-1.30 (m, 2H), 1.35-1.54 (m, 2H), 1.64-1.78 (m, 3H), 2.05-2.16 (m, 1H), 2.30 (s, 3H), 2.56-2.63 (m, 2H), 2.92 (dd, 1H), 3.10 (dd, 1H), 4.64-4.77 (m, 1H), 6.86 (d, 1H), 7.13-7.19 (m, 1H), 7.25 (s, 1H), 7.29-7.40 (m, 2H), 7.77 (m, 8H), 7.97 (d, 2H), 8.22 (d, 1H), 10.49 (s, 1H).

LC-MS (Method 4): Rₜ=0.58 min; MS (ESIpos): m/z=554 [M+H−HCl]⁺.

### Example 67

### N-alpha-{[trans-4-(Aminomethyl)cyclohexyl]carbonyl}-N-(2-oxo-2,3-dihydro-1H-benzimidazol-5-yl)-3-[2-(piperazin-1-yl)pyrimidin-5-yl]-L-phenylalaninamide hydrochloride

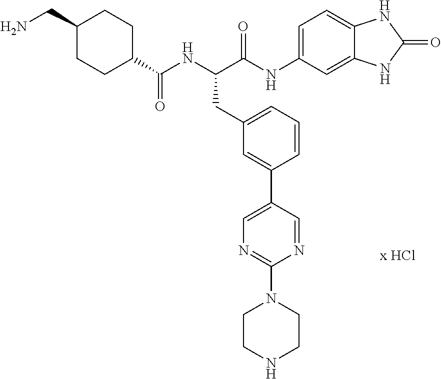

0.26 ml (1.05 mmol) of 4M hydrogen chloride in 1,4-dioxane was added to a suspension of 70 mg (0.088 mmol) of tert-butyl 4-[5-(3-{(2S)-2-{[(trans-4-{[(tert-butoxycarbonyl)amino]methyl}cyclohexyl)carbonyl]amino}-3-oxo-3-[(2-oxo-2,3-dihydro-1H-benzimidazol-5-yl)amino]propyl}phenyl)pyrimidin-2-yl]piperazine-1-carboxylate in 4 ml of 1,4-dioxane, and the mixture was stirred at 40° C. for 5 h. 5 ml of acetonitrile were added to the reaction mixture and the resulting precipitate was filtered off with suction, washed with acetonitrile and then dried under high vacuum. This gave 55 mg (92% of theory) of the title compound.

¹H NMR (300 MHz, DMSO-d₆): δ=ppm 0.75-0.95 (m, 2H), 1.00-1.29 (m, 2H), 1.36-1.50 (m, 2H), 1.60-1.77 (m, 3H), 2.05-2.16 (m, 1H), 2.54-2.62 (m, 2H), 2.89 (dd, 1H), 3.02-3.19 (m, 5H), 3.93-4.03 (m, 4H), 4.61-4.72 (m, 1H), 6.78 (d, 1H), 6.99 (dd, 1H), 7.23 (d, 1H), 7.31 (t, 1H), 7.40 (d, 1H), 7.45 (d, 1H), 7.55 (s, 1H), 7.78-7.92 (m, 3H), 8.18 (d, 1H), 8.68 (s, 2H), 9.25-9.38 (m, 2H), 10.05 (s, 1H), 10.46-10.49 (m, 1H), 10.50-10.55 (m, 1H).

LC-MS (Method 4): Rₜ=0.55 min; MS (ESIpos): m/z=598 [M+H−HCl]⁺.

### Example 68

### N-alpha-{[trans-4-(Aminomethyl)cyclohexyl]carbonyl}-3-[2-(piperazin-1-yl)pyrimidin-5-yl]-N-[4-(1H-tetrazol-5-yl)phenyl]-L-phenylalaninamide hydrochloride

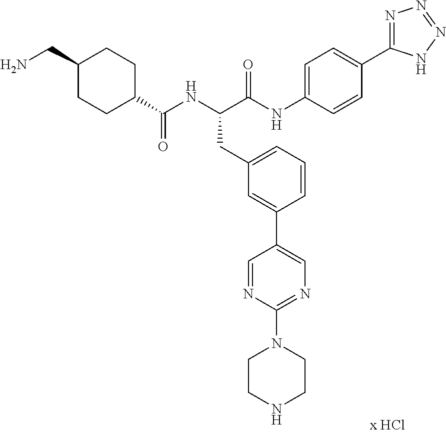

0.32 ml (1.3 mmol) of 4M hydrogen chloride in 1,4-dioxane was added to a suspension of 70 mg (0.086 mmol) of tert-butyl 4-(5-{3-[(2S)-2-{[(trans-4-{[(tert-butoxycarbonyl)amino]methyl}cyclohexyl)carbonyl]amino}-3-oxo-3-{[4-(1H-tetrazol-5-yl)-phenyl]amino}propyl]phenyl}pyrimidin-2-yl)piperazine-1-carboxylate in 3.8 ml of 1,4-dioxane, and the mixture was stirred at 40° C. overnight. 5 ml of acetonitrile were added to the reaction mixture and the resulting precipitate was filtered off with suction, washed with acetonitrile and then dried under high vacuum. This gave 61 mg (100% of theory) of the title compound.

¹H NMR (400 MHz, DMSO-d₆): δ=ppm 0.78-0.96 (m, 2H), 1.04-1.31 (m, 2H), 1.37-1.54 (m, 2H), 1.60-1.76 (m, 3H), 2.05-2.16 (m, 1H), 2.55-2.61 (m, 2H), 2.88-2.99 (m, 1H), 3.14 (m, 5H), 3.94-3.99 (m, 4H), 4.68-4.79 (m, 1H), 7.25 (d, 1H), 7.33 (t, 1H), 7.46 (d, 1H), 7.58 (s, 1H), 7.80 (m, 5H), 7.98 (d, 2H), 8.24 (d, 1H), 8.68 (s, 2H), 9.24 (br. s, 2H), 10.56 (s, 1H).

LC-MS (Method 4): Rₜ=0.63 min; MS (ESIpos): m/z=610 [M+H−HCl]⁺.

### Example 69

### N-alpha-{[trans-4-(Aminomethyl)cyclohexyl]carbonyl}-3-[2-(4-methylpiperazin-1-yl)pyrimidin-5-yl]-N-[4-(1H-tetrazol-5-yl)phenyl]-L-phenylalaninamide hydrochloride

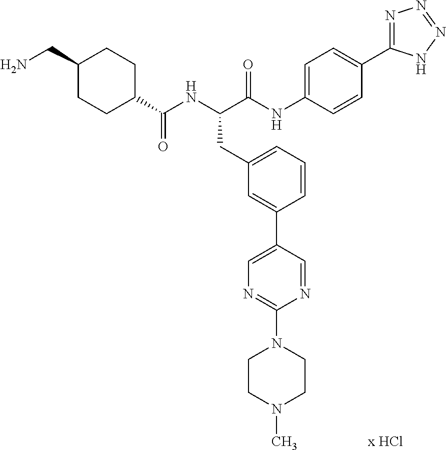

0.55 ml (2.3 mmol) of 4M hydrogen chloride in 1,4-dioxane was added to a suspension of 229 mg (0.32 mmol) of N-alpha-[(trans-4-{[(tert-butoxycarbonyl)amino]methyl}cyclohexyl)carbonyl]-3-[2-(4-methylpiperazin-1-yl)pyrimidin-5-yl]-N-[4-(1H-tetrazol-5-yl)phenyl]-L-phenylalaninamide in 9 ml of dichloromethane, and the mixture was stirred at 50° C. for 3 h. 5 ml of acetonitrile were added to the reaction mixture and the resulting precipitate was filtered off with suction, washed with acetonitrile, dried under high vacuum and then chromatographed by HPLC (Method 7). This gave 12 mg (5% of theory) of the title compound.

¹H NMR (300 MHz, DMSO-d₆): δ=ppm 0.71-0.97 (m, 2H), 0.99-1.30 (m, 3H), 1.31-1.52 (m, 2H), 1.56-1.77 (m, 3H), 2.04-2.16 (m, 1H), 2.19 (s, 3H), 2.34 (t, 4H), 2.59 (d, 2H), 2.83-2.94 (m, 1H), 3.05-3.13 (m, 1H), 3.71-3.78 (m, 4H), 4.65-4.77 (m, 1H), 7.19-7.25 (m, 1H), 7.31 (t, 1H), 7.37-7.47 (m, 1H), 7.56 (d, 4H), 7.86 (d, 2H), 8.15 (s, 1H), 8.64 (s, 2H), 10.11 (s, 1H).

LC-MS (Method 4): Rₜ=0.59 min; MS (ESIpos): m/z=623 [M+H−HCl]⁺.

### Example 70

### N-alpha-{[trans-4-(Aminomethyl)cyclohexyl]carbonyl}-3-[2-(dimethylamino)pyrimidin-5-yl]-N-[4-(1H-tetrazol-5-yl)phenyl]-L-phenylalaninamide hydrochloride

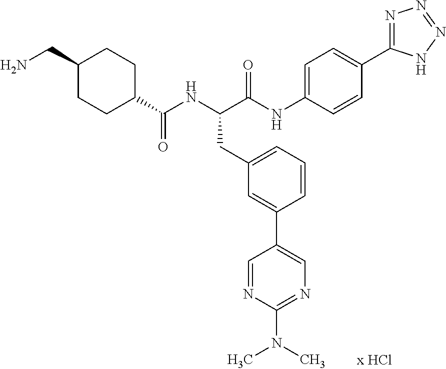

0.4 ml (1.6 mmol) of 4M hydrogen chloride in 1,4-dioxane was added to a suspension of 247 mg (0.32 mmol) of N-alpha-[(trans-4-{[(tert-butoxycarbonyl)amino]methyl}cyclohexyl)carbonyl]-3-[2-(dimethylamino)pyrimidin-5-yl]-N-[4-(1H-tetrazol-5-yl)phenyl]-L-phenylalaninamide in 8 ml of dichloromethane, and the mixture was stirred at RT for 72 h. After addition of a further 0.24 ml of 4M hydrogen chloride in 1,4-dioxane and 3 h at 35° C., 5 ml of acetonitrile were added to the reaction mixture and the resulting precipitate was filtered off with suction, washed with acetonitrile, dried under high vacuum and then chromatographed by HPLC (Method 7). This gave 75 mg (35% of theory) of the title compound.

¹H NMR (300 MHz, DMSO-d₆): δ=ppm 0.87 (m, 2H), 1.00-1.30 (m, 2H), 1.44 (m, 2H), 1.70 (d, 3H), 2.03-2.18 (m, 1H), 2.60 (d, 2H), 2.89 (dd, 1H), 3.07 (dd, 1H), 4.66-4.77 (m, 1H), 7.21 (d, 1H), 7.30 (t, 1H), 7.42 (d, 1H), 7.53 (s, 1H), 7.59 (d, 2H), 7.87 (d, 2H), 8.12-8.21 (m, 2H), 8.63 (s, 2H), 10.17 (s, 1H).

LC-MS (Method 4): Rₜ=0.74 min; MS (ESIpos): m/z=569 [M+H−HCl]⁺.

### Example 71

### N-alpha-{[trans-4-(Aminomethyl)cyclohexyl]carbonyl}-3-[6-(dimethylamino)pyridin-3-yl]-N-[4-(1H-tetrazol-5-yl)phenyl]-L-phenylalaninamide hydrochloride

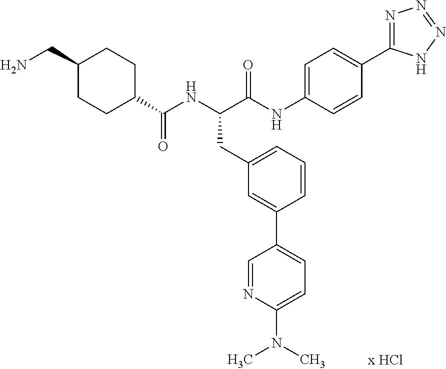

0.31 ml (1.3 mmol) of 4M hydrogen chloride in 1,4-dioxane was added to a suspension of 168 mg (0.25 mmol) of N-alpha-[(trans-4-{[(tert-butoxycarbonyl)amino]methyl}cyclohexyl)carbonyl]-3-[6-(dimethylamino)pyridin-3-yl]-N-[4-(1H-tetrazol-5-yl)phenyl]-L-phenylalaninamide in 6 ml of dichloromethane, and the mixture was stirred at RT overnight. 5 ml of acetonitrile were added to the reaction mixture and the resulting precipitate was filtered off with suction, washed with acetonitrile, dried under high vacuum and then chromatographed by HPLC (Method 7). This gave 41 mg (25% of theory) of the title compound.

¹H NMR (300 MHz, DMSO-d₆): δ=ppm 0.75-0.97 (m, 2H), 1.03-1.30 (m, 2H), 1.31-1.55 (m, 2H), 1.58-1.77 (m, 3H), 2.04-2.18 (m, 1H), 2.59 (d, 2H), 2.88 (dd, 1H), 3.02 (s, 6H), 3.09 (dd, 1H), 4.65-4.77 (m, 1H), 6.67 (d, 1H), 7.17 (d, 1H), 7.27 (t, 1H), 7.39 (d, 1H), 7.50 (s, 1H), 7.57 (d, 2H), 7.73 (dd, 1H), 7.86 (d, 2H), 8.08 (d, 1H), 8.38 (d, 1H), 10.09 (s, 1H).

LC-MS (Method 4): Rₜ=0.57 min; MS (ESIpos): m/z=568 [M+H−HCl]⁺.

### Example 72

### N-alpha-{[trans-4-(Aminomethyl)cyclohexyl]carbonyl}-3-(5-aminopyridin-3-yl)-N-[4-(1H-tetrazol-5-yl)phenyl]-L-phenylalaninamide hydrochloride

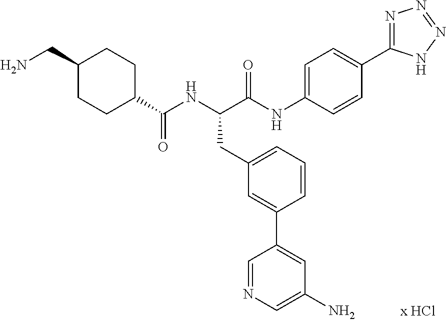

0.08 ml (0.3 mmol) of 4M hydrogen chloride in 1,4-dioxane was added to a suspension of 28.6 mg (0.045 mmol) of 3-(5-aminopyridin-3-yl)-N-alpha-[(trans-4-{[(tert-butoxycarbonyl)amino]methyl}cyclohexyl)carbonyl]-N-[4-(1H-tetrazol-5-yl)phenyl]-L-phenylalaninamide in 2 ml of 1,4-dioxane, and the mixture was stirred at 40° C. overnight. After addition of a further 0.034 ml (0.13 mmol) of 4M hydrogen chloride in 1,4-dioxane and at 40° C. for 3 h, 5 ml of acetonitrile were added to the reaction mixture and the resulting precipitate was filtered off with suction, washed with acetonitrile and dried under high vacuum. This gave 24 mg (84% of theory) of the title compound.

¹H NMR (300 MHz, DMSO-d₆): δ=ppm 0.75-0.98 (m, 2H), 1.01-1.31 (m, 2H), 1.33-1.53 (m, 2H), 1.58-1.78 (m, 4H), 2.05-2.17 (m, 1H), 2.54-2.63 (m, 2H), 2.95 (dd, 1H), 3.14 (dd, 1H), 4.66-4.78 (m, 1H), 7.39-7.45 (m, 2H), 7.48-7.56 (m, 1H), 7.67-7.72 (m, 1H), 7.78 (m, 6H), 7.97 (m, 3H), 8.25-8.28 (m, 1H), 10.53 (s, 1H).

LC-MS (Method 4): Rₜ=0.59 min; MS (ESIpos): m/z=540 [M+H−HCl]⁺.

### Example 73

### N-alpha-{[trans-4-(Aminomethyl)cyclohexyl]carbonyl}-3-[2-(dimethylamino)pyrimidin-5-yl]-N-(2-oxo-2,3-dihydro-1H-benzimidazol-5-yl)-L-phenylalaninamide hydrochloride

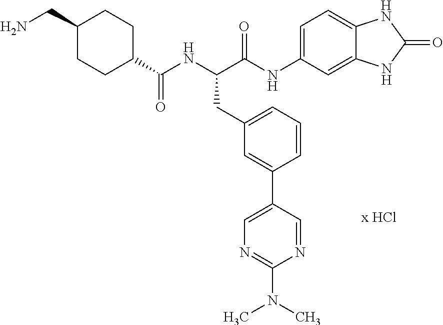

0.125 ml (0.5 mmol) of 4M hydrogen chloride in 1,4-dioxane was added to a suspension of 47 mg (0.072 mmol) of N-alpha-[(trans-4-{[(tert-butoxycarbonyl)amino]methyl}cyclohexyl)carbonyl]-3-[2-(dimethylamino)pyrimidin-5-yl]-N-(2-oxo-2,3-dihydro-1H-benzimidazol-5-yl)-L-phenylalaninamide in 2.4 ml of 1,4-dioxane, and the mixture was stirred at 40° C. for 5 h. 5 ml of acetonitrile were added to the reaction mixture and the resulting precipitate was filtered off with suction, washed with acetonitrile and dried under high vacuum. This gave 37 mg (80% of theory) of the title compound.

¹H NMR (400 MHz, DMSO-d₆): δ=ppm 0.77-0.94 (m, 2H), 1.01-1.15 (m, 1H), 1.15-1.29 (m, 1H), 1.35-1.51 (m, 2H), 1.60-1.76 (m, 3H), 2.05-2.15 (m, 1H), 2.54-2.61 (m, 2H), 2.88 (dd, 1H), 3.06 (dd, 1H), 3.14 (s, 6H), 4.62-4.72 (m, 1H), 6.79 (d, 1H), 6.98 (dd, 1H), 7.20 (d, 1H), 7.29 (t, 1H), 7.37-7.39 (m, 1H), 7.42 (d, 1H), 7.51 (s, 1H), 7.72 (br. s, 3H), 8.11 (d, 1H), 8.61 (s, 2H), 9.96 (s, 1H), 10.44 (s, 1H), 10.50 (s, 1H).

LC-MS (Method 4): Rₜ=0.74 min; MS (ESIpos): m/z=557 [M+H−HCl]⁺.

### Example 74

### N-alpha-{[trans-4-(Aminomethyl)cyclohexyl]carbonyl}-3-[2-(morpholin-4-yl)pyrimidin-5-yl]-N-[4-(1H-tetrazol-5-yl)phenyl]-L-phenylalaninamide hydrochloride

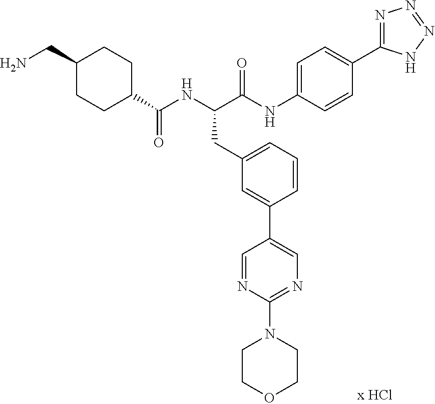

0.42 ml (1.7 mmol) of 4M hydrogen chloride in 1,4-dioxane was added to a solution of 170 mg (0.24 mmol) of N-alpha-[(trans-4-{[(tert-butoxycarbonyl)amino]methyl}cyclohexyl)carbonyl]-3-[2-(morpholin-4-yl)pyrimidin-5-yl]-N-[4-(1H-tetrazol-5-yl)phenyl]-L-phenylalaninamide in 1.8 ml of DMSO, and the mixture was stirred at 40° C. for 5 h and at RT overnight. After the addition of a further 0.9 ml (3.5 mmol) of 4M hydrogen chloride in 1,4-dioxane, the mixture was stirred at 40° C. overnight. The reaction mixture was concentrated, passed through a Milipore filter and chromatographed by HPLC (Method 10). This gave 32 mg (19% of theory) of the title compound.

¹H NMR (400 MHz, DMSO-d₆): δ=ppm 0.77-0.95 (m, 2H), 1.02-1.15 (m, 1H), 1.16-1.29 (m, 1H), 1.33-1.43 (m, 1H), 1.44-1.51 (m, 1H), 1.59-1.66 (m, 1H), 1.67-1.76 (m, 2H), 2.06-2.15 (m, 1H), 2.60 (d, 2H), 2.89 (dd, 1H), 3.10 (dd, 1H), 3.65 (d, 4H), 3.71 (m, 4H), 4.68-4.76 (m, 1H), 7.23 (d, 1H), 7.32 (t, 1H), 7.44 (d, 1H), 7.53-7.59 (m, 4H), 7.86 (d, 2H), 8.11 (d, 1H), 8.67 (s, 2H), 10.10 (s, 1H).

LC-MS (Method 4): Rₜ=0.80 min; MS (ESIpos): m/z=611 [M+H−HCl]⁺.

### Example 75

### N-alpha-{[trans-4-(Aminomethyl)cyclohexyl]carbonyl}-3-(3-methylpyridin-4-yl)-N-(2-oxo-2,3-dihydro-1H-benzimidazol-5-yl)-L-phenylalaninamide hydrochloride

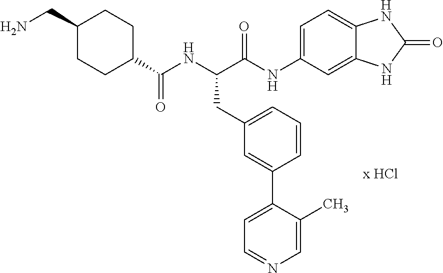

0.1 ml (0.38 mmol) of 4M hydrogen chloride in 1,4-dioxane was added to a suspension of 34 mg (0.055 mmol) of N-alpha-[(trans-4-{[(tert-butoxycarbonyl)amino]methyl}cyclohexyl)carbonyl]-3-(3-methylpyridin-4-yl)-N-(2-oxo-2,3-dihydro-1H-benzimidazol-5-yl)-L-phenylalaninamide in 2.7 ml of 1,4-dioxane, and the mixture was stirred at 40° C. for 5 h. 5 ml of acetonitrile were added to the reaction mixture and the resulting precipitate was filtered off with suction, washed with acetonitrile and dried under high vacuum. This gave 30 mg (85% of theory) of the title compound.

¹H NMR (400 MHz, DMSO-d₆): δ=ppm 0.78-0.94 (m, 2H), 1.02-1.27 (m, 2H), 1.36-1.51 (m, 2H), 1.62-1.76 (m, 3H), 2.04-2.12 (m, 1H), 2.27 (s, 3H), 2.55-2.61 (m, 2H), 2.91 (dd, 1H), 3.09 (dd, 1H), 4.63-4.72 (m, 1H), 6.79 (d, 1H), 6.97 (dd, 1H), 7.27-7.31 (m, 1H), 7.33-7.45 (m, 5H), 7.52-7.59 (m, 1H), 7.77 (br. s, 3H), 8.14 (d, 1H), 8.64 (d, 1H), 8.72 (s, 1H), 9.97 (s, 1H), 10.45 (s, 1H), 10.51 (s, 1H).

LC-MS (Method 4): Rₜ=0.52 min; MS (ESIpos): m/z=527 [M+H−HCl]⁺.

### Example 76

### N-alpha-{[trans-4-(Aminomethyl)cyclohexyl]carbonyl}-3-[2-(4-methylpiperazin-1-yl)pyridin-4-yl]-N-(2-oxo-2,3-dihydro-1H-benzimidazol-5-yl)-L-phenylalaninamide hydrochloride

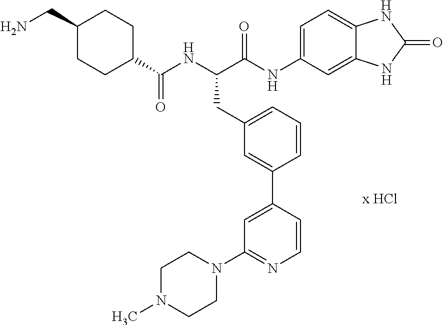

0.24 ml (0.97 mmol) of 4M hydrogen chloride in 1,4-dioxane was added to a suspension of 98 mg (0.14 mmol) of N-alpha-[(trans-4-{[(tert-butoxycarbonyl)amino]methyl}cyclohexyl)carbonyl]-3-[2-(4-methylpiperazin-1-yl)pyridin-4-yl]-N-(2-oxo-2,3-dihydro-1H-benzimidazol-5-yl)-L-phenylalaninamide in 7 ml of 1,4-dioxane, and the mixture was stirred at 40° C. for 5 h. 5 ml of acetonitrile were added to the reaction mixture and the resulting precipitate was filtered off with suction, washed with acetonitrile and dried under high vacuum. This gave 85 mg (94% of theory) of the title compound.

¹H NMR (400 MHz, DMSO-d₆): δ=ppm 0.75-0.94 (m, 2H), 0.98-1.11 (m, 1H), 1.15-1.28 (m, 1H), 1.37-1.48 (m, 2H), 1.60-1.76 (m, 3H), 2.05-2.14 (m, 1H), 2.54-2.61 (m, 2H), 2.79 (d, 3H), 2.88-2.97 (m, 1H), 3.08-3.20 (m, 2H), 3.43-3.51 (m, 3H), 3.53 (s, 3H), 4.60 (d, 2H), 4.67-4.75 (m, 1H), 6.81 (d, 1H), 7.06 (dd, 1H), 7.20 (br. d, 1H), 7.37-7.46 (m, 4H), 7.64-7.70 (m, 1H), 7.81-7.94 (m, 4H), 8.16 (d, 1H), 8.21 (d, 1H), 10.18 (s, 1H), 10.46 (s, 1H), 10.52 (s, 1H).

LC-MS (Method 4): Rₜ=0.55 min; MS (ESIpos): m/z=611 [M+H−HCl]⁺.

### Example 77

### N-alpha-{[trans-4-(Aminomethyl)cyclohexyl]carbonyl}-3-[6-(4-methylpiperazin-1-yl)pyridin-3-yl]-N-[4-(1H-tetrazol-5-yl)phenyl]-L-phenylalaninamide hydrochloride

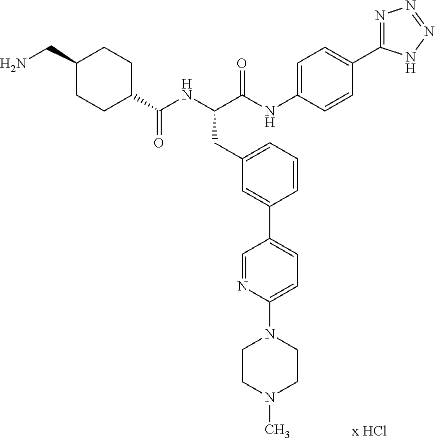

0.17 ml (0.67 mmol) of 4M hydrogen chloride in 1,4-dioxane was added to a suspension of 70 mg (0.1 mmol) of N-alpha-[(trans-4-{[(tert-butoxycarbonyl)amino]methyl}cyclohexyl)carbonyl]-3-[6-(4-methylpiperazin-1-yl)pyridin-3-yl]-N-[4-(1H-tetrazol-5-yl)phenyl]-L-phenylalaninamide in 5 ml of 1,4-dioxane, and the mixture was stirred at 40° C. overnight. 5 ml of acetonitrile were added to the reaction mixture and the resulting precipitate was filtered off with suction, washed with acetonitrile and dried under high vacuum. This gave 64 mg (100% of theory) of the title compound.

¹H NMR (300 MHz, DMSO-d₆): δ=ppm 0.74-0.96 (m, 2H), 1.01-1.30 (m, 2H), 1.36-1.53 (m, 2H), 1.70 (m, 3H), 2.06-2.17 (m, 1H), 2.54-2.63 (m, 2H), 2.77 (m, 3H), 2.86-2.97 (m, 1H), 2.99-3.16 (m, 4H), 3.23-3.38 (m, 3H), 3.41-3.50 (m, 2H), 3.53 (s, 3H), 4.43 (d, 2H), 4.67-4.77 (m, 1H), 7.10 (d, 1H), 7.24 (d, 1H), 7.31 (t, 1H), 7.45 (d, 1H), 7.59 (s, 1H), 7.81 (m, 5H), 7.91-8.03 (m, 3H), 8.26 (d, 1H), 8.40 (d, 1H), 10.61 (s, 1H), 10.88-11.03 (m, 1H).

LC-MS (Method 4): Rₜ=0.61 min; MS (ESIpos): m/z=623 [M+H−HCl]⁺.

### Example 78

### N-alpha-{[trans-4-(Aminomethyl)cyclohexyl]carbonyl}-3-[4-(benzyloxy)-2-(dimethylamino)pyrimidin-5-yl]-N-[4-(1H-tetrazol-5-yl)phenyl]-L-phenylalaninamide hydrochloride

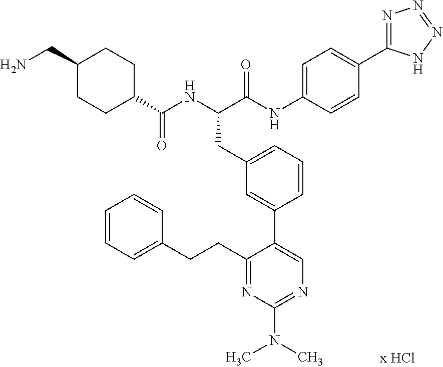

0.16 ml (0.66 mmol) of 4M hydrogen chloride in 1,4-dioxane was added to a suspension of 73 mg (0.093 mmol) of 3-[4-(benzyloxy)-2-(dimethylamino)pyrimidin-5-yl]-N-alpha-[(trans-4-{[(tert-butoxycarbonyl)amino]methyl}cyclohexyl)carbonyl]-N-[4-(1H-tetrazol-5-yl)phenyl]-L-phenylalaninamide in 3 ml of 1,4-dioxane, and the mixture was stirred at 40° C. for 5 h. 5 ml of acetonitrile were added to the reaction mixture and the resulting precipitate was filtered off with suction, washed with acetonitrile and dried under high vacuum. This gave 64 mg (89% of theory) of the title compound.

¹H NMR (300 MHz, DMSO-d₆): δ=ppm 0.74-0.97 (m, 2H), 1.01-1.30 (m, 2H), 1.33-1.54 (m, 2H), 1.59-1.77 (m, 3H), 2.04-2.15 (m, 1H), 2.55-2.62 (m, 2H), 2.88 (dd, 1H), 3.06 (dd, 1H), 3.14 (s, 6H), 4.63-4.75 (m, 1H), 5.45 (s, 2H), 7.19-7.37 (m, 7H), 7.38-7.44 (m, 3H), 7.77 (m, 5H), 7.94 (d, 2H), 8.12 (s, 1H), 8.20 (d, 1H), 10.49 (s, 1H).

LC-MS (Method 4): Rₜ=0.90 min; MS (ESIpos): m/z=675 [M+H−HCl]⁺.

### Example 79

### N-alpha-{[trans-4-(Aminomethyl)cyclohexyl]carbonyl}-3-(2-aminopyrimidin-5-yl)-N-[4-(1H-tetrazol-5-yl)phenyl]-L-phenylalaninamide hydrochloride

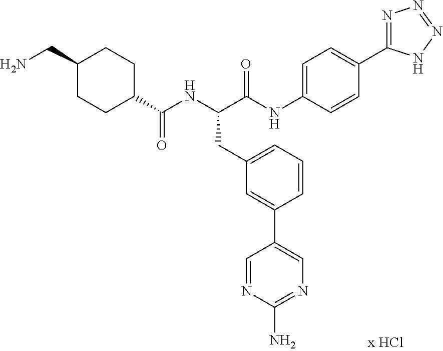

0.5 ml (2 mmol) of 4M hydrogen chloride in 1,4-dioxane was added to a suspension of 457 mg (0.28 mmol) of 3-(2-aminopyrimidin-5-yl)-N-alpha-[(trans-4-{[(tert-butoxycarbonyl)amino]methyl}cyclohexyl)carbonyl]-N-[4-(1H-tetrazol-5-yl)phenyl]-L-phenylalaninamide in 8 ml of dichloromethane, and the mixture was stirred at RT overnight. After addition of a further 0.2 ml (0.085 mmol) of 4M hydrogen chloride in 1,4-dioxane and at 40° C., 5 ml of acetonitrile were added to the reaction mixture and the resulting precipitate was filtered off with suction, washed with acetonitrile and dried under high vacuum. The crude product was purified by chromatography via HPLC (Method 7). This gave 65 mg (36% of theory) of the title compound.

¹H NMR (300 MHz, DMSO-d₆): δ=ppm 0.75-0.96 (m, 2H), 1.00-1.30 (m, 2H), 1.33-1.52 (m, 2H), 1.58-1.77 (m, 3H), 2.05-2.16 (m, 1H), 2.59 (d, 2H), 2.87 (dd, 1H), 3.08 (dd, 1H), 4.64-4.77 (m, 1H), 6.74 (s, 2H), 7.21 (d, 1H), 7.30 (t, 1H), 7.41 (d, 1H), 7.52 (s, 1H), 7.57 (d, 2H), 7.86 (d, 2H), 8.14 (d, 1H), 8.51 (s, 2H), 10.11 (s, 1H).

LC-MS (Method 4): Rₜ=0.63 min; MS (ESIpos): m/z=541 [M+H−HCl]⁺.

### Example 80

### N-alpha-{[trans-4-(Aminomethyl)cyclohexyl]carbonyl}-3-(3-methylpyridin-4-yl)-N-[4-(1H-tetrazol-5-yl)phenyl]-L-phenylalaninamide hydrochloride

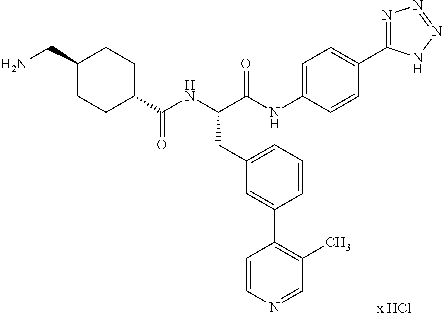 [SMILES: Cc1cnccc1-c1cccc(C[C@H](NC(=O)[C@H]2CC[C@H](CN)CC2)C(=O)Nc2ccc(-c3nnn[nH]3)cc2)c1]

0.4 ml (1.66 mmol) of 4M hydrogen chloride in 1,4-dioxane was added to a solution of 152 mg (0.24 mmol) of N-alpha-[(trans-4-{[(tert-butoxycarbonyl)amino]methyl}cyclohexyl)carbonyl]-3-(3-methylpyridin-4-yl)-N-[4-(1H-tetrazol-5-yl)phenyl]-L-phenylalaninamide in 1.8 ml of DMSO, and the mixture was stirred at RT overnight. After addition of a further 0.9 ml (3.5 mmol) of 4M hydrogen chloride in 1,4-dioxane and stirring at 40° C. overnight, the reaction mixture was concentrated and purified chromatographically by HPLC (Method 10). This gave 50 mg (33% of theory) of the title compound.

¹H NMR (400 MHz, DMSO-d₆): δ=ppm 0.77-0.95 (m, 2H), 1.03-1.29 (m, 2H), 1.34-1.52 (m, 2H), 1.61-1.76 (m, 3H), 2.04-2.13 (m, 1H), 2.17 (s, 3H), 2.60 (d, 2H), 2.91 (dd, 1H), 3.10 (dd, 1H), 4.67-4.75 (m, 1H), 7.12 (d, 1H), 7.18-7.23 (m, 1H), 7.28 (s, 1H), 7.31-7.40 (m, 2H), 7.54-7.68 (m, 4H), 7.89 (d, 2H), 8.13 (d, 1H), 8.38 (d, 1H), 8.45 (s, 1H), 10.21 (s, 1H).

LC-MS (Method 4): Rₜ=0.60 min; MS (ESIpos): m/z=539 [M+H−HCl]⁺.

### Example 81

### N-alpha-{[trans-4-(Aminomethyl)cyclohexyl]carbonyl}-N-[4-(1H-tetrazol-5-yl)phenyl]-3-[2-(trifluoromethyl)pyridin-3-yl]-L-phenylalaninamide hydrochloride

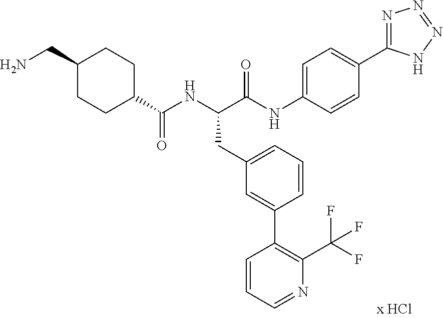

0.06 ml (0.245 mmol) of 4M hydrogen chloride in 1,4-dioxane was added to a suspension of 24 mg (0.035 mmol) of N-alpha-[(trans-4-{[(tert-butoxycarbonyl)amino]methyl}cyclohexyl)carbonyl]-N-[4-(1H-tetrazol-5-yl)phenyl]-3-[2-(trifluoromethyl)pyridin-3-yl]-L-phenylalaninamide in 1.8 ml of 1,4-dioxane, and the mixture was stirred at RT overnight. After addition of a further 0.02 ml (0.07 mmol) of 4M hydrogen chloride in 1,4-dioxane and 4 h at 50° C., 5 ml of acetonitrile were added to the reaction mixture and the resulting precipitate was filtered off with suction, washed with acetonitrile and dried under high vacuum. This gave 50 mg (33% of theory) of the title compound.

¹H NMR (400 MHz, DMSO-d₆): δ=ppm 0.77-0.94 (m, 2H), 1.04-1.29 (m, 2H), 1.36-1.54 (m, 2H), 1.62-1.76 (m, 3H), 2.01-2.13 (m, 1H), 2.56-2.62 (m, 2H), 2.92 (dd, 1H), 3.10 (dd, 1H), 4.65-4.74 (m, 1H), 7.13-7.19 (m, 1H), 7.25 (s, 1H), 7.33-7.41 (m, 2H), 7.61-7.80 (m, 7H), 7.95 (d, 2H), 8.14 (d, 1H), 8.71-8.76 (m, 1H), 10.43 (s, 1H).

LC-MS (Method 4): Rₜ=0.86 min; MS (ESIpos): m/z=593 [M+H−HCl]⁺.

### Example 82

### N-alpha-{[trans-4-(Aminomethyl)cyclohexyl]carbonyl}-3-[2-(cyclopropylamino)pyrimidin-5-yl]-N-[4-(1H-tetrazol-5-yl)phenyl]-L-phenylalaninamide hydrochloride

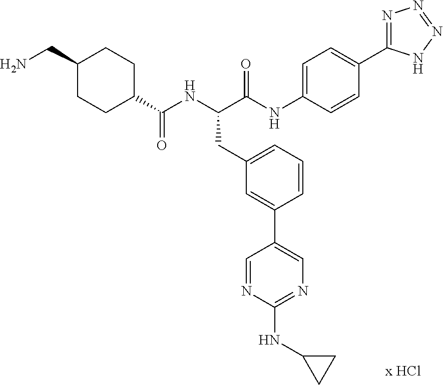

0.11 ml (0.45 mmol) of 4M hydrogen chloride in 1,4-dioxane was added to a suspension of 44 mg (0.065 mmol) of N-alpha-[(trans-4-{[(tert-butoxyycarbonyl)amino]methyl}cyclohexyl)carbonyl]-3-[2-(cyclopropylamino)pyrimidin-5-yl]-N-[4-(1H-tetrazol-5-yl)phenyl]-L-phenylalaninamide in 3.2 ml of 1,4-dioxane, and the mixture was stirred at 50° C. for 4 h and then at RT for 72 h. After addition of a further 0.33 ml (1.3 mmol) of 4M hydrogen chloride in 1,4-dioxane and 96 h at RT and 3 h at 60° C., 5 ml of acetonitrile were added to the reaction mixture and the precipitate formed was filtered off with suction. The precipitate was suspended in water and lyophilized. This gave 33 mg (80% of theory) of the title compound.

¹H NMR (300 MHz, MeOH-d₄): δ=ppm 0.64-0.72 (m, 2H), 0.89-0.98 (m, 2H), 0.99-1.14 (m, 2H), 1.27-1.66 (m, 4H), 1.68-1.95 (m, 5H), 2.20-2.32 (m, 1H), 2.68-2.74 (m, 1H), 2.77 (d, 2H), 3.10 (dd, 1H), 3.20-3.26 (m, 1H), 4.73-4.78 (m, 1H), 7.32-7.51 (m, 5H), 7.69 (d, 2H), 7.90 (d, 2H), 8.61 (s, 2H).

LC-MS (Method 4): Rₜ=0.77 min; MS (ESIpos): m/z=581 [M+H−HCl]⁺.

### Example 83

### N-alpha-{[trans-4-(Aminomethyl)cyclohexyl]carbonyl}-3-(5-methyl-1H-indazol-4-yl)-N-[4-(1H-tetrazol-5-yl)phenyl]-L-phenylalaninamide hydrochloride

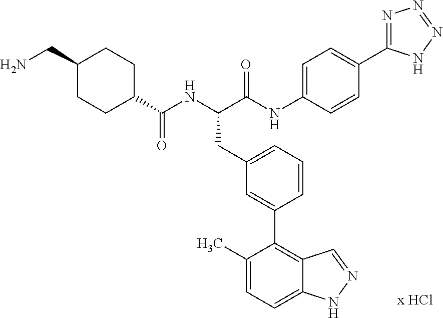

0.1 ml (0.4 mmol) of 4M hydrogen chloride in 1,4-dioxane was added to a suspension of 39 mg (0.006 mmol) of N-alpha-[(trans-4-{[(tert-butoxycarbonyl)amino]methyl}cyclohexyl)carbonyl]-3-(5-methyl-1H-indazol-4-yl)-N-[4-(1H-tetrazol-5-yl)phenyl]-L-phenylalaninamide in 2 ml of 1,4-dioxane, and the mixture was stirred at RT for 4 h. 5 ml of acetonitrile were added to the reaction mixture and the resulting precipitate was filtered off with suction, washed with acetonitrile and then dried under high vacuum. This gave 32 mg (90% of theory) of the title compound.

¹H NMR (300 MHz, MeOH-d₄): δ=ppm 0.93-1.12 (m, 2H), 1.26-1.48 (m, 2H), 1.49-1.62 (m, 1H), 1.64-1.93 (m, 4H), 2.17-2.30 (m, 4H), 2.75 (d, 2H), 3.04-3.15 (m, 1H), 4.84-4.88 (m, 1H), 7.22-7.28 (m, 1H), 7.31 (s, 1H), 7.35-7.48 (m, 4H), 7.69-7.75 (m, 3H), 7.87-7.93 (m, 2H).

LC-MS (Method 4): Rₜ=0.86 min; MS (ESIpos): m/z=578 [M+H−HCl]⁺.

### Example 84

### trans-4-(Aminomethyl)-N-{(25)-1-oxo-1-{[4-(1H-tetrazol-5-yl)phenyl]amino}-3-[2′-(trifluoromethyl)biphenyl-3-yl]propan-2-yl}cyclohexanecarboxamide hydrochloride

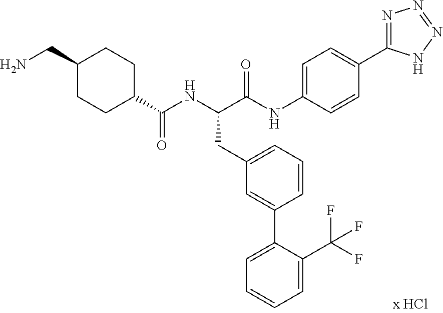

0.6 ml (2.3 mmol) of 4M hydrogen chloride in 1,4-dioxane was added to a suspension of 229 mg (0.332 mmol) of tert-butyl {[trans-4-({(2S)-1-oxo-1-{[4-(1H-tetrazol-5-yl)phenyl]amino}-3-[2′-(trifluoromethyl)biphenyl-3-yl]propan-2-yl}carbamoyl)cyclohexyl]methyl}carbamate in 10 ml of dichloromethane, and the mixture was stirred at 50° C. for 3 h and then at RT overnight. 5 ml of acetonitrile were added to the reaction mixture and the resulting precipitate was filtered off with suction, washed with acetonitrile, dried under high vacuum and then chromatographed by HPLC (Method 8). This gave 16 mg (7% of theory) of the title compound.

¹H NMR (300 MHz, DMSO-d₆): δ=ppm 0.72-0.97 (m, 2H), 1.02-1.30 (m, 2H), 1.31-1.55 (m, 2H), 1.58-1.79 (m, 3H), 1.98-2.14 (m, 1H), 2.56-2.63 (m, 2H), 2.83-2.95 (m, 1H), 3.04-3.12 (m, 1H), 4.61-4.76 (m, 1H), 7.11-7.18 (m, 1H), 7.21-7.28 (m, 1H), 7.31-7.40 (m, 2H), 7.49-7.57 (m, 3H), 7.68-7.77 (m, 2H), 7.81-7.88 (m, 2H), 8.04-8.16 (m, 1H), 8.69-8.76 (m, 1H), 10.06 (s, 1H).

LC-MS (Method 4): Rₜ=0.82 min; MS (ESIpos): m/z=593 [M+H−HCl]⁺.

### Example 85

### N-alpha-{[trans-4-(Aminomethyl)cyclohexyl]carbonyl}-3-[2-(morpholin-4-yl)pyridin-4-yl]-N-[4-(2H-tetrazol-5-yl)phenyl]-L-phenylalaninamide hydrochloride

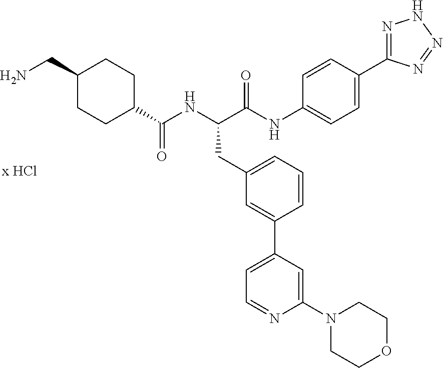

0.29 ml (1.15 mmol) of 4M hydrogen chloride in 1,4-dioxane was added to a solution of 63 mg (0.08 mmol) of N-alpha-[(trans-4-{[(tert-butoxycarbonyl)amino]methyl}cyclohexyl)carbonyl]-3-[2-(morpholin-4-yl)pyridin-4-yl]-N-[4-(2H-tetrazol-5-yl)phenyl]-L-phenylalaninamide in 3 ml of dioxane, and the mixture was stirred at RT overnight. The solid formed was filtered off, washed with acetonitrile and dried under high vacuum. 43 mg (80% of theory) of the title compound were obtained.

¹H NMR (400 MHz, DMSO-d₆): δ=ppm 0.78-0.98 (m, 2H), 1.02-1.32 (m, 2H), 1.38-1.53 (m, 2H), 1.61-1.80 (m, 3H), 2.07-2.20 (m, 1H), 2.57-2.69 (m, 2H), 2.97 (dd, 1H), 3.21 (dd, 1H), 3.3-4.0 (m, 8H), 4.75-4.85 (m, 1H), 7.22 (bs, 1H), 7.35-7.57 (m, 3H), 7.70-7.95 (m, 7H), 8.02 (d, 2H), 8.13 (d, 1H), 8.32 (d, 1H), 10.70 (s, 1H).

LC-MS (Method 1): Rₜ=0.59 min; MS (ESIneg): m/z=608 [M−H−HCl]⁻.

### Example 86

### trans-N-[(2S)-3-[5′-(Acetylamino)-2′-chlorobiphenyl-3-yl]-1-oxo-1-{[4-(1H-tetrazol-5-yl)phenyl]amino}propan-2-yl]-4-(aminomethyl)cyclohexanecarboxamide hydrochloride

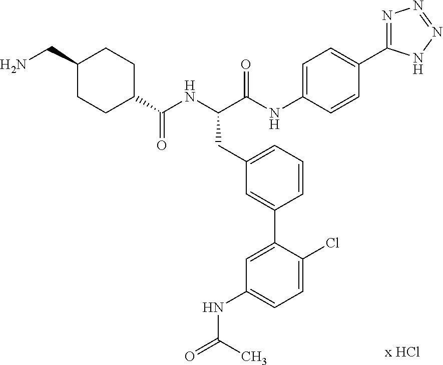

0.18 ml (0.71 mmol) of 4M hydrogen chloride in 1,4-dioxane was added to a suspension of 73 mg (0.1 mmol) of tert-butyl [(trans-4-{[(2S)-3-[5′-(acetylamino)-2′-chlorobiphenyl-3-yl]-1-oxo-1-{[4-(1H-tetrazol-5-yl)phenyl]amino}propan-2-yl]carbamoyl}cyclohexyl)methyl]carbamate in 5 ml of 1,4-dioxane, and the mixture was stirred at RT overnight. 5 ml of acetonitrile were added to the reaction mixture and the resulting precipitate was filtered off with suction, washed with acetonitrile, suspended in water and lyophilized. This gave 48 mg (71% of theory) of the title compound.

¹H NMR (300 MHz, DMSO-d₆): δ=ppm 0.74-0.95 (m, 2H), 1.01-1.29 (m, 2H), 1.34-1.56 (m, 2H), 1.70 (s, 3H), 2.01 (s, 3H), 2.03-2.14 (m, 1H), 2.55-2.63 (m, 2H), 2.90 (dd, 1H), 3.10 (dd, 1H), 4.61-4.73 (m, 1H), 7.16-7.22 (m, 1H), 7.32 (d, 3H), 7.42 (d, 1H), 7.51 (dd, 1H), 7.62-7.64 (m, 1H), 7.65-7.74 (m, 2H), 7.77 (d, 2H), 7.96 (d, 2H), 8.19 (d, 1H), 10.14 (s, 1H), 10.49 (s, 1H).

LC-MS (Method 4): Rₜ=0.83 min; MS (ESIpos): m/z=615 [M+H−HCl]⁺.

### Example 87

### N-alpha-{[trans-4-(Aminomethyl)cyclohexyl]carbonyl}-3-(5-methyl-1H-indazol-4-yl)-N-[4-(5-oxo-4,5-dihydro-1,2,4-oxadiazol-3-yl)phenyl]-L-phenylalaninamide hydrochloride

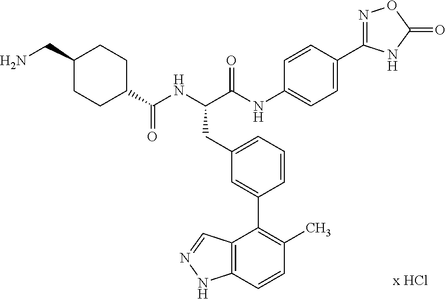

0.12 ml (0.47 mmol) of 4M hydrogen chloride in 1,4-dioxane was added to a suspension of 47 mg (0.07 mmol) of N-alpha-[(trans-4-{[(tert-butoxycarbonyl)amino]methyl}cyclohexyl)carbonyl]-3-(5-methyl-1H-indazol-4-yl)-N-[4-(5-oxo-4,5-dihydro-1,2,4-oxadiazol-3-yl)phenyl]-L-phenylalaninamide in 7 ml of 1,4-dioxane, and the mixture was stirred at RT overnight. 5 ml of acetonitrile were added to the reaction mixture and the resulting precipitate was filtered off with suction and dried under high vacuum. This gave 33 mg (70% of theory) of the title compound.

¹H NMR (300 MHz, DMSO-d₆): δ=ppm 0.76-0.95 (m, 2H), 1.03-1.30 (m, 2H), 1.35-1.53 (m, 2H), 1.59-1.77 (m, 3H), 2.04-2.15 (m, 1H), 2.18 (s, 3H), 2.55-2.63 (m, 2H), 2.92 (dd, 1H), 3.13 (dd, 1H), 4.68-4.80 (m, 1H), 7.17-7.21 (m, 1H), 7.24 (d, 1H), 7.29-7.36 (m, 3H), 7.38 (d, 2H), 7.55 (s, 1H), 7.73 (m, 7H), 8.21 (d, 1H), 10.52 (s, 1H), 12.85 (s, 1H).

LC-MS (Method 4): Rₜ=0.79 min; MS (ESIpos): m/z=594 [M+H−HCl]⁺.

### Example 88

### N-alpha-{[trans-4-(Aminomethyl)cyclohexyl]carbonyl}-3-[2-(morpholin-4-yl)pyrimidin-5-yl]-N-[4-(5-oxo-4,5-dihydro-1H-1,2,4-triazol-3-yl)phenyl]-L-phenylalaninamide hydrochloride

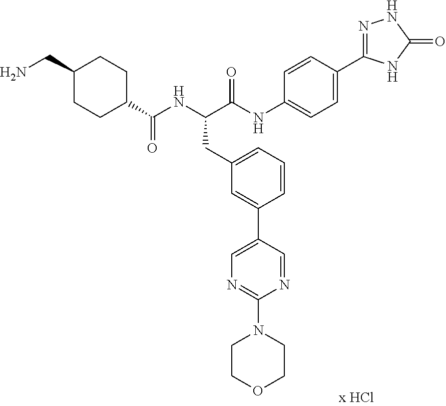

0.1 ml (0.4 mmol) of 4M hydrogen chloride in 1,4-dioxane was added to a suspension of 15 mg (0.02 mmol) of N-alpha-[(trans-4-{[(tert-butoxycarbonyl)amino]methyl}cyclohexyl)carbonyl]-3-[2-(morpholin-4-yl)pyrimidin-5-yl]-N-[4-(5-oxo-4,5-dihydro-1H-1,2,4-triazol-3-yl)phenyl]-L-phenylalaninamide in 0.8 ml of 1,4-dioxane, and the mixture was stirred at 50° C. for 1 h. 5 ml of acetonitrile were added to the reaction mixture and the resulting precipitate was filtered off with suction and dried under high vacuum. This gave 10 mg (66% of theory) of the title compound.

¹H NMR (300 MHz, MeOH-d₄): δ=ppm 0.94-1.16 (m, 2H), 1.28-1.49 (m, 2H), 1.49-1.66 (m, 1H), 1.68-1.95 (m, 4H), 2.19-2.33 (m, 1H), 2.72-2.79 (m, 2H), 3.06 (dd, 1H), 3.21 (dd, 1H), 3.71-3.85 (m, 8H), 4.70-4.78 (m, 1H), 7.27-7.33 (m, 1H), 7.35-7.46 (m, 3H), 7.54-7.61 (m, 2H), 7.63-7.70 (m, 2H), 8.52 (s, 2H).

LC-MS (Method 4): Rₜ=0.87 min; MS (ESIpos): m/z=626 [M+H−HCl]⁺.

### Example 89

### N-alpha-{[trans-4-(Aminomethyl)cyclohexyl]carbonyl}-3-(3-methylpyridin-4-yl)-N-[4-(5-oxo-4,5-dihydro-1H-1,2,4-triazol-3-yl)phenyl]-L-phenylalaninamide hydrochloride

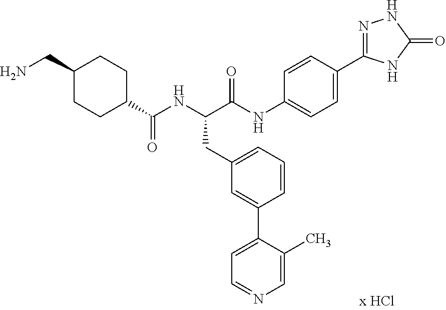

0.11 ml (0.4 mmol) of 4M hydrogen chloride in 1,4-dioxane was added to a suspension of 58 mg (0.09 mmol) of N-alpha-[(trans-4-{[(tert-butoxycarbonyl)amino]methyl}cyclohexyl)carbonyl]-3-(3-methylpyridin-4-yl)-N-[4-(5-oxo-4, 5-dihydro-1H-1,2,4-triazol-3-yl)phenyl]-L-phenylalaninamide in 2 ml of 1,4-dioxane, and the mixture was stirred at 40° C. for 2 h. After further addition of 0.11 ml (0.4 mmol) of 4M hydrogen chloride in 1,4-dioxane and 3 h at 45-50° C., 5 ml of acetonitrile were added to the reaction mixture and the precipitate formed was filtered off with suction and purified chromatographically by HPLC (Method 6). This gave 23 mg (40% of theory) of the title compound.

¹H NMR (300 MHz, DMSO-d₆): δ=ppm 0.70-0.92 (m, 2H), 0.99-1.37 (m, 3H), 1.39-1.51 (m, 1H), 1.58-1.76 (m, 3H), 1.99-2.12 (m, 1H), 2.16 (s, 3H), 2.89 (dd, 1H), 3.07 (dd, 1H), 4.60-4.73 (m, 1H), 4.95-5.10 (m, 1H), 7.06-7.15 (m, 1H), 7.16-7.22 (m, 1H), 7.24-7.29 (m, 1H), 7.34 (s, 2H), 7.66 (m, 4H), 8.23-8.31 (m, 1H), 8.34-8.41 (m, 2H), 8.42-8.48 (m, 1H), 10.34-10.46 (m, 1H).

LC-MS (Method 4): Rₜ=0.55 min; MS (ESIpos): m/z=554 [M+H−HCl]⁺.

### Example 90

### N-alpha-{[trans-4-(Aminomethyl)cyclohexyl]carbonyl}-N-[4-(3-chloro-4H-1,2,4-triazol-5-yl)phenyl]-3-{6-[3-(dimethylamino)propoxy]pyridin-3-yl}-L-phenylalaninamide hydrochloride

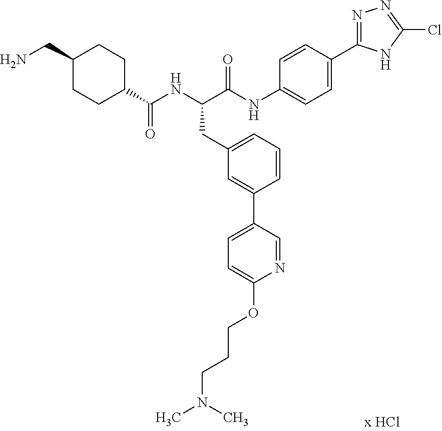

0.19 ml (0.75 mmol) of 4M hydrogen chloride in 1,4-dioxane was added to a suspension of 81 mg (0.1 mmol) of N-alpha-[(trans-4-{[(tert-butoxycarbonyl)amino]methyl}cyclohexyl)carbonyl]-N-[4-(3-chloro-4H-1,2,4-triazol-5-yl)phenyl]-3-{6-[3-(dimethylamino)propoxy]pyridin-3-yl}-L-phenylalaninamide in 3.5 ml of 1,4-dioxane, and the mixture was stirred at RT for 48 h. 5 ml of acetonitrile were then added to the reaction mixture and the resulting precipitate was filtered off with suction and dried under high vacuum. This gave 73 mg (96% of theory) of the title compound.

¹H NMR (400 MHz, DMSO-d₆): δ=ppm 0.81-0.98 (m, 2H), 1.05-1.18 (m, 1H), 1.18-1.31 (m, 1H), 1.40-1.56 (m, 2H), 1.64-1.80 (m, 3H), 2.09-2.19 (m, 3H), 2.57-2.64 (m, 2H), 2.77 (s, 3H), 2.78 (s, 3H), 2.95 (dd, 1H), 3.10-3.24 (m, 4H), 4.33-4.38 (m, 2H), 4.70-4.79 (m, 1H), 6.90 (d, 1H), 7.29 (d, 1H), 7.35 (t, 1H), 7.47 (d, 1H), 7.60 (s, 1H), 7.78 (d, 2H), 7.84 (br. s, 3H), 7.91 (d, 2H), 7.95-8.00 (m, 1H), 8.26 (d, 1H), 8.40-8.46 (m, 1H), 10.23-10.37 (m, 1H), 10.56 (s, 1H).

LC-MS (Method 4): Rₜ=0.77 min; MS (ESIpos): m/z=659.5 [M+H−HCl]⁺.

### Example 91

### N-alpha-{[trans-4-(Aminomethyl)cyclohexyl]carbonyl}-N-[4-(3-chloro-4H-1,2,4-triazol-5-yl)phenyl]-3-[2-(4-methylpiperazin-1-yl)pyrimidin-5-yl]-L-phenylalaninamide hydrochloride

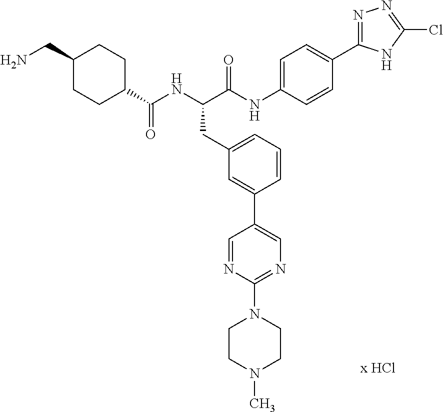

0.12 ml (0.5 mmol) of 4M hydrogen chloride in 1,4-dioxane was added to a suspension of 53 mg (0.07 mmol) of N-alpha-[(trans-4-{[(tert-butoxycarbonyl)amino]methyl}cyclohexyl)carbonyl]-N-[4-(3-chloro-4H-1,2,4-triazol-5-yl)phenyl]-3-[2-(4-methylpiperazin-1-yl)pyrimidin-5-yl]-L-phenylalaninamide in 2.0 ml of 1,4-dioxane, and the mixture was stirred at RT overnight. 5 ml of acetonitrile were then added to the reaction mixture and the resulting precipitate was filtered off with suction and dried under high vacuum. This gave 52 mg (100% of theory) of the title compound.

¹H NMR (300 MHz, DMSO-d₆): δ=ppm 0.74-0.96 (m, 2H), 1.00-1.29 (m, 2H), 1.34-1.54 (m, 2H), 1.60-1.78 (m, 3H), 2.05-2.16 (m, 1H), 2.54-2.62 (m, 2H), 2.76 (d, 3H), 2.85-3.16 (m, 4H), 3.30-3.49 (m, 4H), 4.65-4.76 (m, 3H), 7.26 (d, 1H), 7.33 (t, 1H), 7.46 (d, 1H), 7.60 (s, 1H), 7.75 (d, 2H), 7.80-7.93 (m, 5H), 8.26 (d, 1H), 8.71 (s, 2H), 10.59 (s, 1H), 10.92-11.08 (m, 1H).

LC-MS (Method 4): Rₜ=0.64 min; MS (ESIpos): m/z=657.6 [M+H−HCl]⁺.

### Example 92

### N-alpha-{[trans-4-(Aminomethyl)cyclohexyl]carbonyl}-N-[4-(3-chloro-4H-1,2,4-triazol-5-yl)phenyl]-3-[2-(morpholin-4-yl)pyrimidin-5-yl]-L-phenylalaninamide hydrochloride

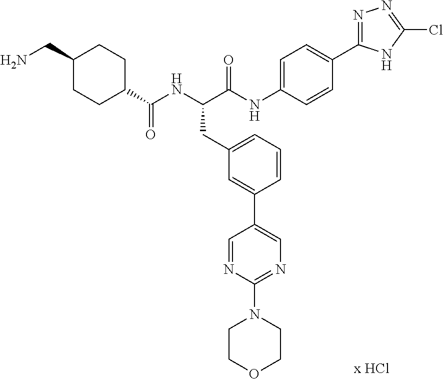

0.1 ml (0.37 mmol) of 4M hydrogen chloride in 1,4-dioxane was added to a suspension of 40 mg (0.05 mmol) of N-alpha-[(trans-4-{[(tert-butoxycarbonyl)amino]methyl}cyclohexyl)carbonyl]-N-[4-(3-chloro-4H-1,2,4-triazol-5-yl)phenyl]-3-[2-(morpholin-4-yl)pyrimidin-5-yl]-L-phenylalaninamide in 1.6 ml of 1,4-dioxane, and the mixture was stirred at RT overnight. 5 ml of acetonitrile were then added to the reaction mixture and the resulting precipitate was filtered off with suction and dried under high vacuum. This gave 35 mg (94% of theory) of the title compound.

¹H NMR (300 MHz, DMSO-d₆): δ=ppm 0.74-0.96 (m, 2H), 1.01-1.29 (m, 2H), 1.34-1.54 (m, 2H), 1.58-1.77 (m, 3H), 2.05-2.16 (m, 1H), 2.55-2.63 (m, 2H), 2.83-2.96 (m, 1H), 3.08 (dd, 1H), 3.67 (m, 9H), 4.65-4.76 (m, 1H), 7.23 (d, 1H), 7.31 (t, 1H), 7.43 (d, 1H), 7.55 (s, 1H), 7.67-7.81 (m, 5H), 7.86 (d, 2H), 8.23 (d, 1H), 8.64 (s, 2H), 10.48 (s, 1H).

LC-MS (Method 4): Rₜ=0.85 min; MS (ESIpos): m/z=644.5 [M+H−HCl]⁺.

### Example 93

### N-alpha-{[trans-4-(Aminomethyl)cyclohexyl]carbonyl}-N-[4-(3-chloro-4H-1,2,4-triazol-5-yl)phenyl]-3-[2-(dimethylamino)pyrimidin-5-yl]-L-phenylalaninamide hydrochloride

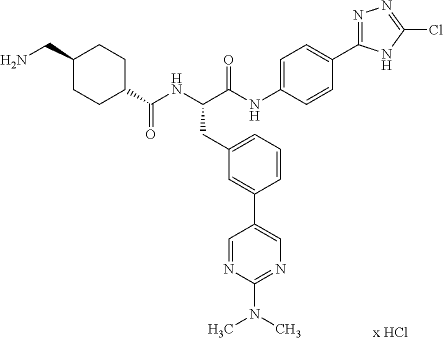

0.19 ml (0.76 mmol) of 4M hydrogen chloride in 1,4-dioxane was added to a suspension of 76 mg (0.11 mmol) of N-alpha-[(trans-4-{[(tert-butoxycarbonyl)amino]methyl}cyclohexyl)carbonyl]-N-[4-(3-chloro-4H-1,2,4-triazol-5-yl)phenyl]-3-[2-(dimethylamino)pyrimidin-5-yl]-L-phenylalaninamide in 3.3 ml of 1,4-dioxane, and the mixture was stirred at RT overnight. 5 ml of acetonitrile were then added to the reaction mixture and the resulting precipitate was filtered off with suction and dried under high vacuum. This gave 70 mg (98% of theory) of the title compound.

¹H NMR (400 MHz, DMSO-d₆): δ=ppm 0.77-0.95 (m, 2H), 1.03-1.29 (m, 2H), 1.35-1.53 (m, 2H), 1.61-1.75 (m, 3H), 2.05-2.15 (m, 1H), 2.55-2.61 (m, 2H), 2.91 (dd, 1H), 3.09 (dd, 1H), 3.13 (s, 6H), 4.67-4.76 (m, 1H), 7.23 (d, 1H), 7.31 (t, 1H), 7.43 (d, 1H), 7.52-7.57 (m, 1H), 7.70-7.80 (m, 5H), 7.86 (d, 2H), 8.20 (d, 1H), 8.62 (s, 2H), 10.47 (s, 1H).

LC-MS (Method 4): Rt=0.85 min; MS (ESIpos): m/z=602.5 [M+H−HCl]⁺.

### Example 94

### 3-[2-(Acetylamino)-4-methylpyrimidin-5-yl]-N-alpha-{[trans-4-(aminomethyl)cyclohexyl]-carbonyl}-N-[4-(1H-tetrazol-5-yl)phenyl]-L-phenylalaninamide hydrochloride

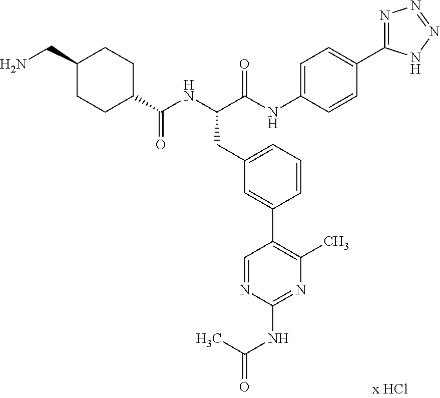

0.06 ml (0.25 mmol) of 4M hydrogen chloride in 1,4-dioxane was added to a suspension of 25 mg (0.04 mmol) of 3-[2-(Acetylamino)-4-methylpyrimidin-5-yl]-N-alpha-[(trans-4-{[(tert-butoxycarbonyl)amino]methyl}cyclohexyl)carbonyl]-N-[4-(1H-tetrazol-5-yl)phenyl]-L-phenylalaninamide in 1.2 ml of 1,4-dioxane, and the mixture was stirred at RT overnight. 5 ml of acetonitrile were then added to the reaction mixture and the resulting precipitate was filtered off with suction and dried under high vacuum. This gave 20 mg (78% of theory) of the title compound.

¹H NMR (300 MHz, DMSO-d₆): δ=ppm 0.75-0.97 (m, 2H), 1.01-1.30 (m, 2H), 1.34-1.51 (m, 2H), 1.60-1.77 (m, 3H), 2.07-2.11 (m, 1H), 2.16 (s, 3H), 2.31 (s, 3H), 2.56-2.63 (m, 2H), 2.91 (dd, 1H), 3.11 (dd, 1H), 4.67-4.77 (m, 1H), 7.21-7.26 (m, 1H), 7.30-7.40 (m, 3H), 7.78 (m, 5H), 7.95 (d, 2H), 8.24 (d, 1H), 8.35 (s, 1H), 10.51 (s, 1H), 10.55 (s, 1H).

LC-MS (Method 4): Rₜ=0.69 min; MS (ESIpos): m/z=597.5 [M+H−HCl]⁺.

### Example 95

### N-alpha-{[trans-4-(Aminomethyl)cyclohexyl]carbonyl}-3-(2-amino-4-methylpyrimidin-5-yl)-N-[4-(1H-tetrazol-5-yl)phenyl]-L-phenylalaninamide hydrochloride

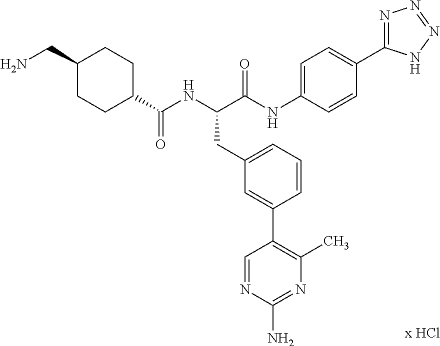

0.04 ml (0.17 mmol) of 4M hydrogen chloride in 1,4-dioxane was added to a suspension of 16 mg (0.02 mmol) of 3-(2-amino-4-methylpyrimidin-5-yl)-N-alpha-[(trans-4-{[(tert-butoxycarbonyl)amino]methyl}cyclohexyl)carbonyl]-N-[4-(1H-tetrazol-5-yl)phenyl]-L-phenylalaninamide in 0.5 ml of 1,4-dioxane, and the mixture was stirred at RT overnight. 5 ml of acetonitrile were then added to the reaction mixture and the resulting precipitate was filtered off with suction and dried under high vacuum. This gave 16 mg (97% of theory) of the title compound.

¹H NMR (300 MHz, DMSO-d₆): δ=ppm 0.72-0.97 (m, 2H), 1.01-1.30 (m, 2H), 1.36-1.52 (m, 2H), 1.61-1.79 (m, 3H), 2.04-2.15 (m, 1H), 2.26 (s, 3H), 2.56-2.64 (m, 2H), 2.90 (dd, 1H), 3.10 (dd, 2H), 4.65-4.77 (m, 1H), 7.16-7.23 (m, 1H), 7.27-7.38 (m, 3H), 7.78 (d, 6H), 7.96 (dd, 2H), 8.17-8.29 (m, 2H), 10.53 (s, 1H).

LC-MS (Method 4): Rₜ=0.66 min; MS (ESIpos): m/z=555.4 [M+H−HCl]⁺.

### Example 96

### 3-(3-{4-[(N-{[trans-4-(Aminomethyl)cyclohexyl]carbonyl}-3-[2-(morpholin-4-yl)pyrimidin-5-yl]-L-phenylalanyl)amino]phenyl}-1H-1,2,4-triazol-5-yl)-2,2,3,3-tetrafluoropropanoic acid hydrochloride

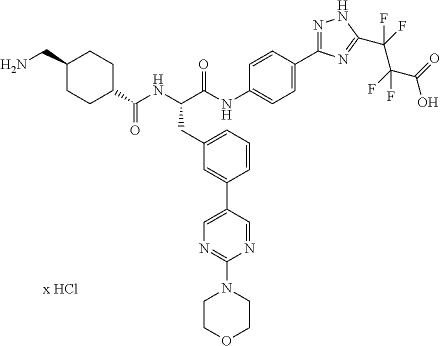

0.04 ml (0.16 mmol) of 4M hydrogen chloride in 1,4-dioxane was added to a solution of 44 mg (0.052 mmol) of 3-{3-[4-({N-[(trans-4-{[(tert-butoxycarbonyl)amino]methyl}cyclohexyl)carbonyl]-3-[2-(morpholin-4-yl)pyrimidin-5-yl]-L-phenylalanyl}amino)phenyl]-1H-1,2,4-triazol-5-yl}-2,2,3,3-tetrafluoropropanoic acid in 1 ml of dichloromethane, and the mixture was stirred at RT for 48 h. After addition of a further 0.026 ml (0.10 mmol) of 4M hydrogen chloride in 1,4-dioxane and stirring at 40° C. for 2 h, acetonitrile was added, the precipitate was filtered off, washed with a little acetonitrile and dried. The residue was purified by chromatography via HPLC (Method 8). This gave 15 mg (35% of theory) of the title compound.

¹H NMR (300 MHz, DMSO-d₆): δ=ppm 0.77-0.97 (m, 2H), 1.03-1.28 (m, 2H), 1.31-1.44 (m, 1H), 1.45-1.58 (m, 1H), 1.61-1.78 (m, 3H), 2.08-2.18 (m, 1H), 2.57-2.60 (m, 2H), 2.93 (dd, 1H), 3.12 (dd, 1H), 3.64-3.69 (m, 4H), 3.71-3.76 (m, 4H), 4.67-4.81 (m, 1H), 7.23-7.28 (m, 1H), 7.35 (t, 1H), 7.44-7.49 (m, 1H), 7.56 (s, 1H), 7.73 (d, 2H), 7.93 (d, 2H), 8.14-8.20 (m, 1H), 8.67 (s, 2H), 10.33 (s, 1H).

LC-MS (Method 4): Rₜ=0.80 min; MS (ESIpos): m/z=754.4 [M+H−HCl]⁺.

### Example 97

### trans-4-(Aminomethyl)-N-[(2S)-3-(4′-amino-2′-methylbiphenyl-3-yl)-1-oxo-1-{[4-(1H-tetrazol-5-yl)phenyl]amino}propan-2-yl]cyclohexanecarboxamide hydrochloride

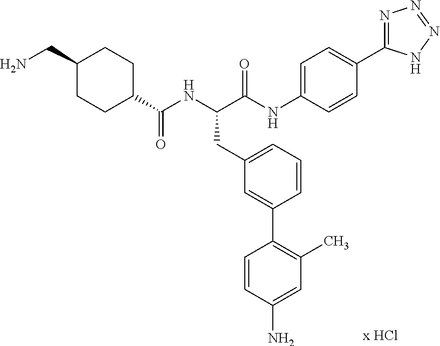

0.42 ml (1.7 mmol) of 4M hydrogen chloride in 1,4-dioxane was added to a suspension of 156 mg (0.3 mmol) of tert-butyl [(trans-4-{[(2S)-3-(4′-amino-2′-methylbiphenyl-3-yl)-1-oxo-1-{[4-(1H-tetrazol-5-yl)phenyl]amino}propan-2-yl]carbamoyl}cyclohexyl)methyl]carbamate in 1.8 ml of dimethyl sulphoxide, and the mixture was stirred at 40° C. for 5 h and at RT overnight. After adding a further 0.9 ml (3.6 mmol) of 4M hydrogen chloride in 1,4-dioxane and 48 h of stirring at RT and 24 h of stirring at 40° C., the reaction mixture was concentrated under reduced pressure and filtered using a Millipore filter. The solution that remained was purified by chromatography by HPLC (Method 10). This gave 33 mg (21% of theory) of the title compound.

¹H NMR (300 MHz, DMSO-d₆): δ=ppm 0.77-0.96 (m, 2H), 1.07-1.27 (m, 2H), 1.37-1.54 (m, 2H), 1.62-1.77 (m, 3H), 2.02 (s, 3H), 2.05-2.15 (m, 1H), 2.58-2.64 (m, 2H), 2.84 (dd, 1H), 3.05 (dd, 1H), 4.63-4.73 (m, 1H), 6.35-6.43 (m, 2H), 6.78 (d, 1H), 7.02 (d, 1H), 7.13-7.18 (m, 2H), 7.21 (d, 1H), 7.53-7.69 (m, 5H), 7.89 (d, 2H), 8.11 (d, 1H), 10.21 (s, 1H).

LC-MS (Method 4): Rₜ=0.70 min; MS (ESIpos): m/z=553.1 [M+H−HCl]⁺

### Example 98

### N-alpha-{[trans-4-(Aminomethyl)cyclohexyl]carbonyl)}-3-(6-hydroxy-2-methylpyridin-3-yl)-N-[4-(1H-tetrazol-5-yl)phenyl]-L-phenylalaninamide hydrochloride

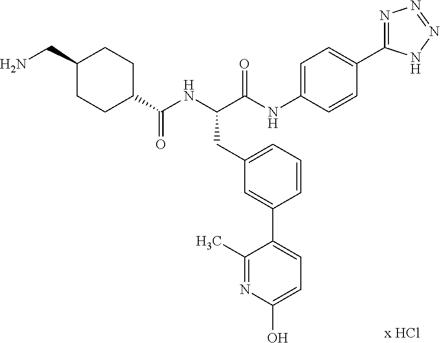

0.13 ml (0.53 mmol) of 4M hydrogen chloride in 1,4-dioxane was added to a suspension of 50 mg (0.08 mmol) of N-alpha-[(trans-4-{[(tert-butoxycarbonyl)amino]methyl}cyclohexyl)carbonyl]-3-(6-hydroxy-2-methylpyridin-3-yl)-N-[4-(1H-tetrazol-5-yl)phenyl]-L-phenylalaninamide in 3.0 ml of 1,4-dioxane, and the mixture was stirred at RT overnight. 5 ml of acetonitrile were then added to the reaction mixture and the resulting precipitate was filtered off with suction and dried under high vacuum. This gave 43 mg (91% of theory) of the title compound.

¹H NMR (400 MHz, DMSO-d₆): δ=ppm 0.80-0.98 (m, 2H), 1.05-1.17 (m, 1H), 1.18-1.31 (m, 1H), 1.40-1.54 (m, 2H), 1.66-1.79 (m, 3H), 2.08-2.16 (m, 4H), 2.59-2.64 (m, 2H), 2.91 (dd, 1H), 3.10 (dd, 1H), 4.67-4.77 (m, 1H), 6.22-6.28 (m, 1H), 7.09-7.16 (m, 1H), 7.21 (s, 1H), 7.32 (m, 3H), 7.80 (m, 5H), 8.00 (d, 2H), 8.21 (d, 1H), 10.51 (s, 1H).

LC-MS (Method 4): Rₜ=0.69 min; MS (ESIpos): m/z=555.4 [M+H−HCl]⁺

### Example 99

### N-alpha-{[trans-4-(Aminomethyl)cyclohexyl]carbonyl}-3-(2-hydroxypyrimidin-5-yl)-N-[4-(1H-tetrazol-5-yl)phenyl]-L-phenylalaninamide hydrochloride

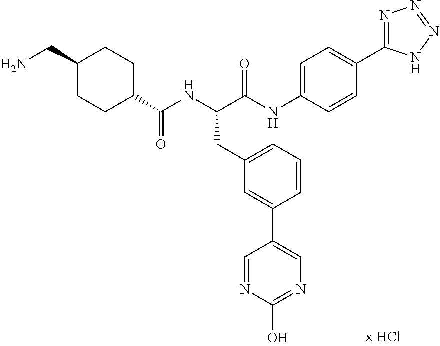

0.14 ml (0.47 mmol) of 4M hydrogen chloride in 1,4-dioxane was added to a suspension of 50 mg (0.08 mmol) of N-alpha-[(trans-4-{[(tert-butoxycarbonyl)amino]methyl}cyclohexyl)carbonyl]-3-(2-hydroxypyrimidin-5-yl)-N-[4-(1H-tetrazol-5-yl)phenyl]-L-phenylalaninamide in 2.7 ml of 1,4-dioxane, and the mixture was stirred at 40° C. for 4 h and at RT overnight. 5 ml of acetonitrile were then added to the reaction mixture and the resulting precipitate was filtered off with suction and dried under high vacuum. This gave 42 mg (91% of theory) of the title compound.

¹H NMR (400 MHz, DMSO-d₆): δ=ppm 0.78-0.96 (m, 2H), 1.03-1.16 (m, 1H), 1.16-1.28 (m, 1H), 1.36-1.52 (m, 2H), 1.61-1.77 (m, 3H), 2.07-2.16 (m, 1H), 2.55-2.62 (m, 2H), 2.90 (dd, 1H), 3.10 (dd, 1H), 4.68-4.76 (m, 1H), 7.25 (d, 1H), 7.31 (t, 1H), 7.45 (d, 1H), 7.60 (s, 1H), 7.73 (br. s, 3H), 7.79 (d, 2H), 7.96 (d, 2H), 8.20 (d, 1H), 8.67 (s, 2H), 10.52 (s, 1H), 10.51 (s, 1H).

LC-MS (Method 4): Rₜ=0.57 min; MS (ESIpos): m/z=664.6 [M+H−HCl]⁺

### Example 100

### N-alpha-{[trans-4-(Aminomethyl)cyclohexyl]carbonyl}-3-{6-[3-(dimethylamino)propoxy]pyridin-3-yl}-N-(2-oxo-2,3-dihydro-1H-benzimidazol-5-yl)-L-phenylalaninamide hydrochloride

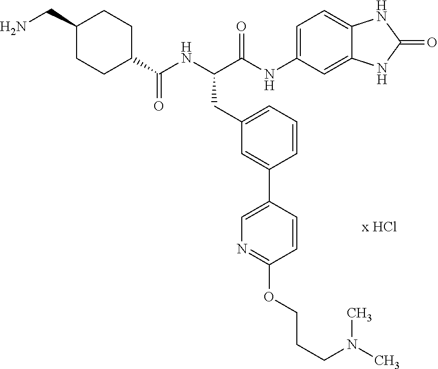

0.23 ml (0.9 mmol) of 4M hydrogen chloride in 1,4-dioxane was added to a suspension of 92 mg (0.13 mmol) of N-alpha-[(trans-4-{[(tert-butoxycarbonyl)amino]methyl}cyclohexyl)carbonyl]-3-{6-[3-(dimethylamino)propoxy]pyridin-3-yl}-N-(2-oxo-2,3-dihydro-1H-benzimidazol-5-yl)-L-phenylalaninamide in 6.4 ml of 1,4-dioxane, and the mixture was stirred at RT overnight. After adding a further 0.1 ml (0.39 mmol) of 4M hydrogen chloride in 1,4-dioxane and 4 h of stirring at 50° C., 5 ml of acetonitrile were added to the reaction mixture and the precipitate formed was filtered off with suction. The precipitate was purified chromatographically by HPLC (Method 18). This gave 37 mg (41% of theory) of the title compound.

¹H NMR (400 MHz, MeOH-d₄): δ=ppm 0.93-1.10 (m, 2H), 1.29-1.49 (m, 2H), 1.50-1.61 (m, 1H), 1.68-1.75 (m, 1H), 1.77-1.91 (m, 3H), 2.08 (m, 2H), 2.19-2.29 (m, 1H), 2.55 (s, 6H), 2.74 (d, 2H), 2.85 (dd, 2H), 3.04 (dd, 1H), 3.19 (dd, 1H), 4.34 (dd, 2H), 4.72 (dd, 1H), 6.77 (d, 1H), 6.86-6.94 (m, 2H), 7.27 (d, 1H), 7.32-7.42 (m, 4H), 7.77 (dd, 1H), 8.19 (d, 1H), 8.52 (s, 1H).

LC-MS (Method 4): Rₜ=0.61 min; MS (ESIpos): m/z=614.3 [M+H−HCl]⁺

### Example 101

### N-alpha-{[trans-4-(Aminomethyl)cyclohexyl]carbonyl}-3-{6-[3-(dimethylamino)propoxy]pyridin-3-yl}-N-{4-[3-(trifluoromethyl)-4H-1,2,4-triazol-5-yl]phenyl}-L-phenylalaninamide hydrochloride

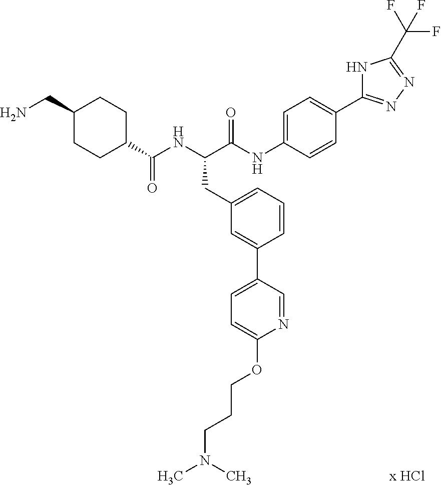

0.47 ml (0.65 mmol) of 4M hydrogen chloride in 1,4-dioxane was added to a suspension of 214 mg (0.27 mmol) of N-alpha-[(trans-4-{[(tert-butoxycarbonyl)amino]methyl}cyclohexyl)carbonyl]-3-{6-[3-(dimethylamino)propoxy]pyridin-3-yl}-N-{4-[3-(trifluoromethyl)-4H-1,2,4-triazol-5-yl]phenyl}-L-phenylalaninamide in 8.8 ml of 1,4-dioxane, and the mixture was stirred at RT for 24 h. The reaction mixture was then concentrated. The residue was purified by chromatography via HPLC (Method 7). This gave 40 mg (19% of theory) of the title compound.

¹H NMR (300 MHz, DMSO-d₆): δ=ppm 0.75-0.95 (m, 2H), 1.01-1.28 (m, 2H), 1.32-1.42 (m, 1H), 1.44-1.54 (m, 1H), 1.58-1.76 (m, 3H), 1.83 (quin, 2H), 2.08-2.12 (m, 1H), 2.14 (s, 6H), 2.35 (t, 2H), 2.55-2.60 (m, 2H), 2.91 (dd, 1H), 3.10 (dd, 1H), 4.27 (t, 2H), 4.65-4.76 (m, 1H), 6.84 (d, 1H), 7.25 (d, 1H), 7.32 (t, 1H), 7.44 (d, 1H), 7.54 (s, 1H), 7.64 (d, 2H), 7.91 (m, 3H), 8.16 (d, 1H), 8.21 (s, 1H), 8.36-8.40 (m, 1H), 10.24 (s, 1H).

LC-MS (Method 4): Rₜ=0.71 min; MS (ESIpos): m/z=693.4 [M+H−HCl]⁺

### Example 102

### N-alpha-{[trans-4-(Aminomethyl)cyclohexyl]carbonyl}-N-[4-(3-chloro-4H-1,2,4-triazol-5-yl)phenyl]-3-[4-methyl-6-(morpholin-4-yl)pyridin-3-yl]-L-phenylalaninamide hydrochloride

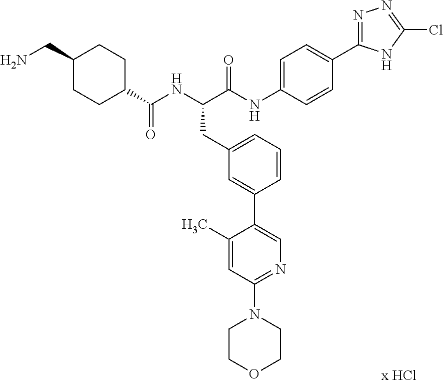

0.04 ml (0.16 mmol) of 4M hydrogen chloride in 1,4-dioxane was added to a suspension of 17 mg (0.02 mmol) of N-alpha-[(trans-4-{[(tert-butoxycarbonyl)amino]methyl}cyclohexyl)carbonyl]-N-[4-(3-chloro-4H-1,2,4-triazol-5-yl)phenyl]-3-[4-methyl-6-(morpholin-4-yl)pyridin-3-yl]-L-phenylalaninamide in 0.7 ml of 1,4-dioxane, and the mixture was stirred at RT for 24 h. 5 ml of acetonitrile were then added to the reaction mixture and the resulting precipitate was filtered off with suction and dried under high vacuum. This gave 10 mg (62% of theory) of the title compound.

¹H NMR (300 MHz, DMSO-d₆): δ=ppm 0.77-0.96 (m, 2H), 1.01-1.30 (m, 2H), 1.36-1.53 (m, 2H), 1.62-1.78 (m, 3H), 2.02-2.13 (m, 1H), 2.16 (s, 3H), 2.54-2.64 (m, 2H), 2.91 (dd, 1H), 3.10 (dd, 1H), 3.56-3.60 (m, 4H), 3.71 (m, 4H), 4.65-4.77 (m, 1H), 7.05-7.19 (m, 2H), 7.24 (s, 1H), 7.28-7.38 (m, 2H), 7.75 (m, 7H), 7.85 (d, 3H), 8.20 (d, 1H), 10.46 (s, 1H).

LC-MS (Method 4): Rₜ=0.71 min; MS (ESIpos): m/z=657.5 [M+H−HCl]⁺

### Example 103

### trans-4-(Aminomethyl)-N-[(2S)-3-(5′-amino-2′-methylbiphenyl-3-yl)-1-oxo-1-{[4-(1H-tetrazol-5-yl)phenyl]amino}propan-2-yl]cyclohexanecarboxamide hydrochloride

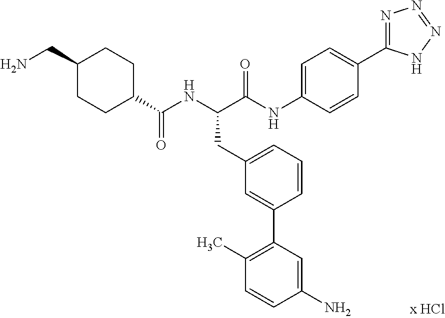

0.6 ml (2.3 mmol) of 4M hydrogen chloride in 1,4-dioxane was added to a suspension of 433 mg (0.3 mmol, 49% pure) of tert-butyl [(trans-4-{[(2S)-3-(5′-amino-2′-methylbiphenyl-3-yl)-1-oxo-1-{[4-(1H-tetrazol-5-yl)phenyl]amino}propan-2-yl]carbamoyl}cyclohexyl)methyl]carbamate in 8.0 ml of dichloromethane, and the mixture was stirred at RT overnight. After adding a further 0.16 ml (0.6 mmol) of 4M hydrogen chloride in 1,4-dioxane and 4 h of stirring at 50° C., 5 ml of acetonitrile were added to the reaction mixture and the precipitate formed was filtered off with suction. The residue was purified by chromatography via HPLC (Method 7). This gave 82 mg (39% of theory) of the title compound.

¹H NMR (300 MHz, DMSO-d₆): δ=ppm 0.76-0.95 (m, 2H), 1.04-1.31 (m, 2H), 1.32-1.54 (m, 2H), 1.61-1.77 (m, 3H), 1.95 (s, 3H), 2.03-2.14 (m, 1H), 2.60 (d, 2H), 2.85 (dd, 1H), 3.06 (dd, 1H), 4.61-4.72 (m, 1H), 6.35 (d, 1H), 6.44 (dd, 1H), 6.86 (d, 1H), 7.01-7.07 (m, 1H), 7.18-7.30 (m, 3H), 7.56 (d, 2H), 7.85 (d, 2H), 8.10 (d, 1H), 10.10 (s, 1H).

LC-MS (Method 4): Rₜ=0.67 min; MS (ESIpos): m/z=553.3 [M+H−HCl]⁺

### Example 104

### trans-4-(Aminomethyl)-N-[(2S)-3-(2′-isopropylbiphenyl-3-yl)-1-({4-[3-(methoxymethyl)-4H-1,2,4-triazol-5-yl]phenyl}amino)-1-oxopropan-2-yl]cyclohexanecarboxamide hydrochloride

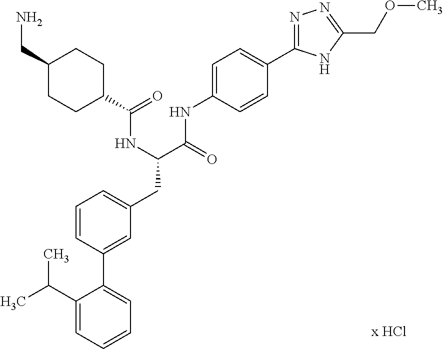

1.00 ml (4.00 mmol) of 4M hydrogen chloride in 1,4-dioxane was added to a solution of 49 mg (0.07 mmol) of tert-butyl [(trans-4-{[(2S)-3-(2′-isopropylbiphenyl-3-yl)-1-({4-[3-(methoxymethyl)-4H-1,2,4-triazol-5-yl]phenyl}amino)-1-oxopropan-2-yl]carbamoyl}cyclohexyl)methyl]carbamate in 2.5 ml of tetrahydrofuran, and the mixture was stirred at RT for 16 h. The solvent was removed on a rotary evaporator, and the residue was stirred in acetonitrile and filtered. The solid formed was washed with acetonitrile and dried under high vacuum. 38 mg (85% of theory) of the title compound were obtained.

¹H NMR (400 MHz, DMSO-d₆): δ=ppm 0.72-0.97 (m, 2H), 1.02-1.37 (m, 8H), 1.39-1.59 (m, 2H), 1.73 (m, 3H), 2.02-2.19 (m, 1H), 2.62 (t, 2H), 2.86-3.02 (m, 2H), 3.07-3.21 (m, 1H), 3.35 (s, 3H), 4.47-4.59 (m, 2H), 4.66-4.79 (m, 1H), 7.03 (dd, 1H), 7.06-7.14 (m, 1H), 7.15-7.25 (m, 2H), 7.28-7.43 (m, 4H), 7.74 (d, 2H), 7.84-8.01 (m, 5H), 8.25 (d, 1H), 10.47 (s, 1H).

LC-MS (Method 1): Rₜ=0.89 min; MS (ESIneg): m/z=607 [M−H−HCl]⁻.

### Example 105

### N-alpha-{[trans-4-(Aminomethyl)cyclohexyl]carbonyl}-3-quinolin-5-yl-N-{4-[5-(methoxymethyl)-4H-1,2,4-triazol-3-yl]phenyl}-L-phenylalaninamide hydrochloride

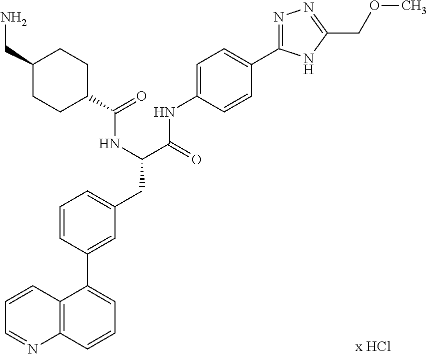

1.00 ml (4.00 mmol) of 4M hydrogen chloride in 1,4-dioxane was added to a solution of 75 mg (0.10 mmol) of tert-butyl [(trans-4-{[(2S)-3-[3-(quinolin-5-yl)phenyl]-1-({4-[3-(methoxymethyl)-4H-1,2,4-triazol-5-yl]phenyl}amino)-1-oxopropan-2-yl]carbamoyl}cyclohexyl)methyl]carbamate in 2.5 ml of tetrahydrofuran, and the mixture was stirred at RT for 4 h. The solvent was removed on a rotary evaporator, and the residue was stirred in acetonitrile and filtered. The solid formed was washed with acetonitrile and dried under high vacuum. 51 mg (72% of theory) of the title compound were obtained.

¹H NMR (400 MHz, DMSO-d₆): δ=ppm 0.73-0.99 (m, 2H), 1.02-1.20 (m, 1H), 1.20-1.35 (m, 1H), 1.40-1.56 (m, 2H), 1.59-1.82 (m, 4H), 2.04-2.21 (m, 1H), 2.57-2.64 (m, 2H), 2.93-3.08 (m, 1H), 3.13-3.27 (m, 1H), 4.53 (s, 3H), 4.77-4.87 (m, 1H), 7.33-7.42 (m, 1H), 7.49 (m, 3H), 7.73 (m, 3H), 7.84-8.03 (m, 6H), 8.06-8.18 (m, 1H), 8.27-8.40 (m, 2H), 8.71 (d, 1H), 9.24 (d, 1H), 10.51 (s, 1H).

LC-MS (Method 1): Rₜ=0.68 min; MS (ESIneg): m/z=616 [M−H−HCl]⁻.

### Example 106

### N-alpha-{[trans-4-(Aminomethyl)cyclohexyl]carbonyl}-N-[4-(3-chloro-4H-1,2,4-triazol-5-yl)phenyl]-3-[5-(piperidin-4-ylsulphamoyl)pyridin-3-yl]-L-phenylalaninamide hydrochloride

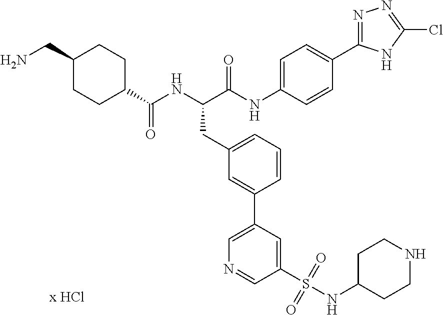

0.081 ml (0.32 mmol) of 4M hydrogen chloride in 1,4-dioxane was added to a suspension of 30 mg (0.03 mmol) of tert-butyl 4-{[(5-{3-[(2S)-2-{[(trans-4-{[(tert-butoxycarbonyl)amino]methyl}cyclohexyl)carbonyl]amino}-3-{[4-(3-chloro-4H-1,2,4-triazol-5-yl)phenyl]amino}-3-oxopropyl]phenyl}pyridin-3-yl)sulphonyl]amino}piperidine-1-carboxylate in 1.5 ml of dioxane, and the mixture was stirred at 40° C. for 4 h and at RT overnight. 5 ml of acetonitrile were added to the reaction mixture and the resulting precipitate was filtered off with suction. The residue was purified chromatographically by HPLC (Method 7). This gave 4.6 mg (17% of theory) of the title compound.

¹H NMR (300 MHz, DMSO-d₆): δ=ppm 0.73-0.95 (m, 2H), 0.98-1.28 (m, 3H), 1.31-1.51 (m, 2H), 1.54-1.81 (m, 8H), 2.03-2.18 (m, 1H), 2.55-2.62 (m, 2H), 2.77-3.01 (m, 4H), 3.07-3.21 (m, 3H), 4.67-4.81 (m, 1H), 7.37-7.44 (m, 2H), 7.58-7.66 (m, 1H), 7.70-7.83 (m, 6H), 7.84-7.92 (m, 2H), 8.26 (d, 1H), 8.32 (br. d, 1H), 8.45 (s, 1H), 8.51-8.66 (m, 1H), 8.68-8.83 (m, 1H), 8.94 (s, 1H), 9.11 (s, 1H), 10.56 (s, 1H).

LC-MS (Method 4): Rₜ=0.65 min; MS (ESIpos): m/z=720.5 [M+H−HCl]⁺

### Example 107

### N-alpha-{[trans-4-(Aminomethyl)cyclohexyl]carbonyl}-3-(4-chloropyridin-3-yl)-N-[4-(5-oxo-4,5-dihydro-1,2,4-oxadiazol-3-yl)phenyl]-L-phenylalaninamide hydrochloride

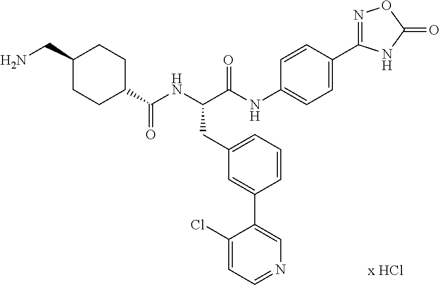

0.04 ml (0.17 mmol) of 4M hydrogen chloride in 1,4-dioxane was added to a suspension of 17 mg (0.025 mmol) of N-alpha-[(trans-4-{[(tert-butoxycarbonyl)amino]methyl}cyclohexyl)carbonyl]-3-(4-chloropyridin-3-yl)-N-[4-(5-oxo-4, 5-dihydro-1,2,4-oxadiazol-3-yl)phenyl]-L-phenylalaninamide in 0.5 ml of 1,4-dioxane, and the mixture was stirred at RT overnight. 5 ml of acetonitrile were then added to the reaction mixture and the resulting precipitate was filtered off with suction and purified chromatographically by HPLC (Method 10). This gave 4 mg (25% of theory) of the title compound.

¹H NMR (300 MHz, DMSO-d₆): δ=ppm 0.75-0.95 (m, 2H), 1.00-1.29 (m, 2H), 1.33-1.52 (m, 2H), 1.58-1.76 (m, 3H), 2.02-2.14 (m, 1H), 2.60 (d, 2H), 2.90 (dd, 1H), 3.11 (dd, 1H), 4.64-4.76 (m, 1H), 7.29-7.32 (m, 1H), 7.35-7.40 (m, 3H), 7.56 (d, 2H), 7.62 (d, 1H), 7.67 (d, 2H), 8.11 (d, 1H), 8.48-8.51 (m, 2H), 10.17 (s, 1H).

LC-MS (Method 4): Rt=0.79 min; MS (ESIpos): m/z=575.2 [M+H−HCl]⁺.

### Example 108

### 4-(5-{4-[(N-{[trans-4-(Aminomethyl)cyclohexyl]carbonyl}-3-{6-[3-(dimethylamino)propoxy]pyridin-3-yl}-L-phenylalanyl)amino]phenyl}-1H-1,2,4-triazol-3-yl)-2,2,3,3,4,4-hexafluorobutanoic acid hydrochloride

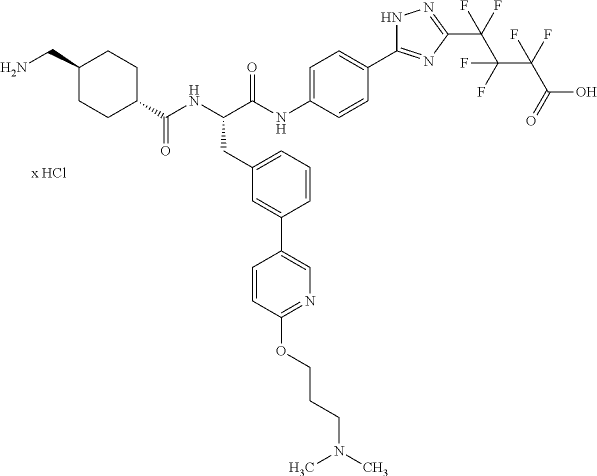 [SMILES: CN(C)CCCOc1ccc(-c2cccc(C[C@H](NC(=O)[C@H]3CC[C@H](CN)CC3)C(=O)Nc3ccc(-c4nc(C(F)(F)C(F)(F)C(F)(F)C(=O)O)n[nH]4)cc3)c2)cn1]

0.11 ml (0.46 mmol) of 4M hydrogen chloride in 1,4-dioxane was added to a solution of 28 mg (0.03 mmol) of 4-(5-{4-[(N-[(trans-4-{[(tert-butoxycarbonyl)amino]methyl}cyclohexyl)carbonyl]-3-{6-[3-(dimethylamino)propoxy]pyridin-3-yl}-L-phenylalanyl)amino]phenyl}-1H-1,2,4-triazol-3-yl)-2,2,3,3,4,4-hexafluorobutanoic acid in 1.5 ml of dioxane, and the mixture was stirred at RT for 3 days. The solid was filtered off with suction, washed with dioxane and acetonitrile, then dried under high vacuum. This gave 22 mg (81% of theory) of the title compound.

¹H NMR (400 MHz, DMSO-d₆): δ=ppm 0.79-0.99 (m, 2H), 1.06-1.33 (m, 2H), 1.39-1.49 (m, 1H), 1.50-1.59 (m, 1H), 1.63-1.85 (m, 3H), 2.09-2.24 (m, 3H), 2.59-2.66 (m, 2H), 2.79 (m, 6H), 2.91-3.01 (m, 1H), 3.10-3.26 (m, 3H), 4.33-4.41 (m, 2H), 4.69-4.81 (m, 1H), 6.90 (d, 1H), 7.27-7.33 (m, 1H), 7.34-7.40 (m, 1H), 7.48 (d, 1H), 7.56-7.63 (m, 1H), 7.80 (m, 5H), 7.93-8.04 (m, 3H), 8.27 (d, 1H), 8.38-8.50 (m, 1H), 10.01-10.31 (m, 1H), 10.47-10.62 (m, 1H), 15.14-15.38 (m, 1H).

LC-MS (Method 1): Rₜ=0.60 min; MS (ESIneg): m/z=817 [M−HCl]⁻.

### Example 109

### 3-(5-{4-[(N-[(trans-4-{[(tert-Butoxycarbonyl)amino]methyl}cyclohexyl)carbonyl]-3-{6-[3-(dimethylamino)propoxy]pyridin-3-yl}-L-phenylalanyl)amino]phenyl}-1H-1,2,4-triazol-3-yl)-2,2,3,3-tetrafluoropropanoic acid hydrochloride

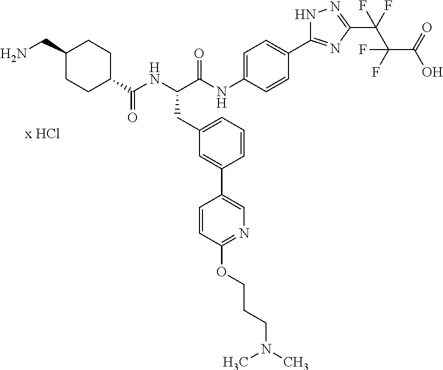 [SMILES: CN(C)CCCOc1ccc(-c2cccc(C[C@H](NC(=O)[C@H]3CC[C@H](CN)CC3)C(=O)Nc3ccc(-c4nc(C(F)(F)C(F)(F)C(=O)O)n[nH]4)cc3)c2)cn1]

0.22 ml (0.86 mmol) of 4M hydrogen chloride in 1,4-dioxane was added to a solution of 50 mg (0.06 mmol) of 3-(5-{4-[(N-[(trans-4-{[(tert-butoxycarbonyl)amino]methyl}cyclohexyl)carbonyl]-3-{6-[3-(dimethylamino)propoxy]pyridin-3-yl}-L-phenylalanyl)amino]phenyl}-1H-1,2,4-triazol-3-yl)-2,2,3,3-tetrafluoropropanoic acid in 2 ml of dioxane, and the mixture was stirred at RT for 4 days. The solid was filtered off with suction, washed with dioxane and acetonitrile, then dried under high vacuum. This gave 37 mg (73% of theory) of the title compound.

¹H NMR (400 MHz, DMSO-d₆): δ=ppm 0.80-0.99 (m, 2H), 1.06-1.33 (m, 2H), 1.39-1.58 (m, 2H), 1.63-1.83 (m, 3H), 2.11-2.22 (m, 3H), 2.58-2.67 (m, 2H), 2.79 (d, 6H), 2.90-3.02 (m, 1H), 3.12-3.25 (m, 3H), 4.36 (m, 1H), 4.71-4.81 (m, 1H), 6.91 (d, 1H), 7.27-7.33 (m, 1H), 7.34-7.41 (m, 1H), 7.49 (d, 1H), 7.58-7.64 (m, 1H), 7.80 (m, 5H), 7.99 (m, 3H), 8.28 (d, 1H), 8.40-8.48 (m, 1H), 10.05-10.34 (m, 1H), 10.46-10.63 (m, 1H), 14.99-15.40 (m, 1H).

LC-MS (Method 1): Rₜ=0.55 min; MS (ESIneg): m/z=767 [M−HCl].

### Example 110

### trans-4-(Aminomethyl)-N-{(2S)-3-[2′-methyl-5′-(morpholin-4-ylsulphonyl)biphenyl-3-yl]-1-oxo-1-[(3-oxo-2,3-dihydro-1H-indazol-6-yl)amino]propan-2-yl}cyclohexanecarboxamide hydrochloride

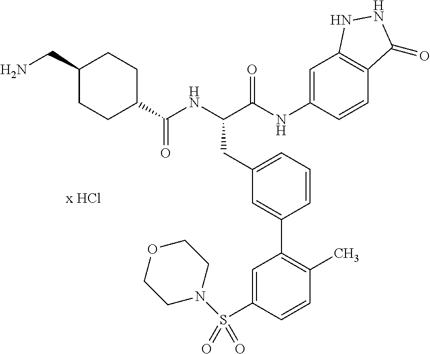

4M hydrogen chloride in 1,4-dioxane is added to a suspension of tert-butyl {[trans-4-({(2S)-3-[2′-methyl-5′-(morpholin-4-ylsulphonyl)biphenyl-3-yl]-1-oxo-1-[(3-oxo-2,3-dihydro-1H-indazol-6-yl)amino]propan-2-yl}carbamoyl)cyclohexyl]methyl}carbamate in 1,4-dioxane, and the mixture is stirred at RT for 3 to 4 days. The reaction mixture is worked up by methods known to those skilled in the art and the residue is separated by means of preparative HPLC. This gives the title compound.

### Example 111

### trans-4-(Aminomethyl)-N-{(2S)-3-[2′-methyl-5′-(pyrrolidin-1-ylsulphonyl)biphenyl-3-yl]-1-oxo-1-[(3-oxo-2,3-dihydro-1H-indazol-6-yl)amino]propan-2-yl}cyclohexanecarboxamide hydrochloride

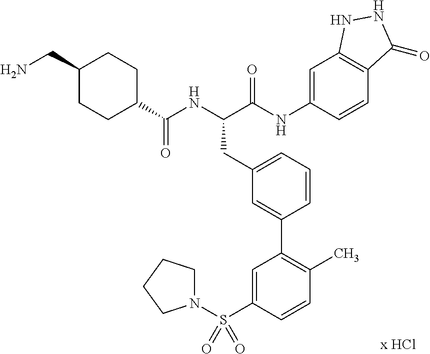

4M hydrogen chloride in 1,4-dioxane is added to a suspension of tert-butyl {[trans-4-({(2S)-3-[2′-methyl-5′-(pyrrolidin-1-ylsulphonyl)biphenyl-3-yl]-1-oxo-1-[(3-oxo-2,3-dihydro-1H-indazol-6-yl)amino]propan-2-yl}carbamoyl)cyclohexyl]methyl}carbamate in 1,4-dioxane, and the mixture is stirred at RT for 3 to 4 days. The reaction mixture is worked up by methods known to those skilled in the art and the residue is separated by means of preparative HPLC. This gives the title compound.

### Example 112

### trans-4-(Aminomethyl)-N-{(2S)-3-[5′-(benzylsulphamoyl)-2′-methylbiphenyl-3-yl]-1-oxo-1-[(3-oxo-2,3-dihydro-1H-indazol-6-yl)amino]propan-2-yl}cyclohexanecarboxamide hydrochloride

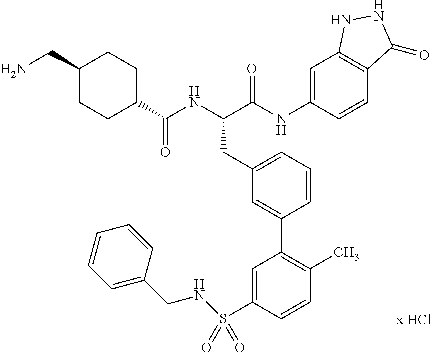

4M hydrogen chloride in 1,4-dioxane is added to a suspension of tert-butyl {[trans-4-({(25)-3-[5′-(benzylsulphamoyl)-2′-methylbiphenyl-3-yl]-1-oxo-1-[(3-oxo-2,3-dihydro-1H-indazol-6-yl)amino]propan-2-yl}carbamoyl)cyclohexyl]methyl}carbamate in 1,4-dioxane, and the mixture is stirred at RT for 3 to 4 days. The reaction mixture is worked up by methods known to those skilled in the art and the residue is separated by means of preparative HPLC. This gives the title compound.

### Example 113

### trans-4-(Aminomethyl)-N-{(2S)-3-[5′-(dimethylsulphamoyl)-2′-methylbiphenyl-3-yl]-1-oxo-1-[(3-oxo-2,3-dihydro-1H-indazol-6-yl)amino]propan-2-yl}cyclohexanecarboxamide hydrochloride

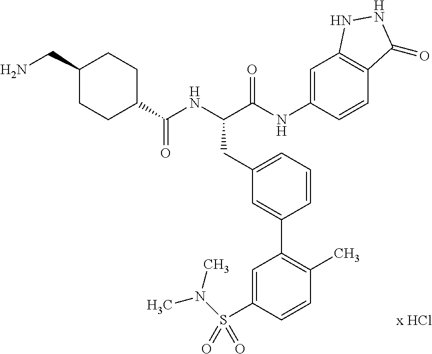

4M hydrogen chloride in 1,4-dioxane is added to a suspension of tert-butyl {[trans-4-({(2S)-3-[5′-(dimethylsulphamoyl)-2′-methylbiphenyl-3-yl]-1-oxo-1-[(3-oxo-2,3-dihydro-1H-indazol-6-yl)amino]propan-2-yl}carbamoyl)cyclohexyl]methyl}carbamate in 1,4-dioxane, and the mixture is stirred at RT for 3 to 4 days. The reaction mixture is worked up by methods known to those skilled in the art and the residue is separated by means of preparative HPLC. This gives the title compound.

### Example 114

### N-alpha-{[trans-4-(Aminomethyl)cyclohexyl]carbonyl}-N-(3-oxo-2,3-dihydro-1H-indazol-6-yl)-3-[2-(piperazin-1-yl)pyrimidin-5-yl]-L-phenylalaninamide hydrochloride

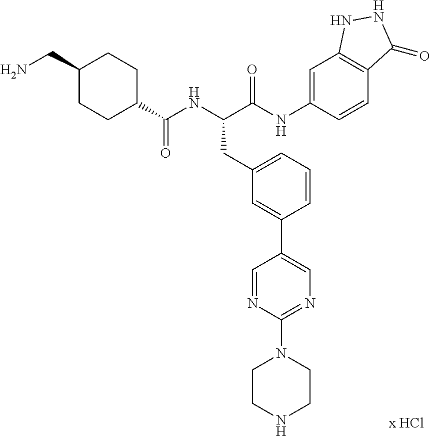

4M hydrogen chloride in 1,4-dioxane is added to a suspension of tert-butyl 4-[5-(3-{(25)-2-{[(trans-4-{[(tert butoxycarbonyl)amino]methyl}cyclohexyl)carbonyl]amino}-3-oxo-3-[(3-oxo-2,3-dihydro-1H-indazol-6-yl)amino]propyl}phenyl)pyrimidin-2-yl]piperazine-1-carboxylate in 1,4-dioxane, and the mixture is stirred at RT for 3 to 4 days. The reaction mixture is worked up by methods known to those skilled in the art and the residue is separated by means of preparative HPLC. This gives the title compound.

### Example 115

### N-alpha-{[trans-4-(Aminomethyl)cyclohexyl]carbonyl}-3-[2-(dimethylamino)pyrimidin-5-yl]-N-(3-oxo-2,3-dihydro-1H-indazol-6-yl)-L-phenylalaninamide hydrochloride

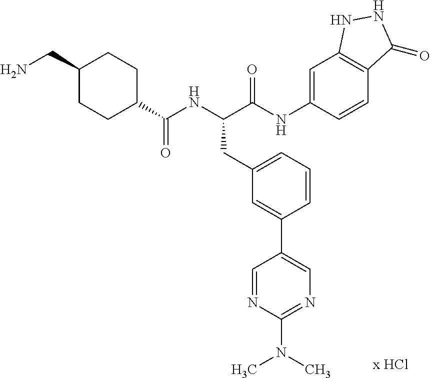

4M hydrogen chloride in 1,4-dioxane is added to a suspension of N-alpha-[(trans-4-{[(tert-butoxycarbonyl)amino]methyl}cyclohexyl)carbonyl]-3-[2-(dimethylamino)pyrimidin-5-yl]-N-(3-oxo-2,3-dihydro-1H-indazol-6-yl)-L-phenylalaninamide in 1,4-dioxane, and the mixture is stirred at RT for 3 to 4 days. The reaction mixture is worked up by methods known to those skilled in the art and the residue is separated by means of preparative HPLC. This gives the title compound.

### Example 116

### N-alpha-{[trans-4-(Aminomethyl)cyclohexyl]carbonyl}-3-(3-methylpyridin-4-yl)-N-(3-oxo-2,3-dihydro-1H-indazol-6-yl)-L-phenylalaninamide hydrochloride

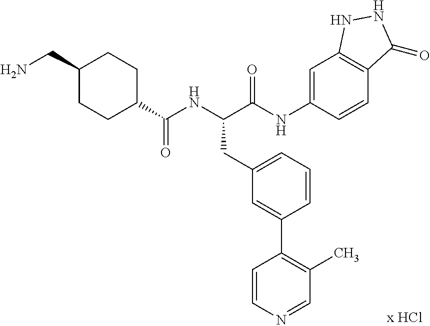

4M hydrogen chloride in 1,4-dioxane is added to a suspension of N-alpha-[(trans-4-{[(tert-butoxycarbonyl)amino]methyl}cyclohexyl)carbonyl]-3-(3-methylpyridin-4-yl)-N-(3-oxo-2,3-dihydro-1H-indazol-6-yl)-L-phenylalaninamide in 1,4-dioxane, and the mixture is stirred at RT for 3 to 4 days. The reaction mixture is worked up by methods known to those skilled in the art and the residue is separated by means of preparative HPLC. This gives the title compound.

### Example 117

### N-alpha-{[trans-4-(Aminomethyl)cyclohexyl]carbonyl}-3-[2-(4-methylpiperazin-1-yl)pyridin-4-yl]-N-(3-oxo-2,3-dihydro-1H-indazol-6-yl)-L-phenylalaninamide hydrochloride

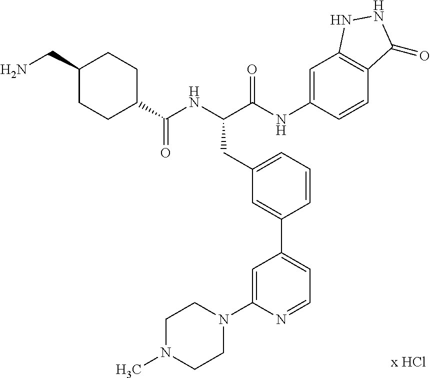

A suspension of N-alpha-[(trans-4-{[(tert-butoxycarbonyl)amino]methyl}cyclohexyl)carbonyl]-3-[2-(4-methylpiperazin-1-yl)pyridin-4-yl]-N-(3-oxo-2,3-dihydro-1H-indazol-6-yl)-L-phenylalaninamide in 1,4-dioxane is admixed with 4M hydrogen chloride in 1,4-dioxane and stirred at RT for 3 to 4 days. The reaction mixture is worked up by methods known to those skilled in the art and the residue is separated by means of preparative HPLC. This gives the title compound.

### Example 118

### N-alpha-{[trans-4-(Aminomethyl)cyclohexyl]carbonyl}-3-{6-[3-(dimethylamino)propoxy]pyridin-3-yl}-N-(3-oxo-2,3-dihydro-1H-indazol-6-yl)-L-phenylalaninamide hydrochloride

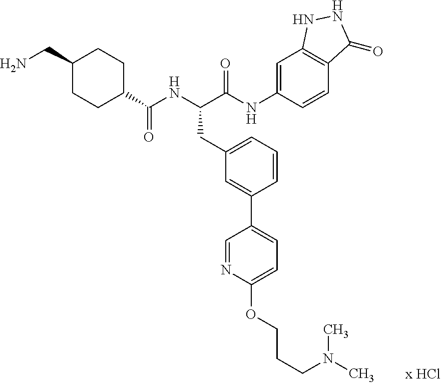

4M hydrogen chloride in 1,4-dioxane is added to a suspension of N-alpha-[(trans-4-{[(tert-butoxycarbonyl)amino]methyl}cyclohexyl)carbonyl]-3-{6-[3-(dimethylamino)propoxy]pyridin-3-yl}-N-(3-oxo-2,3-dihydro-1H-indazol-6-yl)-L-phenylalaninamide in 1,4-dioxane, and the mixture is stirred at RT for 3 to 4 days. The reaction mixture is worked up by methods known to those skilled in the art and the residue is separated by means of preparative HPLC. This gives the title compound.

### B) ASSESSMENT OF PHYSIOLOGICAL EFFICACY

The suitability of the compounds according to the invention for treating thromboembolic or hyperfibrinolytic disorders can be demonstrated in the following assay systems:

### a) Test Descriptions (In Vitro)

### a.1) Measurement of FXIa Inhibition

The factor XIa inhibition of the substances according to the invention is determined using a biochemical test system which utilizes the reaction of a peptidic factor XIa substrate to determine the enzymatic activity of human factor XIa. Here, factor XIa cleaves from the peptic factor XIa substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions is placed into the wells of white microtitre plates from Greiner (384 wells). Subsequently, the following are added successively: 20 μl of assay buffer (50 mmol/l Tris buffer pH 7.4; 100 mmol/l sodium chloride; 5 mmol/l calcium chloride; 0.1% bovine serum albumin) and 20 μl of factor XIa from Kordia (0.45 nM in assay buffer). After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulphoxide instead of test substance in dimethyl sulphoxide), and IC₅₀ values are calculated from the concentration/activity relationships. Activity data from this test are listed in Table A below:

TABLE A

| Example No. | IC₅₀ [nM] | Example No. | IC₅₀ [nM] |
|---|---|---|---|
| 1 | 0.4 | 2 | 1.1 |
| 3 | 0.9 | 4 | 1.5 |
| 5 | 3.4 | 6 | 1.9 |
| 7 | 1.7 | 8 | 3.8 |
| 9 | 9.6 | 10 | 13 |
| 11 | 7.9 | 12 | 3.2 |
| 13 | 9.2 | 14 | 4.8 |
| 15 | 7.7 | 16 | 5.5 |
| 17 | 4.1 | 18 | 5.7 |
| 19 | 6.0 | 20 | 1.8 |
| 21 | 2.3 | 22 | 2.4 |
| 23 | 3.6 | 24 | 4.3 |
| 25 | 4.4 | 26 | 4.5 |
| 27 | 4.7 | 28 | 5.2 |
| 29 | 5.5 | 30 | 5.8 |
| 31 | 7.0 | 32 | 6.8 |
| 33 | 7.6 | 34 | 8.6 |
| 35 | 10 | 36 | 2.2 |
| 37 | 5.6 | 38 | 4.8 |
| 39 | 8.9 | 40 | 8.3 |
| 41 | 8.4 | 42 | 11 |
| 43 | 5.2 | 44 | 13 |
| 45 | 5.8 | 46 | 7.9 |
| 47 | 5.2 | 48 | 6.9 |
| 49 | 21 | 50 | 55 |
| 51 | 36 | 52 | 1.8 |
| 53 | 17 | 54 | 1.9 |
| 55 | 4.3 | 56 | 15 |
| 57 | 17 | 58 | 17 |
| 59 | 7.2 | 60 | 1.4 |
| 61 | 12 | 62 | 6.2 |
| 63 | 52 | 64 | 5.2 |
| 65 | 12 | 66 | 3.3 |
| 67 | 37 | 68 | 7.3 |
| 69 | 4.9 | 70 | 2.9 |
| 71 | 3.1 | 72 | 5.3 |
| 73 | 25 | 74 | 2.1 |
| 75 | 35 | 76 | 59 |
| 77 | 7.3 | 78 | 2.3 |
| 79 | 2.3 | 80 | 2.4 |
| 81 | 2.5 | 82 | 3.4 |
| 83 | 1.3 | 84 | 3.1 |
| 85 | 2.4 | 86 | 2.5 |
| 87 | 3.5 | 88 | 29 |
| 89 | 21 | 90 | 5.8 |
| 91 | 3.5 | 92 | 3.6 |
| 93 | 5.3 | 94 | 5.4 |
| 95 | 5.4 | 96 | 3.3 |
| 97 | 2.1 | 98 | 5.4 |
| 99 | 8.9 | 100 | 59 |
| 101 | 14 | 102 | 2.2 |
| 103 | 3.3 | 104 | 8.6 |
| 105 | 13 | 106 | 5.7 |
| 107 | 6.2 | 108 | 5.5 |
| 109 | 6.0 | | |

### a.2) Determination of the Selectivity

To demonstrate the selectivity of the substances with respect to FXIa inhibition, the test substances are examined for their inhibition of other human serine proteases, such as factor Xa, trypsin and plasmin. To determine the enzymatic activity of factor Xa (1.3 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of sodium chloride, 0.1% BSA [bovine serum albumin], 5 mmol/l of calcium chloride, pH 7.4) and incubated for 15 min with test substance in various concentrations in dimethyl sulphoxide and also with dimethyl sulphoxide without test substance. The enzymatic reaction is then started by addition of the appropriate substrates (5 μmol/l of Boc-Ile-Glu-Gly-Arg-AMC from Bachem for factor Xa and trypsin, 50 μmol/1 of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulphoxide instead of test substance in dimethyl sulphoxide) and IC₅₀ values are calculated from the concentration/activity relationships.

### a.3) Thrombin Generation Assay (Thrombogram)

The effect of the test substances on the thrombogram (thrombin generation assay according to Hemker) is determined in vitro in human plasma (Octaplas® from Octapharma).

In the thrombin generation assay according to Hemker, the activity of thrombin in coagulating plasma is determined by measuring the fluorescent cleavage products of the substrate I-1140 (Z-Gly-Gly-Arg-AMC, Bachem). The reactions are carried out in the presence of varying concentrations of test substance or the corresponding solvent. To start the reaction, reagents from Thrombinoscope (30 pM or 0.1 pM recombinant tissue factor, 24 μM phospholipids in HEPES) are used. In addition, a thrombin calibrator from Thrombinoscope is used whose amidolytic activity is required for calculating the thrombin activity in a sample containing an unknown amount of thrombin. The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM Hepes, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser.

Using the Thrombinoscope software, the thrombogram is calculated and represented graphically. The following parameters are calculated: lag time, time to peak, peak, ETP (endogenous thrombin potential) and start tail.

### a.4) Determination of Anticoagulatory Activity

The anticoagulatory activity of the test substances is determined in vitro in human and animal plasma (for example mouse, rat, rabbit, pig and dog plasma). To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

The activated partial thromboplastin time (aPTT) is determined in the presence of varying concentrations of test substance or the corresponding solvent using a commercial test kit (C.K. Prest from Diagnostica Stago). The test compounds are incubated with the plasma and the PTT reagent (cephalin, kaolin) at 37° C. for 3 minutes. Coagulation is then started by addition of a 25 mM aqueous calcium chloride solution, and the time when coagulation occurs is determined. The concentration of test substance which brings about a 1.5-fold extension of the aPTT is determined. Activity data from this test are listed in Table B below:

TABLE B

| | aPTT | | aPTT |
|---|---|---|---|
| Example No. | [μmol/l] | Example No. | [μmol/l] |
| 1 | 0.1 | 2 | 0.08 |
| 3 | 0.09 | 4 | 0.21 |
| 5 | 0.23 | 6 | 0.27 |
| 7 | 0.33 | 8 | 0.41 |
| 9 | 0.14 | 10 | 0.23 |
| 11 | 0.3 | 12 | 0.3 |
| 13 | 0.47 | 14 | 0.15 |
| 15 | 0.19 | 16 | 0.25 |
| 17 | 0.19 | 18 | 0.4 |
| 19 | 0.14 | 20 | 0.24 |
| 21 | 0.19 | 22 | 0.34 |
| 24 | 0.15 | 27 | 0.3 |
| 29 | 0.37 | 30 | 0.34 |
| 31 | 0.36 | 32 | 0.18 |
| 33 | 0.08 | 34 | 0.26 |
| 35 | 0.37 | 36 | 0.12 |
| 37 | 0.19 | 38 | 0.2 |
| 39 | 0.22 | 40 | 0.19 |
| 41 | 0.32 | 42 | 0.35 |
| 43 | 0.26 | 44 | 0.28 |
| 45 | 0.3 | 46 | 0.32 |
| 47 | 0.34 | 48 | 0.44 |
| 49 | 0.47 | 51 | 0.33 |
| 52 | 0.06 | 54 | 0.28 |
| 55 | 0.14 | 56 | 0.28 |
| 57 | 0.3 | 58 | 0.3 |
| 59 | 0.19 | 60 | 0.21 |
| 61 | 0.37 | 62 | 0.48 |
| 63 | | 64 | 0.12 |
| 65 | 0.13 | 66 | 0.12 |
| 67 | 0.15 | 68 | 0.18 |
| 70 | 0.36 | 78 | 0.19 |
| 79 | 0.14 | 80 | 0.25 |
| 81 | 0.3 | 82 | 0.14 |
| 83 | 0.17 | 84 | 0.28 |
| 86 | 0.24 | 87 | 0.49 |
| 89 | 0.46 | 90 | 0.08 |
| 91 | 0.06 | 92 | 0.37 |
| 93 | 0.48 | 94 | 0.23 |
| 95 | 0.24 | 96 | 0.19 |
| 97 | 0.12 | 98 | 0.2 |
| 99 | 0.25 | 100 | 0.34 |
| 101 | 0.43 | 102 | 0.43 |
| 103 | 0.1 | 105 | 0.14 |
| 106 | 0.13 | 107 | 0.7 |
| 108 | 0.25 | 109 | 0.16 |

### a.5) Determination of Fibrinolvtic Activity

Antifibrinolytic activity in vitro is assessed in human, platelet-free plasma. Tissue factor (TF) (1 pM) and tissue plasminogen activator (tPA) (40 nM) are pipetted into plasma together with 12.5 mM aqueous calcium chloride solution and substance. On occurrence of clotting, the subsequent clot lysis is determined photometrically over a period of 30 minutes.

### b) Determination of Antithrombotic Activity (In Vivo)

b.1) Arterial Thrombosis Model (Iron(II) Chloride-Induced Thrombosis) in Combination with Ear Bleeding Time in Rabbits

The antithrombotic activity of the FXIa inhibitors is tested in an arterial thrombosis model. Thrombus formation is triggered here by causing chemical injury to a region in the carotid artery in rabbits. Simultaneously, the ear bleeding time is determined.

Male rabbits (Crl:KBL (NZW)BR, Charles River) receiving a normal diet and having a body weight of 2.2-2.5 kg are anaesthetized by intramuscular administration of xylazine and ketamine (Rompun, Bayer, 5 mg/kg and Ketavet, Pharmacia & Upjohn GmbH, 40 mg/kg body weight). Anaesthesia is furthermore maintained by intravenous administration of the same preparations (bolus: continuous infusion) via the right auricular vein.

The right carotid artery is exposed and the vessel injury is then caused by wrapping a piece of filter paper (10 mm×10 mm) on a Parafilm® strip (25 mm×12 mm) around the carotid artery without disturbing the blood flow. The filter paper contains 100 μL of a 13% strength solution of iron(II) chloride (Sigma) in water. After 5 min, the filter paper is removed and the vessel is rinsed twice with aqueous 0.9% strength sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h, respectively, before the injury.

Ear bleeding time is determined 2 min after injury to the carotid artery. To this end, the left ear is shaved and a defined 3-mm-long incision (blade Art. Number 10-150-10, Martin, Tuttlingen, Germany) is made parallel to the longitudinal axis of the ear. Care is taken here not to damage any visible vessels. Any blood that extravasates is taken up in 15 second intervals using accurately weighed filter paper pieces, without touching the wound directly. Bleeding time is calculated as the time from making the incision to the point in time where no more blood can be detected on the filter paper. The volume of the extravasated blood is calculated after weighing of the filter paper pieces.

### c) Determination of Fibrinolvtic Activity (In Vivo)

### c.1) Hyper-Fibrinolytic Rats

The determination of antifibrinolytic activity in vivo is conducted in hyperfibrinolytic rats. After anaesthetization and catheterization of the animals, hyperfibrinolysis is triggered by infusion of tissue plasminogen activator (tPA) (8 mg/kg/h). 10 minutes after commencement of tPA infusion, the substances are administered as an i.v. bolus. After a further 15 minutes, tPA infusion is ended and a transsection of the tail is conducted. Subaqual bleeding (in physiological saline at 37° C.) is observed over 30 minutes and the bleed time is determined.

### C) WORKING EXAMPLES OF PHARMACEUTICAL PREPARATIONS

The substances according to the invention can, for example, be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP) and 2 mg of magnesium stearate.

Tablet weight 212 mg. Diameter 8 mm, radius of curvature 12 mm.

### Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tabletting press (see above for format of the tablet).

### Oral Suspension:

### Composition:

1000 mg of the compound of Example 1, 1000 mg of ethanol (96%), 400 mg of Rhodigel and 99 g of water.

10 ml of oral suspension correspond to a single dose of 100 mg of the compound of the invention.

### Production:

The Rhodigel is suspended in ethanol, and the compound of Example 1 is added to the suspension. The water is added while stirring. The mixture is stirred for about 6 h until swelling of the Rhodigel is complete.

### Solution for Oral Administration:

### Composition:

500 mg of the compound of the invention, 2.5 g of polysorbate and 97 g of polyethylene glycol 400. 20 g of oral solution correspond to a single dose of 100 mg of the compound of the invention.

### Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or polyethylene glycol 400/water 30% m/m). The solution is subjected to sterile filtration and dispensed into sterile and pyrogen-free injection vessels.

1. A compound of the formula 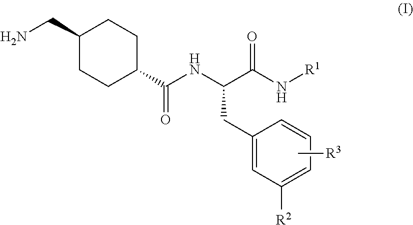
in which
R¹ represents a group of the formula 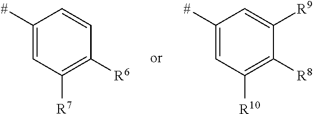
where # is the point of attachment to the nitrogen atom,
R⁶ represents 5-membered heteroaryl,
where heteroaryl may be substituted by a substituent selected from the group consisting of oxo, chlorine, cyano, hydroxy and C₁-C₃-alkyl,
in which alkyl may be substituted by 1 to 3 substituents independently of one another selected from the group consisting of hydroxy, amino, hydroxycarbonyl and methoxy, or
in which alkyl may be substituted by 1 to 7 fluorine substituents, or
in which alkyl is substituted by a substituent selected from the group consisting of hydroxy, amino, hydroxycarbonyl and methoxy, and in which alkyl is additionally substituted by 1 to 6 fluorine substituents,
R⁷ represents hydrogen, fluorine or chlorine,
R⁸ and R⁹ together with the carbon atoms to which they are attached form a 5-membered heterocycle,
where the heterocycle may be substituted by 1 to 2 substituents independently of one another selected from the group consisting of oxo, chlorine, cyano, hydroxy, C₁-C₃-alkyl, pyrazolyl and pyridyl,
in which alkyl may be substituted by 1 to 3 substituents independently of one another selected from the group consisting of hydroxy, amino, hydroxycarbonyl and methoxy, or
in which alkyl may be substituted by 1 to 7 fluorine substituents, or
in which alkyl is substituted by a substituent selected from the group consisting of hydroxy, amino, hydroxycarbonyl and methoxy, and in which alkyl is additionally substituted by 1 to 6 fluorine substituents,
R¹⁰ represents hydrogen, fluorine or chlorine,
R² represents a group of the formula 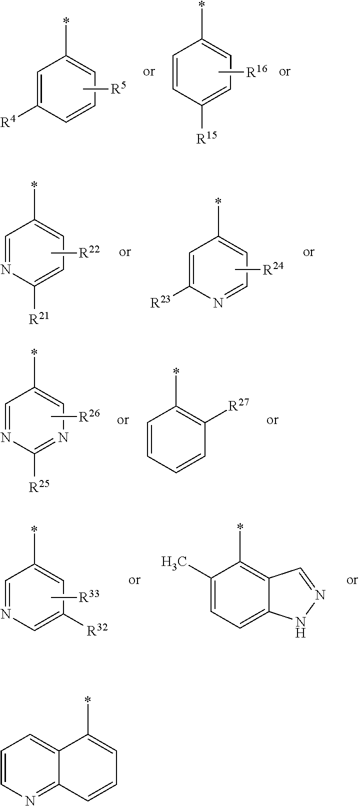
where * is the point of attachment to the phenyl ring,
R⁴ represents amino, C₁-C₄-alkylcarbonylamino, —S(O)₂NR¹¹R¹² or —C(O)NR¹³R¹⁴,
where
R¹¹ represents hydrogen, C₁-C₃-alkyl, C₃-C₆-cycloalkyl, benzyl or 4- to 8-membered heterocyclyl which is attached via a carbon atom,
R¹² represents hydrogen or C₁-C₃-alkyl, or
R¹¹ and R¹² together with the nitrogen atom to which they are attached form a 4- to 7-membered heterocycle,
R¹³ represents hydrogen, C₁-C₃-alkyl, C₁-C₃-alkoxy, C₃-C₆-cycloalkyl, benzyl or 4- to 8-membered heterocyclyl which is attached via a carbon atom,
in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
in which n is a number from 1 to 6,
in which m is a number from 1 to 6, and
in which cycloalkyl may be substituted by 1 to 2 substituents independently of one another selected from the group consisting of oxo, fluorine, hydroxy, amino, C₁-C₄-alkyl and C₁-C₃-alkylamino,
in which alkyl and alkylamino for their part may be substituted by 1 to 5 fluorine substituents, and
in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
in which alkyl and alkylamino for their part may be substituted by 1 to 5 fluorine substituents, and
in which heterocyclyl may additionally be substituted by 1 to 4 substituents independently of one another selected from the group consisting of fluorine and methyl,
R¹⁴ represents hydrogen or C₁-C₃-alkyl,
R¹³ and R¹⁴ together with the nitrogen atom to which they are attached form a 4- to 7-membered heterocycle,
in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
in which alkyl and alkylamino for their part may be substituted by 1 to 5 fluorine substituents, and
in which heterocyclyl may additionally be substituted by 1 to 4 substituents independently of one another selected from the group consisting of fluorine and methyl,
R⁵ represents hydrogen, fluorine, chlorine, C₁-C₄-alkyl, methoxy or trifluoromethyl,
R¹⁵ represents amino, —S(O)₂NR¹⁷R¹⁸ or —C(O)NR¹⁹R²⁰,
where
R¹⁷ represents hydrogen, C₁-C₃-alkyl, C₃-C₆-cycloalkyl, benzyl or 4- to 8-membered heterocyclyl which is attached via a carbon atom,
R¹⁸ represents hydrogen or C₁-C₃-alkyl, or
R¹⁷ and R¹⁸ together with the nitrogen atom to which they are attached form a 4- to 7-membered heterocycle,
R¹⁹ represents hydrogen, C₁-C₃-alkyl, C₁-C₃-alkoxy, C₃-C₆-cycloalkyl, benzyl or 4- to 8-membered heterocyclyl which is attached via a carbon atom,
in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
in which n is a number from 1 to 6,
in which m is a number from 1 to 6, and
in which cycloalkyl may be substituted by 1 to 2 substituents independently of one another selected from the group consisting of oxo, fluorine, hydroxy, amino, C₁-C₄-alkyl and C₁-C₃-alkylamino,
in which alkyl and alkylamino for their part may be substituted by 1 to 5 fluorine substituents, and
in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
in which alkyl and alkylamino for their part may be substituted by 1 to 5 fluorine substituents, and
in which heterocyclyl may additionally be substituted by 1 to 4 substituents independently of one another selected from the group consisting of fluorine and methyl,
R²⁰ represents hydrogen or C₁-C₃-alkyl,
R¹⁹ and R²⁰ together with the nitrogen atom to which they are attached form a 4- to 7-membered heterocycle,
in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
where alkyl and alkylamino for their part may be substituted by 1 to 5 substituents independently of one another selected from the group consisting of hydroxy and fluorine, and
in which heterocyclyl may additionally be substituted by 1 to 4 substituents independently of one another selected from the group consisting of fluorine and methyl,
R¹⁶ represents hydrogen, fluorine, chlorine, C₁-C₄-alkyl, methoxy or trifluoromethyl,
R²¹ represents hydrogen, hydroxy, amino, C₁-C₄-alkoxy, C₁-C₃-alkylamino or 5- or 6-membered heterocyclyl which is attached via a nitrogen atom,
where alkoxy may be substituted by 1 to 2 substituents independently of one another selected from the group consisting of amino and C₁-C₃-alkylamino, and
where heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R²² represents hydrogen, fluorine, chlorine, amino, C₁-C₄-alkyl, methoxy or trifluoromethyl,
R²³ represents hydrogen, hydroxy, amino, C₁-C₄-alkoxy, C₁-C₃-alkylamino or 5- or 6-membered heterocyclyl which is attached via a nitrogen atom,
where alkoxy may be substituted by 1 to 2 substituents independently of one another selected from the group consisting of amino and C₁-C₃-alkylamino, and
where heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R²⁴ represents hydrogen, fluorine, chlorine, C₁-C₄-alkyl, methoxy or trifluoromethyl,
R²⁵ represents hydrogen, hydroxy, amino, C₁-C₄-alkoxy, C₁-C₃-alkylamino, C₁-C₄-alkylcarbonylamino, C₃-C₆-cycloalkylamino or 5- or 6-membered heterocyclyl which is attached via a nitrogen atom,
where alkoxy may be substituted by 1 to 2 substituents independently of one another selected from the group consisting of amino and C₁-C₃-alkylamino, and
where heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R²⁶ represents hydrogen, C₁-C₄-alkyl, methoxy, trifluoromethyl or benzyloxy,
R²⁷ represents C₁-C₄-alkyl, trifluoromethyl, —S(O)₂NR²⁸R²⁹ or —C(O)NR³⁰R³¹,
where
R²⁸ represents hydrogen, C₁-C₃-alkyl, C₃-C₆-cycloalkyl, benzyl or 4- to 8-membered heterocyclyl which is attached via a carbon atom,
R²⁹ represents hydrogen or C₁-C₃-alkyl, or
R²⁸ and R²⁹ together with the nitrogen atom to which they are attached form a 4- to 7-membered heterocycle,
R³⁰ represents hydrogen, C₁-C₃-alkyl, C₁-C₃-alkoxy, C₃-C₆-cycloalkyl, benzyl or 4- to 8-membered heterocyclyl which is attached via a carbon atom,
in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
in which n is a number from 1 to 6,
in which m is a number from 1 to 6, and
in which cycloalkyl may be substituted by 1 to 2 substituents independently of one another selected from the group consisting of oxo, fluorine, hydroxy, amino, C₁-C₄-alkyl and C₁-C₃-alkylamino,
in which alkyl and alkylamino for their part may be substituted by 1 to 5 fluorine substituents, and
in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
in which alkyl and alkylamino for their part may be substituted by 1 to 5 fluorine substituents, and
in which heterocyclyl may additionally be substituted by 1 to 4 substituents independently of one another selected from the group consisting of fluorine and methyl,
R³¹ represents hydrogen or C₁-C₃-alkyl,
R³⁰ and R³¹ together with the nitrogen atom to which they are attached form a 4- to 7-membered heterocycle,
in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
in which alkyl and alkylamino for their part may be substituted by 1 to 5 fluorine substituents, and
in which heterocyclyl may additionally be substituted by 1 to 4 substituents independently of one another selected from the group consisting of fluorine and methyl,
R³² represents —S(O)₂NR³⁴R³⁵ or —C(O)NR³⁶R³⁷,
where
R³⁴ represents hydrogen, C₁-C₃-alkyl, C₃-C₆-cycloalkyl, benzyl or 4- to 8-membered heterocyclyl which is attached via a carbon atom,
R³⁵ represents hydrogen or C₁-C₃-alkyl, or
R³⁴ and R³⁵ together with the nitrogen atom to which they are attached form a 4- to 7-membered heterocycle,
R³⁶ represents hydrogen, C₁-C₃-alkyl, C₁-C₃-alkoxy, C₃-C₆-cycloalkyl, benzyl or 4- to 8-membered heterocyclyl which is attached via a carbon atom,
in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
in which n is a number from 1 to 6,
in which m is a number from 1 to 6, and
in which cycloalkyl may be substituted by 1 to 2 sustituents independently of one another selected from the group consisting of oxo, fluorine, hydroxy, amino, C₁-C₄-alkyl and C₁-C₃-alkylamino,
in which alkyl and alkylamino for their part may be substituted by 1 to 5 fluorine substituents, and
in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
in which alkyl and alkylamino for their part may be substituted by 1 to 5 fluorine substituents, and
in which heterocyclyl may additionally be substituted by 1 to 4 substituents independently of one another selected from the group consisting of fluorine and methyl,
R³⁷ represents hydrogen or C₁-C₃-alkyl,
R³⁶ and R³⁷ together with the nitrogen atom to which they are attached form a 4- to 7-membered heterocycle,
in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkylaminocarbonyl,
in which alkyl and alkylamino for their part may be substituted by 1 to 5 fluorine substituents, and
in which heterocyclyl may additionally be substituted by 1 to 4 substituents independently of one another selected from the group consisting of fluorine and methyl,
R³³ represents hydrogen, C₁-C₄-alkyl, methoxy or trifluoromethyl,
R³ represents hydrogen, fluorine, chlorine, methyl or methoxy,
or one of the salts thereof, solvates thereof or solvates of the salts thereof.

2. The compound according to claim 1, characterized in that
R¹ represents a group of the formula 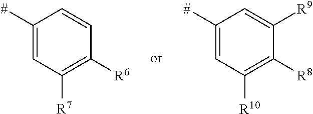
where # is the point of attachment to the nitrogen atom,
R⁶ represents 5-membered heteroaryl,
where heteroaryl may be substituted by a substituent selected from the group consisting of C₁-C₃-alkyl,
in which alkyl is substituted by a hydroxycarbonyl substituent and in which alkyl is additionally substituted by 1 to 6 fluorine substituents,
R⁷ represents hydrogen or fluorine,
R⁸ and R⁹ together with the carbon atoms to which they are attached form a 5-membered heterocycle,
where the heterocycle may be substituted by a substituent independently selected from the group consisting of oxo, methyl, ethyl and n-propyl,
where ethyl may be substituted by 4 or 5 fluorine substituents,
R¹⁰ represents hydrogen,
R² represents a group of the formula 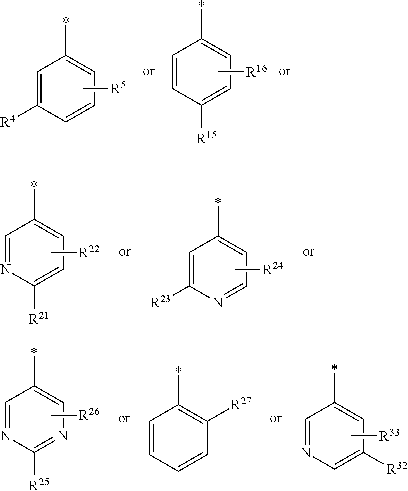
where * is the point of attachment to the phenyl ring,
R⁴ represents —S(O)₂NR¹¹R¹² or —C(O)NR¹³R¹⁴,
where
R¹¹ represents hydrogen, methyl, cyclopropyl or benzyl,
R¹² represents hydrogen or methyl, or
R¹¹ and R¹² together with the nitrogen atom to which they are attached form a 4- to 7-membered heterocycle,
R¹³ represents hydrogen, C₁-C₃-alkyl, methoxy or 4- to 8-membered heterocyclyl which is attached via a carbon atom,
in which alkyl may be substituted by 1 to 2 substituents independently of one another selected from the group consisting of C₁-C₃-alkylamino, —(OCH₂CH₂)ₙ—OCH₃, —(OCH₂CH₂)ₘ—OH, morpholinyl and pyrrolidinyl,
in which n is a number from 1 to 6,
in which m is a number from 1 to 6, and
in which heterocyclyl may be substituted by 1 to 2 substituents independently of one another selected from the group consisting of C₁-C₄-alkyl,
R¹⁴ represents hydrogen or methyl,
R¹³ and R¹⁴ together with the nitrogen atom to which they are attached form a 4- to 7-membered heterocycle,
in which the heterocycle may be substituted by 1 to 2 substituents independently of one another selected from the group consisting of C₁-C₄-alkyl,
R⁵ represents hydrogen, fluorine, chlorine, methyl or methoxy,
R¹⁵ represents amino, —S(O)₂NR¹⁷R¹⁸ or —C(O)NR¹⁹R²⁰,
where
R¹⁷ represents methyl or 4- to 8-membered heterocyclyl which is attached via a carbon atom,
R¹⁸ represents methyl,
R¹⁹ represents C₁-C₃-alkyl, C₃-C₆-cycloalkyl or 4- to 8-membered heterocyclyl which is attached via a carbon atom,
in which alkyl may be substituted by 1 to 2 substituents independently of one another selected from the group consisting of amino, C₁-C₃-alkylamino, —(OCH₂CH₂)ₘ—OH, piperidinyl and pyrrolidinyl,
in which m is a number from 1 to 6, and
in which cycloalkyl may be substituted by a C₁-C₃-alkylamino substituent, and
in which heterocyclyl may be substituted by an oxo substituent,
R²⁰ represents hydrogen,
R¹⁹ and R²⁰ together with the nitrogen atom to which they are attached form a 4- to 7-membered heterocycle,
in which the heterocycle may be substituted by 1 to 2 substituents independently of one another selected from the group consisting of C₁-C₄-alkyl,
in which alkyl for its part may be substituted by a hydroxy substituent,
R¹⁶ represents hydrogen or methyl,
R²¹ represents hydrogen, hydroxy, amino, C₁-C₄-alkoxy, C₁-C₃-alkylamino or 5- or 6-membered heterocyclyl which is attached via a nitrogen atom,
where alkoxy may be substituted by a C₁-C₃-alkylamino substituent and
where heterocyclyl may be substituted by 1 to 2 methyl substituents,
R²² represents hydrogen, amino, methyl or trifluoromethyl,
R²³ represents hydrogen or 5- or 6-membered heterocyclyl which is attached via a nitrogen atom,
where heterocyclyl may be substituted by 1 to 2 methyl substituents,
R²⁴ represents hydrogen or methyl,
R²⁵ represents hydroxy, amino, C₁-C₃-alkylamino, C₁-C₄-alkylcarbonylamino, C₃-C₆-cycloalkylamino or 5- or 6-membered heterocyclyl which is attached via a nitrogen atom,
where heterocyclyl may be substituted by 1 to 2 methyl substituents,
R²⁶ represents hydrogen, methyl or benzyloxy,
R²⁷ represents C₁-C₄-alkyl, trifluoromethyl or —S(O)₂NR²⁸R²⁹,
where
R²⁸ and R²⁹ together with the nitrogen atom to which they are attached form a 4- to 7-membered heterocycle,
R³² represents —S(O)₂NR³⁴R³⁵,
where
R³⁴ represents 4- to 8-membered heterocyclyl which is attached via a carbon atom,
R³⁵ represents hydrogen,
R³³ represents hydrogen,
R³ represents hydrogen, methyl or methoxy,
or one of the salts thereof, solvates thereof or solvates of the salts thereof.

3. The compound of claim 1, characterized in that
R¹ represents a group of the formula 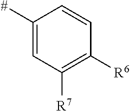
where # is the point of attachment to the nitrogen atom,
R⁶ represents oxadiazolyl, triazolyl and tetrazolyl,
where oxadiazolyl, triazolyl and tetrazolyl may be substituted by a substituent selected from the group consisting of C₁-C₃-alkyl,
in which alkyl is substituted by a hydroxycarbonyl substituent and in which alkyl is additionally substituted by 1 to 6 fluorine sub stituents,
R⁷ represents hydrogen, or
R¹ represents 2,3-dihydro-1H-indazol-6-yl, 1H-benzimidazol-6-yl or 1H-indazol-6-yl,
where 2,3-dihydro-1H-indazol-6-yl, 1H-benzimidazol-6-yl und 1H-indazol-6-yl may be substituted by a substituent independently selected from the group consisting of oxo, methyl, ethyl and n-propyl,
in which ethyl may be substituted by 4 or 5 fluorine substituents,
R² represents a group of the formula 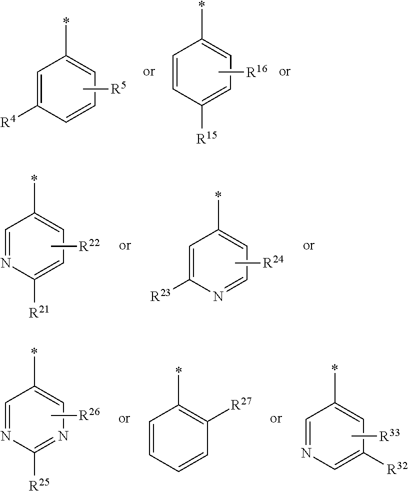
where * is the point of attachment to the phenyl ring,
R⁴ represents —S(O)₂NR¹¹R¹² or —C(O)NR¹³R¹⁴,
where
R¹¹ represents hydrogen, methyl, cyclopropyl or benzyl,
R¹² represents hydrogen or methyl, or
R¹¹ and R¹² together with the nitrogen atom to which they are attached form a pyrrolidinyl, morpholinyl or piperazinyl,
R¹³ represents hydrogen, C₁-C₃-alkyl, methoxy or heterocyclyl which is attached via a carbon atom and selected from the group consisting of pyrrolidinyl and piperidinyl,
in which alkyl may be substituted by 1 to 2 substituents independently of one another selected from the group consisting of C₁-C₃-alkylamino, —(OCH₂CH₂)ₙ—OCH₃, —(OCH₂CH₂)ₘ—OH, morpholinyl and pyrrolidinyl,
in which n is a number from 1 to 6,
in which m is a number from 1 to 6, and
in which pyrrolidinyl and piperidinyl may be substituted by 1 to 2 substituents independently of one another selected from the group consisting of C₁-C₄-alkyl,
R¹⁴ represents hydrogen or methyl,
R¹³ and R¹⁴ together with the nitrogen atom to which they are attached form a pyrrolidinyl, morpholinyl or piperazinyl,
in which pyrrolidinyl, morpholinyl and piperazinyl may be substituted by 1 to 2 substituents independently of one another selected from the group consisting of C₁-C₄-alkyl,
R⁵ represents hydrogen, fluorine, chlorine, methyl or methoxy,
R¹⁵ represents amino, —S(O)₂NR¹⁷R¹⁸ or —C(O)NR¹⁹R²⁰,
where
R¹⁷ represents methyl or heterocyclyl which is attached via a carbon atom and selected from the group consisting of pyrrolidinyl and piperidinyl,
R¹⁸ represents methyl,
R¹⁹ represents C₁-C₃-alkyl, C₃-C₆-cycloalkyl or heterocyclyl which is attached via a carbon atom and selected from the group consisting of pyrrolidinyl and piperidinyl,
in which alkyl may be substituted by 1 to 2 substituents independently of one another selected from the group consisting of amino, C₁-C₃-alkylamino, —(OCH₂CH₂)ₘ—OH, piperidinyl and pyrrolidinyl,
in which m is a number from 1 to 6, and
in which cycloalkyl may be substituted by a C₁-C₃-alkylamino substituent and
in which pyrrolidinyl and piperidinyl may be substituted by an oxo substituent,
R²⁰ represents hydrogen,
R¹⁹ and R²⁰ together with the nitrogen atom to which they are attached form a pyrrolidinyl, morpholinyl or piperazinyl,
in which pyrrolidinyl, morpholinyl and piperazinyl may be substituted by 1 to 2 substituents independently of one another selected from the group consisting of C₁-C₄-alkyl,
in which alkyl for its part may be substituted by a hydroxy substituent,
R¹⁶ represents hydrogen or methyl,
R²¹ represents hydrogen, hydroxy, amino, C₁-C₄-alkoxy, C₁-C₃-alkylamino or heterocyclyl which is attached via a nitrogen atom and selected from the group consisting of morpholinyl and piperazinyl,
where alkoxy may be substituted by a C₁-C₃-alkylamino substituent, and
where morpholinyl and piperazinyl may be substituted by 1 to 2 methyl substituents,
R²² represents hydrogen, amino, methyl or trifluoromethyl,
R²³ represents hydrogen or heterocyclyl which is attached via a nitrogen atom and selected from the group consisting of morpholinyl and piperazinyl,
where morpholinyl and piperazinyl may be substituted by 1 to 2 methyl substituents,
R²⁴ represents hydrogen or methyl,
R²⁵ represents hydroxy, amino, C₁-C₃-alkylamino, C₁-C₄-alkylcarbonylamino, C₃-C₆-cycloalkylamino or heterocyclyl which is attached via a nitrogen atom and selected from the group consisting of morpholinyl and piperazinyl,
where morpholinyl and piperazinyl may be substituted by 1 to 2 methyl sub stituents,
R²⁶ represents hydrogen, methyl or benzyloxy,
R²⁷ represents C₁-C₄-alkyl, trifluoromethyl or —S(O)₂NR²⁸R²⁹,
where
R²⁸ and R²⁹ together with the nitrogen atom to which they are attached form a pyrrolidinyl, morpholinyl or piperazinyl,
R³² represents —S(O)₂NR³⁴R³⁵,
where
R³⁴ represents heterocyclyl which is attached via a carbon atom and selected from the group consisting of pyrrolidinyl and piperidinyl,
R³⁵ represents hydrogen,
R³³ represents hydrogen,
R³ represents hydrogen, methyl or methoxy,
or one of the salts thereof, solvates thereof or solvates of the salts thereof.

4. A method of making the compound of claim 1 of the formula (I) or one of the salts thereof, solvates thereof or solvates of the salts thereof, characterized in that a compound of the formula 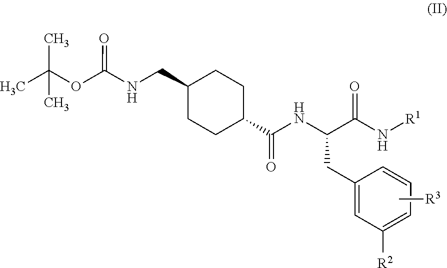
in which R¹, R² and R³ are each as defined in claim 1, is reacted with an acid.

5. A method for treating and/or prophylaxis of diseases using the compound of claim 1.

6. A method of making a medicament for the treatment and/or prophylaxis of diseases using the compound of claim 1.

7. A method of making a medicament for the treatment and/or prophylaxis of thrombotic or thromboembolic disorders using the compound of claim 1.

8. A medicament comprising a compound according to claim 1 in combination with an inert, nontoxic, pharmaceutically suitable excipient.

9. The medicament of claim 8 for the treatment and/or prophylaxis of thrombotic or thromboembolic disorders.

10. A method for the control of thrombotic or thromboembolic disorders in humans and animals by administration of a therapeutically effective amount of the compound of claim 1.

11. A method for the control of thrombotic or thromboembolic disorders in humans and animals by administration of a therapeutically effective amount of the medicament of claim 8.

12. A method for the control of thrombotic or thromboembolic disorders in humans and animals by administration of a therapeutically effective amount of the medicament of claim 6.
